(19) United States
(12) Patent Application Publication
(10) Pub. No.: US 2005/0042664 A1
(43) Pub. Date: Feb. 24, 2005

(54) Humanization of antibodies

(75) Inventors: Herren Wu, Boyds, MD (US); William Dall-Acqua, Gaithersburg, MD (US); Melissa Damschroder, Germantown, MD (US)

Correspondence Address: JOHNATHAN KLEIN-EVANS, ONE MEDIMMUNE WAY, GAITHERSBURG, MD, 20878, US

(73) Assignee: MEDIMMUNE, INC., Gaithersburg, MD (US)

(21) Appl. No.: 10/923,068

(22) Filed: Aug. 20, 2004

Related U.S. Application Data
(60) Provisional application No. 60/497,213, filed on Aug. 22, 2003.
(60) Provisional application No. 60/510,741, filed on Oct. 13, 2003.

(51) Int. Cl. C12Q 1/68

(52) U.S. Cl. 435/6

(57) ABSTRACT

The present invention provides methods of re-engineering or re-shaping an antibody from a first species, wherein the re-engineered or re-shaped antibody does not elicit undesired immune response in a second species, and the re-engineered or re-shaped antibody retains substantially the same antigen binding-ability of the antibody from the first species. In accordance with the present invention, a combinatorial library comprising the CDRs of the antibody from the first species fused in frame with framework regions derived from a second species can be constructed and screened for the desired modified antibody. In particular, the present invention provides methods utilizing low homology acceptor antibody frameworks for efficiently humanizing an antibody or a fragment thereof. The present invention also provides antibodies produced by the methods of the invention.

This application claims the benefit under 35 U.S.C. § 119(e) of U.S. provisional application Ser. No. 60/497,213, filed Aug. 22, 2003 and 60/510,741, filed Oct. 13, 2003, both of which are incorporated by reference herein in their entireties.

### 1. FIELD OF THE INVENTION

The present invention relates to methods of reengineering or reshaping antibodies to reduce the immunogenicity of the antibodies, while maintaining the immunospecificity of the antibodies for an antigen. In particular, the present invention provides methods utilizing low homology acceptor antibody framework regions for efficiently humanizing an antibody or a fragment thereof. The present invention also provides antibodies produced by the methods of the invention.

### 2. BACKGROUND OF THE INVENTION

Antibodies play a vital role in our immune responses. They can inactivate viruses and bacterial toxins, and are essential in recruiting the complement system and various types of white blood cells to kill invading microorganisms and large parasites. Antibodies are synthesized exclusively by B lymphocytes, and are produced in millions of forms, each with a different amino acid sequence and a different binding site for an antigen. Antibodies, collectively called immunoglobulins (Ig), are among the most abundant protein components in the blood. Alberts et al., Molecular Biology of the Cell, 2nd ed., 1989, Garland Publishing, Inc.

A typical antibody is a Y-shaped molecule with two identical heavy (H) chains (each containing about 440 amino acids) and two identical light (L) chains (each containing about 220 amino acids). The four chains are held together by a combination of noncovalent and covalent (disulfide) bonds. The proteolytic enzymes, such as papain and pepsin, can split an antibody molecule into different characteristic fragments. Papain produces two separate and identical Fab fragments, each with one antigen-binding site, and one Fc fragment. Pepsin produces one F(ab′)₂ fragment. Alberts et al., Molecular Biology of the Cell, 2nd ed., 1989, Garland Publishing, Inc.

Both L and H chains have a variable sequence at their amino-terminal ends but a constant sequence at their carboxyl-terminal ends. The L chains have a constant region about 110 amino acids long and a variable region of the same size. The H chains also have a variable region about 110 amino acids long, but the constant region of the H chains is about 330 or 440 amino acid long, depending on the class of the H chain. Alberts et al., Molecular Biology of the Cell, 2nd ed., 1989, Garland Publishing, Inc. at pp1019.

Only part of the variable region participates directly in the binding of antigen. Studies have shown that the variability in the variable regions of both L and H chains is for the most part restricted to three small hypervariable regions (also called complementarity-determining regions, or CDRs) in each chain. The remaining parts of the variable region, known as framework regions (FR), are relatively constant. Alberts et al., Molecular Biology of the Cell, 2nd ed., 1989, Garland Publishing, Inc. at pp 1019-1020.

Natural immunoglobulins have been used in assays, diagnosis and, to a more limited extent, therapy. However, such uses, especially in therapy, have been hindered by the polyclonal nature of natural immunoglobulins. The advent of monoclonal antibodies of defined specificity increased the opportunities for therapeutic use. However, most monoclonal antibodies are produced following immunization of a rodent host animal with the target protein, and subsequent fusion of a rodent spleen cell producing the antibody of interest with a rodent myeloma cell. They are, therefore, essentially rodent proteins and as such are naturally immunogenic in humans, frequently giving rise to an undesirable immune response termed the HAMA (Human Anti-Mouse Antibody) response.

Many groups have devised techniques to decrease the immunogenicity of therapeutic antibodies. Traditionally, a human template is selected by the degree of homology to the donor antibody, i.e., the most homologous human antibody to the non-human antibody in the variable region is used as the template for humanization. The rationale is that the framework sequences serve to hold the CDRs in their correct spacial orientation for interaction with an antigen, and that framework residues can sometimes even participate in antigen binding. Thus, if the selected human framework sequences are most similar to the sequences of the donor frameworks, it will maximize the likelihood that affinity will be retained in the humanized antibody. Winter (EP No. 0239400), for instance, proposed generating a humanized antibody by site-directed mutagenesis using long oligonucleotides in order to graft three complementarity determining regions (CDR1, CDR2 and CDR3) from each of the heavy and light chain variable regions. Although this approach has been shown to work, it limits the possibility of selecting the best human template supporting the donor CDRs.

Although a humanized antibody is less immunogenic than its natural or chimeric counterpart in a human, many groups find that a CDR grafted humanized antibody may demonstrate a significantly decreased binding affinity (e.g., Riechmann et al., 1988, Nature 3 32:323-327). For instance, Reichmann and colleagues found that transfer of the CDR regions alone was not sufficient to provide satisfactory antigen binding activity in the CDR-grafted product, and that it was also necessary to convert a serine residue at position 27 of the human sequence to the corresponding rat phenylalanine residue. These results indicated that changes to residues of the human sequence outside the CDR regions may be necessary to obtain effective antigen binding activity. Even so, the binding affinity was still significantly less than that of the original monoclonal antibody.

For example, Queen et al (U.S. Pat. No. 5,530,101) described the preparation of a humanized antibody that binds to the interleukin-2 receptor, by combining the CDRs of a murine monoclonal (anti-Tac MAb) with human immunoglobulin framework and constant regions. The human framework regions were chosen to maximize homology with the anti-Tac MAb sequence. In addition, computer modeling was used to identify framework amino acid residues which were likely to interact with the CDRs or antigen, and mouse amino acids were used at these positions in the humanized antibody. The humanized anti-Tac antibody obtained was reported to have an affinity for the interleukin-2 receptor (p55) of 3×10⁹ M⁻¹, which was still only about one-third of that of the murine MAb.

Other groups identified further positions within the framework of the variable regions (i.e., outside the CDRs and structural loops of the variable regions) at which the amino acid identities of the residues may contribute to obtaining CDR-grafted products with satisfactory binding affinity. See, e.g., U.S. Pat. Nos. 6,054,297 and 5,929,212. Still, it is impossible to know beforehand how effective a particular CDR grafting arrangement will be for any given antibody of interest.

Leung (U.S. Patent Application Publication No. US 2003/0040606) describes a framework patching approach, in which the variable region of the immunoglobulin is compartmentalized into FR1, CDR1, FR2, CDR2, FR3, CDR3 and FR4, and the individual FR sequence is selected by the best homology between the non-human antibody and the human antibody template. This approach, however, is labor intensive, and the optimal framework regions may not be easily identified.

As more therapeutic antibodies are being developed and are holding more promising results, it is important to be able to reduce or eliminate the body's immune response elicited by the administered antibody. Thus, new approaches allowing efficient and rapid engineering of antibodies to be human-like, and/or allowing a reduction in labor to humanize an antibody provide great benefits and medical value.

Citation or discussion of a reference herein shall not be construed as an admission that such is prior art to the present invention.

### 3. SUMMARY OF THE INVENTION

The invention is based, in part, on synthesis of a combinatorial library of antibodies comprising a variable heavy chain region and/or a variable light chain region with the variable chain region(s) produced by fusing together in frame complementarity determining regions (CDRs) derived from a donor antibody and framework regions derived from a low homology framework region of an acceptor antibody, wherein said donor antibody and acceptor antibody are from different species (e.g., a donor antibody from mouse, and an acceptor antibody from human). The acceptor frameworks can be derived from germline sequences, mature antibody gene sequences, or other known functional antibody sequences. The combinatorial libraries are created by introducing limited diversity in both the light and heavy chain variable regions using wobble codons that encode for either donor or acceptor residues at several key positions (i.e., key residues). The resulting libraries are screened for antigen-binding activity and/or function of the antibodies. The synthesis of combinatorial libraries of antibodies (with or without constant regions) using low homology acceptor frameworks allows for fast, less labor intensive production of antibodies (with or without constant regions) which can be readily screened for their immunospecificity for an antigen of interest, as well as their immunogenicity in an organism of interest. The methods of the invention are exemplified herein for the production of humanized antibodies for use in human beings. However, the methods of the invention can readily be applied to the production of antibodies for use in any organism of interest.

The present invention provides a library of nucleic acid sequences comprising a plurality of nucleotide sequences, each nucleotide sequence encoding an acceptor heavy chain framework region (e.g., human heavy chain framework region 1, human heavy chain framework 2, human heavy chain framework region 3, or human heavy chain framework region 4) that is less than 65% (preferably, less than 60%, less than 55%, less than 50%, less than 45%, or less than 40%) identical to the corresponding framework region of a donor antibody at the amino acid level. In some embodiments, the acceptor heavy chain framework region contains at least one amino acid residue (preferably, at least two, or at least three amino acid residues) at amino acid residues 6, 23, 24 and/or 49 according to the Kabat numbering system that is (are) not identical to the corresponding residue(s) in the donor antibody. In certain embodiments, the acceptor heavy chain variable framework regions contain one or more mutations introduced at amino acid residues designated key residues, said key residues not including amino acid residues 2, 4, 24, 35, 36, 39, 43, 45, 64, 69, 70, 73, 74, 75, 76, 78, 92 and 93 according to the Kabat numbering system. In certain embodiments, the residues designated key are one or more of the following: adjacent to a CDR, a potential glycosylation site, a rare residue, a residue capable of interacting with the antigen, a residue capable of interacting with a CDR, canonical residues, contact residues between the variable heavy domain and variable light domain, a residue within the vernier zone, and/or a residue within the region which overlaps between the Chothia definition of the heavy chain variable region CDR1 and the Kabat definition of the first heavy chain framework. In some embodiments, the mutations introduced at amino acid residues designated key are substitutions. In particular embodiments, the amino acid residues designated key are not heavy chain variable framework region amino acid residues 6, 23, 24 and 49 as a group according to the Kabat numbering system.

The present invention provides a library of nucleic acid sequences comprising a plurality of nucleotide sequences, each nucleotide sequence encoding an acceptor light chain framework region (e.g., a human light chain framework region 1, human light chain framework region 2, human light chain framework region 3, or human light chain framework region 4) that is less than 65% (preferably, less than 60%, less than 55%, less than 50%, less than 45%, or less than 40%) identical to the corresponding framework region of a donor antibody at the amino acid level. In some embodiments, the acceptor light chain variable framework regions contain one or more mutations introduced at amino acid residues designated key residues, said key residues not including amino acid residues 4, 38, 43, 44, 46, 58, 62, 65, 66, 67, 68, 69, 73, 85, and 98 according to the Kabat numbering system. In some embodiments, the mutations introduced at amino acid residues designated key are substitutions. In specific embodiments, the substitutions replace the acceptor amino acid residues in the light chain variable framework region with the corresponding amino acid residues in the donor light chain variable framework region. In some embodiments, the residues designated key are one or more of the following: adjacent to a CDR, a potential glycosylation site, a rare residue, a residue capable of interacting with the antigen, a residue capable of interacting with a CDR, canonical residues, contact residues between the variable heavy domain and variable light domain, and/or a residue within the vernier zone.

The present invention provides a library of nucleic acid sequences comprising a plurality of nucleotide sequences, each nucleotide sequence encoding a humanized heavy chain variable region produced by fusing together in frame nucleic acid sequences encoding CDRs from a donor antibody heavy chain variable region and nucleic acid sequences encoding acceptor heavy chain variable framework regions selected as described herein. In some embodiments, the humanized heavy chian variable region further comprises one or more constant regions in addition to the variable region. The library of nucleic acid sequences comprising a plurality of nucleotide sequences encoding humanized heavy chain variable regions can be expressed in host cells (which host cells may or may not contain or comprise a nucleic acid sequence comprising a nucleotide sequence encoding a light chain or light chain variable region), which can be used to screen, identify and/or select a humanized antibody that immunospecifically binds to an antigen of interest.

The present invention provides a library of nucleic acid sequences comprising a plurality of nucleotide sequences, each nucleotide sequence encoding a humanized light chain variable region produced by fusing together in frame nucleic acid sequences encoding CDRs from a donor antibody light chain variable region and nucleic acid sequences encoding acceptor light chain variable framework regions selected as described herein. In some embodiments, the humanized light chain variable region further comprises one or more constant regions in addition to the variable region. The library of nucleic acid sequences comprising a plurality of nucleotide sequences encoding humanized light chain variable regions can be expressed in host cells (which host cells may or may not contain or comprise a nucleic acid sequence comprising a nucleotide sequence encoding a heavy chain or heavy chain variable region), which can be used to screen, identify and/or select a humanized antibody that immunospecifically binds to an antigen of interest.

The present invention provides a library of nucleic acid sequences comprising (i) a first set of nucleotide sequences, and (ii) a second set nucleotide sequences, wherein each nucleotide sequence in the first set of nucleotide sequences encodes a humanized heavy chain variable region produced by fusing together in frame nucleic acid sequences encoding CDRs from a donor antibody and nucleic acid sequences encoding acceptor heavy chain variable framework regions selected as described herein, and wherein each nucleotide sequence in the second set of nucleotide sequences encodes a humanized light chain variable region produced by fusing together in frame nucleic acid sequences encoding CDRs from a donor antibody and nucleic acid sequences encoding acceptor light chain variable framework regions selected as described herein. In some embodiments, the humanized antibody comprises one or more constant regions in addition to the variable regions. The library of nucleic acid sequences comprising a first set of nucleotide sequences encoding humanized heavy chain variable regions and a second set of nucleotide sequences encoding humanized light chain variable region can be expressed in host cells, which can be used to screen, identify, and/or select a humanized antibody that immunospecifically binds to an antigen of interest.

The present invention provides a nucleic acid sequence encoding a humanized antibody that immunospecifically binds to an antigen, said nucleic acid sequence produced by the process comprising: (a) selecting an acceptor heavy chain framework region 1, an acceptor heavy chain framework region 2, an acceptor heavy chain framework region 3, and an acceptor heavy chain framework region 4, wherein at least one (preferably, at least two, at least three, or all four) of the framework regions is less than 65% (preferably, less than 60%, less than 55%, less than 50%, less than 45%, or less than 40%) identical to the corresponding framework region(s) of a donor antibody at the amino acid level; and (b) synthesizing a nucleic acid sequence comprising a nucleotide sequence encoding a humanized heavy chain variable region, said nucleotide sequence comprising nucleic acid sequences encoding complementarity determining regions (CDRs) from the donor antibody heavy chain variable region and nucleic acid sequences encoding the acceptor heavy chain variable framework regions. In certain embodiments, a donor antibody amino acid residue in the humanized heavy chain variable framework region is not within 6 Å, 6.5 Å, 7 Å, 7.5 Å or 8 Å of a CDR. The present invention also provides a cell containing a nucleic acid sequence encoding a humanized antibody that immunospecifically binds to an antigen, said cell is produced by introducing the nucleic acid sequence comprising the nucleotide sequence encoding the humanized heavy chain variable region described herein into the cell. In some embodiments, the cell further contains a nucleic acid sequence comprising a nucleotide sequence encoding a light chain variable region, preferably, a human or humanized light chain variable region. The present invention further provides a method of producing a humanized antibody that immunospecifically binds to an antigen, said method comprising expressing the nucleic acid sequence encoding the humanized antibody contained in the cell described herein. The present invention also provides optional screening methods for identification and/or selection of a humanized antibody of interest.

The present invention provides a nucleic acid sequence encoding a humanized antibody that immunospecifically binds to an antigen, said nucleic acid sequence produced by the process comprising (a) selecting an acceptor heavy chain framework region 1, an acceptor heavy chain framework region 2, an acceptor heavy chain framework region 3, and an acceptor heavy chain framework region 4, wherein at least one (preferably, at least two, at least three, or all four) of the framework regions is less than 65% (preferably, less than 60%, less than 55%, less than 50%, less than 45%, or less than 40%) identical to the corresponding framework region(s) of a donor antibody at the amino acid level; and (b) synthesizing a nucleic acid sequence comprising a nucleotide sequence encoding a humanized heavy chain variable region with at least one (preferably at least two, at least three, or all four) framework region(s) that remains less than 65% (preferably less than 60%, less than 55%, less than 50%, less than 45%, or less than 40%) identical to the corresponding donor antibody heavy chain variable framework region(s) at the amino acid level, said nucleotide sequence comprising nucleic acid sequences encoding CDRs from the donor antibody heavy chain variable region and nucleic acid sequences encoding the acceptor heavy chain variable framework regions with one or more mutations introduced at amino acid residues designated key residues, said key residues not including amino acid residues 2, 4, 24, 35, 36, 39, 43, 45, 64, 69, 70, 73, 74, 75, 76, 78, 92 and 93 according to the Kabat numbering system. In certain embodiments, the residues designated key are one or more of the following: adjacent to a CDR, a potential glycosylation site, a rare residue, a residue capable of interacting with the antigen, a residue capable of interacting with a CDR, canonical residues, contact residues between the variable heavy domain and variable light domain, a residue within the vernier zone, and/or a residue within the region which overlaps between the Chothia definition of the heavy chain variable region CDR1 and the Kabat definition of the first heavy chain framework. In some embodiments, the mutations introduced at amino acid residues designated key are substitutions. In particular embodiments, the amino acid residues designated key are not heavy chain variable framework region amino acid residues 6, 23, 24 and 49 as a group according to the Kabat numbering system. In some embodiments, a donor antibody amino acid residue in the humanized heavy chain variable framework region is not within 6 Å, preferably 6.5 Å, 7 Å, 7.5 Å or 8 Å of a CDR. In accordance with the invention, the donor antibody and acceptor antibody are from different species (e.g., a donor antibody from mouse, and an acceptor antibody from human). The present invention also provides a cell containing or comprising a nucleic acid sequence encoding a humanized antibody that immunospecifically binds to an antigen, said cell produced by introducing the nucleic acid sequence comprising the nucleotide sequence encoding the humanized heavy chain variable region described herein into the cell. In some embodiments, the cell further contains or comprises a nucleic acid sequence comprising a nucleotide sequence encoding a light chain variable region, preferably, a human or humanized light chain variable region. The present invention also provides a method of producing a humanized antibody that immunospecifically binds to an antigen, said method comprising expressing the nucleic acid sequence encoding the humanized antibody contained in the cell described herein. The present invention further provides optional screening methods for identification and/or selection of a humanized antibody of interest.

The present invention provides a nucleic acid sequence encoding a humanized antibody that immunospecifically binds to an antigen, said nucleic acid sequence produced by the process comprising (a) selecting an acceptor heavy chain framework region 1, an acceptor heavy chain framework region 2, an acceptor heavy chain framework region 3, and an acceptor heavy chain framework region 4, wherein at least one (preferably, at least two, at least three, or all four) of the framework regions is less than 65% (preferably, less than 60%, less than 55%, less than 50%, less than 45%, or less than 40%) identical to the corresponding framework region(s) of a donor antibody at the amino acid level, and the acceptor heavy chain framework region contains at least one amino acid residue (preferably, at least two, or at least three amino acid residues) at amino acid residues 6, 23, 24 and/or 49 according to the Kabat numbering system that is (are) not identical to the corresponding residue(s) in the donor antibody; and (b) synthesizing a nucleic acid sequence comprising a nucleotide sequence encoding a humanized heavy chain variable region with at least one (preferably at least two, at least three, or all four) framework region(s) that remains less than 65% (preferably less than 60%, less than 55%, less than 50%, less than 45%, or less than 40%) identical to the corresponding donor antibody heavy chain variable framework region(s) at the amino acid level, said nucleotide sequence comprising nucleic acid sequences encoding CDRs from the donor antibody heavy chain variable region and nucleic acid sequences encoding the acceptor heavy chain variable framework regions with one or more mutations introduced at amino acid residues designated key residues, said key residues not including amino acid residues 2, 4, 24, 35, 36, 39, 43, 45, 64, 69, 70, 73, 74, 75, 76, 78, 92 and 93 according to the Kabat numbering system.

The present invention provides a nucleic acid sequence encoding a humanized antibody that immunospecifically binds to an antigen, said nucleic acid sequence produced by the process comprising: (a) selecting an acceptor light chain framework region 1, an acceptor light chain framework region 2, an acceptor light chain framework region 3, and an acceptor light chain framework region 4, wherein at least one (preferably, at least two, at least three, or all four) of the framework regions is less than 65% (preferably, less than 60%, less than 55%, less than 50%, less than 45%, or less than 40%) identical to the corresponding framework region(s) of a donor antibody at the amino acid level; and (b) synthesizing a nucleic acid sequence comprising a nucleotide sequence encoding a humanized light chain variable region, said nucleotide sequence comprising nucleic acid sequences encoding complementarity determining regions (CDRs) from the donor antibody light chain variable region and nucleic acid sequences encoding the acceptor light chain variable framework regions. The present invention also provides a cell containing or comprising a nucleic acid sequence encoding a humanized antibody that immunospecifically binds to an antigen, said cell produced by introducing the nucleic acid sequence comprising the nucleotide sequence encoding the humanized light chain variable region described herein into the cell. In some embodiments, the cell further contains or comprises a nucleic acid sequence comprising a nucleotide sequence encoding a heavy chain variable region, preferably, a human or humanized heavy chain variable region. The present invention also provides a method of producing a humanized antibody that immunospecifically binds to an antigen, said method comprising expressing the nucleic acid sequence encoding the humanized antibody contained in the cell described herein. The present invention further provides optional screening methods for identification and/or selection of a humanized antibody of interest.

The present invention provides a nucleic acid sequence encoding a humanized antibody that immunospecifically binds to an antigen, said nucleic acid sequence produced by the process comprising: (a) selecting an acceptor light chain framework region 1, an acceptor light chain framework region 2, an acceptor light chain framework region 3, and an acceptor light chain framework region 4, wherein at least one (preferably, at least two, at least three, or all four) of the framework regions is less than 65% (preferably, less than 60%, less than 55%, less than 50%, less than 45%, or less than 40%) identical to the corresponding framework region(s) of a donor antibody at the amino acid level; and (b) synthesizing a nucleic acid sequence comprising a nucleotide sequence encoding a humanized light chain variable region, said nucleotide sequence comprising nucleic acid sequences encoding CDRs from the donor antibody light chain variable region and nucleic acid sequences encoding the acceptor light chain variable framework regions with one or more mutations introduced at amino acid residues designated key residues, said key residues not including amino acid residues 4, 38, 43, 44, 46, 58, 62, 65, 66, 67, 68, 69, 73, 85, and 98 according to the Kabat numbering system. In some embodiments, a donor antibody amino acid residue in the humanized light chain variable framework region is not within 6 Å, preferably, 6.5 Å, 7 Å, 7.5 Å, or 8 Å of a CDR. In some embodiments, the mutations introduced at amino acid residues designated key are substitutions. In specific embodiments, the substitutions replace the acceptor amino acid residues in the light chain variable framework region with the corresponding amino acid residues in the donor light chain variable framework region. In some embodiments, the residues designated key are one or more of the following: adjacent to a CDR, a potential glycosylation site, a rare residue, a residue capable of interacting with the antigen, a residue capable of interacting with a CDR, canonical residues, contact residues between the variable heavy domain and variable light domain, and/or a residue within the vernier zone. The present invention also provides a cell containing or comprising a nucleic acid sequence encoding a humanized antibody that immunospecifically binds to an antigen, said cell produced by introducing the nucleic acid sequence comprising the nucleotide sequence encoding the humanized light chain variable region described herein into the cell. In some embodiments, the cell further contains or comprising a nucleic acid sequence comprising a nucleotide sequence encoding a heavy chain variable region, preferably, a human or humanized heavy chain variable region. The present invention also provides a method of producing a humanized antibody that immunospecifically binds to an antigen, said method comprising expressing the nucleic acid sequence encoding the humanized antibody contained in the cell described herein. The present invention further provides optional screening methods for identification and/or selection of a humanized antibody of interest.

The present invention provides a nucleic acid sequence encoding a humanized antibody that immunospecifically binds to an antigen, said nucleic acid sequence produced by the process comprising: (a) selecting an acceptor heavy chain framework region 1, an acceptor heavy chain framework region 2, an acceptor heavy chain framework region 3, and an acceptor heavy chain framework region 4, wherein at least one (preferably, at least two, at least three, or all four) of the framework regions is less than 65% (preferably, less than 60%, less than 55%, less than 50%, less than 45%, or less than 40%) identical to the corresponding framework region(s) of a donor antibody at the amino acid level; and (b) synthesizing a nucleic acid sequence comprising: (i) a first nucleotide sequence encoding a light chain variable region, and (ii) a second nucleotide sequence encoding a humanized heavy chain variable region with at least one (preferably, at least two, at least three, or all four) framework region(s) that remains less than 65% (preferably less than 60%, less than 55%, less than 50%, less than 45%, or less than 40%) identical to the corresponding donor antibody heavy chain variable framework region(s) at the amino acid level, said second nucleotide sequence comprising nucleic acid sequences encoding complementarity determining regions (CDRs) from the donor antibody heavy chain variable region and nucleic acid sequences encoding the acceptor heavy chain variable framework regions. The present invention provides a cell containing or comprising a nucleic acid sequence encoding a humanized antibody that immunospecifically binds to an antigen, said cell produced by introducing the nucleic acid sequence comprising the first nucleotide sequence and second nucleotide sequence described herein into the cell. In some embodiments, the light chain is humanized. In certain embodiments, a donor antibody amino acid residue in the humanized heavy chain variable framework region is not within 6 Å, preferably not within 6.5 Å, 7 Å, 7.5 Å or 8 Å of a CDR. The present invention also provides a method of producing a humanized antibody that immunospecifically binds to an antigen, said method comprising expressing the nucleic acid sequence encoding the humanized antibody contained in the cell described herein. The present invention further provides optional screening methods for identification and/or selection of a humanized antibody of interest.

The present invention provides a nucleic acid sequence encoding a humanized antibody that immunospecifically binds to an antigen, said nucleic acid sequence produced by the process comprising: (a) selecting an acceptor heavy chain framework region 1, an acceptor heavy chain framework region 2, an acceptor heavy chain framework region 3, and an acceptor heavy chain framework region 4, wherein at least one (preferably, at least two, at least three, or all four) of the framework regions is less than 65% (preferably, less than 60%, less than 55%, less than 50%, less than 45%, or less than 40%) identical to the corresponding framework region(s) of a donor antibody at the amino acid level; and (b) synthesizing a nucleic acid sequence comprising: (i) a first nucleotide sequence encoding a light chain variable region, and (ii) a second nucleotide sequence encoding a humanized heavy chain variable region with at least one (preferably, at least two, at least three, or all four) framework region(s) that remains less than 65% (preferably less than 60%, less than 55%, less than 50%, less than 45%, or less than 40%) identical to the corresponding donor antibody heavy chain variable framework region(s) at the amino acid level, said second nucleotide sequence comprising nucleic acid sequences encoding CDRs from the donor antibody heavy chain variable region and nucleic acid sequences encoding the acceptor heavy chain variable framework regions with one or more mutations introduced at amino acid residues designated key residues, said key residues not including amino acid residues 2, 4, 24, 35, 36, 39, 43, 45, 64, 69, 70, 73, 74, 75, 76, 78, 92 and 93 according to the Kabat numbering system. In some embodiments, the light chain is humanized. In certain embodiments, the residues designated key are one or more of the following: adjacent to a CDR, a potential glycosylation site, a rare residue, a residue capable of interacting with the antigen, a residue capable of interacting with a CDR, canonical residues, contact residues between the variable heavy domain and variable light domain, a residue within the vernier zone, and/or a residue within the region which overlaps between the Chothia definition of the heavy chain CDR1 and the Kabat definition of the first heavy chain framework. In some embodiments, the mutations introduced at amino acid residues designated key are substitutions. In specific embodiments, the substitutions replace the acceptor amino acid residues in the heavy chain variable framework region with the corresponding amino acid residues in the donor heavy chain variable framework region. In some embodiments, a donor antibody amino acid residue in the humanized heavy chain and/or light chain variable framework region is not within 6 Å, preferably not within 6.5 Å, 7 Å, 7.5 Å or 8 Å of a CDR. The present invention also provides a cell containing or comprising a nucleic acid sequence encoding a humanized antibody that immunospecifically binds to an antigen, said cell produced by introducing the nucleic acid sequence comprising the first nucleotide sequence and the second nucleotide sequence described herein into the cell. The present invention also provides a method of producing a humanized antibody that immunospecifically binds to an antigen, said method comprising expressing the nucleic acid sequence encoding the humanized antibody contained in the cell described herein. The present invention further provides optional screening methods for identification and/or selection of a humanized antibody of interest.

The present invention provides a nucleic acid sequence encoding a humanized antibody that immunospecifically binds to an antigen, said nucleic acid sequence produced by the process comprising: (a) selecting an acceptor heavy chain framework region 1, an acceptor heavy chain framework region 2, an acceptor heavy chain framework region 3, and an acceptor heavy chain framework region 4, wherein at least one (preferably, at least two, at least three, or all four) of the framework regions is less than 65% (preferably, less than 60%, less than 55%, less than 50%, less than 45%, or less than 40%) identical to the corresponding framework region(s) of a donor antibody at the amino acid level; (b) selecting an acceptor light chain variable framework region less than 65% (preferably less than 60%, less than 55%, less than 50%, less than 45%, or less than 40%) identical to a donor antibody light chain variable framework region at the amino acid level; and (c) synthesizing a nucleic acid sequence comprising: (i) a first nucleotide sequence encoding a humanized light chain variable region, said first nucleotide sequence comprising nucleic acid sequences encoding CDRs from the donor antibody light chain variable region and nucleic acid sequences encoding the acceptor light chain variable framework regions with one or more mutations introduced at amino acid residues designated key residues, said key residues not including amino acid residues 4, 38, 43, 44, 46, 58, 62, 65, 66, 67, 68, 69, 73, 85, and 98 according to the Kabat numbering system, and (ii) a second nucleotide sequence encoding a humanized heavy chain variable region with at least one (preferably, at least two, at least three, or all four) framework region(s) that remains less than 65% (preferably less than 60%, less than 55%, less than 50%, less than 45%, or less than 40%) identical to the corresponding donor antibody heavy chain variable framework region(s) at the amino acid level, said second nucleotide sequence comprising nucleic acid sequences encoding complementarity determining regions (CDRs) from the donor antibody heavy chain variable region and nucleic acid sequences encoding the acceptor heavy chain variable framework regions. In some embodiments, the residues designated key are one or more of the following: adjacent to a CDR, a potential glycosylation site, a rare residue, a residue capable of interacting with the antigen, a residue capable of interacting with a CDR, canonical residues, contact residues between the variable heavy domain and variable light domain, a residue within the vernier zone, and/or a residue within the region which overlaps between the Chothia definition of the heavy chain CDR1 and the Kabat definition of the first heavy chain framework. In some embodiments, the mutations introduced at the residues designated key are substitutions. In specific embodiments, the substitutions replace the acceptor amino acid residues in the light chain variable framework region with the corresponding amino acid residues in the donor light chain variable framework region. In some embodiments, a donor antibody amino acid residue in the humanized heavy chain and/or humanized light chain variable framework region is not within 6 Å, preferably not within 6.5 Å, 7 Å, 7.5 Å or 8 Å of a CDR. The present invention also provides a cell containing or comprising a nucleic acid sequence encoding a humanized antibody that immunospecifically binds to an antigen, said cell produced by introducing the nucleic acid sequence comprising the first nucleotide sequence and second nucleotide sequence described herein into the cell. The present invention also provides a method of producing a humanized antibody that immunospecifically binds to an antigen, said method comprising expressing the nucleic acid sequence encoding the humanized antibody contained in the cell described herein. The present invention also provides optional screening methods for identification and/or selection of a humanized antibody of interest.

The present invention provides a nucleic acid sequence encoding a humanized antibody that immunospecifically binds to an antigen, said nucleic acid sequence produced by the process comprising: (a) selecting an acceptor heavy chain framework region 1, an acceptor heavy chain framework region 2, an acceptor heavy chain framework region 3, and an acceptor heavy chain framework region 4, wherein at least one (preferably, at least two, at least three, or all four) of the framework regions is less than 65% (preferably, less than 60%, less than 55%, less than 50%, less than 45%, or less than 40%) identical to the corresponding framework region(s) of a donor antibody at the amino acid level; (b) selecting an acceptor light chain variable framework region less than 65% (preferably less than 60%, less than 55%, less than 50%, less than 45%, or less than 40%) identical to a donor antibody light chain variable framework region at the amino acid level; and (c) synthesizing a nucleic acid sequence comprising: (i) a first nucleotide sequence encoding a humanized light chain variable region, said first nucleotide sequence comprising nucleic acid sequences encoding CDRs from the donor antibody light chain variable region and nucleic acid sequences encoding the acceptor light chain variable framework regions with one or more mutations introduced at amino acid residues designated key residues, said key residues not including amino acid residues 4, 38, 43, 44, 46, 58, 62, 65, 66, 67, 68, 69, 73, 85, and 98 according to the Kabat numbering system, and (ii) a second nucleotide sequence encoding a humanized heavy chain variable region with at least one (preferably, at least two, at least three, or all four) framework region(s) that remains less than 65% (preferably less than 60%, less than 55%, less than 50%, less than 45%, or less than 40%) identical to the corresponding donor antibody heavy chain variable framework region(s) at the amino acid level, said second nucleotide sequence comprising nucleic acid sequences encoding CDRs from the donor antibody heavy chain variable region and nucleic acid sequences encoding the acceptor heavy chain variable framework regions with one or more mutations introduced at amino acid residues designated key residues, said key residues not including amino acid residues 2, 4, 24, 35, 36, 39, 43, 45, 64, 69, 70, 73, 74, 75, 76, 78, 92 and 93 according to the Kabat numbering system. In some embodiments, the residues designated key are one or more of the following: adjacent to a CDR, a potential glycosylation site, a rare residue, a residue capable of interacting with the antigen, a residue capable of interacting with a CDR, canonical residues, contact residues between the variable heavy domain and variable light domain, or a residue within the vernier zone. In certain embodiments, the mutations introduced at the amino acid residues designated key are substitutions. In specific embodiments, the substitutions replace the acceptor amino acid residues in the heavy and/or light chain variable framework region with the corresponding amino acid residues in the donor heavy and/or light chain variable framework region. In some embodiments, a donor antibody amino acid residue in the humanized heavy and/or light chain variable framework region is not within 6 Å, preferably not within 6.5 Å, 7 Å, 7.5 Å or 8 Å of a CDR. The present invention also provides a cell containing or comprising a nucleic acid sequence encoding a humanized antibody that immunospecifically binds to an antigen, said cell produced by introducing the nucleic acid sequence comprising the first nucleotide sequence and the second nucleotide sequence described herein into the cell. The present invention also provides a method of producing a humanized antibody that immunospecifically binds to an antigen, said method comprising expressing the nucleic acid sequence encoding the humanized antibody contained in the cell described herein. The present invention further provides optional screening methods for identification and/or selection of a humanized antibody of interest.

The present invention provides a nucleic acid sequence encoding a humanized antibody that immunospecifically binds to an antigen, said nucleic acid sequence produced by the process comprising: (a) selecting an acceptor heavy chain framework region 1, an acceptor heavy chain framework region 2, an acceptor heavy chain framework region 3, and an acceptor heavy chain framework region 4, wherein at least one (preferably, at least two, at least three, or all four) of the framework regions is less than 65% (preferably, less than 60%, less than 55%, less than 50%, less than 45%, or less than 40%) identical to the corresponding framework region(s) of a donor antibody at the amino acid level, and wherein the acceptor heavy chain variable framework region contains at least one amino acid residue (preferably, at least two, or at least three amino acid residues) at amino acid residues 6, 23, 24 and/or 49 according to the Kabat numbering system that is (are) not identical to the corresponding residue(s) in the donor antibody; (b) selecting an acceptor light chain variable framework region less than 65% (preferably less than 60%, less than 55%, less than 50%, less than 45%, or less than 40%) identical to a donor antibody light chain variable framework region at the amino acid level; and (c) synthesizing a nucleic acid sequence comprising: (i) a first nucleotide sequence encoding a humanized light chain variable region, said first nucleotide sequence comprising nucleic acid sequences encoding CDRs from the donor antibody light chain variable region and nucleic acid sequences encoding the acceptor light chain variable framework regions with one or more mutations introduced at amino acid residues designated key residues, said key residues not including amino acid residues 4, 38, 43, 44, 46, 58, 62, 65, 66, 67, 68, 69, 73, 85, and 98 according to the Kabat numbering system, and (ii) a second nucleotide sequence encoding a humanized heavy chain variable region with at least one (preferably, at least two, at least three, or all four) framework region(s) that remains less than 65% (preferably less than 60%, less than 55%, less than 50%, less than 45%, or less than 40%) identical to the corresponding donor antibody heavy chain variable framework region(s) at the amino acid level, said second nucleotide sequence comprising nucleic acid sequences encoding CDRs from the donor antibody heavy chain variable region and nucleic acid sequences encoding the acceptor heavy chain variable framework regions with one or more mutations introduced at amino acid residues designated key residues, said key residues not including amino acid residues 2, 4, 24, 35, 36, 39, 43, 45, 64, 69, 70, 73, 74, 75, 76, 78, 92 and 93 according to the Kabat numbering system.

The present invention provides a nucleic acid sequence encoding a humanized antibody that immunospecifically binds to an antigen, said nucleic acid sequence produced by the process comprising: (a) selecting an acceptor heavy chain variable framework region (which preferably comprises framework region 1, framework region 2, framework region 3 and framework region 4) globally or overall less than 65% (preferably less than 60%, less than 55%, less than 50%, less than 45%, or less than 40%) identical to a donor antibody heavy chain variable framework region at the amino acid level, which acceptor heavy chain variable framework region contains at least one amino acid residue (preferably, at least two, or at least three amino acid residues) at amino acid residues 6, 23, 24 and/or 49 according to the Kabat numbering system that is (are) not identical to the corresponding residue(s) in the donor antibody; and (b) synthesizing a nucleic acid sequence comprising a nucleotide sequence encoding a humanized heavy chain variable region, said nucleotide sequence comprising nucleic acid sequences encoding complementarity determining regions (CDRs) from the donor antibody heavy chain variable region and nucleic acid sequences encoding the acceptor heavy chain variable framework regions. In certain embodiments, a donor antibody amino acid residue in the humanized heavy chain variable framework region is not within 6 Å, 6.5 Å, 7 Å, 7.5 Å or 8 Å of a CDR. The present invention also provides a cell containing or comprising a nucleic acid sequence encoding a humanized antibody that immunospecifically binds to an antigen, said cell is produced by introducing the nucleic acid sequence comprising the nucleotide sequence encoding the humanized heavy chain variable region described herein into the cell. In some embodiments, the cell further contains a nucleic acid sequence comprising a nucleotide sequence encoding a light chain variable region, preferably, a human or humanized light chain variable region. The present invention further provides a method of producing a humanized antibody that immunospecifically binds to an antigen, said method comprising expressing the nucleic acid sequence encoding the humanized antibody contained in the cell described herein. The present invention also provides optional screening methods for identification and/or selection of a humanized antibody of interest.

The present invention provides a nucleic acid sequence encoding a humanized antibody that immunospecifically binds to an antigen, said nucleic acid sequence produced by the process comprising: (a) selecting an acceptor heavy chain variable framework region (which preferably comprises framework region 1, framework region 2, framework region 3 and framework region 4) globally or overall less than 65% (preferably less than 60%, less than 55%, less than 50%, less than 45%, or less than 40%) identical to a donor antibody heavy chain variable framework region at the amino acid level, which acceptor heavy chain variable framework region contains at least one amino acid residue (preferably, at least two, or at least three amino acid residues) at amino acid residues 6, 23, 24 and/or 49 according to the Kabat numbering system, wherein the amino acid residue is (are) not identical to the corresponding residue(s) in the donor antibody; and (b) synthesizing a nucleic acid sequence comprising a nucleotide sequence encoding a humanized heavy chain variable region with a framework region (which preferably comprises framework region 1, framework region 2, framework region 3 and framework region 4) that globally or overall remains less than 65% (preferably less than 60%, less than 55%, less than 50%, less than 45%, or less than 40%) identical to the donor antibody heavy chain variable framework region at the amino acid level, said nucleotide sequence comprising nucleic acid sequences encoding CDRs from the donor antibody heavy chain variable region and nucleic acid sequences encoding the acceptor heavy chain variable framework regions with one or more mutations introduced at amino acid residues designated key residues, said key residues not including amino acid residues 2, 4, 24, 35, 36, 39, 43, 45, 64, 69, 70, 73, 74, 75, 76, 78, 92 and 93 according to the Kabat numbering system. In certain embodiments, the residues designated key are one or more of the following: adjacent to a CDR, a potential glycosylation site, a rare residue, a residue capable of interacting with the antigen, a residue capable of interacting with a CDR, canonical residues, contact residues between the variable heavy domain and variable light domain, a residue within the vernier zone, and/or a residue within the region which overlaps between the Chothia definition of the heavy chain variable region CDR1 and the Kabat definition of the first heavy chain framework. In some embodiments, the mutations introduced at amino acid residues designated key are substitutions. In particular embodiments, the amino acid residues designated key are not heavy chain variable framework region amino acid residues 6, 23, 24 and 49 as a group according to the Kabat numbering system. In a further embodiment, the amino acid residues designated key are not heavy chain variable framework region amino acid residues 6, 24, 48, 49, 71, 73, and 78 as a group according to the Kabat numbering system. In a further embodiment, the amino acid residues designated key are not heavy chain variable framework region amino acid residues 23, 24, 26 to 30, and 49 as a group according to the Kabat numbering system. In some embodiments, a donor antibody amino acid residue in the humanized heavy chain and/or light chain variable framework region is not within 6 Å, preferably 6.5 Å, 7 Å, 7.5 Å or 8 Å of a CDR. In accordance with the invention, the donor antibody and acceptor antibody are from different species (e.g., a donor antibody from mouse, and an acceptor antibody from human). The present invention also provides a cell containing a nucleic acid sequence encoding a humanized antibody that immunospecifically binds to an antigen, said cell produced by introducing the nucleic acid sequence comprising the nucleotide sequence encoding the humanized heavy chain variable region described herein into the cell. In some embodiments, the cell further contains a nucleic acid sequence comprising a nucleotide sequence encoding a light chain variable region, preferably, a human or humanized light chain variable region. The present invention also provides a method of producing a humanized antibody that immunospecifically binds to an antigen, said method comprising expressing the nucleic acid sequence encoding the humanized antibody contained in the cell described herein. The present invention further provides optional screening methods for identification and/or selection of a humanized antibody of interest.

The present invention provides a nucleic acid sequence encoding a humanized antibody that immunospecifically binds to an antigen, said nucleic acid sequence produced by the process comprising: (a) selecting an acceptor light chain variable framework region (which preferably comprises framework region 1, framework region 2, framework region 3 and framework region 4) globally or overall less than 65% (preferably less than 60%, less than 55%, less than 50%, less than 45%, or less than 40%) identical to a donor antibody light chain variable framework region at the amino acid level; and (b) synthesizing a nucleic acid sequence comprising a nucleotide sequence encoding a humanized light chain variable region, said nucleotide sequence comprising nucleic acid sequences encoding complementarity determining regions (CDRs) from the donor antibody light chain variable region and nucleic acid sequences encoding the acceptor light chain variable framework regions. The present invention also provides a cell containing or comprising a nucleic acid sequence encoding a humanized antibody that immunospecifically binds to an antigen, said cell produced by introducing the nucleic acid sequence comprising the nucleotide sequence encoding the humanized light chain variable region described herein into the cell. In some embodiments, the cell further contains or comprises a nucleic acid sequence comprising a nucleotide sequence encoding a heavy chain variable region, preferably, a human or humanized heavy chain variable region. The present invention also provides a method of producing a humanized antibody that immunospecifically binds to an antigen, said method comprising expressing the nucleic acid sequence encoding the humanized antibody contained in the cell described herein. The present invention further provides optional screening methods for identification and/or selection of a humanized antibody of interest.

The present invention provides a nucleic acid sequence encoding a humanized antibody that immunospecifically binds to an antigen, said nucleic acid sequence produced by the process comprising: (a) selecting an acceptor light chain variable framework region (which preferably comprises framework region 1, framework region 2, framework region 3 and framework region 4) globally or overall less than 65% (preferably less than 60%, less than 55%, less than 50%, less than 45%, or less than 40%) identical to a donor antibody heavy chain variable framework region at the amino acid level; and (b) synthesizing a nucleic acid sequence comprising a nucleotide sequence encoding a humanized light chain variable region, said nucleotide sequence comprising nucleic acid sequences encoding CDRs from the donor antibody light chain variable region and nucleic acid sequences encoding the acceptor light chain variable framework regions with one or more mutations introduced at amino acid residues designated key residues, said key residues not including amino acid residues 4, 38, 43, 44, 46, 58, 62, 65, 66, 67, 68, 69, 73, 85, and 98 according to the Kabat numbering system. In some embodiments, the mutations introduced at amino acid residues designated key are substitutions. In specific embodiments, the substitutions replace the acceptor amino acid residues in the light chain variable framework region with the corresponding amino acid residues in the donor light chain variable framework region. In some embodiments, the residues designated key are one or more of the following: adjacent to a CDR, a potential glycosylation site, a rare residue, a residue capable of interacting with the antigen, a residue capable of interacting with a CDR, canonical residues, contact residues between the variable heavy domain and variable light domain, and/or a residue within the vernier zone. The present invention also provides a cell containing or comprising a nucleic acid sequence encoding a humanized antibody that immunospecifically binds to an antigen, said cell produced by introducing the nucleic acid sequence comprising the nucleotide sequence encoding the humanized light chain variable region described herein into the cell. In some embodiments, the cell further contains or comprises a nucleic acid sequence comprising a nucleotide sequence encoding a heavy chain variable region, preferably, a human or humanized heavy chain variable region. The present invention also provides a method of producing a humanized antibody that immunospecifically binds to an antigen, said method comprising expressing the nucleic acid sequence encoding the humanized antibody contained in the cell described herein. The present invention further provides optional screening methods for identification and/or selection of a humanized antibody of interest.

The present invention provides a nucleic acid sequence encoding a humanized antibody that immunospecifically binds to an antigen, said nucleic acid sequence produced by the process comprising: (a) selecting an acceptor heavy chain variable framework region (which preferably comprises framework region 1, framework region 2, framework region 3 and framework region 4) globally or overall less than 65% (preferably less than 60%, less than 55%, less than 50%, less than 45%, or less than 40%) identical to a donor antibody heavy chain variable framework region at the amino acid level, which acceptor heavy chain variable framework region contains at least one amino acid residue (preferably, at least two, or at least three amino acid residues) at amino acid residues 6, 23, 24 and/or 49 according to the Kabat numbering system that is (are) not identical to the corresponding residues(s) in the donor antibody; and (b) synthesizing a nucleic acid sequence comprising: (i) a first nucleotide sequence encoding a light chain variable region, and (ii) a second nucleotide sequence encoding a humanized heavy chain variable region with a framework region (which preferably comprises framework region 1, framework region 2, framework region 3 and framework region 4) that globally or overall remains less than 65% (preferably less than 60%, less than 55%, less than 50%, less than 45%, or less than 40%) identical to the donor antibody heavy chain variable framework region at the amino acid level, said second nucleotide sequence comprising nucleic acid sequences encoding complementarity determining regions (CDRs) from the donor antibody heavy chain variable region and nucleic acid sequences encoding the acceptor heavy chain variable framework regions. The present invention provides a cell containing or comprising a nucleic acid sequence encoding a humanized antibody that immunospecifically binds to an antigen, said cell produced by introducing the nucleic acid sequence comprising the first nucleotide sequence and second nucleotide sequence described herein into the cell. In some embodiments, the light chain is humanized. In certain embodiments, a donor antibody amino acid residue in the humanized heavy chain variable framework region is not within 6 Å, preferably not within 6.5 Å, 7 Å, 7.5 Å or 8 Å of a CDR. The present invention also provides a method of producing a humanized antibody that immunospecifically binds to an antigen, said method comprising expressing the nucleic acid sequence encoding the humanized antibody contained in the cell described herein. The present invention further provides optional screening methods for identification and/or selection of a humanized antibody of interest.

The present invention provides a nucleic acid sequence encoding a humanized antibody that immunospecifically binds to an antigen, said nucleic acid sequence produced by the process comprising: (a) selecting an acceptor heavy chain variable framework region (which preferably comprises framework region 1, framework region 2, framework region 3 and framework region 4) globally or overall less than 65% (preferably less than 60%, less than 55%, less than 50%, less than 45%, or less than 40%) identical to a donor antibody heavy chain variable framework region at the amino acid level, which acceptor heavy chain variable framework region contains at least one amino acid residue (preferably at least two, or at least three amino acid residues) at amino acid residues 6, 23, 24 and/or 49 according to the Kabat numbering system that is (are) not identical to the corresponding residue(s) in the donor antibody; and (b) synthesizing a nucleic acid sequence comprising: (i) a first nucleotide sequence encoding a light chain variable region, and (ii) a second nucleotide sequence encoding a humanized heavy chain variable region with a framework region (which preferably comprises framework region 1, framework region 2, framework region 3 and framework region 4) that globally or overall remains less than 65% (preferably less than 60%, less than 55%, less than 50%, less than 45%, or less than 40%) identical to the donor antibody heavy chain variable framework region at the amino acid level, said second nucleotide sequence comprising nucleic acid sequences encoding CDRs from the donor antibody heavy chain variable region and nucleic acid sequences encoding the acceptor heavy chain variable framework regions with one or more mutations introduced at amino acid residues designated key residues, said key residues not including amino acid residues 2, 4, 24, 35, 36, 39, 43, 45, 64, 69, 70, 73, 74, 75, 76, 78, 92 and 93 according to the Kabat numbering system. In some embodiments, the light chain is humanized. In certain embodiments, the residues designated key are one or more of the following: adjacent to a CDR, a potential glycosylation site, a rare residue, a residue capable of interacting with the antigen, a residue capable of interacting with a CDR, canonical residues, contact residues between the variable heavy domain and variable light domain, a residue within the vernier zone, and/or a residue within the region which overlaps between the Chothia definition of the heavy chain CDR1 and the Kabat definition of the first heavy chain framework. In some embodiments, the mutations introduced at amino acid residues designated key are substitutions. In specific embodiments, the substitutions replace the acceptor amino acid residues in the heavy chain variable framework region with the corresponding amino acid residues in the donor heavy chain variable framework region. In some embodiments, a donor antibody amino acid residue in the humanized heavy chain and/or light chain variable framework region is not within 6 Å, preferably not within 6.5 Å, 7 Å, 7.5 Å or 8 Å of a CDR. The present invention also provides a cell containing or comprising a nucleic acid sequence encoding a humanized antibody that immunospecifically binds to an antigen, said cell produced by introducing the nucleic acid sequence comprising the first nucleotide sequence and the second nucleotide sequence described herein into the cell. The present invention also provides a method of producing a humanized antibody that immunospecifically binds to an antigen, said method comprising expressing the nucleic acid sequence encoding the humanized antibody contained in the cell described herein. The present invention further provides optional screening methods for identification and/or selection of a humanized antibody of interest.

The present invention provides a nucleic acid sequence encoding a humanized antibody that immunospecifically binds to an antigen, said nucleic acid sequence produced by the process comprising: (a) selecting an acceptor heavy chain variable framework region (which preferably comprises framework region 1, framework region 2, framework region 3 and framework region 4) globally or overall less than 65% (preferably less than 60%, less than 55%, less than 50%, less than 45%, or less than 40%) identical to a donor antibody heavy chain variable framework region at the amino acid level, which acceptor heavy chain variable framework region contains at least one amino acid residue (preferably, at least two, or at least three amino acid residues) at amino acid residues 6, 23, 24 and/or 49 according to the Kabat numbering system that is (are) not identical to the corresponding residue(s) in the donor antibody; (b) selecting an acceptor light chain variable framework region (which preferably comprises framework region 1, framework region 2, framework region 3 and framework region 4) globally or overall less than 65% (preferably less than 60%, less than 55%, less than 50%, less than 45%, or less than 40%) identical to a donor antibody light chain variable framework region at the amino acid level; and (c) synthesizing a nucleic acid sequence comprising: (i) a first nucleotide sequence encoding a humanized light chain variable region, said first nucleotide sequence comprising nucleic acid sequences encoding CDRs from the donor antibody light chain variable region and nucleic acid sequences encoding the acceptor light chain variable framework regions with one or more mutations introduced at amino acid residues designated key residues, said key residues not including amino acid residues 4, 38, 43, 44, 46, 58, 62, 65, 66, 67, 68, 69, 73, 85, and 98 according to the Kabat numbering system, and (ii) a second nucleotide sequence encoding a humanized heavy chain variable region with a framework region (which preferably comprises framework region 1, framework region 2, framework region 3 and framework region 4) that globally or overall remains less than 65% (preferably less than 60%, less than 55%, less than 50%, less than 45%, or less than 40%) identical to the corresponding donor antibody heavy chain variable framework region at the amino acid level, said second nucleotide sequence comprising nucleic acid sequences encoding complementarity determining regions (CDRs) from the donor antibody heavy chain variable region and nucleic acid sequences encoding the acceptor heavy chain variable framework regions. In some embodiments, the residues designated key are one or more of the following: adjacent to a CDR, a potential glycosylation site, a rare residue, a residue capable of interacting with the antigen, a residue capable of interacting with a CDR, canonical residues, contact residues between the variable heavy domain and variable light domain, a residue within the vernier zone, and/or a residue within the region which overlaps between the Chothia definition of the heavy chain CDR1 and the Kabat definition of the first heavy chain framework. In some embodiments, the mutations introduced at the residues designated key are substitutions. In specific embodiments, the substitutions replace the acceptor amino acid residues in the light chain variable framework region with the corresponding amino acid residues in the donor light chain variable framework region. In some embodiments, a donor antibody amino acid residue in the humanized heavy chain and/or light chain variable framework region is not within 6 Å, preferably not within 6.5 Å, 7 Å, 7.5 Å or 8 Å of a CDR. The present invention also provides a cell containing a nucleic acid sequence encoding a humanized antibody that immunospecifically binds to an antigen, said cell produced by introducing the nucleic acid sequence comprising the first nucleotide sequence and second nucleotide sequence described herein into the cell. The present invention also provides a method of producing a humanized antibody that immunospecifically binds to an antigen, said method comprising expressing the nucleic acid sequence encoding the humanized antibody contained in the cell described herein. The present invention also provides optional screening methods for identification and/or selection of a humanized antibody of interest.

The present invention provides a nucleic acid sequence encoding a humanized antibody that immunospecifically binds to an antigen, said nucleic acid sequence produced by the process comprising: (a) selecting an acceptor heavy chain variable framework region (which preferably comprises framework region 1, framework region 2, framework region 3 and framework region 4) globally or overall less than 65% (preferably less than 60%, less than 55%, less than 50%, less than 45%, or less than 40%) identical to a donor antibody heavy chain variable framework region at the amino acid level, which acceptor heavy chain variable framework region contains at least one amino acid residue (preferably, at least two, or at least three amino acid residues) at amino acid residues 6, 23, 24 and/or 49 according to the Kabat numbering system that is (are) not identical to the corresponding residues in the donor antibody; (b) selecting an acceptor light chain variable framework region (which preferably comprises framework region 1, framework region 2, framework region 3 and framework region 4) globally or overall less than 65% (preferably less than 60%, less than 55%, less than 50%, less than 45%, or less than 40%) identical to a donor antibody light chain variable framework region at the amino acid level; and (c) synthesizing a nucleic acid sequence comprising: (i) a first nucleotide sequence encoding a humanized light chain variable region, said first nucleotide sequence comprising nucleic acid sequences encoding CDRs from the donor antibody light chain variable region and nucleic acid sequences encoding the acceptor light chain variable framework regions with one or more mutations introduced at amino acid residues designated key residues, said key residues not including amino acid residues 4, 38, 43, 44, 46, 58, 62, 65, 66, 67, 68, 69, 73, 85, and 98 according to the Kabat numbering system, and (ii) a second nucleotide sequence encoding a humanized heavy chain variable region with a framework region (which preferably comprises framework region 1, framework region 2, framework region 3 and framework region 4) that globally or overall remains less than 65% (preferably less than 60%, less than 55%, less than 50%, less than 45%, or less than 40%) identical to the donor antibody heavy chain variable framework region at the amino acid level, said second nucleotide sequence comprising nucleic acid sequences encoding CDRs from the donor antibody heavy chain variable region and nucleic acid sequences encoding the acceptor heavy chain variable framework regions with one or more mutations introduced at amino acid residues designated key residues, said key residues not including amino acid residues 2, 4, 24, 35, 36, 39, 43, 45, 64, 69, 70, 73, 74, 75, 76, 78, 92 and 93 according to the Kabat numbering system. In some embodiments, the residues designated key are one or more of the following: adjacent to a CDR, a potential glycosylation site, a rare residue, a residue capable of interacting with the antigen, a residue capable of interacting with a CDR, canonical residues, contact residues between the variable heavy domain and variable light domain, or a residue within the vernier zone, and/or a residue within the region which overlaps between the Chothia definition of the heavy chain variable region CDR1 and the Kabat definition of the first heavy chain framework. In certain embodiments, the mutations introduced at the amino acid residues designated key are substitutions. In specific embodiments, the substitutions replace the acceptor amino acid residues in the heavy and/or light chain variable framework region with the corresponding amino acid residues in the donor heavy and/or light chain variable framework region. In some embodiments, a donor antibody amino acid residue in the humanized heavy and/or light chain variable framework region is not within 6 Å, preferably not within 6.5 Å, 7 Å, 7.5 Å or 8 Å of a CDR. The present invention also provides a cell containing or comprising a nucleic acid sequence encoding a humanized antibody that immunospecifically binds to an antigen, said cell produced by introducing the nucleic acid sequence comprising the first nucleotide sequence and the second nucleotide sequence described herein into the cell. The present invention also provides a method of producing a humanized antibody that immunospecifically binds to an antigen, said method comprising expressing the nucleic acid sequence encoding the humanized antibody contained in the cell described herein. The present invention further provides optional screening methods for identification and/or selection of a humanized antibody of interest.

The present invention provides a population of cells engineered to contain or comprise nucleic acid sequences encoding a plurality of humanized antibodies produced by a process comprising: (a) selecting acceptor heavy chain variable framework regions (which preferably comprises framework region 1, framework region 2, framework region 3 and framework region 4) globally or overall less than 65% (preferably less than 60%, less than 55%, less than 50%, less than 45%, or less than 40%) identical to a donor antibody heavy chain variable framework region at the amino acid level, which acceptor heavy chain variable framework regions contain at least one amino acid residue (preferably, at least two, or at least three amino acid residues) at amino acid residues 6, 23, 24 and/or 49 according to the Kabat numbering system that is (are) not identical to the corresponding residues in the donor antibody; (b) synthesizing a nucleic acid sequences comprising nucleotide sequences encoding humanized heavy chain variable regions, said nucleotide sequences comprising nucleic acid sequences encoding complementarity determining regions (CDRs) from the donor antibody heavy chain variable region and nucleic acid sequences encoding the acceptor heavy chain variable framework regions; and (c) introducing the nucleic acid sequences comprising the nucleotide sequences encoding the humanized heavy chain variable regions into cells. In some embodiments, the cells further contain a nucleic acid sequence comprising a nucleotide sequence encoding a light chain variable region. In specific embodiments, the light chain is humanized. In certain embodiments, the residues designated key are one or more of the following: adjacent to a CDR, a potential glycosylation site, a rare residue, a residue capable of interacting with the antigen, a residue capable of interacting with a CDR, canonical residues, contact residues between the variable heavy domain and variable light domain, a residue within the vernier zone, and/or a residue within a region which overlaps between the Chothia definition of the heavy chain CDR1 and the Kabat definition of the first heavy chain framework. The population of the cells can be used to screen, identify and/or select a humanized antibody that immunospecifically binds to an antigen of interest.

The present invention provides a population of cells engineered to contain or comprise nucleic acid sequences encoding a plurality of humanized antibodies produced by a process comprising: (a) selecting acceptor heavy chain variable framework regions (which preferably comprises framework region 1, framework region 2, framework region 3 and framework region 4) globally or overall less than 65% (preferably less than 60%, less than 55%, less than 50%, less than 45%, or less than 40%) identical to a donor antibody heavy chain variable framework region at the amino acid level, which acceptor heavy chain variable framework regions contain at least one amino acid residue (preferably, at least two, or at least three amino acid residues) at amino acid residues 6, 23, 24 and/or 49 according to the Kabat numbering system that is (are) not identical to the corresponding residues in the donor antibody; (b) synthesizing nucleic acid sequences comprising nucleotide sequences encoding humanized heavy chain variable regions with framework regions that remain less than 65% (preferably less than 60%, less than 55%, less than 50%, less than 45%, or less than 40%) identical to the donor antibody heavy chain variable framework region at the amino acid level, said nucleotide sequences comprising nucleic acid sequences encoding CDRs from the donor antibody heavy chain variable region and nucleic acid sequences encoding the acceptor heavy chain variable framework regions with one or more mutations introduced at amino acid residues designated key residues, said key residues not including amino acid residues 2, 4, 24, 35, 36, 39, 43, 45, 64, 69, 70, 73, 74, 75, 76, 78, 92 and 93 according to the Kabat numbering system; and (c) introducing the nucleic acid sequences comprising the nucleotide sequences encoding the humanized heavy chain variable regions into cells. In some embodiments, the cells further contain a nucleotide sequence encoding a light chain variable region, preferably a human or humanized light chain variable region. In certain embodiments, the residues designated key are one or more of the following: adjacent to a CDR, a potential glycosylation site, a rare residue, a residue capable of interacting with the antigen, a residue capable of interacting with a CDR, canonical residues, contact residues between the variable heavy domain and variable light domain, a residue within the vernier zone, and/or a residue within the region which overlaps between the Chothia definition of the heavy chain CDR1 and the Kabat definition of the first heavy chain framework. In some embodiments, the mutations introduced at the amino acid residues designated key are substitutions. In specific embodiments, the substitutions replace the acceptor amino acid residues in the heavy chain variable framework regions with the corresponding amino acid residues in the donor heavy chain variable framework region. The population of the cells can be used to screen, identify and/or select a humanized antibody that immunospecifically binds to an antigen of interest.

The present invention provides a population of cells engineered to contain or comprise nucleic acid sequences encoding a plurality of humanized antibodies produced by a process comprising: (a) selecting acceptor light chain variable framework regions (which preferably comprises framework region 1, framework region 2, framework region 3 and framework region 4) globally or overall less than 65% (preferably less than 60%, less than 55%, less than 50%, less than 45%, or less than 40%) identical to a donor antibody light chain variable framework region at the amino acid level; (b) synthesizing nucleic acid sequences comprising nucleotide sequences encoding humanized light chain variable regions, said nucleotide sequences comprising nucleic acid sequences encoding complementarity determining regions (CDRs) from the donor antibody light chain variable region and nucleic acid sequences encoding the acceptor light chain variable framework regions; and (c) introducing the nucleic acid sequences comprising the nucleotide sequences encoding the humanized light chain variable regions into cells. The population of the cells can be used to screen, identify and/or select a humanized antibody that immunospecifically binds to an antigen of interest

The present invention provides a population of cells engineered to contain or comprise nucleic acid sequences encoding a plurality of humanized antibodies produced by a process comprising: (a) selecting acceptor light chain variable framework regions (which preferably comprises framework region 1, framework region 2, framework region 3 and framework region 4) globally or overall less than 65% (preferably less than 60%, less than 55%, less than 50%, less than 45%, or less than 40%) identical to a donor antibody heavy chain variable framework region at the amino acid level; (b) synthesizing nucleic acid sequences comprising nucleotide sequences encoding humanized light chain variable regions, said nucleotide sequences comprising nucleic acid sequences encoding CDRs from the donor antibody light chain variable region and nucleic acid sequences encoding the acceptor light chain variable framework regions with one or more mutations introduced at amino acid residues designated key residues, said key residues not including amino acid residues 4, 38, 43, 44, 46, 58, 62, 65, 66, 67, 68, 69, 73, 85, and 98 according to the Kabat numbering system; and (c) introducing the nucleic acid sequences comprising the nucleotide sequences encoding the humanized light chain variable regions into cells. In some embodiments, the residues designated key are one or more of the following: adjacent to a CDR, a potential glycosylation site, a rare residue, a residue capable of interacting with the antigen, a residue capable of interacting with a CDR, canonical residues, contact residues between the variable heavy domain and variable light domain, a residue within the vernier zone, and/or a residue within the region which overlaps between the Chothia definition of the heavy chain CDR1 and the Kabat definition of the first heavy chain framework. In certain embodiments, the mutations introduced at the amino acid residues designated key are substitutions. In specific embodiments, the substitutions replace the acceptor amino acid residues in the light chain variable framework regions with the corresponding amino acid residues in the donor light chain variable framework region. The population of the cells can be used to screen, identify and/or select a humanized antibody that immunospecifically binds to an antigen of interest

The present invention provides a population of cells engineered to contain or comprise nucleic acid sequences encoding a plurality of humanized antibodies produced by a process comprising: (a) selecting acceptor heavy chain variable framework regions (which preferably comprises framework region 1, framework region 2, framework region 3 and framework region 4) globally or overall less than 65% (preferably less than 60%, less than 55%, less than 50%, less than 45%, or less than 40%) identical to a donor antibody heavy chain variable framework region at the amino acid level, which acceptor heavy chain variable framework regions contain at least one amino acid residue (preferably, at least two, or at least three amino acid residues) at amino acid residues 6, 23, 24 and/or 49 according to the Kabat numbering system that is (are) not identical to the corresponding amino acid residue(s) in the donor antibody; (b) synthesizing nucleic acid sequences comprising: (i) a first set of nucleotide sequences encoding light chain variable regions, and (ii) a second set of nucleotide sequences encoding humanized heavy chain variable regions with framework regions (which preferably comprises framework region 1, framework region 2, framework region 3 and framework region 4) that globally or overall remain less than 65% (preferably less than 60%, less than 55%, less than 50%, less than 45%, or less than 40%) identical to the donor antibody heavy chain variable framework region at the amino acid level, said second set of nucleotide sequences comprising nucleic acid sequences encoding complementarity determining regions (CDRs) from the donor antibody heavy chain variable region and nucleic acid sequences encoding the acceptor heavy chain variable framework regions; and (c) introducing the nucleic acid sequences comprising the first set of nucleotide sequences and second set of nucleotide sequences into a cell. Preferably, the light chain is humanized. The population of the cells can be used to screen, identify and/or select a humanized antibody that immunospecifically binds to an antigen of interest.

The present invention provides a population of cells engineered to contain or comprise nucleic acid sequences encoding a plurality of humanized antibodies produced by a process comprising: (a) selecting acceptor heavy chain variable framework regions (which preferably comprises framework region 1, framework region 2, framework region 3 and framework region 4) globally or overall less than 65% (preferably less than 60%, less than 55%, less than 50%, less than 45%, or less than 40%) identical to a donor antibody heavy chain variable framework region at the amino acid level, which acceptor heavy chain variable framework regions contain at least one amino acid residue (preferably, at least two, or at least three amino acid residues) at amino acid residues 6, 23, 24 and/or 49 according to the Kabat numbering system that is (are) not identical to the corresponding amino acid residue(s) in the donor antibody; (b) synthesizing nucleic acid sequences comprising: (i) a first set of nucleotide sequences encoding light chain variable regions, and (ii) a second set of nucleotide sequences encoding humanized heavy chain variable regions with framework regions (which preferably comprises framework region 1, framework region 2, framework region 3 and framework region 4) that globally or overall remain less than 65% (preferably less than 60%, less than 55%, less than 50%, less than 45%, or less than 40%) identical to the donor antibody heavy chain variable framework region at the amino acid level, said second set of nucleotide sequence comprising nucleic acid sequences encoding CDRs from the donor antibody heavy chain variable region and nucleic acid sequences encoding the acceptor heavy chain variable framework regions with one or more mutations introduced at amino acid residues designated key residues, said key residues not including amino acid residues 2, 4, 24, 35, 36, 39, 43, 45, 64, 69, 70, 73, 74, 75, 76, 78, 92 and 93 according to the Kabat numbering system; and (c) introducing the nucleic acid sequences comprising the first set of nucleotide sequences and the second set of nucleotide sequences into cells. In some embodiments, the light chain is humanized. In some embodiments, the residues designated key are one or more of the following: adjacent to a CDR, a potential glycosylation site, a rare residue, a residue capable of interacting with the antigen, a residue capable of interacting with a CDR, canonical residues, contact residues between the variable heavy domain and variable light domain, a residue within the vernier zone, and/or a residue within the region which overlaps between the Chothia definition of the heavy chain CDR1 and the Kabat definition of the first heavy chain framework. In certain embodiments, the mutations introduced at the amino acid residues designated key are substitutions. In specific embodiments, the substitutions replace the acceptor amino acid residues in the heavy chain variable framework regions with the corresponding amino acid residues in the donor heavy chain variable framework region. The population of the cells can be used to screen, identify and/or select a humanized antibody that immunospecifically binds to an antigen of interest.

The present invention provides a population of cells engineered to contain or comprise nucleic acid sequences encoding a plurality of humanized antibodies produced by a process comprising: (a) selecting acceptor heavy chain variable framework regions (which preferably comprises framework region 1, framework region 2, framework region 3 and framework region 4) globally or overall less than 65% (preferably less than 60%, less than 55%, less than 50%, less than 45%, or less than 40%) identical to a donor antibody heavy chain variable framework region at the amino acid level, which acceptor heavy chain variable framework regions contain at least one amino acid residue (preferably, at least two, or at least three amino acid residues) at amino acid residues 6, 23, 24 and/or 49 according to the Kabat numbering system that is (are) not identical to the corresponding amino acid residue(s) in the donor antibody; (b) selecting acceptor light chain variable framework regions (which preferably comprises framework region 1, framework region 2, framework region 3 and framework region 4) globally or overall less than 65% (preferably less than 60%, less than 55%, less than 50%, less than 45%, or less than 40%) identical to a donor antibody light chain variable framework region at the amino acid level; (c) synthesizing nucleic acid sequences comprising: (i) a first set of nucleotide sequences encoding humanized light chain variable regions, said first set of nucleotide sequences comprising nucleic acid sequences encoding CDRs from the donor antibody light chain variable region and nucleic acid sequences encoding the acceptor light chain variable framework regions with one or more mutations introduced at amino acid residues designated key residues, said key residues not including amino acid residues 4, 38, 43, 44, 46, 58, 62, 65, 66, 67, 68, 69, 73, 85, and 98 according to the Kabat numbering system, and (ii) a second set of nucleotide sequences encoding humanized heavy chain variable regions with framework regions that remain less than 65% (preferably less than 60%, less than 55%, less than 50%, less than 45%, or less than 40%) identical to the donor antibody heavy chain variable framework region at the amino acid level, said second set of nucleotide sequences comprising nucleic acid sequences encoding complementarity determining regions (CDRs) from the donor antibody heavy chain variable region and nucleic acid sequences encoding the acceptor heavy chain variable framework regions; and (d) introducing the nucleic acid sequences comprising the first set of nucleotide sequences and second set of nucleotide sequences into cells. In some embodiments, the residues designated key are one or more of the following: adjacent to a CDR, a potential glycosylation site, a rare residue, a residue capable of interacting with the antigen, a residue capable of interacting with a CDR, canonical residues, contact residues between the variable heavy domain and variable light domain, a residue within the vernier zone, and/or a residue within the region which overlaps between the Chothia definition of the heavy chain CDR1 and the Kabat definition of the first heavy chain framework. In certain embodiments, the mutations introduced at the amino acid residues designated key are substitutions. In specific embodiments, the substitutions replace the acceptor amino acid residues in the light chain variable framework regions with the corresponding amino acid residues in the donor light chain variable framework region. The population of the cells can be used to screen, identify and/or select a humanized antibody that immunospecifically binds to an antigen of interest.

The present invention provides a population of cells engineered to contain or comprise nucleic acid sequences encoding a plurality of humanized antibodies produced by a process comprising: (a) selecting acceptor heavy chain variable framework regions (which preferably comprises framework region 1, framework region 2, framework region 3 and framework region 4) globally or overall less than 65% (preferably less than 60%, less than 55%, less than 50%, less than 45%, or less than 40%) identical to a donor antibody heavy chain variable framework region at the amino acid level, which acceptor heavy chain variable framework regions contain at least one amino acid residue (preferably, at least two, or at least three amino acid residues) at amino acid residues 6, 23, 24 and/or 49 according to the Kabat numbering system that is (are) not identical to the corresponding amino acid residue(s) in the donor antibody; (b) selecting acceptor light chain variable framework regions (which preferably comprises framework region 1, framework region 2, framework region 3 and framework region 4) globally or overall less than 65% (preferably less than 60%, less than 55%, less than 50%, less than 45%, or less than 40%) identical to a donor antibody light chain variable framework region at the amino acid level; (c) synthesizing nucleic acid sequences comprising: (i) a first set of nucleotide sequences encoding humanized light chain variable regions, said first set of nucleotide sequences comprising nucleic acid sequences encoding CDRs from the donor antibody light chain variable region and nucleic acid sequences encoding the acceptor light chain variable framework regions with one or more mutations introduced at amino acid residues designated key residues, said key residues not including amino acid residues 4, 38, 43, 44, 46, 58, 62, 65, 66, 67, 68, 69, 73, 85, and 98 according to the Kabat numbering system, and (ii) a second set of nucleotide sequences encoding humanized heavy chain variable regions with framework regions (which preferably comprises framework region 1, framework region 2, framework region 3 and framework region 4) that globally or overall remain less than 65% (preferably less than 60%, less than 55%, less than 50%, less than 45%, or less than 40%) identical to the donor antibody heavy chain variable framework region at the amino acid level, said second set of nucleotide sequences comprising nucleic acid sequences encoding CDRs from the donor antibody heavy chain variable region and nucleic acid sequences encoding the acceptor heavy chain variable framework regions with one or more mutations introduced at amino acid residues designated key residues, said key residues not including amino acid residues 2, 4, 24, 35, 36, 39, 43, 45, 64, 69, 70, 73, 74, 75, 76, 78, 92 and 93 according to the Kabat numbering system; and (d) introducing the nucleic acid sequences comprising the first set of nucleotide sequences and the second set of nucleotide sequences into cells. In some embodiments, the residues designated key are one or more of the following: adjacent to a CDR, a potential glycosylation site, a rare residue, a residue capable of interacting with the antigen, a residue capable of interacting with a CDR, canonical residues, contact residues between the variable heavy domain and variable light domain, a residue within the vernier zone, and/or a residue within the region which overlaps between the Chothia definition of the heavy chain CDR1 and the Kabat definition of the first heavy chain framework. In certain embodiments, the mutations introduced at the amino acid residues designated key are substitutions. In specific embodiments, the substitutions replace the acceptor amino acid residues in the heavy and/or light chain variable framework regions with the corresponding amino acid residues in the donor heavy and/or light chain variable framework region. The population of the cells can be used to screen, identify and/or select a humanized antibody that immunospecifically binds to an antigen of interest.

In accordance with the present invention, the cells described herein may contain a heavy chain variable region, a light chain variable region, a heavy chain variable region and a constant region, a light chain variable region and a constant region, or a combination thereof (e.g., a light chain and a heavy chain with constant region, a heavy chain variable region and a light chain variable region, etc).

The present invention provides a method of producing a humanized antibody that immunospecifically binds to an antigen, said method comprising providing a cell containing or comprising nucleic acid sequences comprising nucleotide sequences encoding humanized heavy chain and light chain variable regions and expressing the nucleic acid sequences, wherein said cell containing or comprising the nucleic acid sequences is produced by: (a) comparing the nucleic acid sequence of a donor antibody heavy chain variable region against a collection of sequences of acceptor heavy chain variable regions; (b) selecting an acceptor heavy chain variable framework region (which preferably comprises framework region 1, framework region 2, framework region 3 and framework region 4) globally or overall less than 65% (preferably less than 60%, less than 55%, less than 50%, less than 45%, or less than 40%) identical to the donor antibody heavy chain variable framework region at the amino acid level, which acceptor heavy chain variable framework region contains at least one (preferably, at least two, or at least three amino acid residues) at amino acid residues 6, 23, 24 and/or 49 according to the Kabat numbering system that is (are) not identical to the corresponding amino acid residue(s) in the donor antibody; (c) synthesizing a nucleic acid sequence encoding a humanized heavy chain variable region, said nucleic acid sequence comprising nucleotide sequences encoding complementarity determining regions (CDRs) from the donor antibody heavy chain variable region and nucleotide sequences encoding the acceptor heavy chain variable framework regions; and (d) introducing the nucleic acid sequence encoding the humanized heavy chain variable region into a cell. In some embodiments, the residues designated key are one or more of the following: adjacent to a CDR, a potential glycosylation site, a rare residue, a residue capable of interacting with the antigen, a residue capable of interacting with a CDR, canonical residues, contact residues between the variable heavy domain and variable light domain, a residue within the vernier zone, and or a residue within the region which overlaps between the Chothia definition of the heavy chain CDR1 and the Kabat definition of the first heavy chain framework. In certain embodiments, the mutations introduced at the amino acid residues designated key are substitutions. In specific embodiments, the substitutions replace the acceptor amino acid residues in the heavy chain variable framework region with the corresponding amino acid residues in the donor heavy chain variable framework region.

The present invention provides a method of producing a humanized antibody that immunospecifically binds to an antigen, said method comprising providing a cell containing or comprising nucleic acid sequences comprising nucleotide sequences encoding humanized heavy chain and light chain variable regions and expressing the nucleic acid sequences, wherein said cell containing or comprising the nucleic acid sequences is produced by: (a) comparing the nucleic acid sequence of a donor antibody heavy chain variable region against a collection of sequences of acceptor heavy chain variable regions; (b) selecting an acceptor heavy chain variable framework region (which preferably comprises framework region 1, framework region 2, framework region 3 and framework region 4) globally or overall less than 65% (preferably less than 60%, less than 55%, less than 50%, less than 45%, or less than 40%) identical to the donor antibody heavy chain variable framework region at the amino acid level, which acceptor heavy chain variable framework region contains at least one amino acid (preferably, at least two, or at least three amino acid residues) at amino acid residues 6, 23, 24 and/or 49 according to the Kabat numbering system that is (are) not identical to the corresponding amino acid residue(s) in the donor antibody; (c) synthesizing a nucleic acid sequence comprising nucleotide sequence encoding a humanized heavy chain variable region, said nucleotide sequence comprising nucleic acid sequences encoding complementarity determining regions (CDRs) from the donor antibody heavy chain variable region and nucleic acid sequences encoding the acceptor heavy chain variable framework regions with one or more mutations introduced at residues designated key residues; and (d) introducing the nucleic acid sequence comprising the nucleotide sequence encoding the humanized heavy chain variable region into a cell. In some embodiments, the residues designated key are one or more of the following: adjacent to a CDR, a potential glycosylation site, a rare residue, a residue capable of interacting with the antigen, a residue capable of interacting with a CDR, canonical residues, contact residues between the variable heavy domain and variable light domain, a residue within the vernier zone, and/or a residue within the region which overlaps between the Chothia definition of the heavy chain CDR1 and the Kabat definition of the first heavy chain framework. In some embodiments, the mutations introduced at amino acid residues designated key are substitutions. In specific embodiments, the substitutions replace the acceptor amino acid residues in the heavy chain variable framework region with the corresponding amino acid residues in the donor heavy chain variable framework region.

The present invention provides a method of producing a humanized antibody that immunospecifically binds to an antigen, said method comprising providing a cell containing or comprising nucleic acid sequences comprising nucleotide sequences encoding humanized heavy chain and light chain variable regions and expressing the nucleic acid sequences, wherein said cell containing or comprising the nucleic acid sequences is produced by: (a) comparing the nucleic acid sequence of a donor antibody light chain variable region against a collection of sequences of acceptor light chain variable regions; (b) selecting an acceptor light chain variable framework region (which preferably comprises framework region 1, framework region 2, framework region 3 and framework region 4) globally or overall less than 65% (preferably less than 60%, less than 55%, less than 50%, less than 45%, or less than 40%) identical to the donor antibody light chain variable framework region at the amino acid level; (c) synthesizing a nucleic acid sequence comprising nucleotide sequence encoding a humanized light chain variable region, said nucleotide sequence comprising nucleic acid sequences encoding complementarity determining regions (CDRs) from the donor antibody light chain variable region and nucleic acid sequences encoding the acceptor light chain variable framework regions with one or more mutations introduced at residues designated key residues; and (d) introducing the nucleic acid sequence comprising the nucleotide sequence encoding the humanized light chain variable region into a cell. In some embodiments, the residues designated key are one or more of the following: adjacent to a CDR, a potential glycosylation site, a rare residue, a residue capable of interacting with the antigen, a residue capable of interacting with a CDR, canonical residues, contact residues between the variable heavy domain and variable light domain, and/or a residue within the vernier zone. In some embodiments, the mutations introduced at amino acid residues designated key are substitutions. In specific embodiments, the substitutions replace the acceptor amino acid residues in the light chain variable framework region with the corresponding amino acid residues in the donor light chain variable framework region.

The present invention provides a method of producing a humanized antibody that immunospecifically binds to an antigen, said method comprising providing a cell containing or comprising nucleic acid sequences comprising nucleotide sequences encoding humanized heavy chain and light chain variable regions and expressing the nucleic acid sequences, wherein said cell containing or comprising the nucleic acid sequences is produced by: (a) comparing the nucleic acid sequence of a donor antibody heavy chain variable region against a collection of sequences of acceptor heavy chain variable regions; (b) selecting an acceptor heavy chain variable framework region (which preferably comprises framework region 1, framework region 2, framework region 3 and framework region 4) globally or overall less than 65% (preferably less than 60%, less than 55%, less than 50%, less than 45%, or less than 40%) identical to the donor antibody heavy chain variable framework region at the amino acid level, which acceptor heavy chain variable framework region preferably contains at least one amino acid (preferably, at least two, or at least three amino acid residues) at amino acid residues 6, 23, 24 and/or 49 according to the Kabat numbering system that is (are) not identical to the corresponding amino acid residue(s) in the donor antibody; (c) synthesizing a nucleic acid sequence comprising nucleotide sequence encoding a humanized heavy chain variable region, said nucleotide sequence comprising nucleic acid sequences encoding complementarity determining regions (CDRs) from the donor antibody heavy chain variable region and nucleic acid sequences encoding the acceptor heavy chain variable framework regions with one or more mutations introduced at residues designated key residues; (d) comparing the nucleic acid sequence of a donor antibody light chain variable region against a collection of sequences of acceptor light chain variable regions; (e) selecting an acceptor light chain variable framework region (which preferably comprises framework region 1, framework region 2, framework region 3 and framework region 4) globally or overall less than 65% (preferably less than 60%, less than 55%, less than 50%, less than 45%, or less than 40%) identical to the donor antibody light chain variable framework region at the amino acid level; (f) synthesizing a nucleic acid sequence comprising nucleotide sequence encoding a humanized light chain variable region, said nucleotide sequence comprising nucleic acid sequences encoding complementarity determining regions (CDRs) from the donor antibody light chain variable region and nucleic acid sequences encoding the acceptor light chain variable framework regions with one or more mutations introduced at residues designated key residues; and (d) introducing the nucleic acid sequence comprising the nucleotide sequence encoding the humanized heavy chain variable region and the humanized light chain variable region into a cell.

The present invention provides optional screening methods for identification and/or selection of a humanized antibody of interest. The present invention also provides a method of identifying a humanized antibody that immunospecifically binds to an antigen of interest, said method comprising expressing the nucleic acid sequences in the cells described hereinabove and screening for a humanized antibody that has an affinity of at least 1×10⁶ M⁻¹, preferably at least 1×10⁷ M⁻¹, at least 1×10⁸ M⁻¹, or at least 1×10⁹ M⁻¹ or above for said antigen.

In accordance with the present invention, the antibodies generated as described herein (e.g., a humanized antibody) comprise a light chain variable region and/or a heavy chain variable region. In some embodiments, the antibodies generated as described herein further comprise a constant region(s).

The present invention provides antibodies (preferably, humanized antibodies) generated in accordance with the invention conjugated or fused to a moiety (e.g., a therapeutic agent or drug). In a specific embodiment, the invention provides humanized anti-interleukin-9 (anti-IL-9) antibody and/or a humanized anti-EphA2 antibody generated in accordance with the present invention conjugated or fused to a moiety. The present invention also provides compositions, preferably pharmaceutical compositions, comprising an antibody generated and/or identified in accordance with the present invention and a carrier, diluent or excipient. In a specific embodiment, the present invention provides compositions, preferably pharmaceutical compositions, comprising a humanized anti-IL-9 antibody and/or a humanized anti-EphA2 antibody generated and/or identified in accordance with the present invention and a carrier, diluent or excipient. In certain preferred embodiments, the present invention provides compositions, preferably pharmaceutical compositions, comprising a humanized antibody as described herein and a carrier, diluent or excipient. The present invention also provides compositions, preferably pharmaceutical compositions, comprising an antibody generated and/or identified in accordance with the present invention conjugated or fused to a moiety (e.g., a therapeutic agent or drug), and a carrier, diluent or excipient. In certain preferred embodiments, the present invention provides compositions comprising a humanized antibody (or fragment thereof) conjugated or fused to a moiety (e.g., a therapeutic agent or drug), and a carrier, diluent or excipient. The present invention further provides uses of an antibody generated and/or identified in accordance with the present invention (e.g., a humanized antibody) alone or in combination with other therapies to prevent, treat, manage or ameliorate a disorder or a symptom thereof.

The pharmaceutical compositions of the invention may be used for the prevention, management, treatment or amelioration of a disease or one or more symptoms thereof. Preferably, the pharmaceutical compositions of the invention are sterile and in suitable form for a particular method of administration to a subject with a disease. In a specific embodiment, the compositions of the invention comprising a humanized anti-IL-9 antibody are used for the prevention, management, treatment or amelioration of a respiratory disorder or a symptom thereof. In another embodiment, the compositions of the invention comprising a humanized anti-EphA2 antibody are used for the prevention, management, treatment or amelioration of a hyperproliferative cell disease.

The invention further provides methods of detecting, diagnosing and/or monitoring the progression of a disorder utilizing one or more antibodies (preferably, one or more humanized antibodies) generated and/or identified in accordance with the methods of the invention.

The present invention provides a pharmaceutical pack or kit comprising one or more containers filled with a humanized antibody of the invention. The pharmaceutical pack or kit may further comprises one or more other prophylactic or therapeutic agents useful for the treatment of a particular disease. The invention also provides a pharmaceutical pack or kit comprising one or more containers filled with one or more of the ingredients of the pharmaceutical compositions of the invention. Optionally associated with such container(s) can be a notice in the form prescribed by a governmental agency regulating the manufacture, use or sale of pharmaceuticals or biological products, which notice reflects approval by the agency of manufacture, use or sale for human administration.

The present invention also provides articles of manufacture.

### 3.1. Terminology

As used herein, the terms “acceptor” and “acceptor antibody” refer to the antibody or nucleic acid sequence providing or encoding at least 80%, at least 85%, at least 90%, at least 95%, at least 98% or 100% of the amino acid sequences of one or more of the framework regions. In some embodiments, the term “acceptor” refers to the antibody or nucleic acid sequence providing or encoding the constant region(s). In yet another embodiment, the term “acceptor” refers to the antibody or nucleic acid sequence providing or encoding one or more of the framework regions and the constant region(s). In a specific embodiment, the term “acceptor” refers to a human antibody or nucleic acid sequence that provides or encodes at least 80%, preferably, at least 85%, at least 90%, at least 95%, at least 98%, or 100% of the amino acid sequences of one or more of the framework regions. In accordance with this embodiment, an acceptor may contain at least 1, at least 2, at least 3, least 4, at least 5, or at least 10 amino acid residues that does (do) not occur at one or more specific positions of a human antibody. An acceptor framework region and/or acceptor constant region(s) may be, e.g., derived or obtained from a germline antibody gene, a mature antibody gene, a functional antibody (e.g., antibodies well-known in the art, antibodies in development, or antibodies commercially available).

As used herein, the terms “antibody” and “antibodies” refer to monoclonal antibodies, multispecific antibodies, human antibodies, humanized antibodies, camelised antibodies, chimeric antibodies, single-chain Fvs (scFv), single chain antibodies, single domain antibodies, Fab fragments, F(ab) fragments, disulfide-linked Fvs (sdFv), anti-idiotypic (anti-Id) antibodies, and epitope-binding fragments of any of the above. In particular, antibodies include immunoglobulin molecules and immunologically active fragments of immunoglobulin molecules, i.e., molecules that contain an antigen binding site. Immunoglobulin molecules can be of any type (e.g., IgG, IgE, IgM, IgD, IgA and IgY), class (e.g., IgG₁, IgG₂, IgG₃, IgG₄, IgA₁ and IgA₂) or subclass.

A typical antibody contains two heavy chains paired with two light chains. A full-length heavy chain is about 50 kD in size (approximately 446 amino acids in length), and is encoded by a heavy chain variable region gene (about 116 amino acids) and a constant region gene. There are different constant region genes encoding heavy chain constant region of different isotypes such as alpha, gamma (IgG1, IgG2, IgG3, IgG4), delta, epsilon, and mu sequences. A full-length light chain is about 25 Kd in size (approximately 214 amino acids in length), and is encoded by a light chain variable region gene (about 110 amino acids) and a kappa or lambda constant region gene. The variable regions of the light and/or heavy chain are responsible for binding to an antigen, and the constant regions are responsible for the effector functions typical of an antibody.

As used herein, the term “analog” in the context of a proteinaceous agent (e.g., proteins, polypeptides, and peptides, such as antibodies) refers to a proteinaceous agent that possesses a similar or identical function as a second proteinaceous agent but does not necessarily comprise a similar or identical amino acid sequence of the second proteinaceous agent, or possess a similar or identical structure of the second proteinaceous agent. A proteinaceous agent that has a similar amino acid sequence refers to a second proteinaceous agent that satisfies at least one of the following: (a) a proteinaceous agent having an amino acid sequence that is at least 30%, at least 35%, at least 40%, at least 45%, at least 50%, at least 55%, at least 60%, at least 65%, at least 70%, at least 75%, at least 80%, at least 85%, at least 90%, at least 95% or at least 99% identical to the amino acid sequence of a second proteinaceous agent; (b) a proteinaceous agent encoded by a nucleotide sequence that hybridizes under stringent conditions to a nucleotide sequence encoding a second proteinaceous agent of at least 5 contiguous amino acid residues, at least 10 contiguous amino acid residues, at least 15 contiguous amino acid residues, at least 20 contiguous amino acid residues, at least 25 contiguous amino acid residues, at least 40 contiguous amino acid residues, at least 50 contiguous amino acid residues, at least 60 contiguous amino residues, at least 70 contiguous amino acid residues, at least 80 contiguous amino acid residues, at least 90 contiguous amino acid residues, at least 100 contiguous amino acid residues, at least 125 contiguous amino acid residues, or at least 150 contiguous amino acid residues; and (c) a proteinaceous agent encoded by a nucleotide sequence that is at least 30%, at least 35%, at least 40%, at least 45%, at least 50%, at least 55%, at least 60%, at least 65%, at least 70%, at least 75%, at least 80%, at least 85%, at least 90%, at least 95% or at least 99% identical to the nucleotide sequence encoding a second proteinaceous agent. A proteinaceous agent with similar structure to a second proteinaceous agent refers to a proteinaceous agent that has a similar secondary, tertiary or quaternary structure to the second proteinaceous agent. The structure of a proteinaceous agent can be determined by methods known to those skilled in the art, including but not limited to, peptide sequencing, X-ray crystallography, nuclear magnetic resonance, circular dichroism, and crystallographic electron microscopy.

To determine the percent identity of two amino acid sequences or of two nucleic acid sequences, the sequences are aligned for optimal comparison purposes (e.g., gaps can be introduced in the sequence of a first amino acid or nucleic acid sequence for optimal alignment with a second amino acid or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=number of identical overlapping positions/total number of positions×100%). In one embodiment, the two sequences are the same length.

The determination of percent identity between two sequences can also be accomplished using a mathematical algorithm. A preferred, non-limiting example of a mathematical algorithm utilized for the comparison of two sequences is the algorithm of Karlin and Altschul, 1990, Proc. Natl. Acad. Sci. U.S.A. 87:2264-2268, modified as in Karlin and Altschul, 1993, Proc. Natl. Acad. Sci. U.S.A. 90:5873-5877. Such an algorithm is incorporated into the NBLAST and XBLAST programs of Altschul et al., 1990, J. Mol. Biol. 215:403. BLAST nucleotide searches can be performed with the NBLAST nucleotide program parameters set, e.g., for score=100, wordlength=12 to obtain nucleotide sequences homologous to a nucleic acid molecules of the present invention. BLAST protein searches can be performed with the XBLAST program parameters set, e.g., to score-50, wordlength=3 to obtain amino acid sequences homologous to a protein molecule of the present invention. To obtain gapped alignments for comparison purposes, Gapped BLAST can be utilized as described in Altschul et al., 1997, Nucleic Acids Res. 25:3389-3402. Alternatively, PSI-BLAST can be used to perform an iterated search which detects distant relationships between molecules (Id.). When utilizing BLAST, Gapped BLAST, and PSI-Blast programs, the default parameters of the respective programs (e.g., of XBLAST and NBLAST) can be used (see, e.g., the NCBI website). Another preferred, non-limiting example of a mathematical algorithm utilized for the comparison of sequences is the algorithm of Myers and Miller, 1988, CABIOS 4:11-17. Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used.

The percent identity between two sequences can be determined using techniques similar to those described above, with or without allowing gaps. In calculating percent identity, typically only exact matches are counted.

As used herein, the term “CDR” refers to the complement determining region within antibody variable sequences. There are three CDRs in each of the variable regions of the heavy chain and the light chain, which are designated CDR1, CDR2 and CDR3, for each of the variable regions. The exact boundaries of these CDRs have been defined differently according to different systems. The system described by Kabat (Kabat et al., Sequences of Proteins of Immunological Interest (National Institutes of Health, Bethesda, Md. (1987) and (1991)) not only provides an unambiguous residue numbering system applicable to any variable region of an antibody, but also provides precise residue boundaries defining the three CDRs. These CDRs may be referred to as Kabat CDRs. Chothia and coworkers (Chothia & Lesk, J. Mol. Biol. 196:901-917 (1987) and Chothia et al., Nature 342:877-883 (1989)) found that certain sub-portions within Kabat CDRs adopt nearly identical peptide backbone conformations, despite having great diversity at the level of amino acid sequence. These sub-portions were designated as L1, L2 and L3 or H1, H2 and H3 where the “L” and the “H” designates the light chain and the heavy chains regions, respectively. These regions may be referred to as Chothia CDRs, which have boundaries that overlap with Kabat CDRs. Other boundaries defining CDRs overlapping with the Kabat CDRs have been described by Padlan (FASEB J. 9:133-139 (1995)) and MacCallum (J Mol Biol 262(5):732-45 (1996)). Still other CDR boundary definitions may not strictly follow one of the above systems, but will nonetheless overlap with the Kabat CDRs, although they may be shortened or lengthened in light of prediction or experimental findings that particular residues or groups of residues or even entire CDRs do not significantly impact antigen binding. The methods used herein may utilize CDRs defined according to any of these systems, although preferred embodiments use Kabat or Chothia defined CDRs.

As used herein, the term “canonical” residue refers to a residue in a CDR or framework that defines a particular canonical CDR structure as defined by Chothia et al. (J. Mol. Biol. 196:901-907 (1987); Chothia et al., J. Mol. Biol. 227:799 (1992), both are incorporated herein by reference). According to Chothia et al., critical portions of the CDRs of many antibodies have nearly identical peptide backbone confirmations despite great diversity at the level of amino acid sequence. Each canonical structure specifies primarily a set of peptide backbone torsion angles for a contiguous segment of amino acid residues forming a loop.

As used herein, the term “derivative” in the context of proteinaceous agent (e.g., proteins, polypeptides, and peptides, such as antibodies) refers to a proteinaceous agent that comprises an amino acid sequence which has been altered by the introduction of amino acid residue substitutions, deletions, and/or additions. The term “derivative” as used herein also refers to a proteinaceous agent which has been modified, i.e., by the covalent attachment of any type of molecule to the proteinaceous agent. For example, but not by way of limitation, an antibody may be modified, e.g., by glycosylation, acetylation, pegylation, phosphorylation, amidation, derivatization by known protecting/blocking groups, proteolytic cleavage, linkage to a cellular ligand or other protein, etc. A derivative of a proteinaceous agent may be produced by chemical modifications using techniques known to those of skill in the art, including, but not limited to specific chemical cleavage, acetylation, formylation, metabolic synthesis of tunicamycin, etc. Further, a derivative of a proteinaceous agent may contain one or more non-classical amino acids. A derivative of a proteinaceous agent possesses a similar or identical function as the proteinaceous agent from which it was derived.

As used herein, the terms “disorder” and “disease” are used interchangeably for a condition in a subject.

As used herein, the terms “donor” and “donor antibody” refer to an antibody providing one or more CDRs. In a preferred embodiment, the donor antibody is an antibody from a species different from the antibody from which the framework regions are obtained or derived. In the context of a humanized antibody, the term “donor antibody” refers to a non-human antibody providing one or more CDRs.

As used herein, the term “effective amount” refers to the amount of a therapy which is sufficient to reduce or ameliorate the severity and/or duration of a disorder or one or more symptoms thereof, prevent the advancement of a disorder, cause regression of a disorder, prevent the recurrence, development, onset or progression of one or more symptoms associated with a disorder, detect a disorder, or enhance or improve the prophylactic or therapeutic effect(s) of another therapy (e.g., prophylactic or therapeutic agent).

As used herein, the term “epitopes” refers to fragments of a polypeptide or protein having antigenic or immunogenic activity in an animal, preferably in a mammal, and most preferably in a human. An epitope having immunogenic activity is a fragment of a polypeptide or protein that elicits an antibody response in an animal. An epitope having antigenic activity is a fragment of a polypeptide or protein to which an antibody immunospecifically binds as determined by any method well-known to one of skill in the art, for example by immunoassays. Antigenic epitopes need not necessarily be immunogenic.

As used herein, the term “fusion protein” refers to a polypeptide or protein (including, but not limited to an antibody) that comprises an amino acid sequence of a first protein or polypeptide or functional fragment, analog or derivative thereof, and an amino acid sequence of a heterologous protein, polypeptide, or peptide (i.e., a second protein or polypeptide or fragment, analog or derivative thereof different than the first protein or fragment, analog or derivative thereof). In one embodiment, a fusion protein comprises a prophylactic or therapeutic agent fused to a heterologous protein, polypeptide or peptide. In accordance with this embodiment, the heterologous protein, polypeptide or peptide may or may not be a different type of prophylactic or therapeutic agent. For example, two different proteins, polypeptides or peptides with immunomodulatory activity may be fused together to form a fusion protein. In a preferred embodiment, fusion proteins retain or have improved activity relative to the activity of the original protein, polypeptide or peptide prior to being fused to a heterologous protein, polypeptide, or peptide.

As used herein, the term “fragment” refers to a peptide or polypeptide (including, but not limited to an antibody) comprising an amino acid sequence of at least 5 contiguous amino acid residues, at least 10 contiguous amino acid residues, at least 15 contiguous amino acid residues, at least 20 contiguous amino acid residues, at least 25 contiguous amino acid residues, at least 40 contiguous amino acid residues, at least 50 contiguous amino acid residues, at least 60 contiguous amino residues, at least 70 contiguous amino acid residues, at least contiguous 80 amino acid residues, at least contiguous 90 amino acid residues, at least contiguous 100 amino acid residues, at least contiguous 125 amino acid residues, at least 150 contiguous amino acid residues, at least contiguous 175 amino acid residues, at least contiguous 200 amino acid residues, or at least contiguous 250 amino acid residues of the amino acid sequence of another polypeptide or protein. In a specific embodiment, a fragment of a protein or polypeptide retains at least one function of the protein or polypeptide.

As used herein, the term “functional fragment” refers to a peptide or polypeptide (including, but not limited to an antibody) comprising an amino acid sequence of at least 5 contiguous amino acid residues, at least 10 contiguous amino acid residues, at least 15 contiguous amino acid residues, at least 20 contiguous amino acid residues, at least 25 contiguous amino acid residues, at least 40 contiguous amino acid residues, at least 50 contiguous amino acid residues, at least 60 contiguous amino residues, at least 70 contiguous amino acid residues, at least contiguous 80 amino acid residues, at least contiguous 90 amino acid residues, at least contiguous 100 amino acid residues, at least contiguous 125 amino acid residues, at least 150 contiguous amino acid residues, at least contiguous 175 amino acid residues, at least contiguous 200 amino acid residues, or at least contiguous 250 amino acid residues of the amino acid sequence of second, different polypeptide or protein, wherein said polypeptide or protein retains at least one function of the second, different polypeptide or protein. In a specific embodiment, a fragment of a polypeptide or protein retains at least two, three, four, or five functions of the protein or polypeptide. Preferably, a fragment of an antibody that immunospecifically binds to a particular antigen retains the ability to immunospecifically bind to the antigen.

As used herein, the term “framework” or “framework sequence” refers to the remaining sequences of a variable region minus the CDRs. Because the exact definition of a CDR sequence can be determined by different systems, the meaning of a framework sequence is subject to correspondingly different interpretations. The six CDRs (CDR1, 2, and 3 of light chain and CDR1, 2, and 3 of heavy chain) also divide the framework regions on the light chain and the heavy chain into four sub-regions (FR1, FR2, FR3 and FR4) on each chain, in which CDR1 is positioned between FR1 and FR2, CDR2 between FR2 and FR3, and CDR3 between FR3 and FR4. Without specifying the particular sub-regions as FR1, FR2, FR3 or FR4, a framework region, as referred by others, represents the combined FR's within the variable region of a single, naturally occurring immunoglobulin chain. As used herein, a FR represents one of the four sub-regions, and FRs represents two or more of the four sub-regions constituting a framework region.

As used herein, the term “germline antibody gene” or “gene fragment” refers to an immunoglobulin sequence encoded by non-lymphoid cells that have not undergone the maturation process that leads to genetic rearrangement and mutation for expression of a particular immunoglobulin. (See, e.g., Shapiro et al., Crit. Rev. Immunol. 22(3): 183-200 (2002); Marchalonis et al., Adv Exp Med Biol. 484:13-30 (2001)). One of the advantages provided by various embodiments of the present invention stems from the recognition that germline antibody genes are more likely than mature antibody genes to conserve essential amino acid sequence structures characteristic of individuals in the species, hence less likely to be recognized as from a foreign source when used therapeutically in that species.

As used herein, the term “key” residues refer to certain residues within the variable region that have more impact on the binding specificity and/or affinity of an antibody, in particular a humanized antibody. A key residue includes, but is not limited to, one or more of the following: a residue that is adjacent to a CDR, a potential glycosylation site (can be either N- or O-glycosylation site), a rare residue, a residue capable of interacting with the antigen, a residue capable of interacting with a CDR, a canonical residue, a contact residue between heavy chain variable region and light chain variable region, a residue within the Vernier zone, and a residue in the region that overlaps between the Chothia definition of a variable heavy chain CDR1 and the Kabat definition of the first heavy chain framework. In a specific embodiment, key residues are not heavy chain variable framework region amino acid residues 6, 23, 24 and 49 as a group according to the Kabat numbering system. In a specific embodiment, a key residue is not heavy chain variable framework region amino acid residue 2, 4, 24, 35, 36, 39, 43, 45, 64, 69, 70, 73, 74, 75, 76, 78, 92 and 93 according to the Kabat numbering system. In a specific embodiment, a key residue is not light chain variable framework region amino acid residue 4, 38, 43, 44, 46, 58, 62, 65, 66, 67, 68, 69, 73, 85 or 98 according to the Kabat numbering system.

As used herein, the term “hyperproliferative cell disorder” refers to a disorder in which cellular hyperproliferation causes or contributes to the pathological state or symptoms of the disorder. In some embodiments, the hyperproliferative cell disorder is cancer. In some embodiments, the hyperproliferative cell disorder is a non-neoplastic disorder in which cellular hyperproliferation causes or contributes to the pathological state or symptoms of the disorder. In some embodiments, the hyperproliferative cell disorder is characterized by hyperproliferating epithelial cells. Hyperproliferative epithelial cell disorders include, but are not limited to, asthma, COPD, lung fibrosis, bronchial hyper responsiveness, psoriasis, seborrheic dermatitis, and cystic fibrosis. In other embodiments, the hyperproliferative cell disorder is characterized by hyperproliferating endothelial cells. Hyperproliferative endothelial cell disorders include, but are not limited to restenosis, hyperproliferative vascular disease, Behcet's Syndrome, atherosclerosis, and macular degeneration.

As used herein, the term “humanized antibody” is an antibody or a variant, derivative, analog or fragment thereof which immunospecifically binds to an antigen of interest and which comprises a framework (FR) region having substantially the amino acid sequence of a human antibody and a complementary determining region (CDR) having substantially the amino acid sequence of a non-human antibody. As used herein, the term “substantially” in the context of a CDR refers to a CDR having an amino acid sequence at least 80%, preferably at least 85%, at least 90%, at least 95%, at least 98% or at least 99% identical to the amino acid sequence of a non-human antibody CDR. A humanized antibody comprises substantially all of at least one, and typically two, variable domains (Fab, Fab′, F(ab′)₂, FabC, Fv) in which all or substantially all of the CDR regions correspond to those of a non-human immunoglobulin (i.e., donor antibody) and all or substantially all of the framework regions are those of a human immunoglobulin consensus sequence. Preferably, a humanized antibody also comprises at least a portion of an immunoglobulin constant region (Fc), typically that of a human immunoglobulin. In some embodiments, a humanized antibody contains both the light chain as well as at least the variable domain of a heavy chain. The antibody also may include the CH1, hinge, CH2, CH3, and CH4 regions of the heavy chain. In some embodiments, a humanized antibody only contains a humanized light chain. In some embodiments, a humanized antibody only contains a humanized heavy chain. In specific embodiments, a humanized antibody only contains a humanized variable domain of a light chain and/or humanized heavy chain.

The humanized antibody can be selected from any class of immunoglobulins, including IgM, IgG, IgD, IgA and IgE, and any isotype, including without limitation IgG₁, IgG₂, IgG₃ and IgG₄. The humanized antibody may comprise sequences from more than one class or isotype, and particular constant domains may be selected to optimize desired effector functions using techniques well-known in the art.

The framework and CDR regions of a humanized antibody need not correspond precisely to the parental sequences, e.g., the donor antibody CDR or the consensus framework may be mutagenized by substitution, insertion and/or deletion of at least one amino acid residue so that the CDR or framework residue at that site does not correspond to either the donor antibody or the consensus framework. In a preferred embodiment, such mutations, however, will not be extensive. Usually, at least 80%, preferably at least 85%, more preferably at least 90%, and most preferably at least 95% of the humanized antibody residues will correspond to those of the parental FR and CDR sequences. As used herein, the term “consensus framework” refers to the framework region in the consensus immunoglobulin sequence. As used herein, the term “consensus immunoglobulin sequence” refers to the sequence formed from the most frequently occurring amino acids (or nucleotides) in a family of related immunoglobulin sequences (See e.g., Winnaker, From Genes to Clones (Verlagsgesellschaft, Weinheim, Germany 1987). In a family of immunoglobulins, each position in the consensus sequence is occupied by the amino acid occurring most frequently at that position in the family. If two amino acids occur equally frequently, either can be included in the consensus sequence.

As used herein, the term “host cell” includes a particular subject cell transfected or transformed with a nucleic acid molecule and the progeny or potential progeny of such a cell. Progeny of such a cell may not be identical to the parent cell transfected with the nucleic acid molecule due to mutations or environmental influences that may occur in succeeding generations or integration of the nucleic acid molecule into the host cell genome.

As used herein, the term “immunospecifically binds to an antigen” and analogous terms refer to peptides, polypeptides, proteins (including, but not limited to fusion proteins and antibodies) or fragments thereof that specifically bind to an antigen or a fragment and do not specifically bind to other antigens. A peptide, polypeptide, or protein that immunospecifically binds to an antigen may bind to other antigens with lower affinity as determined by, e.g., immunoassays, BIAcore, or other assays known in the art. Antibodies or fragments that immunospecifically bind to an antigen may be cross-reactive with related antigens. Preferably, antibodies or fragments that immunospecifically bind to an antigen do not cross-react with other antigens.

As used herein, the term “isolated” in the context of a proteinaceous agent (e.g., a peptide, polypeptide, or protein (such as a fusion protein or an antibody)) refers to a proteinaceous agent which is substantially free of cellular material or contaminating proteins, polypeptides, peptides and antibodies from the cell or tissue source from which it is derived, or substantially free of chemical precursors or other chemicals when chemically synthesized. The language “substantially free of cellular material” includes preparations of a proteinaceous agent in which the proteinaceous agent is separated from cellular components of the cells from which it is isolated or recombinantly produced. Thus, a proteinaceous agent that is substantially free of cellular material includes preparations of a proteinaceous agent having less than about 30%, 20%, 10%, or 5% (by dry weight) of heterologous protein, polypeptide or peptide (also referred to as a “contaminating protein”). When the proteinaceous agent is recombinantly produced, it is also preferably substantially free of culture medium, i.e., culture medium represents less than about 20%, 10%, or 5% of the volume of the proteinaceous agent preparation. When the proteinaceous agent is produced by chemical synthesis, it is preferably substantially free of chemical precursors or other chemicals, i.e., it is separated from chemical precursors or other chemicals which are involved in the synthesis of the proteinaceous agent. Accordingly, such preparations of a proteinaceous agent have less than about 30%, 20%, 10%, 5% (by dry weight) of chemical precursors or compounds other than the proteinaceous agent of interest. In a specific embodiment, proteinaceous agents disclosed herein are isolated. In a preferred embodiment, an antibody of the invention is isolated.

As used herein, the term “isolated” in the context of nucleic acid molecules refers to a nucleic acid molecule which is separated from other nucleic acid molecules which are present in the natural source of the nucleic acid molecule. Moreover, an “isolated” nucleic acid molecule, such as a cDNA molecule, is preferably substantially free of other cellular material, or culture medium when produced by recombinant techniques, or substantially free of chemical precursors or other chemicals when chemically synthesized. In a specific embodiment, nucleic acid molecules are isolated. In a preferred embodiment, a nucleic acid molecule encoding an antibody of the invention is isolated. As used herein, the term “substantially free” refers to the preparation of the “isolated” nucleic acid having less than about 30%, 20%, 10%, or 5% (by dry weight) of heterologous nucleic acids, and preferably other cellular material, culture medium, chemical precursors, or other chemicals.

As used herein, the term “in combination” refers to the use of more than one therapies (e.g., more than one prophylactic agent and/or therapeutic agent). The use of the term “in combination” does not restrict the order in which therapies (e.g., prophylactic and/or therapeutic agents) are administered to a subject. A first therapy (e.g., a first prophylactic or therapeutic agent) can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapy (e.g., a second prophylactic or therapeutic agent) to a subject.

As used herein, the terms “manage,” “managing,” and “management” refer to the beneficial effects that a subject derives from a therapy (e.g., a prophylactic or therapeutic agent), which does not result in a cure of the disease. In certain embodiments, a subject is administered one or more therapies (e.g., one or more prophylactic or therapeutic agents) to “manage” a disease so as to prevent the progression or worsening of the disease.

As used herein, the term “mature antibody gene” refers to a genetic sequence encoding an immunoglobulin that is expressed, for example, in a lymphocyte such as a B cell, in a hybridoma or in any antibody producing cell that has undergone a maturation process so that the particular immunoglobulin is expressed. The term includes mature genomic DNA, cDNA and other nucleic acid sequences that encode such mature genes, which have been isolated and/or recombinantly engineered for expression in other cell types. Mature antibody genes have undergone various mutations and rearrangements that structurally distinguish them from antibody genes encoded in all cells other than lymphocytes. Mature antibody genes in humans, rodents, and many other mammals are formed by fusion of V and J gene segments in the case of antibody light chains and fusion of V, D, and J gene segments in the case of antibody heavy chains. Many mature antibody genes acquire point mutations subsequent to fusion, some of which increase the affinity of the antibody protein for a specific antigen.

As used herein, the term “pharmaceutically acceptable” refers approved by a regulatory agency of the federal or a state government, or listed in the U.S. Pharmacopeia, European Pharmacopeia, or other generally recognized pharmacopeia for use in animals, and more particularly, in humans.

As used herein, the terms “prevent,” “preventing,” and “prevention” refer to the inhibition of the development or onset of a disorder or the prevention of the recurrence, onset, or development of one or more symptoms of a disorder in a subject resulting from the administration of a therapy (e.g., a prophylactic or therapeutic agent), or the administration of a combination of therapies (e.g., a combination of prophylactic or therapeutic agents).

As used herein, the terms “prophylactic agent” and “prophylactic agents” refer to any agent(s) which can be used in the prevention of a disorder or one or more of the symptoms thereof. In certain embodiments, the term “prophylactic agent” refers to an antibody of the invention. In certain other embodiments, the term “prophylactic agent” refers to an agent other than an antibody of the invention. Preferably, a prophylactic agent is an agent which is known to be useful to or has been or is currently being used to the prevent or impede the onset, development, progression and/or severity of a disorder or one or more symptoms thereof.

As used herein, the term “prophylactically effective amount” refers to the amount of a therapy (e.g., prophylactic agent) which is sufficient to result in the prevention of the development, recurrence, or onset of a disorder or one or more symptoms thereof, or to enhance or improve the prophylactic effect(s) of another therapy (e.g., a prophylactic agent).

As used herein, the phrase “protocol” refers to a regimen for dosing and timing the administration of one or more therapies (e.g., therapeutic agents) that has a therapeutic effective.

As used herein, the phrase “side effects” encompasses unwanted and adverse effects of a prophylactic or therapeutic agent. Side effects are always unwanted, but unwanted effects are not necessarily adverse. An adverse effect from a therapy (e.g., a prophylactic or therapeutic agent) might be harmful, uncomfortable, or risky.

As used herein, the term “small molecules” and analogous terms include, but are not limited to, peptides, peptidomimetics, amino acids, amino acid analogs, polynucleotides, polynucleotide analogs, nucleotides, nucleotide analogs, organic or inorganic compounds (i.e., including heteroorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such agents.

As used herein, the terms “subject” and “patient” are used interchangeably. As used herein, the terms “subject” and “subjects” refer to an animal, preferably a mammal including a non-primate (e.g., a cow, pig, horse, cat, dog, rat, and mouse) and a primate (e.g., a monkey, such as a cynomolgous monkey, a chimpanzee, and a human), and most preferably a human. In one embodiment, the subject is a non-human animal such as a bird (e.g., a quail, chicken, or turkey), a farm animal (e.g., a cow, horse, pig, or sheep), a pet (e.g., a cat, dog, or guinea pig), or laboratory animal (e.g., an animal model for a disorder). In a preferred embodiment, the subject is a human (e.g., an infant, child, adult, or senior citizen).

As used herein, the term “synergistic” refers to a combination of therapies (e.g., prophylactic or therapeutic agents) which is more effective than the additive effects of any two or more single therapies (e.g., one or more prophylactic or therapeutic agents). A synergistic effect of a combination of therapies (e.g., a combination of prophylactic or therapeutic agents) permits the use of lower dosages of one or more of therapies (e.g., one or more prophylactic or therapeutic agents) and/or less frequent administration of said therapies to a subject with a disorder. The ability to utilize lower dosages of therapies (e.g., prophylactic or therapeutic agents) and/or to administer said therapies less frequently reduces the toxicity associated with the administration of said therapies to a subject without reducing the efficacy of said therapies in the prevention or treatment of a disorder. In addition, a synergistic effect can result in improved efficacy of therapies (e.g., prophylactic or therapeutic agents) in the prevention or treatment of a disorder. Finally, synergistic effect of a combination of therapies (e.g., prophylactic or therapeutic agents) may avoid or reduce adverse or unwanted side effects associated with the use of any single therapy.

As used herein, the terms “therapeutic agent” and “therapeutic agents” refer to any agent(s) which can be used in the prevention, treatment, management, or amelioration of a disorder or one or more symptoms thereof. In certain embodiments, the term “therapeutic agent” refers to an antibody of the invention. In certain other embodiments, the term “therapeutic agent” refers an agent other than an antibody of the invention. Preferably, a therapeutic agent is an agent which is known to be useful for, or has been or is currently being used for the prevention, treatment, management, or amelioration of a disorder or one or more symptoms thereof.

As used herein, the term “therapeutically effective amount” refers to the amount of a therapy (e.g., an antibody of the invention), which is sufficient to reduce the severity of a disorder, reduce the duration of a disorder, ameliorate one or more symptoms of a disorder, prevent the advancement of a disorder, cause regression of a disorder, or enhance or improve the therapeutic effect(s) of another therapy.

As used herein, the terms “therapies” and “therapy” can refer to any protocol(s), method(s), and/or agent(s) that can be used in the prevention, treatment, management, and/or amelioration of a disorder or one or more symptoms thereof. In certain embodiments, the terms “therapy” and “therapy” refer to anti-viral therapy, anti-bacterial therapy, anti-fungal therapy, anti-cancer agent, biological therapy, supportive therapy, and/or other therapies useful in treatment, management, prevention, or amelioration of a disorder or one or more symptoms thereof known to one skilled in the art, for example, a medical professional such as a physician.

As used herein, the terms “treat,” “treatment,” and “treating” refer to the reduction or amelioration of the progression, severity, and/or duration of a disorder or amelioration of one or more symptoms thereof resulting from the administration of one or more therapies (including, but not limited to, the administration of one or more prophylactic or therapeutic agents).

As used herein, “Vernier” zone refers to a subset of framework residues that may adjust CDR structure and fine-tune the fit to antigen as described by Foote and Winter (1992, J. Mol. Biol. 224:487-499, which is incorporated herein by reference). Vernier zone residues form a layer underlying the CDRs and may impact on the structure of CDRs and the affinity of the antibody. Non-limiting examples of residues that are within the Vernier zone are listed in Table 1 (see Foote and Winter, 1992, J. Mol. Biol. 224:487-499):

TABLE 1
Residues in the Vernier zone (Kabat numbering):

| Heavy Chain | Light Chain |
|---|---|
| 2 | 2 |
| 27-30 | 4 |
| 47-49 | 35-36 |
| 67 | 46-49 |
| 69 | 64 |
| 71 | 66 |
| 73 | 68-69 |
| 78 | 71 |
| 93-94 | 98 |
| 103 | |

### 4. BRIEF DESCRIPTION OF THE FIGURES

FIG. 1. Nucleic acid and protein sequences of the heavy and light chains of the anti-IL9 monoclonal antibody L1.

FIG. 2. Sequence alignment of the heavy and light chains of the anti-IL9 monoclonal antibody L1 with the corresponding selected acceptor germlines sequences (V_{H}3-23/JH4 and L23/Jκ4, respectively).

FIG. 3. Protein sequences of the combinatorial humanization libraries for the heavy and light chains of the anti-IL9 monoclonal antibody L1. Four positions in the light chain and 4-6 positions in the heavy chain were targeted for introduction of diversity.

FIG. 4. Phage vector used for screening of the combinatorial libraries and expression of Fab fragments.

FIG. 5. Capture-lift screening of library 2. Six clones positive for binding to human IL-9 are circled.

FIG. 6. Representative sequences of humanized clones of the anti-IL9 monoclonal antibody L1 after secondary screening of combinatorial libraries 1 and 2.

FIG. 7. (A) and (B): ELISA titration using supernatant-expressed Fabs on immobilized antigen (IL9). Clones were numbered according to FIG. 6. Negative control was the supernatant-expressed Fab of an anti-RSV monoclonal antibody.

FIG. 8. Nucleic acid and protein sequences of the heavy and light chains of the anti-human EphA2 monoclonal antibody EP101.

FIG. 9. Sequence alignment of the heavy and light chains of the anti-human EphA2 monoclonal antibody EP101 with the corresponding selected acceptor germlines sequences (VH1-58/JH5 and O18/Jκ4, respectively).

FIG. 10. Protein sequences of the combinatorial humanization libraries for the heavy and light chains of the anti-human EphA2 monoclonal antibody EP101. Four positions in the light chain and four positions in the heavy chain were targeted for introduction of diversity.

FIG. 11. Representative sequences of humanized clones of the anti-human EphA2 monoclonal antibody EP101 after secondary screening of combinatorial libraries 1 and 2.

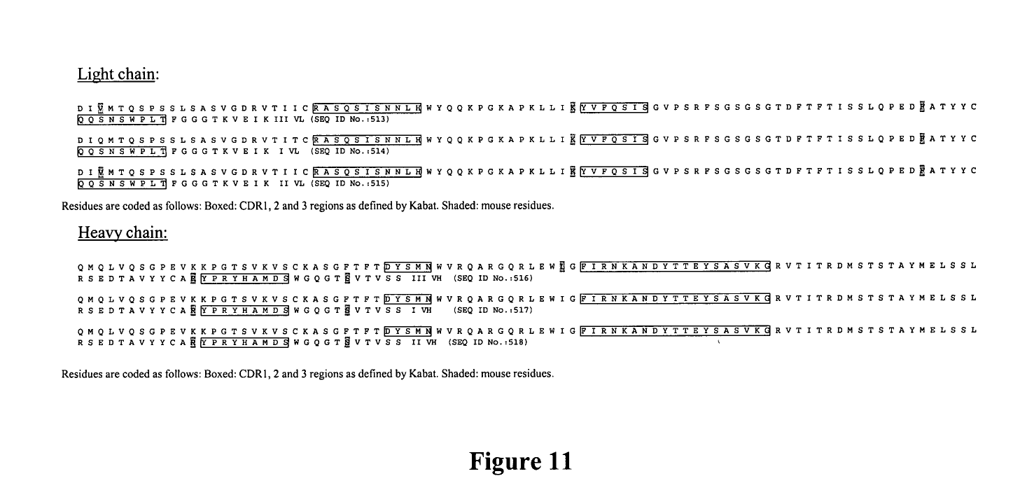

FIG. 12. ELISA titration using periplasm-expressed Fabs on immobilized antigen (human EphA2).

### 5. DETAILED DESCRIPTION OF THE INVENTION

The present invention provides methods of re-engineering or re-shaping an antibody from a first species, wherein the re-engineered or re-shaped antibody does not elicit undesired immune response in a second species, and the re-engineered or re-shaped antibody retains substantially the same antigen binding-ability of the antibody from the first species. In accordance with the present invention, a combinatorial library comprising the CDRs of the antibody from the first species fused in frame with framework regions derived from a second species can be constructed and screened for the desired modified antibody.

The present invention provides nucleic acid sequences encoding a humanized antibody that immunospecifically binds to an antigen. The present invention also provides cells comprising, containing or engineered to express the nucleic acid sequences described herein. The present invention provides a method of producing a heavy chain variable region (preferably, a humanized heavy chain variable region), said method comprising expressing the nucleotide sequence encoding a heavy chain variable region (preferably, a humanized heavy chain variable region) in a cell described herein. The present invention provides a method of producing an light chain variable region (preferably, a humanized light chain variable region), said method comprising expressing the nucleotide sequence encoding a light chain variable region (preferably, a humanized light chain variable region) in a cell described herein. The present invention also provides a method of producing an antibody (preferably, a humanized antibody) that immunospecifically binds to an antigen, said method comprising expressing the nucleic acid sequence(s) encoding the humanized antibody contained in the cell described herein. The present invention further provides optional screening methods for identify and/or selecting a humanized antibody of interest.

The present invention provides antibodies produced by the methods described herein. In a preferred embodiment, the invention provides humanized antibodies produced by the methods described herein. The present invention also provides a composition comprising an antibody produced by the methods described herein and a carrier, diluent or excipient. In a preferred embodiment, the invention provides a composition comprising a humanized antibody produced by the methods described herein and a carrier, diluent or excipient. Preferably, the compositions of the invention are pharmaceutical compositions in a form for its intended use.

For clarity of disclosure, and not by way of limitation, the detailed description of the invention is divided into the following subsections:
(i) selection of acceptor antibody template
(ii) construction of combinatorial libraries
(iii) expression of the combinatorial libraries
(iv) selection of humanized antibodies
(v) production and characterization of humanized antibodies
(vi) antibody conjugates
(vii) uses of the compositions of the invention
(viii) administration and formulations
(ix) dosage and frequency of administration
(x) biological assays
(xi) kits
(xii) article of manufacture

### 5.1. Selection of Acceptor Antibody Template

One acceptor heavy chain framework (preferably a human heavy chain framework) and one acceptor light chain framework (preferably a human light chain framework) are selected according to the following “rules of design”:

(1) Select acceptor framework regions of the heavy and/or light chain using (a) or (b):

(a) For the 1st, 2nd, 3rd and 4th framework regions of the heavy and/or light chains, select corresponding acceptor sequences, such as human germline sequences, human functional antibody sequences, human antibody sequences obtained from databanks or literature, or sequences of human antibodies available to public, with framework homology to the donor antibody sequence of less than 65%, preferably, less than 60%, less than 55%, less than 50%, less than 45%, or less than 40% at the amino acid level. In this case, acceptor FR1, FR2, FR3 or FR4 individually have less than 65%, 60%, 55%, 55% or 45% homology to the corresponding framework region of the donor antibody at the amino acid level. Preferably, both the Chothia and Kabat definitions of the CDRs are applied in determining the framework regions. If no such sequences exist, select sequences with the lowest homology possible. In particular and as an optional consideration, the choice of an acceptor 4th framework for both heavy and light chains can be made according to more refined criteria, e.g., human germline 4th frameworks or functional antibody 4th frameworks exhibiting high homology to the donor antibody sequence in their proximal end of CDR3 and low homology in their distal end of CDR3 can be preferentially selected. As used herein, the “proximal end of CDR3” refers to the N-terminus of the 4^{th} framework, and the “distal end of CDR 3” refers to the C-terminus of the 4^{th} framework.

(b) Alternatively, for the framework region of the heavy chain and/or the framework region of the light chain, select corresponding acceptor sequences, such as human germline sequences, human functional antibody sequences, human antibody sequences obtained from databanks or literature, or sequences of human antibodies available to public, with global framework homology to the donor antibody sequence of less than 65%, preferably, less than 60%, less than 55%, less than 50%, less than 45%, or less than 40% at the amino acid level. In this case, acceptor FR1, FR2, FR3 and FR4 together have less than 65%, 60%, 55%, 50%, 45%, or 40% homology at the amino acid level to donor antibody FR1, FR2, FR3 and FR4 together. Accordingly, one or more of the four acceptor framework regions may individually have a homology to one or more of the donor antibody framework regions that is more than 65%, 60%, 55%, 55% or 45% at the amino acid level. For example, in one embodiment, the global framework homology of the acceptor antibody to the donor antibody sequence is less than 65% at the amino acid level, however, framework region 1 of the acceptor antibody has a homology to the donor antibody sequence that is more than 65% at the amino acid level. Preferably, both the Chothia and Kabat definitions of the CDRs are applied in determining the framework regions. If no such sequences exist, select sequences with the lowest homology possible.

(2) Identify and select those heavy chain frameworks with amino acid residues at one, two, three or all of the following amino acid residues: 6, 23, 24 and 49 (Kabat numbering) that are not identical to the corresponding residues in the donor antibody. Eliminate any acceptor sequence that does not have at least one of these four residues differing from the donor sequence.

(3) Identify the following amino acid residues: 4L, 38L, 43L, 44L, 46L, 58L, 62L, 65L, 66L, 67L, 68L, 69L, 73L, 85L and 98L in the light chain and 2H, 4H, 24H, 36H, 39H, 43H, 45H, 69H, 70H, 73H, 74H, 75H, 76H, 78H, 92H and 93H in the heavy chain. Residues at those positions are fixed as acceptor so that no mutations are introduced in the combinatorial libraries. When applicable, acceptor sequences which vary at more than one of these positions when compared to the donor antibody sequence are eliminated. Acceptor framework sequences that are conserved relative to donor antibody sequences at these positions are preferred. More refined criteria can also be used, leading to the selection of human germline genes or functional antibody sequences that are highly conserved at the above-mentioned positions which are further defined as canonical, vernier or interface packing (see rule (6), infra).

(4) For both the light and heavy chain sequences, determine the canonical class of the CDR loops. When applicable, eliminate acceptor sequences that do not have the same canonical class (as described, e.g., in Chothia & Lesk, 1987, J. Mol. Biol. 196, 901-917, or at the websites: www.rubic.rdg.ac.uk/˜andrew/bioinf.org/abs/chothia.html, and www.rubic.rdg.ac.uk/˜andrew/bioinf.org/abs/chothia.dat.auto) as the donor antibody sequences. Optionally, select acceptor sequences harboring H1, H2, L1, L2 and L3 loops of the same canonical class as the donor antibody. Optionally, further selection among the remaining acceptor sequences can be done by eliminating the acceptor sequences that exhibit the lowest homology to the donor antibody sequences in both CDR1 and CDR2 of the light and heavy chains.

(5) Using known three-dimensional structures of various Fab fragments (available at www.rcsb.org/pdb/) as models, identify specific positions in the selected acceptor heavy and light chains which are (a) not interacting with a CDR residue, (b) not adjacent to a CDR, (c) not a substitute for a rare acceptor framework residue, and/or (d) further than 6 Å, preferably, further than 6.5 Å, 7 Å, 7.5 Å, or 8 Å from a CDR. The donor antibody and the acceptor antibody are derived from different species, e.g., the donor antibody is a non-human antibody, and the acceptor antibody is a human antibody. Preferably, positions corresponding to buried residues are examined. Among the positions fulfilling those requirements, at least one position (at least two, at least three, at least four positions) for the light chain and for the heavy chain whose corresponding residues are different between donor and acceptor will be identified. No substitutions will be introduced at those positions (i.e. no diversity will be introduced in the combinatorial libraries).

(6) Individually align the remaining acceptor antibody sequences with the donor antibody sequence. One or more mutations are preferably introduced at some or all of the following positions designated as key residues, provided they have not been fixed in the preceding steps: (a) rare framework residues that differ between the donor antibody framework and the acceptor antibody framework (as defined, e.g., by Kabat et al., 1991, U.S. Public Health Service, National institutes of Health, Washington, D.C. and the website of people.cryst.bbk.ac.uk/˜ubcg07s/); (b) Vernier zone residues when differing between donor antibody framework and acceptor antibody framework (including, but not limited to the following, according to Kabat numbering: 2H, 27-30H, 47-49H, 67H, 69H, 71H, 73H, 78H, 93H, 94H, 103H, 2L, 4L, 35L, 36L, 46-49L, 64L, 66L, 68L, 69L, 71L and 98L); (c) interchain packing residues at the VL/VH domain interface that differ between the donor antibody and the acceptor antibody framework (including, but not limited to the following, according to Kabat numbering: L36, L38, L44, L46, L87, L98, H37, H39, H45, H47, H91, H93 and H103); (d) Canonical residues which differ between the donor antibody framework and the acceptor antibody framework sequences, particularly the framework positions crucial for the definition of the canonical class of the donor CDR loops (as described for instance in Chothia & Lesk, 1987, J. Mol. Biol. 196, 901-917, websites of www.rubic.rdg.ac.uk/˜andrew/bioinf.org/abs/chothia.html, and www.rubic.rdg.ac.uk/˜andrew/bioinf.org/abs/chothia.dat.auto); (e) residues that encompass both the Chothia-defined CDR1 region and the Kabat-defined 1st framework region of the heavy chain that differ between the donor antibody framework and the acceptor antibody framework (positions 26-30 according to Kabat numbering); (f) residues that are adjacent to a CDR; (g) residues that are potential glycosylation sites; (h) residues that are capable interacting with the antigen; (i) residues that are capable interacting with a CDR; and (j) contact residues between the variable heavy domain and variable light domain. In some embodiments, the mutation(s) introduced into the acceptor antibody framework at a key residue results in the amino acid residue at such position being identified to the corresponding amino acid residue in the donor antibody framework.

In rule (6)(a)-(j), the similarity in the chemical structure between donor antibody framework residues and acceptor antibody framework residues is considered so that the presence of similar residues at a given position might lead to the conservation of the corresponding acceptor residue. The features to take into consideration in determining whether a particular amino acid residue should be conserved include, but are not be limited to, hydrophobicity and charge profiles.

Acceptor frameworks can be obtained or derived from any source known to one of skill in the art. In one embodiment, acceptor antibody frameworks for use in accordance with the present invention are obtained or derived from human germline sequences (Vκ, V_{γ}, and V_{H}). In specific embodiments, 46 human germline kappa chain framework sequences are considered for the 1st, 2nd and 3rd frameworks (A1, A10, A11, A14, A17, A18, A19, A2, A20, A23, A26, A27, A3, A30, A5, A7, B2, B3, L1, L10, L11, L12, L14, L15, L16, L18, L19, L2, L20, L22, L23, L24, L25, L4/18a, L5, L6, L8, L9, O1, O11, O12, O14, O18, O2, O4 and O8 as described in Kawasaki et al., 2001, Eur. J. Immunol., 31:1017-1028, Schable and Zachau, 1993, Biol. Chem. Hoppe Seyler 374:1001-1022 and Brensing-Kuppers et al., 1997, Gene 191:173-181 and summarized at the website: www.ncbi.nlm.nih.gov/igblast/showGermline.cgi?organism=human&chainType=VK&seqType=nucleotide). See Table 2.

TABLE 2

Germline Kappa Chain Framework Sequences (SEQ ID Nos. 1-138)

| | | |
|---|---|---|
| 1st Framework | 2nd Framework | 3rd Framework |

| | | | |
|---|---|---|---|
| DVVMTQSPLSLPVTLGQPASISC | WFQQRPGQSPRRLIY | GVPDRFSGSGSGTDFTLKISRVEAEDVGVYYC | A1 |
| EIVLTQSPDFQSVTPKEKVTITG | WYQQKPDQSPKLLIK | GVPSRFSGSGSGTDFTLTINSLEAEDAATYYC | A10 |
| EIVLTQSPATLSLSPGERATLSC | WYQQKPGLAPRLLIY | GIPDRFSGSGSGTDFTLTISRLEPEDFAVYYC | A11 |
| DVVMTQSPAFLSVTPGEKVTITC | WYQQKPDQAPKLLIK | GVPSRFSGSGSGTDFTFTISSLEAEDAATYYC | A14 |
| DVVMTQSPLSLPVTLGQPASISC | WFQQRPGQSPRRLIY | GVPDRFSGSGSGTDFTLKISRVEAEDVGVYYC | A17 |
| DIVMTQTPLSLSVTPGQPASISC | WYLQKPGQSPQLLIY | GVPDRFSGSGSGTDFTLKISRVEAEDVGVYYC | A18 |
| DIVMTQSPLSLPVTPGEPASISC | WYLQKPGQSPQLLIY | GVPDRFSGSGSGTDFTLKISRVEAEDVGVYYC | A19 |
| DIVMTQTPLSLSVTPGQPASISC | WYLQKPGQPPQLLIY | GVPDRFSGSGSGTDFTLKISRVEAEDVGVYYC | A2 |
| DIQMTQSPSSLSASVGDRVTITC | WYQQKPGKVPKLLIY | GVPSRFSGSGSGTDFTLTISSLQPEDVATYYC | A20 |
| DIVMTQTPLSSPVTLGQPASISC | WLQQRPGQPPRLLIY | GVPDRFSGSGAGTDFTLKISRVEAEDVGVYYC | A23 |
| EIVLTQSPDFQSVTPKEKVTITC | WYQQKPDQSPKLLIK | GVPSRFSGSGSGTDFTLTINSLEAEDAATYYC | A26 |
| EIVLTQSPGTLSLSPGERATLSC | WYQQKPGQAPRLLIY | GIPDRFSGSGSGTDFTLTISRLEPEDFAVYYC | A27 |
| DIVMTQSPLSLPVTPGEPASISC | WYLQKPGQSPQLLIY | GVPDRFSGSGSGTDFTLKISRVEAEDVGVYYC | A3 |
| DIQMTQSPSSLSASVGDRVTITC | WYQQKPGKAPKRLIY | GVPSRFSGSGSGTEFTLTISSLQPEDFATYYC | A30 |
| EIVMTQTPLSLSITPGEQASISC | WFLQKARPVSTLLIY | GVPDRFSGSGSGTDFTLKISRVEAEDFGVYYC | A5 |
| DIVMTQTPLSSPVTLGQPASISF | WLQQRPGQPPRLLIY | GVPDRFSGSGAGTDFTLKISRVEAEDVGVYYC | A7 |
| ETTLTQSPAFMSATPGDKVNISC | WYQQKPGEAAIFIIQ | GIPPRFSGSGYGTDFTLTINNIESEDAAYYFC | B2 |
| DIVMTQSPDSLAVSLGERATINC | WYQQKPGQPPKLLIY | GVPDRFSGSGSGTDFTLTISSLQAEDVAVYYC | B3 |
| DIQMTQSPSSLSASVGDRVTITC | WFQQKPGKAPKSLIY | GVPSRFSGSGSGTDFTLTISSLQPEDFATYYC | L1 |
| EIVMTQSPPTLSLSPGERVTLSC | WYQQKPGQAPRLLIY | SIPARFSGSGSGTDFTLTISSLQPEDFAVYYC | L10 |
| AIQMTQSPSSLSASVGDRVTITC | WYQQKPGKAPKLLIY | GVPSRFSGSGSGTDFTLTISSLQPEDFATYYC | L11 |
| DIQMTQSPSTLSASVGDRVTITC | WYQQKPGKAPKLLIY | GVPSRFSGSGSGTEFTLTISSLQPDDFATYYC | L12 |
| NIQMTQSPSAMSASVGDRVTITC | WFQQKPGKVPKHLIY | GVPSRFSGSGSGTEFTLTISSLQPEDFATYYC | L14 |
| DIQMTQSPSSLSASVGDRVTITC | WYQQKPEKAPKSLIY | GVPSRFSGSGSGTDFTLTISSLQPEDFATYYC | L15 |
| EIVMTQSPATLSVSPGERATLSC | WYQQKPGQAPRLLIY | GIPARFSGSGSGTEFTLTISSLQSEDFAVYYC | L16 |
| AIQLTQSPSSLSASVGDRVTITC | WYQQKPGKAPKLLIY | GVPSRFSGSGSGTDFTLTISSLQPEDFATYYC | L18 |
| DIQMTQSPSSVSASVGDRVTITC | WYQQKPGKAPKLLIY | GVPSRFSGSGSGTDFTLTISSLQPEDFATYYC | L19 |
| EIVMTQSPATLSVSPGERATLSC | WYQQKPGQAPRLLIY | GIPARFSGSGSGTEFTLTISSLQSEDFAVYYC | L2 |
| EIVLTQSPATLSLSPGERATLSC | WYQQKPGQAPRLLIY | GIPARFSGSGPGTDFTLTISSLEPEDFAVYYC | L20 |
| DIQMIQSPSFLSASVGDRVSIIC | WYLQKPGKSPKLFLY | GVSSRFSGRGSGTDFTLTIISLKPEDFAAYYC | L22 |
| AIRMTQSPFSLSASVGDRVTITC | WYQQKPAKAPKLFIY | GVPSRFSGSGSGTDYTLTISSLQPEDFATYYC | L23 |
| VIWMTQSPSLLSASTGDRVTISC | WYQQKPGKAPELLIY | GVPSRFSGSGSGTDFTLTISGLQSEDFATYYC | L24 |
| EIVMTQSPATLSLSPGERATLSC | WYQQKPGQAPRLLIY | GIPARFSGSGSGTDFTLTISSLQPEDFAVYYC | L25 |
| AIQLTQSPSSLSASVGDRVTITC | WYQQKPGKAPKLLIY | GVPSRFSGSGSGTDFTLTISSLQPEDFATYYC | L4/18a |
| DIQMTQSPSSVSASVGDRVTITC | WYQQKPGKAPKLLIY | GVPSRFSGSGSGTDFTLTISSLQPEDFATYYC | L5 |
| EIVLTQSPATLSLSPGERATLSC | WYQQKPGQAPRLLIY | GIPARFSGSGSGTDFTLTISSLEPEDFAVYYC | L6 |
| DIQLTQSPSFLSASVGDRVTITC | WYQQKPGKAPKLLIY | GVPSRFSGSGSGTEFTLTISSLQPEDFATYYC | L8 |
| AIRMTQSPSSFSASTGDRVTITC | WYQQKPGKAPKLLIY | GVPSRFSGSGSGTDFTLTISCLQSEDFATYYC | L9 |
| DIVMTQTPLSLPVTPGEPASISC | WYLQKPGQSPQLLIY | GVPDRFSGSGSGTDFTLKISRVEAEDVGVYYC | O1 |
| DIVMTQTPLSLPVTPGEPASISC | WYLQKPGQSPQLLIY | GVPDRFSGSGSGTDFTLKISRVEAEDVGVYYC | O11 |
| DIQMTQSPSSLSASVGDRVTITC | WYQQKPGKAPKLLIY | GVPSRFSGSGSGTDFTLTISSLQPEDFATYYC | O12 |
| DIQLTQSPSSLSASVGDRVTITC | WYRQKPGKVPKLLIY | GVPSRFSGSGSGTDFTLTISSLQPEDVATYYG | O14 |
| DIQMTQSPSSLSASVGDRVTITC | WYQQKPGKAPKLLIY | GVPSRFSGSGSGTDFTFTISSLQPEDIATYYC | O18 |
| DIQMTQSPSSLSASVGDRVTITC | WYQQKPGKAPKLLIY | GVPSRFSGSGSGTDFTLTISSLQPEDFATYYC | O2 |
| DIQLTQSPSSLSASVGDRVTITG | WYRQKPGKVPKLLIY | GVPSRFSGSGSGTDFTLTISSLQPEDVATYYG | O4 |
| DIQMTQSPSSLSASVGDRVTITC | WYQQKPGKAPKLLIY | GVPSRFSGSGSGTDFTFTISSLQPEDIATYYC | O8 |

In specific embodiments, 5 human germline kappa chain sequences are considered for the 4th framework (Jκ1, Jκ2, Jκ3, Jκ4 and Jκ5 as described in Hieter et al., 1982, J. Biol. Chem. 257:1516-1522 and summarized at the website: www.ncbi.nlm.nih.gov/igblast/showGermline.cgi?organism=human&chainType=JK&seqType=nucleotide). See Table 3.

TABLE 3
4^{th} Framework Sequences of Kappa Chain

| | | |
|---|---|---|
| 139 | WTFGQGTKVEIK | Jκ1 |
| 140 | YTFGQGTKLEIK | Jκ2 |
| 141 | FTFGPGTKVDIK | Jκ3 |
| 142 | LTFGGGTKVEIK | Jκ4 |
| 143 | ITFGQGTRLEIK | Jκ5 |

In other specific embodiments, human germline k chain sequences are considered for the 1^{st}, 2^{nd}, 3^{rd} or 4^{th} framework.

In specific embodiments, 44 human germline heavy chain sequences are considered for the 1st, 2nd and 3rd frameworks (VH1-18, VH1-2, VH1-24, VH1-3, VH1-45, VH1-46, VH1-58, VH1-69, VH1-8, VH2-26, VH2-5, VH2-70, VH3-11, VH3-13, VH3-15, VH3-16, VH3-20, VH3-21, VH3-23, VH3-30, VH3-33, VH3-35, VH3-38, VH3-43, VH3-48, VH3-49, VH3-53, VH3-64, VH3-66, VH3-7, VH3-72, VH3-73, VH3-74, VH3-9, VH4-28, VH4-31, VH4-34, VH4-39, VH4-4, VH4-59, VH4-61, VH5-51, VH6-1 and VH7-81 as described in Matsuda et al., 1998, J. Exp. Med., 188:1973-1975 and summarized at the website: www.ncbi.nlm.nih.gov/igblast/showGermline.cgi?organism=human&chainType=VH&seqType=nucleotide). See Table 4 (according to the Kabat definition) and Table 5 (according to the Chothia definition).

TABLE 4

Frameworks defined according to Kabat (Seq ID Nos: 144-275):

| | | |
|---|---|---|
| 1st Framework | 2nd Framework | 3rd Framework |
| (Kabat definition) | (Kabat definition) | (Kabat definition) |

| | | | |
|---|---|---|---|
| QVQLVQSGAEVKKPGASVKVSCKASGYTFT | WVRQAPGQGLEWMG | RVTMTTDTSTSTAYMELRSLRSDDTAVYYCAR | VH1-18 |
| QVQLVQSGAEVKKPGASVKVSCKASGYTFT | WVRQAPGQGLEWMG | RVTMTRDTSISTAYMELSRLRSDDTAVYYCAR | VH1-2 |
| QVQLVQSGAEVKKPGASVKVSCKVSGYTLT | WVRQAPGKGLEWMG | RVTMTEDTSTDTAYMELSSLRSEDTAVYYCAT | VH1-24 |
| QVQLVQSGAEVKKPGASVKVSCKASGYTFT | WVRQAPGQRLEWMG | RVTITRDTSASTAYMELSSLRSEDMAVYYCAR | VH1-3 |
| QMQLVQSGAEVKKTGSSVKVSCKASGYTFT | WVRQAPGQALEWMG | RVTITRDRSMSTAYMELSSLRSEDTAMYYCAR | VH1-45 |
| QVQLVQSGAEVKKPGASVKVSCKASGYTFT | WVRQAPGQGLEWMG | RVTMTRDTSTSTVYMELSSLRSEDTAVYYCAR | VH1-46 |
| QMQLVQSGPEVKKPGTSVKVSCKASGFTFT | WVRQARGQRLEWIG | RVTITRDMSTSTAYMELSSLRSEDTAVYYCAA | VH1-58 |
| QVQLVQSGAEVKKPGSSVKVSCKASGGTFS | WVRQAPGQGLEWMG | RVTITADKSTSTAYMELSSLRSEDTAVYYCAR | VH1-69 |
| QVQLVQSGAEVKKPGASVKVSCKASGYTFT | WVRQATGQGLEWMG | RVTMTRNTSISTAYMELSSLRSEDTAVYYCAR | VH1-8 |
| QVTLKESGPVLVKPTETLTLTCTVSGFSLS | WIRQPPGKALEWLA | RLTISKDTSKSQVVLTMTNMDPVDTATYYCAR | VH2-26 |
| QITLKESGPTLVKPTQTLTLTCTFSGFSLS | WIRQPPGKALEWLA | RLTITKDTSKNQVVLTMTNMDPVDTATYYCAH | VH2-5 |
| QVTLRESGPALVKPTQTLTLTCTFSGFSLS | WIRQPPGKALEWLA | RLTISKDTSKNQVVLTMTNMDPVDTATYYCAR | VH2-70 |
| QVQLVESGGGLVKPGGSLRLSCAASGFTFS | WIRQAPGKGLEWVS | RFTISRDNAKNSLYLQMNSLRAEDTAVYYCAR | VH3-11 |
| EVQLVESGGGLVQPGGSLRLSCAASGFTFS | WVRQATGKGLEWVS | RFTISRENAKNSLYLQMNSLRAGDTAVYYCAR | VH3-13 |
| EVQLVESGGGLVKPGGSLRLSCAASGFTFS | WVRQAPGKGLEWVG | RFTISRDDSKNTLYLQMNSLKTEDTAVYYCTT | VH3-15 |
| EVQLVESGGGLVQPGGSLRLSCAASGFTFS | WARKAPGKGLEWVS | RFIISRDNSRNSLYLQKNRRRAEDMAVYYCVR | VH3-16 |
| EVQLVESGGGVVRPGGSLRLSCAASGFTFD | WVRQAPGKGLEWVS | RFTISRDNAKNSLYLQMNSLRAEDTALYHCAR | VH3-20 |
| EVQLVESGGGLVKPGGSLRLSCAASGFTFS | WVRQAPGKGLEWVS | RFTISRDNAKNSLYLQMNSLRAEDTAVYYCAR | VH3-21 |
| EVQLLESGGGLVQPGGSLRLSCAASGFTFS | WVRQAPGKGLEWVS | RFTISRDNSKNTLYLQMNSLRAEDTAVYYCAK | VH3-23 |
| QVQLVESGGGVVQPGRSLRLSCAASGFTFS | WVRQAPGKGLEWVA | RFTISRDNSKNTLYLQMNSLRAEDTAVYYCAR | VH3-30 |
| QVQLVESGGGVVQPGRSLRLSCAASGFTFS | WVRQAPGKGLEWVA | RFTISRDNSKNTLYLQMNSLRAEDTAVYYCAR | VH3-33 |
| EVQLVESGGGLVQPGGSLRLSCAASGFTFS | WVHQAPGKGLEWVS | RFIISRDNSRNTLYLQTNSLRAEDTAVYYCVR | VH3-35 |
| EVQLVESGGGLVQPRGSLRLSCAASGFTVS | WIRQAPGKGLEWVS | RFTISRDNSKNTLYLQMNNLRAEGTAVYYCAR | VH3-38 |
| EVQLVESGGVVVQPGGSLRLSCAASGFTFD | WVRQAPGKGLEWVS | RFTISRDNSKNSLYLQMNSLRTEDTALYYCAK | VH3-43 |
| EVQLVESGGGLVQPGGSLRLSCAASGFTFS | WVRQAPGKGLEWVS | RFTISRDNAKNSLYLQMNSLRDEDTAVYYCAR | VH3-48 |
| EVQLVESGGGLVQPGRSLRLSCTASGFTFG | WFRQAPGKGLEWVG | RFTISRDDSKSIAYLQMNSLKTEDTAVYYCTR | VH3-49 |
| EVQLVESGGGLIQPGGSLRLSCAASGFTVS | WVRQAPGKGLEWVS | RFTISRDNSKNTLYLQMNSLRAEDTAVYYCAR | VH3-53 |
| EVQLVESGECLVQPGGSLRLSCAASGFTFS | WVRQAPGKGLEYVS | RFTISRDNSKNTLYLQMGSLRAEDMAVYYCAR | VH3-64 |
| EVQLVESGGGLIQPGGSLRLSCAASGFTVS | WVRQAPGKGLEWVS | RFTISRDNSKNTLYLQMNSLRAEDTAVYYCAR | VH3-66 |
| EVQLVESGGGLVQPGGSLRLSCAASGFTFS | WVRQAPGKGLEWVA | RFTISRDNAKNSLYLQMNSLRAEDTAVYYCAR | VH3-7 |
| EVQLVESGGGLVQPGGSLRLSCAASGFTFS | WVRQAPGKGLEWVG | RFTISRDDSKNSLYLQMNSLKTEDTAVYYCAR | VH3-72 |
| EVQLVESGGGLVQPGGSLKLSCAASGFTFS | WVRQASGKGLEWVG | RFTISRDDSKNTAYLQMNSLKTEDTAVYYCTR | VH3-73 |
| EVQLVESGGGLVQPGGSLRLSCAASGFTFS | WVRQAPGKGLVWVS | RFTISRDNAKNTLYLQMNSLRAEDTAVYYCAR | VH3-74 |
| EVQLVESGGGLVQPGRSLRLSCAASGFTFD | WVRQAPCKGLEWVS | RFTISRDNAKNSLYLQMNSLRAEDTALYYCAK | VH3-9 |
| QVQLQESGPGLVKPSDTLSLTCAVSGYSIS | WIRQPPGKGLEWIG | RVTMSVDTSKNQFSLKLSSVTAVDTAVYYCAR | VH4-28 |
| QVQLQESGPGLVKPSQTLSLTCTVSGGSIS | WIRQHPGKGLEWIG | RVTISVDTSKNQFSLKLSSVTAADTAVYYCAR | VH4-31 |
| QVQLQQWGAGLLKPSETLSLTCAVYGGSFS | WIRQPPGKGLEWIG | RVTISVDTSKNQFSLKLSSVTAADTAVYYCAR | VH4-34 |
| QLQLQESGPGLVKPSETLSLTCTVSGGSIS | WIRQPPGKGLEWIG | RVTISVDTSKNQFSLKLSSVTAADTAVYYCAR | VH4-39 |
| QVQLQESGPGLVKPSETLSLTCTVSGGSIS | WIRQPAGKGLEWIG | RVTMSVDTSKNQFSLKLSSVTAADTAVYYCAR | VH4-4 |
| QVQLQESGPGLVKPSETLSLTCTVSGGSIS | WIRQPPGKGLEWIG | RVTISVDTSKNQFSLKLSSVTAADTAVYYCAR | VH4-59 |
| QVQLQESGPGLVKPSETLSLTCTVSGGSVS | WIRQPPGKGLEWIG | RVTISVDTSKNQFSLKLSSVTAADTAVYYCAR | VH4-61 |
| EVQLVQSGAEVKKPGESLKISCKGSGYSFT | WVRQMPGKGLEWMG | QVTISADKSISTAYLQWSSLKASDTAMYYCAR | VH5-51 |
| QVQLQQSGPGLVKPSQTLSLTCAISGDSVS | WIRQSPSRGLEWLG | RITINPDTSKNQFSLQLNSVTPEDTAVYYCAR | VH6-1 |
| QVQLVQSGHEVKQPGASVKVSCKASGYSFT | WVPQAPGQGLEWMG | RFVFSMDTSASTAYLQISSLKAEDMAMYYCAR | VH7-81 |

TABLE 5

Frameworks defined according to Chothia (Seq ID Nos: 276-407):

| | | |
|---|---|---|
| 1st Framework | 2nd Framework | 3rd Framework |
| (Chothia definition) | (Chothia definition) | (Chothia definition) |

| | | | |
|---|---|---|---|
| QVQLVQSGAEVKKPGASVKVSCKAS | GISWVRQAPGQGLEWMG | RVTMTTDTSTSTAYMELRSLRSDDTAVYYCAR | VH1-18 |
| QVQLVQSGAEVKKPGASVKVSCKAS | YMHWVRQAPGQGLEWMG | RVTMTRDTSISTAYMELSRLRSDDTAVYYCAR | VH1-2 |
| QVQLVQSGAEVKKPGASVKVSCKVS | SMHWVRQAPGKGLEWMG | RVTMTEDTSTDTAYMELSSLRSEDTAVYYCAT | VH1-24 |
| QVQLVQSGAEVKKPGASVKVSCKAS | AMHWVRQAPGQRLEWMG | RVTITRDTSASTAYMELSSLRSEDMAVYYCAR | VH1-3 |
| QMQLVQSGAEVKKTGSSVKVSCKAS | YLHWVRQAPGQALEWMG | RVTITRDRSMSTAYMELSSLRSEDTAMYYCAR | VH1-45 |
| QVQLVQSGAEVKKPGASVKVSCKAS | YMHWVRQAPGQGLEWMG | RVTMTRDTSTSTVYMELSSLRSEDTAVYYCAR | VH1-46 |
| QMQLVQSGPEVKKPGTSVKVSCKAS | AMQWVRQARGQRLEWIG | RVTITRDMSTSTAYMELSSLRSEDTAVYYCAA | VH1-58 |
| QVQLVQSGAEVKKPGSSVKVSCKAS | AISWVRQAPGQGLEWMG | RVTITADKSTSTAYMELSSLRSEDTAVYYCAR | VH1-69 |
| QVQLVQSGAEVKKPGASVKVSCKAS | DINWVRQATGQGLEWMG | RVTMTRNTSISTAYMELSSLRSEDTAVYYCAR | GVH1-8 |
| QVTLKESGPVLVKPTETLTLTCTVS | GVSWIRQPPGKALEWLA | RLTISKDTSKSQVVLTMTNMDPVDTATYYCAR | VH2-26 |
| QITLKESGPTLVKPTQTLTLTCTFS | CVGWIRQPPGKALEWLA | RLTITKDTSKNQVVLTMTNMDPVDTATYYCAH | RVH2-5 |
| QVTLRESGPALVKPTQTLTLTCTFS | CVSWIRQPPGKALEWLA | RLTISKDTSKNQVVLTMTNMDPVDTATYYCAR | VH2-70 |
| QVQLVESGGGLVKPGGSLRLSCAAS | YMSWIRQAPGKGLEWVS | RFTISRDNAKNSLYLQMNSLRAEDTAVYYCAR | VH3-11 |
| EVQLVESGGGLVQPGGSLRLSCAAS | DMHWVRQATGKGLEWVS | RFTISRENAKNSLYLQMNSLRAGDTAVYYCAR | VH3-13 |
| EVQLVESGGGLVKPGGSLRLSCAAS | WMSWVRQAPGKGLEWVG | RFTISRDDSKNTLYLQMNSLKTEDTAVYYCTT | VH3-15 |
| EVQLVESGGGLVQPGGSLRLSCAAS | DMNWARKAPGKGLEWVS | RFIISRDNSRNSLYLQKNRRRAEDMAVYYCVR | VH3-16 |
| EVQLVESGGGVVRPGGSLRLSCAAS | GMSWVRQAPGKGLEWVS | RFTISRDNAKNSLYLQMNSLRAEDTALYHCAR | VH3-20 |
| EVQLVESGGGLVKPGGSLRLSCAAS | SMNWVRQAPGKGLEWVS | RFTISRDNAKNSLYLQMNSLRAEDTAVYYCAR | VH3-21 |
| EVQLLESGGGLVQPGGSLRLSCAAS | AMSWVRQAPGKGLEWVS | RFTISRDNSKNTLYLQMNSLRAEDTAVYYCAK | VH3-23 |
| QVQLVESGGGVVQPGRSLRLSCAAS | GMHWVRQAPGKGLEWVA | RFTISRDNSKNTLYLQMNSLRAEDTAVYYCAR | VH3-30 |
| QVQLVESGGGVVQPGRSLRLSCAAS | CMHWVRQAPGKGLEWVA | RFTISRDNSKNTLYLQMNSLRAEDTAVYYCAR | VH3-33 |
| EVQLVESGGGLVQPGGSLRLSCAAS | DMNWVHQAPGKGLEWVS | RFIISRDNSRNTLYLQTNSLRAEDTAVYYCVR | VH3-35 |
| EVQLVESGGGLVQPRGSLRLSCAAS | EMSWIRQAPGKGLEWVS | RFTISRDNSKNTLYLQMNNLRAEGTAVYYCAR | VH3-38 |
| EVQLVESGGVVVQPGGSLRLSCAAS | TMHWVRQAPGKGLEWVS | RFTISRDNSKNSLYLQMNSLRTEDTALYYCAK | DVH3-43 |
| EVQLVESGGGLVQPGGSLRLSCAAS | SMNWVRQAPGKGLEWVS | RFTISRDNAKNSLYLQMNSLRDEDTAVYYCAR | VH3-48 |
| EVQLVESGGGLVQPGRSLRLSCTAS | AMSWFRQAPGKGLEWVG | RFTISRDDSKSIAYLQMNSLKTEDTAVYYCTR | VH3-49 |
| EVQLVESGGGLIQPGGSLRLSCAAS | YMSWVRQAPGKGLEWVS | RFTISRDNSKNTLYLQMNSLRAEDTAVYYCAR | VH3-53 |
| EVQLVESGEGLVQPGGSLRLSCAAS | AMHWVRQAPGKGLEYVS | RFTISRDNSKNTLYLQMGSLRAEDMAVYYCAR | VH3-64 |
| EVQLVESGGGLIQPGGSLRLSCAAS | YMSWVRQAPGKGLEWVS | RFTISRDNSKNTLYLQMNSLRAEDTAVYYCAR | VH3-66 |
| EVQLVESGGGLVQPGGSLRLSCAAS | WMSWVRQAPGKGLEWVA | RFTISRDNAKNSLYLQMNSLRAEDTAVYYCAR | VH3-7 |
| EVQLVESGGGLVQPGGSLRLSCAAS | YMDWVRQAPGKGLEWVG | RFTISRDDSKNSLYLQMNSLKTEDTAVYYCAR | VH3-72 |
| EVQLVESGGGLVQPCGSLKLSCAAS | AMHWVRQASGKGLEWVG | RFTISRDDSKNTAYLQMNSLKTEDTAVYYCTR | VH3-73 |
| EVQLVESGGGLVQPGGSLRLSCAAS | WMHWVRQAPGKGLVWVS | RFTISRDNAKNTLYLQMNSLRAEDTAVYYCAR | VH3-74 |
| EVQLVESGGGLVQPGRSLRLSCAAS | AMHWVRQAPGKGLEWVS | RFTISRDNAKNSLYLQMNSLRAEDTALYYCAK | VH3-9 |
| QVQLQESGPGLVKPSDTLSLTCAVS | WWGWIRQPPGKGLEWIG | RVTMSVDTSKNQFSLKLSSVTAVDTAVYYCAR | VH4-28 |
| QVQLQESGPGLVKPSQTLSLTCTVS | YWSWIRQHPGKGLEWIG | RVTISVDTSKNQFSLKLSSVTAADTAVYYCAR | VH4-31 |
| QVQLQQWGACLLKPSETLSLTCAVY | YWSWIRQPPGKGLEWIG | RVTISVDTSKNQFSLKLSSVTAADTAVYYCAR | VH4-34 |
| QLQLQESGPGLVKPSETLSLTCTVS | YWGWIRQPPGKGLEWIG | RVTISVDTSKNQFSLKLSSVTAADTAVYYCAR | VH4-39 |
| QVQLQESGPGLVKPSETLSLTCTVS | YWSWIRQPAGKGLEWIG | RVTMSVDTSKNQFSLKLSSVTAADTAVYYCAR | VH4-4 |
| QVQLQESGPGLVKPSETLSLTCTVS | YWSWIRQPPGKGLEWIG | RVTISVDTSKNQFSLKLSSVTAADTAVYYCAR | VH4-59 |
| QVQLQESGPGLVKPSETLSLTCTVS | YWSWIRQPPGKGLEWIG | RVTISVDTSKNQFSLKLSSVTAADTAVYYCAR | VH4-61 |
| EVQLVQSGAEVKKPGESLKISCKGS | WIGWVRQMPGKGLEWMG | QVTISADKSISTAYLQWSSLKASDTAMYYCAR | VH5-51 |
| QVQLQQSGPGLVKPSQTLSLTCAIS | AWNWIRQSPSRGLEWLG | RITINPDTSKNQFSLQLNSVTPEDTAVYYCAR | VH6-1 |
| QVQLVQSGHEVKQPGASVKVSCKAS | GMNWVPQAPGQGLEWMG | RFVFSMDTSASTAYLQISSLKAEDMAMYYCAR | VH7-81 |

In specific embodiments, 6 human germline heavy chain sequences are considered for the fourth framework (JH1, JH2, JH3, JH4, JH5 and JH6 as described in Ravetch et al., 1981, Cell 27(3 Pt 2):583-591 and summarized at the website: www.ncbi.nlm.nih.gov/igblast/showGermline.cgi?organism=human&chainType=JH&seqType=nucleotide). See Table 6.

TABLE 6
4^{th} Framework Sequences of the Heavy Chain

| | | |
|---|---|---|
| 408 | WGQGTLVTVSS | JH1 |
| 409 | WGRGTLVTVSS | JH2 |
| 410 | WGQGTMVTVSS | JH3 |
| 411 | WGQGTLVTVSS | JH4 |
| 412 | WGQGTLVTVSS | JH5 |
| 413 | WGQGTTVTVSS | JH6 |

In another embodiment, human frameworks for use in accordance with the present invention are obtained or derived from any antibodies (preferably mature antibody genes) that are known in the art, such as market approved or in late stage clinical trial antibodies, that do not elicit a significant immune response in human. Non-limiting examples of such antibodies include, but are not limited to, HuMax CD4, MT201, LL2 IgG (for lupus), Xolair, Synagis, Herceptin (anti HER-2), and Zenapax (anti-IL2 receptor). In another embodiment, acceptor antibody frameworks for use in accordance with the present invention are obtained from or derived from humanized antibodies that are known in the art. The amino acid sequences of the frameworks of antibodies known in the art may be obtained from the literature, databases or any other source. Non-limiting examples of antibodies include, but are not limited to, 0.5B (Maeda et al (1991) Hum. Antibod. Hybridomas 2:124 134); 1B4 (Singer et al (1993) J. Immunol. 150:2844 2857); 3a4D10 (Tempest et al (1994) Prot. Engng. 7:1501 1507; 425, Kettleborough et al (1991) Prot. Engng. 4:773 783; 60.3, Hsiao et al (1994) Prot. Engng. 7:815 822); A4.6.1 (Baca et al (1997) J. Biol. Chem. 272:10678 10684); AN100226m (Leger et al (1997) Hum. Antibod. 8:3 16); AT13/5 (Ellis et al (1995) J. Immunol. 155:925 937); AUK12 20 (Sato etal (1994) Mol. Immunol. 31:371-381); B1 8 (Jones et al (1986) Nature 321:522 525); B3 {Fv} PE38 (Benhar et al (1994) P.N.A.S. 91:12051 12055); B72.3 {M4} (Sha and Xiang (1994) Canc. Biother. 9:341 349); BMA 031 (Shearman et al (1991) J. Immunol. 147:4366 4373); BR96 (Rosok et al (1996) J. Biol. Chem. 271:22611 22618); BW431/26 (Gussow & Seemann (1991) Meth. Enzymol. 203:99 121); BrE 3 (Couto et al (1994) Antigen and Antibody Molecular Engineering, pp:55 59); CC49 (Kashmiri et al (1995) Hybridoma 14:461 473); CTM01 (Baker et al (1994) Antigen and Antibody Molecular Engineering, pp:61 82); Campath 1 {YTH34.5HL} (Riechmann et al (1988) Nature 332:323 327); Campath 9 {YNB46.1.8SG2B1.19} (Gorman et al (1991) P.N.A.S. 88:4181 4185); D1.3 (Verhoeyen et al (1988) Science 239:1534 1536); D1.3 {improved} (Foote & Winter (1992) J. Mol. Biol. 224:487-499); DX48 (Lewis & Crowe (1991) Gene 101:297 302); Fd138 80 (Co et al (1991) P.N.A.S. 88:2869 2873); Fd79 (Co et al (1991) P.N.A.S. 88:2869 2873); H17E2 (Verhoeyen et al (1991) Monoclonal Antibodies, pp:37 43); H52 (Eigenbrot et al (1994) Proteins 18: 49 62); HCMV16 (Hamilton et al (1997) J. Infect. Diseases 176:59 68); HCMV37 (Tempest et al (1995) Int. J. Biol. Macromol. 17:37 42); HMFG1 (Verhoeyen et al (1993) Immunol. 78:364 370); JES1 39D10 (Cook et al, (1996) Prot. Engng. 9:623 628); K20 (Poul et al, (1995) Mol. Immunol. 32:101 116); M195 (Co et al (1992) J. Immunol. 148:1149 1154); M22 (Graziano et al (1995) J. Immunol. 155:4996 5002); MaE11 (Presta et al (1993) J. Immunol. 151:2623-2632); MikB1 (Hakimi et al (1993) J. Immunol. 151:1075 1085); N901 (Roguska et al (1996) Prot. Engng. 9:895 904); OKT3 (Adair et al (1994) Hum. Antibod. Hybridomas 5:41-47); PM 1 (Sato et al (1993) Canc. Res. 53:851 856); RSV19 (Tempest et al (1991) Biotech. 9:266 271); SK2 (Sato et al (1996) Hum. Antibod. Hybridomas 7:175 183); TES C21 (Kolbinger et al (1993) Prot. Engng. 6:971 980); UCHT1 (Zhu and Carter (1995) J. Immunol. 155:1903 1910); YFC51.1 (Sims et al (1993) J. Immunol. 151:2296 2308); YTH12.5 (Routledge et al (1991) Eur. J. Immunol. 21:2717 2725); anti B4 (Roguska et al (1996) Prot. Engng. 9:895 904); anti Tac {MAT} (Queen et al (1989) P.N.A.S. 86:10029 10033); and mumAb4D5 (Carter et al (1992) P.N.A.S. 89:4285 4289). Each of which is incorporated herein by reference in its entirety.

In one embodiment, the heavy chain and light chain framework regions for use in accordance with the present invention are obtained or derived from the same source. In alternative embodiment, the light chain framework is obtained or derived from a different source than the heavy chain framework. In another embodiment, the heavy and/or light chain frameworks and one or more of the constant regions are obtained or derived from the same source. In alternative embodiment, the heavy and/or light chain frameworks and one or more of the constant regions are obtained or derived from different sources.

### 5.2. Construction of Combinatorial Libraries

A combinatorial library comprising a population of nucleic acid molecules comprising nucleotide sequences is constructed, wherein each nucleotide sequence comprises the heavy or light chain CDR loops of the donor antibody sequences fused in frame with the tailored frameworks of an acceptor heavy and/or a light chain variable region selected according to the “rules of design” described in Section 5.1. In accordance with the present invention, the nucleotide sequences may further comprise one or more constant regions.

Preferably, three libraries are constructed, wherein one library comprises a heavy chain combinatorial library with CDRs defined according to Kabat numbering system, a second library comprises a light chain combinatorial library with CDRs defined according to both Kabat and Chothia numbering system, and a third library comprises a heavy chain combinatorial library with CDRs defined according to Chothia numbering system.

A library can be constructed using any method known in the art. In a preferred embodiment, the construction of a combinatorial library is carried out using the Polymerase Chain Reaction (PCR) by overlap extension using appropriate oligonucleotides. Alternatively, the CDRs and the frameworks are ligated together by using a ligase.

The heavy and light chain libraries can be assembled by any method known in the art or as described in Wu, 2003, Methods Mol. Biol., 207, 197-212 (which is incorporated herein by reference). The V_{H} and V_{L} genes can be subsequently amplified as described in Wu, 2003, Methods Mol. Biol., 207, 197-212. A chimeric Fab (mouse V_{H} and V_{L} regions fused to the corresponding acceptor constant regions) can also be constructed after amplification of the genes coding for L1-V_{L} and L1-V_{H}.

The PCR product or the ligation product can be purified by any method known in the art. In a preferred embodiment, the minus single-stranded DNA is purified by ethanol precipitation after dissociation of the double-stranded PCR product or a ligation product using sodium hydroxide and elimination of the biotinylated strand by streptavidin-coated magnetic beads as described in Wu & An, 2003, Methods Mol. Biol., 207, 213-233 and Wu, 2003, Methods Mol. Biol., 207, 197-212, both of which are incorporated herein by reference.

The combinatorial libraries constructed in accordance with the present invention can be stored for a later use. The nucleic acids can be stored in a solution, as a dry sterilized lyophilized powder, or a water free concentrate in a hermetically sealed container. In cases where the nucleic acids are not stored in a solution, the nucleic acids can be reconstituted (e.g., with water or saline) to the appropriate concentration for a later use. The combinatorial libraries of the invention are preferably stored at between 2° C. and 8° C. in a container indicating the quantity and concentration of the nucleic acids.

### 5.3. Expression of the Combinatorial Libraries

The combinatorial libraries constructed in accordance with the present invention can be expressed using any methods know in the art, including but not limited to, bacterial expression system, mammalian expression system, and in vitro ribosomal display system.

In preferred embodiments, the present invention encompasses the use of phage vectors to express the combinatorial libraries. Phage vectors have particular advantages of providing a means for screening a very large population of expressed display proteins and thereby locate one or more specific clones that code for a desired binding activity.

The use of phage display vectors to express a large population of antibody molecules are well known in the art and will not be reviewed in detail herein. The method generally involves the use of a filamentous phage (phagemid) surface expression vector system for cloning and expressing antibody species of a library. See, e.g., Kang et al., Proc. Natl. Acad. Sci., USA, 88:4363-4366 (1991); Barbas et al., Proc. Natl. Acad. Sci., USA, 88:7978-7982 (1991); Zebedee et al., Proc. Natl. Acad. Sci., USA, 89:3175-3179 (1992); Kang et al., Proc. Natl. Acad. Sci., USA, 88:11120-11123 (1991); Barbas et al., Proc. Natl. Acad. Sci., USA, 89:4457-4461 (1992); Gram et al., Proc. Natl. Acad. Sci., USA, 89:3576-3580 (1992); Brinkman et al., J. Immunol. Methods 182:41-50 (1995); Ames et al., J. Immunol. Methods 184:177-186 (1995); Kettleborough et al., Eur. J. Immunol. 24:952-958 (1994); Persic et al., Gene 187 9-18 (1997); Burton et al., Advances in Immunology 57:191-280 (1994); PCT application No. PCT/GB91/01134; PCT publication Nos. WO 90/02809; WO 91/10737; WO 92/01047; WO 92/18619; WO 93/11236; WO 95/15982; WO 95/20401; and U.S. Pat. Nos. 5,698,426; 5,223,409; 5,403,484; 5,580,717; 5,427,908; 5,750,753; 5,821,047; 5,571,698; 5,427,908; 5,516,637; 5,780,225; 5,658,727; 5,733,743 and 5,969,108, all of which are incorporated herein by reference in their entireties.

A preferred phagemid vector of the present invention is a recombinant DNA molecule containing a nucleotide sequence that codes for and is capable of expressing a fusion polypeptide containing, in the direction of amino- to carboxy-terminus, (1) a prokaryotic secretion signal domain, (2) a heterologous polypeptide defining an immunoglobulin heavy or light chain variable region, and (3) a filamentous phage membrane anchor domain. The vector includes DNA expression control sequences for expressing the fusion polypeptide, preferably prokaryotic control sequences.

The filamentous phage membrane anchor is preferably a domain of the cpIII or cpVIII coat protein capable of associating with the matrix of a filamentous phage particle, thereby incorporating the fusion polypeptide onto the phage surface.

Preferred membrane anchors for the vector are obtainable from filamentous phage M13, f1, fd, and equivalent filamentous phage. Preferred membrane anchor domains are found in the coat proteins encoded by gene III and gene VIII. (See Ohkawa et al., J. Biol. Chem., 256:9951-9958, 1981). The membrane anchor domain of a filamentous phage coat protein is a portion of the carboxy terminal region of the coat protein and includes a region of hydrophobic amino acid residues for spanning a lipid bilayer membrane, and a region of charged amino acid residues normally found at the cytoplasmic face of the membrane and extending away from the membrane. For detailed descriptions of the structure of filamentous phage particles, their coat proteins and particle assembly, see the reviews by Rached et al., Microbiol. Rev., 50:401-427 (1986); and Model et al., in “The Bacteriophages: Vol. 2”, R. Calendar, ed. Plenum Publishing Co., pp. 375-456 (1988).

The secretion signal is a leader peptide domain of a protein that targets the protein to the periplasmic membrane of gram negative bacteria. A preferred secretion signal is a pelB secretion signal. (Better et al., Science, 240:1041-1043 (1988); Sastry et al., Proc. Natl. Acad. Sci., USA, 86:5728-5732 (1989); and Mullinax et al., Proc. Natl. Acad. Sci., USA, 87:8095-8099 (1990)). The predicted amino acid residue sequences of the secretion signal domain from two pelB gene product variants from Erwinia carotova are described in Lei et al., Nature, 331:543-546 (1988). Amino acid residue sequences for other secretion signal polypeptide domains from E. coli useful in this invention as described in Oliver, Escherichia coli and Salmonella Typhimurium, Neidhard, F. C. (ed.), American Society for Microbiology, Washington, D.C., 1:56-69 (1987).

DNA expression control sequences comprise a set of DNA expression signals for expressing a structural gene product and include both 5′ and 3′ elements, as is well known, operatively linked to the gene. The 5′ control sequences define a promoter for initiating transcription and a ribosome binding site operatively linked at the 5′ terminus of the upstream translatable DNA sequence. The 3′ control sequences define at least one termination (stop) codon in frame with and operatively linked to the heterologous fusion polypeptide.

In preferred embodiments, the vector used in this invention includes a prokaryotic origin of replication or replicon, i.e., a DNA sequence having the ability to direct autonomous replication and maintenance of the recombinant DNA molecule extra-chromosomally in a prokaryotic host cell, such as a bacterial host cell, transformed therewith. Such origins of replication are well known in the art. Preferred origins of replication are those that are efficient in the host organism. A preferred host cell is E. coli. See Sambrook et al., in “Molecular Cloning: a Laboratory Manual”, 2nd edition, Cold Spring Harbor Laboratory Press, New York (1989).

In addition, those embodiments that include a prokaryotic replicon can also include a nucleic acid whose expression confers a selective advantage, such as drug resistance, to a bacterial host transformed therewith. Typical bacterial drug resistance genes are those that confer resistance to ampicillin, tetracycline, neomycin/kanamycin or chloramphenicol. Vectors typically also contain convenient restriction sites for insertion of translatable DNA sequences.

In some embodiments, the vector is capable of co-expression of two cistrons contained therein, such as a nucleotide sequence encoding a variable heavy chain region and a nucleotide sequence encoding a variable light chain region. Co-expression has been accomplished in a variety of systems and therefore need not be limited to any particular design, so long as sufficient relative amounts of the two gene products are produced to allow assembly and expression of functional heterodimer.

In some embodiments, a DNA expression vector is designed for convenient manipulation in the form of a filamentous phage particle encapsulating a genome. In this embodiment, a DNA expression vector further contains a nucleotide sequence that defines a filamentous phage origin of replication such that the vector, upon presentation of the appropriate genetic complementation, can replicate as a filamentous phage in single stranded replicative form and be packaged into filamentous phage particles. This feature provides the ability of the DNA expression vector to be packaged into phage particles for subsequent segregation of the particle, and vector contained therein, away from other particles that comprise a population of phage particles.

A filamentous phage origin of replication is a region of the phage genome, as is well known, that defines sites for initiation of replication, termination of replication and packaging of the replicative form produced by replication (see for example, Rasched et al., Microbiol. Rev., 50:401-427, 1986; and Horiuchi, J. Mol. Biol., 188:215-223, 1986). A preferred filamentous phage origin of replication for use in the present invention is an M13, f1 or fd phage origin of replication (Short et al., Nucl. Acids Res., 16:7583-7600, 1988).

The method for producing a heterodimeric immunoglobulin molecule generally involves (1) introducing a large population of display vectors each capable of expressing different putative binding sites displayed on a phagemid surface display protein to a filamentous phage particle, (3) expressing the display protein and binding site on the surface of a filamentous phage particle, and (3) isolating (screening) the surface-expressed phage particle using affinity techniques such as panning of phage particles against a preselected antigen, thereby isolating one or more species of phagemid containing a display protein containing a binding site that binds a preselected antigen.

The isolation of a particular vector capable of expressing an antibody binding site of interest involves the introduction of the dicistronic expression vector able to express the phagemid display protein into a host cell permissive for expression of filamentous phage genes and the assembly of phage particles. Typically, the host is E. coli. Thereafter, a helper phage genome is introduced into the host cell containing the phagemid expression vector to provide the genetic complementation necessary to allow phage particles to be assembled.

The resulting host cell is cultured to allow the introduced phage genes and display protein genes to be expressed, and for phage particles to be assembled and shed from the host cell. The shed phage particles are then harvested (collected) from the host cell culture media and screened for desirable antibody binding properties. Typically, the harvested particles are “panned” for binding with a preselected antigen. The strongly binding particles are then collected, and individual species of particles are clonally isolated and further screened for binding to the antigen. Phages which produce a binding site of desired antigen binding specificity are selected.

After phage selection, the antibody coding regions from the phage can be isolated and used to generate whole antibodies or any other desired antigen binding fragment, and expressed in any desired host, including mammalian cells, insect cells, plant cells, yeast, and bacteria, e.g., as described in detail below. For example, techniques to recombinantly produce Fab, Fab′ and F(ab′)₂ fragments can also be employed using methods known in the art such as those disclosed in International Publication No. WO 92/22324; Mullinax et al., BioTechniques 12(6):864-869 (1992); and Sawai et al., AJRI 34:26-34 (1995); and Better et al., Science 240:1041-1043 (1988) (said references incorporated by reference in their entireties). Examples of techniques which can be used to produce single-chain Fvs and antibodies include those described in U.S. Pat. Nos. 4,946,778 and 5,258,498; Huston et al., Methods in Enzymology 203:46-88 (1991); Shu et al., PNAS 90:7995-7999 (1993); and Skerra et al., Science 240:1038-1040 (1988).

The invention also encompasses a host cell containing a vector or nucleotide sequence of this invention. In a specific embodiment, the host cell is E. coli.

In a preferred embodiment, a combinatorial library of the invention is cloned into a M13-based phage vector. This vector allows the expression of Fab fragments that contain the first constant domain of the human γ1 heavy chain and the constant domain of the human kappa (K) light chain under the control of the lacZ promoter. This can be carried out by hybridization mutagenesis as described in Wu & An, 2003, Methods Mol. Biol., 207, 213-233; Wu, 2003, Methods Mol. Biol., 207, 197-212; and Kunkel et al., 1987, Methods Enzymol. 154, 367-382; all of which are incorporated herein by reference in their entireties. Briefly, purified minus strands corresponding to the heavy and light chains to be cloned are annealed to two regions containing each one palindromic loop. Those loops contain a unique XbaI site which allows for the selection of the vectors that contain both V_{L} and V_{H} chains fused in frame with the human kappa (κ) constant and first human γ1 constant regions, respectively (Wu & An, 2003, Methods Mol. Biol., 207, 213-233, Wu, 2003, Methods Mol. Biol., 207, 197-212). Synthesized DNA is then electroporated into XL1-blue for plaque formation on XL1-blue bacterial lawn or production of Fab fragments as described in Wu, 2003, Methods Mol. Biol., 207, 197-212.

In addition to bacterial/phage expression systems, other host-vector systems may be utilized in the present invention to express the combinatorial libraries of the present invention. These include, but are not limited to, mammalian cell systems transfected with a vector or infected with virus (e.g., vaccinia virus, adenovirus, etc.); insect cell systems transfected with a vector or infected with virus (e.g., baculovirus); microorganisms such as yeast containing yeast vectors; or bacteria transformed with DNA, plasmid DNA, or cosmid DNA. See e.g., Verma et al., J Immunol Methods. 216(1-2):165-81 (1998), which is incorporated herein by reference.

The expression elements of vectors vary in their strengths and specificities. Depending on the host-vector system utilized, any one of a number of suitable transcription and translation elements may be used. In a preferred aspect, each nucleic acid of a combinatorial library of the invention is part of an expression vector that expresses the humanized heavy and/or light chain or humanized heavy and/or light variable regions in a suitable host. In particular, such nucleic acids have promoters, preferably heterologous promoters, operably linked to the antibody coding region, said promoter being inducible or constitutive, and, optionally, tissue-specific. (See Section 5.7 for more detail.) In another particular embodiment, nucleic acid molecules are used in which the antibody coding sequences and any other desired sequences are flanked by regions that promote homologous recombination at a desired site in the genome, thus providing for intrachromosomal expression of the antibody encoding nucleic acids (Koller and Smithies, 1989, Proc. Natl. Acad. Sci. USA 86:8932-8935; Zijlstra et al., 1989, Nature 342:435-438).

The combinatorial libraries can also be expressed using in vitro systems, such as the ribosomal display systems (see Section 5.6 for detail).

### 5.4. Selection of Humanized Antibodies

The expressed combinatorial libraries can be screened for binding to the antigen recognized by the donor antibody using any methods known in the art. In preferred embodiments, a phage display library constructed and expressed as described in section 5.2. and 5.3, respectively, is screened for binding to the antigen recognized by the donor antibody, and the phage expressing V_{H} and/or V_{L} domain with significant binding to the antigen can be isolated from a library using the conventional screening techniques (e.g. as described in Harlow, E., and Lane, D., 1988, supra Gherardi, E et al. 1990. J. Immunol. meth. 126 p61-68). The shed phage particles from host cells are harvested (collected) from the host cell culture media and screened for desirable antibody binding properties. Typically, the harvested particles are “panned” for binding with a preselected antigen. The strongly binding particles are then collected, and individual species of particles are clonally isolated and further screened for binding to the antigen. Phages which produce a binding site of desired antigen binding specificity are selected. Preferably, a humanized antibody of the invention has affinity of at least 1×10⁶ M⁻¹, preferably at least 1×10⁷ M⁻¹, at least 1×10⁸ M⁻¹, or at least 1×10⁹ M⁻¹ for an antigen of interest.

In a preferred embodiment, a phage library is first screened using a modified plaque lifting assay, termed capture lift. See Watkins et al., 1997, Anal. Biochem., 253:37-45. Briefly, phage infected bacteria are plated on solid agar lawns and subsequently, are overlaid with nitrocellulose filters that have been coated with a Fab-specific reagent (e.g., an anti-Fab antibody). Following the capture of nearly uniform quantities of phage-expressed Fab, the filters are probed with desired antigen-Ig fusion protein at a concentration substantially below the Kd value of the Fab.

In another embodiment, the combinatorial libraries are expressed and screened using in vitro systems, such as the ribosomal display systems (see, e.g., Graddis et al., Curr Pharm Biotechnol. 3(4):285-97 (2002); Hanes and Plucthau PNAS USA 94:4937-4942 (1997); He, 1999, J. Immunol. Methods, 231:105; Jermutus et al. (1998) Current Opinion in Biotechnology, 9:534-548; each of which is incorporated herein by reference). The ribosomal display system works by translating a library of antibody or fragment thereof in vitro without allowing the release of either antibody (or fragment thereof) or the mRNA from the translating ribosome. This is made possible by deleting the stop codon and utilizing a ribosome stabilizing buffer system. The translated antibody (or fragment thereof) also contains a C-terminal tether polypeptide extension in order to facilitate the newly synthesized antibody or fragment thereof to emerge from the ribosomal tunnel and fold independently. The folded antibody or fragment thereof can be screened or captured with a cognate antigen. This allows the capture of the mRNA, which is subsequently enriched in vitro. The E. Coli and rabbit reticulocute systems are commonly used for the ribosomal display.

Other methods know in the art, e.g., PROfusion™ (U.S. Pat. No. 6,281,344, Phylos Inc., Lexington, Mass.), Covalent Display (International Publication No. WO 9837186, Actinova Ltd., Cambridge, U.K.), can also be used in accordance with the present invention.

In another embodiment, an antigen can be bound to a solid support(s), which can be provided by a petri dish, chromatography beads, magnetic beads and the like. As used herein, the term “solid support” is not limited to a specific type of solid support. Rather a large number of supports are available and are known to one skilled in the art. Solid supports include silica gels, resins, derivatized plastic films, glass beads, cotton, plastic beads, polystyrene beads, alumina gels, and polysaccharides. A suitable solid support may be selected on the basis of desired end use and suitability for various synthetic protocols. For example, for peptide synthesis, a solid support can be a resin such as p-methylbenzhydrylamine (pMBHA) resin (Peptides International, Louisville, Ky.), polystyrenes (e.g., PAM-resin obtained from Bachem Inc., Peninsula Laboratories, etc.), including chloromethylpolystyrene, hydroxymethylpolystyrene and aminomethylpolystyrene, poly (dimethylacrylamide)-grafted styrene co-divinyl-benzene (e.g., POLYHIPE resin, obtained from Aminotech, Canada), polyamide resin (obtained from Peninsula Laboratories), polystyrene resin grafted with polyethylene glycol (e.g., TENTAGEL or ARGOGEL, Bayer, Tubingen, Germany) polydimethylacrylamide resin (obtained from Milligen/Biosearch, California), or Sepharose (Pharmacia, Sweden).

The combinatorial library is then passed over the antigen, and those individual antibodies that bind are retained after washing, and optionally detected with a detection system. If samples of bound population are removed under increasingly stringent conditions, the binding affinity represented in each sample will increase. Conditions of increased stringency can be obtained, for example, by increasing the time of soaking or changing the pH of the soak solution, etc.

In another embodiment, enzyme linked immunosorbent assay (ELISA) is used to screen for an antibody with desired binding activity. ELISAs comprise preparing antigen, coating the wells of a microtiter plate with the antigen, washing away antigen that did not bind the wells, adding the antibody of interest conjugated to a detectable compound such as an enzymatic substrate (e.g., horseradish peroxidase or alkaline phosphatase) to the wells and incubating for a period of time, washing away unbound antibodies or non-specifically bound antibodies, and detecting the presence of the antibodies specifically bound to the antigen coating the well. In ELISAs, the antibody of interest does not have to be conjugated to a detectable compound; instead, a second antibody (which recognizes the antibody of interest) conjugated to a detectable compound may be added to the well. Further, instead of coating the well with the antigen, the antibody may be coated to the well. In this case, the detectable molecule could be the antigen conjugated to a detectable compound such as an enzymatic substrate (e.g., horseradish peroxidase or alkaline phosphatase). One of skill in the art would be knowledgeable as to the parameters that can be modified to increase the signal detected as well as other variations of ELISAs known in the art. For further discussion regarding ELISAs see, e.g., Ausubel et al., eds, 1994, Current Protocols in Molecular Biology, Vol. I, John Wiley & Sons, Inc., New York at 11.2.1.

In another embodiment, BIAcore kinetic analysis is used to determine the binding on and off rates (Kd) of antibodies of the invention to a specific antigen. BIAcore kinetic analysis comprises analyzing the binding and dissociation of an antigen from chips with immobilized antibodies of the invention on their surface. See Wu et al., 1999, J. Mol. Biol., 294:151-162, which is incorporated herein by reference in its entirety. Briefly, antigen-Ig fusion protein is immobilized to a (1-ethyl-3-(3-dimethylaminopropyl)-carbodiimide hydrochloride) and N-hydroxy-succinimide-activated sensor chip CM5 by injecting antigen-Ig in sodium acetate. Antigen-Ig is immobilized at a low density to prevent rebinding of Fabs during the dissociation phase. To obtain association rate constant (Kon), the binding rate at six different Fab concentrations is determined at certain flow rate. Dissociation rate constant (Koff) are the average of six measurements obtained by analyzing the dissociation phase. Sensorgrams are analyzed with the BIAevaluation 3.0 program. Kd is calculated from Kd=Koff/Kon. Residual Fab is removed after each measurement by prolonged dissociation. In a more preferred embodiment, positive plaques are picked, re-plated at a lower density, and screened again.

In another embodiment, the binding affinity of an antibody (including a scFv or other molecule comprising, or alternatively consisting of, antibody fragments or variants thereof) to an antigen and the off-rate of an antibody-antigen interaction can be determined by competitive binding assays. One example of a competitive binding assay is a radioimmunoassay comprising the incubation of labeled antigen (e.g., ³H or ¹²¹I with the antibody of interest in the presence of increasing amounts of unlabeled antigen, and the detection of the antibody bound to the labeled antigen. The affinity of the antibody of the present invention and the binding off-rates can be determined from the data by Scatchard plot analysis. Competition with a second antibody can also be determined using radioimmunoassays. In this case, an antigen is incubated with an antibody of the present invention conjugated to a labeled compound (e.g., ³H or ¹²¹I) in the presence of increasing amounts of an unlabeled second antibody.

Other assays, such as immunoassays, including but not limited to, competitive and non-competitive assay systems using techniques such as western blots, radioimmunoassays, ELISA (enzyme linked immunosorbent assay), sandwich immunoassays, immunoprecipitation assays, precipitin reactions, gel diffusion precipitin reactions, immunodiffusion assays, agglutination assays, complement-fixation assays, fluorescent immunoassays, and protein A immunoassays, can also be used to screen or further characterization of the binding specificity of a humanized antibody. Such assays are routine and well known in the art (see, e.g., Ausubel et al., eds, 1994, Current Protocols in Molecular Biology, Vol. 1, John Wiley & Sons, Inc., New York, which is incorporated by reference herein in its entirety). Exemplary immunoassays are described briefly below (which are not intended by way of limitation).

In a preferred embodiment, ELISA is used as a secondary screening on supernatant prepared from bacterial culture expressing Fab fragments in order to confirm the clones identified by the capture lift assay. Two ELISAs can be carried out: (1) Quantification ELISA: this can be carried out essentially as described in Wu, 2003, Methods Mol. Biol., 207, 197-212, which is incorporated herein by reference in its entirety. Briefly, concentrations can be determined by an anti-human Fab ELISA: individual wells of a 96-well Immulon Immunoplate are coated with 50 ng of a goat anti-human Fab antibody and then incubated with samples (supernatant-expressed Fabs) or standard (human IgG Fab). Incubation with a goat anti-human kappa horseradish peroxidase (HRP) conjugate then followed. HRP activity can be detected with TMB substrate and the reaction quenched with 0.2 M H2SO4. Plates are read at 450 nm. Clones that express detactable amount of Fab are then selected for the next part of the secondary screening. (2) Functional ELISA: briefly, a particular antigen binding activity is determined by the antigen-based ELISA: individual wells of a 96-well Maxisorp Immunoplate are coated with 50 ng of the antigen of interest, blocked with 1% BSA/0.1% Tween 20 and then incubated with samples (supernatant-expressed Fabs). Incubation with a goat anti-human kappa horseradish peroxidase (HRP) conjugate then followed. HRP activity is detected with TMB substrate and the reaction quenched with 0.2 M H2SO4. Plates are read at 450 nm.

Immunoprecipitation protocols generally comprise lysing a population of cells in a lysis buffer such as RIPA buffer (1% NP-40 or Triton X-100, 1% sodium deoxycholate, 0.1% SDS, 0.15 M NaCl, 0.01 M sodium phosphate at pH 7.2, 1% Trasylol) supplemented with protein phosphatase and/or protease inhibitors (e.g., EDTA, PMSF, 159 aprotinin, sodium vanadate), adding the antibody of interest to the cell lysate, incubating for a period of time (e.g., to 4 hours) at 40 degrees C., adding protein A and/or protein G sepharose beads to the cell lysate, incubating for about an hour or more at 40 degrees C., washing the beads in lysis buffer and re-suspending the beads in SDS/sample buffer. The ability of the antibody of interest to immunoprecipitate a particular antigen can be assessed by, e.g., western blot analysis. One of skill in the art would be knowledgeable as to the parameters that can be modified to increase the binding of the antibody to an antigen and decrease the background (e.g., pre-clearing the cell lysate with sepharose beads). For further discussion regarding immunoprecipitation protocols see, e.g., Ausubel et al., eds, 1994, Current Protocols in Molecular Biology, Vol. 1, John Wiley & Sons, Inc., New York, at 10.16.1.

Western blot analysis generally comprises preparing protein samples, electrophoresis of the protein samples in a polyacrylamide get (e.g., 8%-20% SDS-PAGE depending on the molecular weight of the antigen), transferring the protein sample from the polyacrylamide get to a membrane such as nitrocellulose, PVDF or nylon, blocking the membrane in blocking solution (e.g., PBS with 3% BSA or non-fat milk), washing the membrane in washing buffer (e.g., PBSTween 20), blocking the membrane with primary antibody (the antibody of interest) diluted in blocking buffer, washing the membrane in washing buffer, blocking the membrane with a secondary antibody (which recognizes the primary antibody, e.g., an anti-human antibody) conjugated to an enzymatic substrate (e.g., horseradish peroxidase or alkaline phosphatase) or radioactive molecule (e.g., 12P or 121I) diluted in blocking buffer, washing the membrane in wash buffer, and detecting the presence of the antigen. One of skill in the art would be knowledgeable as to the parameters that can be modified to increase the signal detected and to reduce the background noise. For further discussion regarding western blot protocols see, e.g., Ausubel et al., eds, 1994, GinTent Protocols in Molecular Biology, Vol. 1, John Wiley & Sons, Inc., New York at 10.8.1.

A nucleic acid encoding a modified (e.g., humanized) antibody or fragment thereof with desired antigen binding activity can be characterized by sequencing, such as dideoxynucleotide sequencing using a ABI300 genomic analyzer. Other immunoassays, such as the two-part secondary ELISA screen described above, can be used to compare the modified (e.g., humanized) antibodies to each other and to the donor antibody in terms of binding to a particular antigen of interest.

### 5.5. Production and Characterization of Humanized Antibodies

Once one or more nucleic acids encoding a humanized antibody or fragment thereof with desired binding activity are selected, the nucleic acid can be recovered by standard techniques known in the art. In a preferred embodiment, the selected phage particles are recovered and used to infect fresh bacteria before recovering the desired nucleic acids.

A phage displaying a protein comprising a humanized variable region with a desired specificity or affinity can be eluted from an affinity matrix by any method known in the art. In one embodiment, a ligand with better affinity to the matrix is used. In a specific embodiment, the corresponding non-humanized antibody is used. In another embodiment, an elution method which is not specific to the antigen-antibody complex is used.

The method of mild elution uses binding of the phage antibody population to biotinylated antigen and binding to streptavidin magnetic beads. Following washing to remove non-binding phage, the phage antibody is eluted and used to infect cells to give a selected phage antibody population. A disulfide bond between the biotin and the antigen molecule allows mild elution with dithiothreitol. In one embodiment, biotinylated antigen can be used in excess but at or below a concentration equivalent to the desired dissociation constant for the antigen-antibody binding. This method is advantageous for the selection of high affinity antibodies (R. E. Hawkins, S. J. Russell and G. Winter J. Mol. Biol. 226 889-896, 1992). Antibodies may also be selected for slower off rates for antigen selection as described in Hawkins et al, 1992, supra. The concentration of biotinylated antigen may gradually be reduced to select higher affinity phage antibodies. As an alternative, the phage antibody may be in excess over biotinylated antigen in order that phage antibodies compete for binding, in an analogous way to the competition of peptide phage to biotinylated antibody described by J. K. Scott & G. P. Smith (Science 249 386-390, 1990).

In another embodiment, a nucleotide sequence encoding amino acids constituting a recognition site for cleavage by a highly specific protease can be introduced between the foreign nucleic acid inserted, e.g., between a nucleic acid encoding an antibody fragment, and the sequence of the remainder of gene III. Non-limiting examples of such highly specific proteases are Factor X and thrombin. After binding of the phage to an affinity matrix and elution to remove non-specific binding phage and weak binding phage, the strongly bound phage would be removed by washing the column with protease under conditions suitable for digestion at the cleavage site. This would cleave the antibody fragment from the phage particle eluting the phage. These phage would be expected to be infective, since the only protease site should be the one specifically introduced. Strongly binding phage could then be recovered by infecting, e.g., E. coli TG1 cells.

An alternative procedure to the above is to take the affinity matrix which has retained the strongly bound pAb and extract the DNA, for example by boiling in SDS solution. Extracted DNA can then be used to directly transform E. coli host cells or alternatively the antibody encoding sequences can be amplified, for example using PCR with suitable primers, and then inserted into a vector for expression as a soluble antibody for further study or a pAb for further rounds of selection.

In another embodiment, a population of phage is bound to an affinity matrix which contains a low amount of antigen. There is competition between phage, displaying high affinity and low affinity proteins, for binding to the antigen on the matrix. Phage displaying high affinity protein is preferentially bound and low affinity protein is washed away. The high affinity protein is then recovered by elution with the ligand or by other procedures which elute the phage from the affinity matrix (International Publication No. WO92/01047 demonstrates this procedure).

The recovered nucleic acid encoding donor CDRs and humanized framework can be used by itself or can be used to construct nucleic acid for a complete antibody molecule by joining them to the constant region of the respective acceptor template. When the nucleic acids encoding antibodies are introduced into a suitable host cell line, the transfected cells can secrete antibodies with all the desirable characteristics of monoclonal antibodies.

Once a nucleic acid encoding an antibody molecule or a heavy or light chain of an antibody, or fragment thereof preferably, containing the heavy or light chain variable region) of the invention has been obtained, the vector for the production of the antibody molecule may be produced by recombinant DNA technology using techniques well known in the art. Thus, methods for preparing a protein by expressing a nucleic acid encoding an antibody are described herein. Methods which are well known to those skilled in the art can be used to construct expression vectors containing antibody coding sequences and appropriate transcriptional and translational control signals. These methods include, for example, in vitro recombinant DNA techniques, synthetic techniques, and in vivo genetic recombination. The invention, thus, provides replicable vectors comprising a nucleotide sequence encoding an antibody molecule of the invention, a heavy or light chain of an antibody, a heavy or light chain variable domain of an antibody or a fragment thereof, or a heavy or light chain CDR, operably linked to a promoter. In a specific embodiment, the expression of an antibody molecule of the invention, a heavy or light chain of an antibody, a heavy or light chain variable domain of an antibody or a fragment thereof, or a heavy or light chain CDR is regulated by a constitutive promoter. In another embodiment, the expression of an antibody molecule of the invention, a heavy or light chain of an antibody, a heavy or light chain variable domain of an antibody or a fragment thereof, or a heavy or light chain CDR is regulated by an inducible promoter. In another embodiment, the expression of an antibody molecule of the invention, a heavy or light chain of an antibody, a heavy or light chain variable domain of an antibody or a fragment thereof, or a heavy or light chain CDR is regulated by a tissue specific promoter. Such vectors may also include the nucleotide sequence encoding the constant region of the antibody molecule (see, e.g., International Publication No. WO 86/05807; International Publication No. WO 89/01036; and U.S. Pat. No. 5,122,464) and the variable domain of the antibody may be cloned into such a vector for expression of the entire heavy, the entire light chain, or both the entire heavy and light chains.

The expression vector is transferred to a host cell by conventional techniques and the transfected cells are then cultured by conventional techniques to produce an antibody of the invention. Thus, the invention includes host cells containing a polynucleotide encoding an antibody of the invention or fragments thereof, or a heavy or light chain thereof, or portion thereof, or a single chain antibody of the invention, operably linked to a heterologous promoter. In preferred embodiments for the expression of double-chained antibodies, vectors encoding both the heavy and light chains may be co-expressed in the host cell for expression of the entire immunoglobulin molecule, as detailed below.

Preferably, the cell line which is transformed to produce the altered antibody is an immortalized mammalian cell line of lymphoid origin, including but not limited to, a myeloma, hybridoma, trioma or quadroma cell line. The cell line may also comprise a normal lymphoid cell, such as a B cell, which has been immortalized by transformation with a virus, such as the Epstein Barr virus. Most preferably, the immortalized cell line is a myeloma cell line or a derivative thereof.

It is known that some immortalized lymphoid cell lines, such as myeloma cell lines, in their normal state, secrete isolated immunoglobulin light or heavy chains. If such a cell line is transformed with the recovered nucleic acid from phage library, it will not be necessary to reconstruct the recovered fragment to a constant region, provided that the normally secreted chain is complementary to the variable domain of the immunoglobulin chain encoded by the recovered nucleic acid from the phage library.

Although the cell line used to produce the antibodies of the invention is preferably a mammalian cell line, any other suitable cell line may alternatively be used. These include, but are not limited to, microorganisms such as bacteria (e.g., E. coli and B. subtilis) transformed with recombinant bacteriophage DNA, plasmid DNA or cosmid DNA expression vectors containing antibody coding sequences; yeast (e.g., Saccharomyces Pichia) transformed with recombinant yeast expression vectors containing antibody coding sequences; insect cell systems infected with recombinant virus expression vectors (e.g., baculovirus) containing antibody coding sequences; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing antibody coding sequences; or mammalian cell systems (e.g., COS, CHO, BHK, 293, NS0, and 3T3 cells) harboring recombinant expression constructs containing promoters derived from the genome of mammalian cells (e.g., metallothionein promoter) or from mammalian viruses (e.g., the adenovirus late promoter; the vaccinia virus 7.5K promoter). Preferably, bacterial cells such as Escherichia coli, and more preferably, eukaryotic cells, especially for the expression of whole recombinant antibody molecule, are used for the expression of a recombinant antibody molecule. For example, mammalian cells such as Chinese hamster ovary cells (CHO), in conjunction with a vector such as the major intermediate early gene promoter element from human cytomegalovirus is an effective expression system for antibodies (Foecking et al., 1986, Gene 45:101; and Cockett et al., 1990, Bio/Technology 8:2).

In bacterial systems, a number of expression vectors may be advantageously selected depending upon the use intended for the antibody molecule being expressed. For example, when a large quantity of such a protein is to be produced, for the generation of pharmaceutical compositions of an antibody molecule, vectors which direct the expression of high levels of fusion protein products that are readily purified may be desirable. Such vectors include, but are not limited to, the E. coli expression vector pUR278 (Ruther et al., 1983, EMBO 12:1791), in which the antibody coding sequence may be ligated individually into the vector in frame with the lac Z coding region so that a fusion protein is produced; pIN vectors (Inouye & Inouye, 1985, Nucleic Acids Res. 13:3101-3109; Van Heeke & Schuster, 1989, J. Biol. Chem. 24:5503-5509); and the like. pGEX vectors may also be used to express foreign polypeptides as fusion proteins with glutathione 5-transferase (GST). In general, such fusion proteins are soluble and can easily be purified from lysed cells by adsorption and binding to matrix glutathione agarose beads followed by elution in the presence of free glutathione. The pGEX vectors are designed to include thrombin or factor Xa protease cleavage sites so that the cloned target can be released from the GST moiety.

In an insect system, Autographa californica nuclear polyhedrosis virus (AcNPV) is used as a vector to express foreign genes. The virus grows in Spodoptera frugiperda cells. The antibody coding sequence may be cloned individually into non-essential regions (for example the polyhedrin gene) of the virus and placed under control of an AcNPV promoter (for example the polyhedrin promoter).

In mammalian host cells, a number of viral-based expression systems may be utilized. In cases where an adenovirus is used as an expression vector, the antibody coding sequence of interest may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing the antibody molecule in infected hosts (e.g., see Logan & Shenk, 1984, Proc. Natl. Acad. Sci. USA 81:355-359). Specific initiation signals may also be required for efficient translation of inserted antibody coding sequences. These signals include the ATG initiation codon and adjacent sequences. Furthermore, the initiation codon must be in phase with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (see, e.g., Bittner et al., 1987, Methods in Enzymol. 153:516-544).

In addition, a host cell strain may be chosen which modulates the expression of the inserted sequences, or modifies and processes the nucleic acid in a specific fashion desired. Such modifications (e.g., glycosylation) and processing (e.g., cleavage) of protein products may be important for the function of the protein. Different host cells have characteristic and specific mechanisms for the post-translational processing and modification of proteins and gene products. Appropriate cell lines or host systems can be chosen to ensure the correct modification and processing of the foreign protein expressed. To this end, eukaryotic host cells which possess the cellular machinery for proper processing of the primary transcript, glycosylation, and phosphorylation of the gene product may be used. Such mammalian host cells include but are not limited to CHO, VERY, BHK, Hela, COS, MDCK, 293, 3T3, W138, BT483, Hs578T, HTB2, BT2O and T47D, NS0 (a murine myeloma cell line that does not endogenously produce any immunoglobulin chains), CRL7O3O and HsS78Bst cells.

For long-term, high-yield production of recombinant proteins, stable expression is preferred. For example, cell lines which stably express the antibody molecule may be engineered. Rather than using expression vectors which contain viral origins of replication, host cells can be transformed with DNA controlled by appropriate expression control elements (e.g., promoter, enhancer, sequences, transcription terminators, polyadenylation sites, etc.), and a selectable marker. Following the introduction of the foreign DNA, engineered cells may be allowed to grow for 1-2 days in an enriched media, and then are switched to a selective media. The selectable marker in the recombinant plasmid confers resistance to the selection and allows cells to stably integrate the plasmid into their chromosomes and grow to form foci which in turn can be cloned and expanded into cell lines. This method may advantageously be used to engineer cell lines which express the antibody molecule. Such engineered cell lines may be particularly useful in screening and evaluation of compositions that interact directly or indirectly with the antibody molecule.

A number of selection systems may be used, including but not limited to, the herpes simplex virus thymidine kinase (Wigler et al., 1977, Cell 11:223), hypoxanthineguanine phosphoribosyltransferase (Szybalska & Szybalski, 1992, Proc. Natl. Acad. Sci. USA 48:202), and adenine phosphoribosyltransferase (Lowy et al., 1980, Cell 22:8-17) genes can be employed in tk−, hgprt− or aprt− cells, respectively. Also, antimetabolite resistance can be used as the basis of selection for the following genes: dhfr, which confers resistance to methotrexate (Wigler et al., 1980, Natl. Acad. Sci. USA 77:357; O'Hare et al., 1981, Proc. Natl. Acad. Sci. USA 78:1527); gpt, which confers resistance to mycophenolic acid (Mulligan & Berg, 1981, Proc. Natl. Acad. Sci. USA 78:2072); neo, which confers resistance to the aminoglycoside G-418 (Wu and Wu, 1991, Biotherapy 3:87-95; Tolstoshev, 1993, Ann. Rev. Pharmacol. Toxicol. 32:573-596; Mulligan, 1993, Science 260:926-932; and Morgan and Anderson, 1993, Ann. Rev. Biochem. 62: 191-217; May, 1993, TIB TECH 11(5):155-215); and hygro, which confers resistance to hygromycin (Santerre et al., 1984, Gene 30:147). Methods commonly known in the art of recombinant DNA technology may be routinely applied to select the desired recombinant clone, and such methods are described, for example, in Ausubel et al. (eds.), Current Protocols in Molecular Biology, John Wiley & Sons, NY (1993); Kriegler, Gene Transfer and Expression, A Laboratory Manual, Stockton Press, NY (1990); and in Chapters 12 and 13, Dracopoli et al. (eds), Current Protocols in Human Genetics, John Wiley & Sons, NY (1994); Colberre-Garapin et al., 1981, J. Mol. Biol. 150:1, which are incorporated by reference herein in their entireties.

The expression levels of an antibody molecule can be increased by vector amplification (for a review, see Bebbington and Hentschel, The use of vectors based on gene amplification for the expression of cloned genes in mammalian cells in DNA cloning, Vol.3. (Academic Press, New York, 1987)). When a marker in the vector system expressing antibody is amplifiable, increase in the level of inhibitor present in culture of host cell will increase the number of copies of the marker gene. Since the amplified region is associated with the antibody gene, production of the antibody will also increase (Crouse et al., 1983, Mol. Cell. Biol. 3:257).

The host cell may be co-transfected with two expression vectors of the invention, the first vector encoding a heavy chain derived polypeptide and the second vector encoding a light chain derived polypeptide. The two vectors may contain identical selectable markers which enable equal expression of heavy and light chain polypeptides. Alternatively, a single vector may be used which encodes, and is capable of expressing, both heavy and light chain polypeptides. In such situations, the light chain should be placed before the heavy chain to avoid an excess of toxic free heavy chain (Proudfoot, 1986, Nature 322:52; and Kohler, 1980, Proc. Natl. Acad. Sci. USA 77:2 197). The coding sequences for the heavy and light chains may comprise cDNA or genomic DNA.

The antibodies of the invention can also be introduced into a transgenic animal (e.g., transgenic mouse). See, e.g., Bruggemann, Arch. Immunol. Ther. Exp. (Warsz). 49(3):203-8 (2001); Bruggemann and Neuberger, Immunol. Today 8:391-7 (1996), each of which is incorporated herein by reference. Transgene constructs or transloci can be obtained by, e.g., plasmid assembly, cloning in yeast artificial chromosomes, and the use of chromosome fragments. Translocus integration and maintenance in transgenic animal strains can be achieved by pronuclear DNA injection into oocytes and various transfection methods using embryonic stem cells.

For example, nucleic acids encoding humanized heavy and/or light chain or humanized heavy and/or light variable regions may be introduced randomly or by homologous recombination into mouse embryonic stem cells. The mouse heavy and light chain immunoglobulin genes may be rendered non-functional separately or simultaneously with the introduction of nucleic acids encoding humanized antibodies by homologous recombination. In particular, homozygous deletion of the JH region prevents endogenous antibody production. The modified embryonic stem cells are expanded and microinjected into blastocysts to produce chimeric mice. The chimeric mice are then be bred to produce homozygous offspring which express humanized antibodies.

Once an antibody molecule of the invention has been produced by recombinant expression, it may be purified by any method known in the art for purification of an immunoglobulin molecule, for example, by chromatography (e.g., ion exchange, affinity, particularly by affinity for the specific antigen after Protein A, and sizing column chromatography), centrifugation, differential solubility, or by any other standard technique for the purification of proteins. Further, the antibodies of the present invention or fragments thereof may be fused to heterologous polypeptide sequences described herein or otherwise known in the art to facilitate purification.

### 5.6. Antibody Conjugates

The present invention encompasses antibodies or fragments thereof that are conjugated or fused to one or more moieties, including but not limited to, peptides, polypeptides, proteins, fusion proteins, nucleic acid molecules, small molecules, mimetic agents, synthetic drugs, inorganic molecules, and organic molecules.

The present invention encompasses antibodies or fragments thereof that are recombinantly fused or chemically conjugated (including both covalent and non-covalent conjugations) to a heterologous protein or polypeptide (or fragment thereof, preferably to a polypepetide of at least 10, at least 20, at least 30, at least 40, at least 50, at least 60, at least 70, at least 80, at least 90 or at least 100 amino acids) to generate fusion proteins. The fusion does not necessarily need to be direct, but may occur through linker sequences. For example, antibodies may be used to target heterologous polypeptides to particular cell types, either in vitro or in vivo, by fusing or conjugating the antibodies to antibodies specific for particular cell surface receptors. Antibodies fused or conjugated to heterologous polypeptides may also be used in in vitro immunoassays and purification methods using methods known in the art. See e.g., International publication No. WO 93/21232; European Patent No. EP 439,095; Naramura et al., 1994, Immunol. Lett. 39:91-99; U.S. Pat. No. 5,474,981; Gillies et al., 1992, PNAS 89:1428-1432; and Fell et al., 1991, J. Immunol. 146:2446-2452, which are incorporated by reference in their entireties.

The present invention further includes compositions comprising heterologous proteins, peptides or polypeptides fused or conjugated to antibody fragments. For example, the heterologous polypeptides may be fused or conjugated to a Fab fragment, Fd fragment, Fv fragment, F(ab)₂ fragment, a VH domain, a VL domain, a VH CDR, a VL CDR, or fragment thereof. Methods for fusing or conjugating polypeptides to antibody portions are well-known in the art. See, e.g., U.S. Pat. Nos. 5,336,603, 5,622,929, 5,359,046, 5,349,053, 5,447,851, and 5,112,946; European Patent Nos. EP 307,434 and EP 367,166; International publication Nos. WO 96/04388 and WO 91/06570; Ashkenazi et al., 1991, Proc. Natl. Acad. Sci. USA 88: 10535-10539; Zheng et al., 1995, J. Immunol. 154:5590-5600; and Vil et al., 1992, Proc. Natl. Acad. Sci. USA 89:11337-11341 (said references incorporated by reference in their entireties).

Additional fusion proteins may be generated through the techniques of gene-shuffling, motif-shuffling, exon-shuffling, and/or codon-shuffling (collectively referred to as “DNA shuffling”). DNA shuffling may be employed to alter the activities of antibodies of the invention or fragments thereof (e.g., antibodies or fragments thereof with higher affinities and lower dissociation rates). See, generally, U.S. Pat. Nos. 5,605,793; 5,811,238; 5,830,721; 5,834,252; and 5,837,458, and Patten et al., 1997, Curr. Opinion Biotechnol. 8:724-33; Harayama, 1998, Trends Biotechnol. 16(2):76-82; Hansson, et al., 1999, J. Mol. Biol. 287:265-76; and Lorenzo and Blasco, 1998, Biotechniques 24(2):308-313 (each of these patents and publications are hereby incorporated by reference in its entirety). Antibodies or fragments thereof, or the encoded antibodies or fragments thereof, may be altered by being subjected to random mutagenesis by error-prone PCR, random nucleotide insertion or other methods prior to recombination. One or more portions of a polynucleotide encoding an antibody or antibody fragment may be recombined with one or more components, motifs, sections, parts, domains, fragments, etc. of one or more heterologous molecules.

Moreover, the antibodies or fragments thereof can be fused to marker sequences, such as a peptide to facilitate purification. In preferred embodiments, the marker amino acid sequence is a hexa-histidine peptide, such as the tag provided in a pQE vector (QIAGEN, Inc., 9259 Eton Avenue, Chatsworth, Calif., 91311), among others, many of which are commercially available. As described in Gentz et al., 1989, Proc. Natl. Acad. Sci. USA 86:821-824, for instance, hexa-histidine provides for convenient purification of the fusion protein. Other peptide tags useful for purification include, but are not limited to, the hemagglutinin “HA” tag, which corresponds to an epitope derived from the influenza hemagglutinin protein (Wilson et al., 1984, Cell 37:767) and the “flag” tag.

In other embodiments, antibodies of the present invention or fragments, analogs or derivatives thereof can be conjugated to a diagnostic or detectable agent. Such antibodies can be useful for monitoring or prognosing the development or progression of a disorder as part of a clinical testing procedure, such as determining the efficacy of a particular therapy. Such diagnosis and detection can be accomplished by coupling the antibody to detectable substances including, but not limited to various enzymes, such as but not limited to horseradish peroxidase, alkaline phosphatase, beta-galactosidase, or acetylcholinesterase; prosthetic groups, such as but not limited to streptavidin/biotin and avidin/biotin; fluorescent materials, such as but not limited to, umbelliferone, fluorescein, fluorescein isothiocynate, rhodamine, dichlorotriazinylamine fluorescein, dansyl chloride or phycoerythrin; luminescent materials, such as but not limited to, luminol; bioluminescent materials, such as but not limited to, luciferase, luciferin, and aequorin; radioactive materials, such as but not limited to iodine (¹³¹I, ¹²⁵I, ¹²³I, ¹²¹I,), carbon (¹⁴C), sulfur (³⁵S), tritium (³H), indium (¹¹⁵In, ¹¹³In, ¹¹²In, ¹¹¹In,), and technetium (⁹⁹Tc), thallium (²⁰¹Ti), gallium (⁶⁸Ga, ⁶⁷Ga), palladium (¹⁰³Pd), molybdenum (⁹⁹Mo), xenon (¹³³Xe), fluorine (¹⁸F), ¹⁵³Sm, ¹⁷⁷Lu, ¹⁵⁹Gd, ¹⁴⁹ Pm, ¹⁴⁰La, ¹⁷⁵Yb, ¹⁶⁶Ho, ⁹⁰Y, ⁴⁷Sc, ¹⁸⁶Re, ¹⁸⁸Re, ¹⁴²Pr, ¹⁰⁵Rh, ⁹⁷Ru, ⁶⁸Ge, ⁵⁷Co, ⁶⁵Zn, ⁸⁵Sr, ³²P, ¹⁵³Gd, 169Yb, ⁵¹Cr, ⁵⁴Mn, ⁷⁵Se, ¹¹³Sn, and ¹¹⁷Tin; positron emitting metals using various positron emission tomographies, noradioactive paramagnetic metal ions, and molecules that are radiolabelled or conjugated to specific radioisotopes.

The present invention further encompasses antibodies or fragments thereof that are conjugated to a therapeutic moiety. An antibody or fragment thereof may be conjugated to a therapeutic moiety such as a cytotoxin, e.g., a cytostatic or cytocidal agent, a therapeutic agent or a radioactive metal ion, e.g., alpha-emitters. A cytotoxin or cytotoxic agent includes any agent that is detrimental to cells. Therapeutic moieties include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BCNU) and lomustine (CCNU), cyclothosphamide, busulfan, dibromomannitol, streptozotocin, mitomycin C, and cisdichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), Auristatin molecules (e.g., auristatin PHE, bryostatin 1, and solastatin 10; see Woyke et al., Antimicrob. Agents Chemother. 46:3802-8 (2002), Woyke et al., Antimicrob. Agents Chemother. 45:3580-4 (2001), Mohammad et al., Anticancer Drugs 12:735-40 (2001), Wall et al., Biochem. Biophys. Res. Commun. 266:76-80 (1999), Mohammad et al., Int. J. Oncol. 15:367-72 (1999), all of which are incorporated herein by reference), hormones (e.g., glucocorticoids, progestins, androgens, and estrogens), DNA-repair enzyme inhibitors (e.g., etoposide or topotecan), kinase inhibitors (e.g., compound ST1571, imatinib mesylate (Kantaijian et al., Clin Cancer Res. 8(7):2167-76 (2002)), cytotoxic agents (e.g., paclitaxel, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, procaine, tetracaine, lidocaine, propranolol, and puromycin and analogs or homologs thereof) and those compounds disclosed in U.S. Pat. Nos. 6,245,759, 6,399,633, 6,383,790, 6,335,156, 6,271,242, 6,242,196, 6,218,410, 6,218,372, 6,057,300, 6,034,053, 5,985,877, 5,958,769, 5,925,376, 5,922,844, 5,911,995, 5,872,223, 5,863,904, 5,840,745, 5,728,868, 5,648,239, 5,587,459), farnesyl transferase inhibitors (e.g., R115777, BMS-214662, and those disclosed by, for example, U.S. Pat. Nos. 6,458,935, 6,451,812, 6,440,974, 6,436,960, 6,432,959, 6,420,387, 6,414,145, 6,410,541, 6,410,539, 6,403,581, 6,399,615, 6,387,905, 6,372,747, 6,369,034, 6,362,188, 6,342,765, 6,342,487, 6,300,501, 6,268,363, 6,265,422, 6,248,756, 6,239,140, 6,232,338, 6,228,865, 6,228,856, 6,225,322, 6,218,406, 6,211,193, 6,187,786, 6,169,096, 6,159,984, 6,143,766, 6,133,303, 6,127,366, 6,124,465, 6,124,295, 6,103,723, 6,093,737, 6,090,948, 6,080,870, 6,077,853, 6,071,935, 6,066,738, 6,063,930, 6,054,466, 6,051,582, 6,051,574, and 6,040,305), topoisomerase inhibitors (e.g., camptothecin; irinotecan; SN-38; topotecan; 9-aminocamptothecin; GG-211 (GI 147211); DX-895 If; IST-622; rubitecan; pyrazoloacridine; XR-5000; saintopin; UCE6; UCE1022; TAN-1518A; TAN-1518B; KT6006; KT6528; ED-110; NB-506; ED-110; NB-506; and rebeccamycin); bulgarein; DNA minor groove binders such as Hoescht dye 33342 and Hoechst dye 33258; nitidine; fagaronine; epiberberine; coralyne; beta-lapachone; BC-4-1; bisphosphonates (e.g., alendronate, cimadronte, clodronate, tiludronate, etidronate, ibandronate, neridronate, olpandronate, risedronate, piridronate, pamidronate, zolendronate) HMG-CoA reductase inhibitors, (e.g., lovastatin, simvastatin, atorvastatin, pravastatin, fluvastatin, statin, cerivastatin, lescol, lupitor, rosuvastatin and atorvastatin) and pharmaceutically acceptable salts, solvates, clathrates, and prodrugs thereof. See, e.g., Rothenberg, M. L., Annals of Oncology 8:837-855(1997); and Moreau, P., et al., J. Med. Chem. 41:1631-1640(1998)), antisense oligonucleotides (e.g., those disclosed in the U.S. Pat. Nos. 6,277,832, 5,998,596, 5,885,834, 5,734,033, and 5,618,709), immunomodulators (e.g., antibodies and cytokines), antibodies, and adenosine deaminase inhibitors (e.g., Fludarabine phosphate and 2-Chlorodeoxyadenosine).

Further, an antibody or fragment thereof may be conjugated to a therapeutic moiety or drug moiety that modifies a given biological response. Therapeutic moieties or drug moieties are not to be construed as limited to classical chemical therapeutic agents. For example, the drug moiety may be a protein or polypeptide possessing a desired biological activity. Such proteins may include, for example, a toxin such as abrin, ricin A, pseudomonas exotoxin, cholera toxin, or diphtheria toxin; a protein such as tumor necrosis factor, α-interferon, β-interferon, nerve growth factor, platelet derived growth factor, tissue plasminogen activator, an apoptotic agent, e.g., TNF-α, TNF-β, AIM I (see, International publication No. WO 97/33899), AIM II (see, International Publication No. WO 97/34911), Fas Ligand (Takahashi et al., 1994, J. Immunol., 6:1567-1574), and VEGI (see, International publication No. WO 99/23105), a thrombotic agent or an anti-angiogenic agent, e.g., angiostatin, endostatin or a component of the coagulation pathway (e.g., tissue factor); or, a biological response modifier such as, for example, a lymphokine (e.g., interleukin-1 (“IL-1”), interleukin-2 (“IL-2”), interleukin-6 (“IL-6”), granulocyte macrophage colony stimulating factor (“GM-CSF”), and granulocyte colony stimulating factor (“G-CSF”)), a growth factor (e.g., growth hormone (“GH”)), or a coagulation agent (e.g., calcium, vitamin K, tissue factors, such as but not limited to, Hageman factor (factor XII), high-molecular-weight kininogen (HMWK), prekallikrein (PK), coagulation proteins-factors II (prothrombin), factor V, XIIa, VIII, XIIIa, XI, XIa, IX, IXa, X, phospholipid. fibrinopeptides A and B from the α and β chains of fibrinogen, fibrin monomer).

Moreover, an antibody can be conjugated to therapeutic moieties such as a radioactive metal ion, such as alph-emiters such as ²¹³Bi or macrocyclic chelators useful for conjugating radiometal ions, including but not limited to, ¹³¹In, ¹³¹LU, ¹³¹Y, ¹³¹Ho, ¹³¹Sm, to polypeptides. In certain embodiments, the macrocyclic chelator is 1,4,7,10-tetraazacyclododecane-N,N′,N″,N′″-tetraacetic acid (DOTA) which can be attached to the antibody via a linker molecule. Such linker molecules are commonly known in the art and described in Denardo et al., 1998, Clin Cancer Res. 4(10):2483-90; Peterson et al., 1999, Bioconjug. Chem. 10(4):553-7; and Zimmerman et al., 1999, Nucl. Med. Biol. 26(8):943-50, each incorporated by reference in their entireties.

Techniques for conjugating therapeutic moieties to antibodies are well known, see, e.g., Arnon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies 84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985), and Thorpe et al., 1982, Immunol. Rev. 62:119-58.

Alternatively, an antibody can be conjugated to a second antibody to form an antibody heteroconjugate as described by Segal in U.S. Pat. No. 4,676,980, which is incorporated herein by reference in its entirety.

The therapeutic moiety or drug conjugated to an antibody or fragment thereof should be chosen to achieve the desired prophylactic or therapeutic effect(s) for a particular disorder in a subject. A clinician or other medical personnel should consider the following when deciding on which therapeutic moiety or drug to conjugate to an antibody or fragment thereof: the nature of the disease, the severity of the disease, and the condition of the subject.

Antibodies may also be attached to solid supports, which are particularly useful for immunoassays or purification of the target antigen. Such solid supports include, but are not limited to, glass, cellulose, polyacrylamide, nylon, polystyrene, polyvinyl chloride or polypropylene.

### 5.7. Uses of the Compositions of the Invention

The present invention provides methods of efficiently humanizing an antibody of interest. The humanized antibodies of the present invention can be used alone or in combination with other prophylactic or therapeutic agents for treating, managing, preventing or ameliorating a disorder or one or more symptoms thereof.

The present invention provides methods for preventing, managing, treating, or ameliorating a disorder comprising administering to a subject in need thereof one or more antibodies of the invention alone or in combination with one or more therapies (e.g., one or more prophylactic or therapeutic agents) other than an antibody of the invention. The present invention also provides compositions comprising one or more antibodies of the invetnion and one or more prophylactic or therapeutic agents other than antibodies of the invention and methods of preventing, managing, treating, or ameliorating a disorder or one or more symptoms thereof utilizing said compositions. Therapeutic or prophylactic agents include, but are not limited to, small molecules, synthetic drugs, peptides, polypeptides, proteins, nucleic acids (e.g., DNA and RNA nucleotides including, but not limited to, antisense nucleotide sequences, triple helices, RNAi, and nucleotide sequences encoding biologically active proteins, polypeptides or peptides) antibodies, synthetic or natural inorganic molecules, mimetic agents, and synthetic or natural organic molecules.

Any therapy which is known to be useful, or which has been used or is currently being used for the prevention, management, treatment, or amelioration of a disorder or one or more symptoms thereof can be used in combination with an antibody of the invention in accordance with the invention described herein. See, e.g., Gilman et al., Goodman and Gilman's: The Pharmacological Basis of Therapeutics, 10th ed., McGraw-Hill, New York, 2001; The Merck Manual of Diagnosis and Therapy, Berkow, M. D. et al. (eds.), 17th Ed., Merck Sharp & Dohme Research Laboratories, Rahway, N.J., 1999; Cecil Textbook of Medicine, 20th Ed., Bennett and Plum (eds.), W. B. Saunders, Philadelphia, 1996 for information regarding therapies (e.g., prophylactic or therapeutic agents) which have been or are currently being used for preventing, treating, managing, or ameliorating a disorder or one or more symptoms thereof. Examples of such agents include, but are not limited to, immunomodulatory agents, anti-inflammatory agents (e.g., adrenocorticoids, corticosteroids (e.g., beclomethasone, budesonide, flunisolide, fluticasone, triamcinolone, methlyprednisolone, prednisolone, prednisone, hydrocortisone), glucocorticoids, steroids, non-steriodal anti-inflammatory drugs (e.g., aspirin, ibuprofen, diclofenac, and COX-2 inhibitors), anti-cancer agents, pain relievers, leukotreine antagonists (e.g., montelukast, methyl xanthines, zafirlukast, and zileuton), beta2-agonists (e.g., albuterol, biterol, fenoterol, isoetharie, metaproterenol, pirbuterol, salbutamol, terbutalin formoterol, salmeterol, and salbutamol terbutaline), anticholinergic agents (e.g., ipratropium bromide and oxitropium bromide), sulphasalazine, penicillamine, dapsone, antihistamines, anti-malarial agents (e.g., hydroxychloroquine), anti-viral agents, and antibiotics (e.g., dactinomycin (formerly actinomycin), bleomycin, erythomycin, penicillin, mithramycin, and anthramycin (AMC)).

In a specific embodiment, the present invention provides administering one or more humanized anti-IL-9 antibodies to a subject, preferably a human subject, for preventing, treating, managing, or ameliorating a respiratory condition or one or more symptoms thereof. In one embodiment, the invention encompasses a method of preventing, treating, managing, or ameliorating a respiratory disorder or one or more symptoms thereof (e.g., an allergy, wheezing, and asthma), said method comprising administering to a subject in need thereof a dose of a prophylactically or therapeutically effective amount of one or more humanized anti-IL-9 antibodies. In another embodiment, the invention provides a method of preventing, treating, managing, or ameliorating a respiratory infection or one or more symptoms thereof, said method comprising administering a prophylactically or therapeutic effective amount of one or more humanized anti-IL-9 antibodies.

In a specific embodiment, the present invention provides administering one or more humanized anti-EphA2 antibodies to a subject, preferably a human subject, for preventing, treating, managing, or ameliorating a hyperproliferative cell disease or one or more symptoms thereof. In one embodiment, one or more humanized anti-EphA2 antibodies are administered alone or in combination with other agents to a subject to prevent, treat, manage, or ameliorate cancer or one or more symptoms thereof (see, e.g., U.S. application Ser. No. 10/436,782, which is incorporated herein by reference in its entirety). In another embodiment, one or more humanized anti-EphA2 antibodies are administered alone or in combination with other agents to a subject to prevent, treat, manage, or ameliorate a disorder involving non-neoplastic hyperproliferative cells, in particular hyperproliferative epithlial and endothelial cells, or one or symptoms thereof (see e.g., U.S. Application Ser. No. 60/462,024, which is incorporated herein by reference in its entirety). In yet another embodiment, one or more humanized anti-EphA2 antibodies are used for diagnostic or screening purposes.

The humanized antibodies of the invention can be used directly against a particular antigen. In some embodiments, antibodies of the invention belong to a subclass or isotype that is capable of mediating the lysis of cells to which the antibody binds. In a specific embodiment, the antibodies of the invention belong to a subclass or isotype that, upon complexing with cell surface proteins, activates serum complement and/or mediates antibody dependent cellular cytotoxicity (ADCC) by activating effector cells such as natural killer cells or macrophages.

The biological activities of antibodies are known to be determined, to a large extent, by the constant domains or Fc region of the antibody molecule (Uananue and Benacerraf, Textbook of Immunology, 2nd Edition, Williams & Wilkins, p. 218 (1984)). This includes their ability to activate complement and to mediate antibody-dependent cellular cytotoxicity (ADCC) as effected by leukocytes. Antibodies of different classes and subclasses differ in this respect, as do antibodies from the same subclass but different species; according to the present invention, antibodies of those classes having the desired biological activity are prepared. Preparation of these antibodies involves the selection of antibody constant domains and their incorporation in the humanized antibody by known technique. For example, mouse immunoglobulins of the IgG3 and IgG2a class are capable of activating serum complement upon binding to the target cells which express the cognate antigen, and therefore humanized antibodies which incorporate IgG3 and IgG2a effector functions are desirable for certain therapeutic applications.

In general, mouse antibodies of the IgG₂ₐ and IgG₃ subclass and occasionally IgG₁ can mediate ADCC, and antibodies of the IgG₃, IgG₂ₐ, and IgM subclasses bind and activate serum complement. Complement activation generally requires the binding of at least two IgG molecules in close proximity on the target cell. However, the binding of only one IgM molecule activates serum complement.

The ability of any particular antibody to mediate lysis of the target cell by complement activation and/or ADCC can be assayed. The cells of interest are grown and labeled in vitro; the antibody is added to the cell culture in combination with either serum complement or immune cells which may be activated by the antigen antibody complexes. Cytolysis of the target cells is detected by the release of label from the lysed cells. In fact, antibodies can be screened using the patient's own serum as a source of complement and/or immune cells. The antibody that is capable of activating complement or mediating ADCC in the in vitro test can then be used therapeutically in that particular patient.

Use of IgM antibodies may be preferred for certain applications, however IgG molecules by being smaller may be more able than IgM molecules to localize to certain types of infected cells.

In some embodiments, the antibodies of this invention are useful in passively immunizing patients.

The antibodies of the invention can also be used in diagnostic assays either in vivo or in vitro for detection/identification of the expression of an antigen in a subject or a biological sample (e.g., cells or tissues). Non-limiting examples of using an antibody, a fragment thereof, or a composition comprising an antibody or a fragment thereof in a diagnostic assay are given in U.S. Pat. Nos. 6,392,020; 6,156,498; 6,136,526; 6,048,528; 6,015,555; 5,833,988; 5,811,310; 8 5,652,114; 5,604,126; 5,484,704; 5,346,687; 5,318,892; 5,273,743; 5,182,107; 5,122,447; 5,080,883; 5,057,313; 4,910,133; 4,816,402; 4,742,000; 4,724,213; 4,724,212; 4,624,846; 4,623,627; 4,618,486; 4,176,174 (all of which are incorporated herein by reference). Suitable diagnostic assays for the antigen and its antibodies depend on the particular antibody used. Non-limiting examples are an ELISA, sandwich assay, and steric inhibition assays. For in vivo diagnostic assays using the antibodies of the invention, the antibodies may be conjugated to a label that can be detected by imaging techniques, such as X-ray, computed tomography (CT), ultrasound, or magnetic resonance imaging (MRI). The antibodies of the invention can also be used for the affinity purification of the antigen from recombinant cell culture or natural sources.

### 5.8. Administration and Formulations

The invention provides for compositions comprising antibodies of the invention for use in diagnosing, detecting, or monitoring a disorder, in preventing, treating, managing, or ameliorating of a disorder or one or more symptoms thereof, and/or in research. In a specific embodiment, a composition comprises one or more antibodies of the invention. In another embodiment, a composition comprises one or more antibodies of the invention and one or more prophylactic or therapeutic agents other than antibodies of the invention. Preferably, the prophylactic or therapeutic agents known to be useful for or having been or currently being used in the prevention, treatment, management, or amelioration of a disorder or one or more symptoms thereof. In accordance with these embodiments, the composition may further comprise of a carrier, diluent or excipient.

The compositions of the invention include, but are not limited to, bulk drug compositions useful in the manufacture of pharmaceutical compositions (e.g., impure or non-sterile compositions) and pharmaceutical compositions (i.e., compositions that are suitable for administration to a subject or patient) which can be used in the preparation of unit dosage forms. Such compositions comprise a prophylactically or therapeutically effective amount of a prophylactic and/or therapeutic agent disclosed herein or a combination of those agents and a pharmaceutically acceptable carrier. Preferably, compositions of the invention are pharmaceutical compositions and comprise an effective amount of one or more antibodies of the invention, a pharmaceutically acceptable carrier, and, optionally, an effective amount of another prophylactic or therapeutic agent.

The pharmaceutical composition can be formulated as an oral or non-oral dosage form, for immediate or extended release. The composition can comprise inactive ingredients ordinarily used in pharmaceutical preparation such as diluents, fillers, disintegrants, sweeteners, lubricants and flavors. The pharmaceutical composition is preferably formulated for intravenous administration, either by bolus injection or sustained drip, or for release from an implanted capsule. A typical formulation for intravenous administration utilizes physiological saline as a diluent.

Fab or Fab′ portions of the antibodies of the invention can also be utilized as the therapeutic active ingredient. Preparation of these antibody fragments is well-known in the art.

The composition of the present invention can also include printed matter that describes clinical indications for which the antibodies can be administered as a therapeutic agent, dosage amounts and schedules, and/or contraindications for administration of the antibodies of the invention to a patient.

The compositions of the invention include, but are not limited to, bulk drug compositions useful in the manufacture of pharmaceutical compositions (e.g., impure or non-sterile compositions) and pharmaceutical compositions (i.e., compositions that are suitable for administration to a subject or patient) which can be used in the preparation of unit dosage forms. Such compositions comprise a prophylactically or therapeutically effective amount of a prophylactic and/or therapeutic agent disclosed herein or a combination of those agents and a pharmaceutically acceptable carrier. Preferably, compositions of the invention are pharmaceutical compositions and comprise an effective amount of one or more antibodies of the invention, a pharmaceutically acceptable carrier, and, optionally, an effective amount of another prophylactic or therapeutic agent.

In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant (e.g., Freund's adjuvant (complete and incomplete)), excipient, or vehicle with which the therapeutic is contained in or administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like.

Generally, the ingredients of compositions of the invention are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The compositions of the invention can be formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

Various delivery systems are known and can be used to administer one or more antibodies of the invention or the combination of one or more antibodies of the invention and a prophylactic agent or therapeutic agent useful for preventing, managing, treating, or ameliorating a disorder or one or more symptoms thereof, e.g., encapsulation in liposomes, microparticles, microcapsules, recombinant cells capable of expressing the antibody or antibody fragment, receptor-mediated endocytosis (see, e.g., Wu and Wu, J. Biol. Chem. 262:4429-4432 (1987)), construction of a nucleic acid as part of a retroviral or other vector, etc. Methods of administering a prophylactic or therapeutic agent of the invention include, but are not limited to, parenteral administration (e.g., intradermal, intramuscular, intraperitoneal, intravenous and subcutaneous), epidurala administration, intratumoral administration, and mucosal adminsitration (e.g., intranasal and oral routes). In addition, pulmonary administration can be employed, e.g., by use of an inhaler or nebulizer, and formulation with an aerosolizing agent. See, e.g., U.S. Pat. Nos. 6,019,968, 5,985, 320, 5,985,309, 5,934,272, 5,874,064, 5,855,913, 5,290,540, and 4,880,078; and PCT Publication Nos. WO 92/19244, WO 97/32572, WO 97/44013, WO 98/31346, and WO 99/66903, each of which is incorporated herein by reference their entireties. In one embodiment, an antibody of the invention, combination therapy, or a composition of the invention is administered using Alkermes AIR™ pulmonary drug delivery technology (Alkermes, Inc., Cambridge, Mass.). In a specific embodiment, prophylactic or therapeutic agents of the invention are administered intramuscularly, intravenously, intratumorally, orally, intranasally, pulmonary, or subcutaneously. The prophylactic or therapeutic agents may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local.

In a specific embodiment, it may be desirable to administer the prophylactic or therapeutic agents of the invention locally to the area in need of treatment; this may be achieved by, for example, and not by way of limitation, local infusion, by injection, or by means of an implant, said implant being of a porous or non-porous material, including membranes and matrices, such as sialastic membranes, polymers, fibrous matrices (e.g., Tissuel®), or collagen matrices. In one embodiment, an effective amount of one or more antibodies of the invention antagonists is administered locally to the affected area to a subject to prevent, treat, manage, and/or ameliorate a disorder or a symptom thereof. In another embodiment, an effective amount of one or more antibodies of the invention is administered locally to the affected area in combination with an effective amount of one or more therapies (e.g., one or more prophylactic or therapeutic agents) other than an antibody of the invention of a subject to prevent, treat, manage, and/or ameliorate a disorder or one or more symptoms thereof.

In another embodiment, the prophylactic or therapeutic agent can be delivered in a controlled release or sustained release system. In one embodiment, a pump may be used to achieve controlled or sustained release (see Langer, supra; Sefton, 1987, CRC Crit. Ref. Biomed. Eng. 14:20; Buchwald et al., 1980, Surgery 88:507; Saudek et al., 1989, N. Engl. J. Med. 321:574). In another embodiment, polymeric materials can be used to achieve controlled or sustained release of the therapies of the invention (see e.g., Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas, 1983, J., Macromol. Sci. Rev. Macromol. Chem. 23:61; see also Levy et al., 1985, Science 228:190; During et al., 1989, Ann. Neurol. 25:351; Howard et al., 1989, J. Neurosurg. 7 1:105); U.S. Pat. No. 5,679,377; U.S. Pat. No. 5,916,597; U.S. Pat. No. 5,912,015; U.S. Pat. No. 5,989,463; U.S. Pat. No. 5,128,326; PCT Publication No. WO 99/15154; and PCT Publication No. WO 99/20253. Examples of polymers used in sustained release formulations include, but are not limited to, poly(2-hydroxy ethyl methacrylate), poly(methyl methacrylate), poly(acrylic acid), poly(ethylene-co-vinyl acetate), poly(methacrylic acid), polyglycolides (PLG), polyanhydrides, poly(N-vinyl pyrrolidone), poly(vinyl alcohol), polyacrylamide, poly(ethylene glycol), polylactides (PLA), poly(lactide-co-glycolides) (PLGA), and polyorthoesters. In a preferred embodiment, the polymer used in a sustained release formulation is inert, free of leachable impurities, stable on storage, sterile, and biodegradable. In yet another embodiment, a controlled or sustained release system can be placed in proximity of the prophylactic or therapeutic target, thus requiring only a fraction of the systemic dose (see, e.g., Goodson, in Medical Applications of Controlled Release, supra, vol. 2, pp. 115-138 (1984)).

Controlled release systems are discussed in the review by Langer (1990, Science 249:1527-1533). Any technique known to one of skill in the art can be used to produce sustained release formulations comprising one or more therapeutic agents of the invention. See, e.g., U.S. Pat. No. 4,526,938, PCT publication WO 91/05548, PCT publication WO 96/20698, Ning et al., 1996, “Intratumoral Radioimmunotheraphy of a Human Colon Cancer Xenograft Using a Sustained-Release Gel,” Radiotherapy & Oncology 39:179-189, Song et al., 1995, “Antibody Mediated Lung Targeting of Long-Circulating Emulsions,” PDA Journal of Pharmaceutical Science & Technology 50:372-397, Cleek et al., 1997, “Biodegradable Polymeric Carriers for a bFGF Antibody for Cardiovascular Application,” Pro. Int'l. Symp. Control. Rel. Bioact. Mater. 24:853-854, and Lam et al., 1997, “Microencapsulation of Recombinant Humanized Monoclonal Antibody for Local Delivery,” Proc. Int'l. Symp. Control Rel. Bioact. Mater. 24:759-760, each of which is incorporated herein by reference in their entireties.

In a specific embodiment, where the composition of the invention is a nucleic acid encoding a prophylactic or therapeutic agent, the nucleic acid can be administered in vivo to promote expression of its encoded prophylactic or therapeutic agent, by constructing it as part of an appropriate nucleic acid expression vector and administering it so that it becomes intracellular, e.g., by use of a retroviral vector (see U.S. Pat. No. 4,980,286), or by direct injection, or by use of microparticle bombardment (e.g., a gene gun; Biolistic, Dupont), or coating with lipids or cell-surface receptors or transfecting agents, or by administering it in linkage to a homeobox-like peptide which is known to enter the nucleus (see, e.g., Joliot et al., 1991, Proc. Natl. Acad. Sci. USA 88:1864-1868). Alternatively, a nucleic acid can be introduced intracellularly and incorporated within host cell DNA for expression by homologous recombination.

A pharmaceutical composition of the invention is formulated to be compatible with its intended route of administration. Examples of routes of administration include, but are not limited to, parenteral, e.g., intravenous, intradermal, subcutaneous, oral, intranasal (e.g., inhalation), transdermal (e.g., topical), transmucosal, and rectal administration. In a specific embodiment, the composition is formulated in accordance with routine procedures as a pharmaceutical composition adapted for intravenous, subcutaneous, intramuscular, oral, intranasal, or topical administration to human beings. Typically, compositions for intravenous administration are solutions in sterile isotonic aqueous buffer. Where necessary, the composition may also include a solubilizing agent and a local anesthetic such as lignocamne to ease pain at the site of the injection.

If the compositions of the invention are to be administered topically, the compositions can be formulated in the form of an ointment, cream, transdermal patch, lotion, gel, shampoo, spray, aerosol, solution, emulsion, or other form well-known to one of skill in the art. See, e.g., Remington's Pharmaceutical Sciences and Introduction to Pharmaceutical Dosage Forms, 19th ed., Mack Pub. Co., Easton, Pa. (1995). For non-sprayable topical dosage forms, viscous to semi-solid or solid forms comprising a carrier or one or more excipients compatible with topical application and having a dynamic viscosity preferably greater than water are typically employed. Suitable formulations include, without limitation, solutions, suspensions, emulsions, creams, ointments, powders, liniments, salves, and the like, which are, if desired, sterilized or mixed with auxiliary agents (e.g., preservatives, stabilizers, wetting agents, buffers, or salts) for influencing various properties, such as, for example, osmotic pressure. Other suitable topical dosage forms include sprayable aerosol preparations wherein the active ingredient, preferably in combination with a solid or liquid inert carrier, is packaged in a mixture with a pressurized volatile (e.g., a gaseous propellant, such as freon) or in a squeeze bottle. Moisturizers or humectants can also be added to pharmaceutical compositions and dosage forms if desired. Examples of such additional ingredients are well-known in the art.

If the method of the invention comprises intranasal administration of a composition, the composition can be formulated in an aerosol form, spray, mist or in the form of drops. In particular, prophylactic or therapeutic agents for use according to the present invention can be conveniently delivered in the form of an aerosol spray presentation from pressurized packs or a nebuliser, with the use of a suitable propellant (e.g., dichlorodifluoromethane, trichlorofluoromethane, dichlorotetrafluoroethane, carbon dioxide or other suitable gas). In the case of a pressurized aerosol the dosage unit may be determined by providing a valve to deliver a metered amount. Capsules and cartridges (composed of, e.g., gelatin) for use in an inhaler or insufflator may be formulated containing a powder mix of the compound and a suitable powder base such as lactose or starch.

If the method of the invention comprises oral administration, compositions can be formulated orally in the form of tablets, capsules, cachets, gelcaps, solutions, suspensions, and the like. Tablets or capsules can be prepared by conventional means with pharmaceutically acceptable excipients such as binding agents (e.g., pregelatinised maize starch, polyvinylpyrrolidone, or hydroxypropyl methylcellulose); fillers (e.g., lactose, microcrystalline cellulose, or calcium hydrogen phosphate); lubricants (e.g., magnesium stearate, talc, or silica); disintegrants (e.g., potato starch or sodium starch glycolate); or wetting agents (e.g., sodium lauryl sulphate). The tablets may be coated by methods well-known in the art. Liquid preparations for oral administration may take the form of, but not limited to, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives, or hydrogenated edible fats); emulsifying agents (e.g., lecithin or acacia); non-aqueous vehicles (e.g., almond oil, oily esters, ethyl alcohol, or fractionated vegetable oils); and preservatives (e.g., methyl or propyl-p-hydroxybenzoates or sorbic acid). The preparations may also contain buffer salts, flavoring, coloring, and sweetening agents as appropriate. Preparations for oral administration may be suitably formulated for slow release, controlled release, or sustained release of a prophylactic or therapeutic agent(s).

The method of the invention may comprise pulmonary administration, e.g., by use of an inhaler or nebulizer, of a composition formulated with an aerosolizing agent. See, e.g., U.S. Pat. Nos. 6,019,968, 5,985, 320, 5,985,309, 5,934,272, 5,874,064, 5,855,913, 5,290,540, and 4,880,078; and PCT Publication Nos. WO 92/19244, WO 97/32572, WO 97/44013, WO 98/31346, and WO 99/66903, each of which is incorporated herein by reference their entireties. In a specific embodiment, an antibody of the invention, combination therapy, and/or composition of the invention is administered using Alkermes AIR™ pulmonary drug delivery technology (Alkermes, Inc., Cambridge, Mass.).

The method of the invention may comprise administration of a composition formulated for parenteral administration by injection (e.g., by bolus injection or continuous infusion). Formulations for injection may be presented in unit dosage form (e.g., in ampoules or in multi-dose containers) with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle (e.g., sterile pyrogen-free water) before use.

The methods of the invention may additionally comprise of administration of compositions formulated as depot preparations. Such long acting formulations may be administered by implantation (e.g., subcutaneously or intramuscularly) or by intramuscular injection. Thus, for example, the compositions may be formulated with suitable polymeric or hydrophobic materials (e.g., as an emulsion in an acceptable oil) or ion exchange resins, or as sparingly soluble derivatives (e.g., as a sparingly soluble salt).

The methods of the invention encompasses administration of compositions formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

Generally, the ingredients of compositions are supplied either separately or mixed together in unit dosage form, for example, as a dry lyophilized powder or water free concentrate in a hermetically sealed container such as an ampoule or sachette indicating the quantity of active agent. Where the mode of administration is infusion, composition can be dispensed with an infusion bottle containing sterile pharmaceutical grade water or saline. Where the mode of administration is by injection, an ampoule of sterile water for injection or saline can be provided so that the ingredients may be mixed prior to administration.

In particular, the invention also provides that one or more of the prophylactic or therapeutic agents, or pharmaceutical compositions of the invention is packaged in a hermetically sealed container such as an ampoule or sachette indicating the quantity of the agent. In one embodiment, one or more of the prophylactic or therapeutic agents, or pharmaceutical compositions of the invention is supplied as a dry sterilized lyophilized powder or water free concentrate in a hermetically sealed container and can be reconstituted (e.g., with water or saline) to the appropriate concentration for administration to a subject. Preferably, one or more of the prophylactic or therapeutic agents or pharmaceutical compositions of the invention is supplied as a dry sterile lyophilized powder in a hermetically sealed container at a unit dosage of at least 5 mg, more preferably at least 10 mg, at least 15 mg, at least 25 mg, at least 35 mg, at least 45 mg, at least 50 mg, at least 75 mg, or at least 100 mg. The lyophilized prophylactic or therapeutic agents or pharmaceutical compositions of the invention should be stored at between 2° C. and 8° C. in its original container and the prophylactic or therapeutic agents, or pharmaceutical compositions of the invention should be administered within 1 week, preferably within 5 days, within 72 hours, within 48 hours, within 24 hours, within 12 hours, within 6 hours, within 5 hours, within 3 hours, or within 1 hour after being reconstituted. In an alternative embodiment, one or more of the prophylactic or therapeutic agents or pharmaceutical compositions of the invention is supplied in liquid form in a hermetically sealed container indicating the quantity and concentration of the agent. Preferably, the liquid form of the administered composition is supplied in a hermetically sealed container at least 0.25 mg/ml, more preferably at least 0.5 mg/ml, at least 1 mg/ml, at least 2.5 mg/ml, at least 5 mg/ml, at least 8 mg/ml, at least 10 mg/ml, at least 15 mg/kg, at least 25 mg/ml, at least 50 mg/ml, at least 75 mg/ml or at least 100 mg/ml. The liquid form should be stored at between 2° C. and 8° C. in its original container.

Generally, the ingredients of the compositions of the invention are derived from a subject that is the same species origin or species reactivity as recipient of such compositions. Thus, in a preferred embodiment, human or humanized antibodies are administered to a human patient for therapy or prophylaxis.

### 5.8.1. Gene Therapy

In a specific embodiment, nucleic acid sequences comprising nucleotide sequences encoding an antibody of the invention or another prophylactic or therapeutic agent of the invention are administered to treat, prevent, manage, or ameliorate a disorder or one or more symptoms thereof by way of gene therapy. Gene therapy refers to therapy performed by the administration to a subject of an expressed or expressible nucleic acid. In this embodiment of the invention, the nucleic acids produce their encoded antibody or prophylactic or therapeutic agent of the invention that mediates a prophylactic or therapeutic effect.

Any of the methods for gene therapy available in the art can be used according to the present invention. For general reviews of the methods of gene therapy, see Goldspiel et al., 1993, Clinical Pharmacy 12:488-505; Wu and Wu, 1991, Biotherapy 3:87-95; Tolstoshev, 1993, Ann. Rev. Pharmacol. Toxicol. 32:573-596; Mulligan, Science 260:926-932 (1993); and Morgan and Anderson, 1993, Ann. Rev. Biochem. 62:191-217; May, 1993, TIBTECH 11(5):155-215. Methods commonly known in the art of recombinant DNA technology which can be used are described in Ausubel et al. (eds.), Current Protocols in Molecular Biology, John Wiley & Sons, NY (1993); and Kriegler, Gene Transfer and Expression, A Laboratory Manual, Stockton Press, NY (1990).

In one embodiment, the method of the invention comprises administration of a composition comprising nucleic acids encoding antibodies or another prophylactic or therapeutic agent of the invention, said nucleic acids being part of an expression vector that expresses the antibody, another prophylactic or therapeutic agent of the invention, or fragments or chimeric proteins or heavy or light chains thereof in a suitable host. In particular, such nucleic acids have promoters, preferably heterologous promoters, operably linked to the antibody coding region, said promoter being inducible or constitutive, and, optionally, tissue-specific. In another embodiment, nucleic acid molecules are used in which the coding sequences of an antibody or another prophylactic or therapeutic agent of the invention and any other desired sequences are flanked by regions that promote homologous recombination at a desired site in the genome, thus providing for intrachromosomal expression of the antibody encoding nucleic acids (Koller and Smithies, 1989, Proc. Natl. Acad. Sci. USA 86:8932-8935; Zijlstra et al., 1989, Nature 342:435-438). In specific embodiments, the expressed antibody or other prophylactic or therapeutic agent is a single chain antibody; alternatively, the nucleic acid sequences include sequences encoding both the heavy and light chains, or fragments thereof, of the antibody or another prophylactic or therapeutic agent of the invention.

Delivery of the nucleic acids into a subject may be either direct, in which case the subject is directly exposed to the nucleic acid or nucleic acid-carrying vectors, or indirect, in which case, cells are first transformed with the nucleic acids in vitro, then transplanted into the subject. These two approaches are known, respectively, as in vivo or ex vivo gene therapy.

In a specific embodiment, the nucleic acid sequences are directly administered in vivo, where it is expressed to produce the encoded product. This can be accomplished by any of numerous methods known in the art, e.g., by constructing them as part of an appropriate nucleic acid expression vector and administering it so that they become intracellular, e.g., by infection using defective or attenuated retrovirals or other viral vectors (see U.S. Pat. No. 4,980,286), or by direct injection of naked DNA, or by use of microparticle bombardment (e.g., a gene gun; Biolistic, Dupont), or coating with lipids or cell-surface receptors or transfecting agents, encapsulation in liposomes, microparticles, or microcapsules, or by administering them in linkage to a peptide which is known to enter the nucleus, by administering it in linkage to a ligand subject to receptor-mediated endocytosis (see, e.g., Wu and Wu, 1987, J. Biol. Chem. 262:4429-4432) (which can be used to target cell types specifically expressing the receptors). In another embodiment, nucleic acid-ligand complexes can be formed in which the ligand comprises a fusogenic viral peptide to disrupt endosomes, allowing the nucleic acid to avoid lysosomal degradation. In yet another embodiment, the nucleic acid can be targeted in vivo for cell specific uptake and expression, by targeting a specific receptor (see, e.g., International Publication Nos. WO 92/06180; WO 92/22635; WO92/20316; WO93/14188; and WO 93/20221). Alternatively, the nucleic acid can be introduced intracellularly and incorporated within host cell DNA for expression, by homologous recombination (Koller and Smithies, 1989, Proc. Natl. Acad. Sci. USA 86:8932-8935; and Zijlstra et al., 1989, Nature 342:435-438).

In a specific embodiment, viral vectors that contains nucleic acid sequences encoding an antibody, another prophylactic or therapeutic agent of the invention, or fragments thereof are used. For example, a retroviral vector can be used (see Miller et al., 1993, Meth. Enzymol. 217:581-599). These retroviral vectors contain the components necessary for the correct packaging of the viral genome and integration into the host cell DNA. The nucleic acid sequences encoding the antibody or another prophylactic or therapeutic agent of the invention to be used in gene therapy are cloned into one or more vectors, which facilitates delivery of the gene into a subject. More detail about retroviral vectors can be found in Boesen et al., 1994, Biotherapy 6:291-302, which describes the use of a retroviral vector to deliver the mdr 1 gene to hematopoietic stem cells in order to make the stem cells more resistant to chemotherapy. Other references illustrating the use of retroviral vectors in gene therapy are: Clowes et al., 1994, J. Clin. Invest. 93:644-651; Klein et al., 1994, Blood 83:1467-1473; Salmons and Gunzberg, 1993, Human Gene Therapy 4:129-141; and Grossman and Wilson, 1993, Curr. Opin. in Genetics and Devel. 3:110-114.

Adenoviruses are other viral vectors that can be used in gene therapy. Adenoviruses are especially attractive vehicles for delivering genes to respiratory epithelia. Adenoviruses naturally infect respiratory epithelia where they cause a mild disease. Other targets for adenovirus-based delivery systems are liver, the central nervous system, endothelial cells, and muscle. Adenoviruses have the advantage of being capable of infecting non-dividing cells. Kozarsky and Wilson, 1993, Current Opinion in Genetics and Development 3:499-503 present a review of adenovirus-based gene therapy. Bout et al., 1994, Human Gene Therapy 5:3-10 demonstrated the use of adenovirus vectors to transfer genes to the respiratory epithelia of rhesus monkeys. Other instances of the use of adenoviruses in gene therapy can be found in Rosenfeld et al., 1991, Science 252:431-434; Rosenfeld et al., 1992, Cell 68:143-155; Mastrangeli et al., 1993, J. Clin. Invest. 91:225-234; PCT Publication WO94/12649; and Wang et al., 1995, Gene Therapy 2:775-783. In a preferred embodiment, adenovirus vectors are used.

Adeno-associated virus (AAV) has also been proposed for use in gene therapy (Walsh et al., 1993, Proc. Soc. Exp. Biol. Med. 204:289-300; and U.S. Pat. No. 5,436,146).

Another approach to gene therapy involves transferring a gene to cells in tissue culture by such methods as electroporation, lipofection, calcium phosphate mediated transfection, or viral infection. Usually, the method of transfer includes the transfer of a selectable marker to the cells. The cells are then placed under selection to isolate those cells that have taken up and are expressing the transferred gene. Those cells are then delivered to a subject.

In this embodiment, the nucleic acid is introduced into a cell prior to administration in vivo of the resulting recombinant cell. Such introduction can be carried out by any method known in the art, including but not limited to transfection, electroporation, microinjection, infection with a viral or bacteriophage vector containing the nucleic acid sequences, cell fusion, chromosome-mediated gene transfer, microcell-mediated gene transfer, spheroplast fusion, etc. Numerous techniques are known in the art for the introduction of foreign genes into cells (see, e.g., Loeffler and Behr, 1993, Meth. Enzymol. 217:599-618; Cohen et al., 1993, Meth. Enzymol. 217:618-644; Clin. Pharma. Ther. 29:69-92 (1985)) and may be used in accordance with the present invention, provided that the necessary developmental and physiological functions of the recipient cells are not disrupted. The technique should provide for the stable transfer of the nucleic acid to the cell, so that the nucleic acid is expressible by the cell and preferably heritable and expressible by its cell progeny.

The resulting recombinant cells can be delivered to a subject by various methods known in the art. Recombinant blood cells (e.g., hematopoietic stem or progenitor cells) are preferably administered intravenously. The amount of cells envisioned for use depends on the several factors including, but not limited to, the desired effects and the patient state, and can be determined by one skilled in the art.

Cells into which a nucleic acid can be introduced for purposes of gene therapy encompass any desired, available cell type, and include but are not limited to epithelial cells, endothelial cells, keratinocytes, fibroblasts, muscle cells, hepatocytes; blood cells such as T lymphocytes, B lymphocytes, monocytes, macrophages, neutrophils, eosinophils, mast cells, megakaryocytes, granulocytes; various stem or progenitor cells, in particular hematopoietic stem or progenitor cells (e.g., as obtained from bone marrow, umbilical cord blood, peripheral blood, fetal liver, etc.). In a preferred embodiment, the cell used for gene therapy is autologous to the subject.

In an embodiment in which recombinant cells are used in gene therapy, nucleic acid sequences encoding an antibody or fragment thereof are introduced into the cells such that they are expressible by the cells or their progeny, and the recombinant cells are then administered in vivo for therapeutic effect. In a specific embodiment, stem or progenitor cells are used. Any stem and/or progenitor cells which can be isolated and maintained in vitro can potentially be used in accordance with this embodiment of the present invention (see e.g., PCT Publication WO 94/08598; Stemple and Anderson, 1992, Cell 7 1:973-985; Rheinwald, 1980, Meth. Cell Bio. 21A:229; and Pittelkow and Scott, 1986, Mayo Clinic Proc. 61:771).

In a specific embodiment, the nucleic acid to be introduced for purposes of gene therapy comprises an inducible promoter operably linked to the coding region, such that expression of the nucleic acid is controllable by controlling the presence or absence of the appropriate inducer of transcription.

### 5.9. Dosage and Frequency of Administration

The amount of a prophylactic or therapeutic agent or a composition of the present invention which will be effective in the treatment, management, prevention, or amelioration of a disorder or one or more symptoms thereof can be determined by standard clinical. The frequency and dosage will vary according to factors specific for each patient depending on the specific therapy or therapies (e.g., the specific therapeutic or prophylactic agent or agents) administered, the severity of the disorder, disease, or condition, the route of administration, as well as age, body, weight, response, the patient's immune status, and the past medical history of the patient. For example, the dosage of a prophylactic or therapeutic agent or a composition of the invention which will be effective in the treatment, prevention, management, or amelioration of a disorder or one or more symptoms thereof can be determined by administering the composition to an animal model such as, e.g., the animal models disclosed herein or known to those skilled in the art. In addition, in vitro assays may optionally be employed to help identify optimal dosage ranges. Suitable regimens can be selected by one skilled in the art by considering such factors and by following, for example, dosages reported in the literature and recommended in the Physician 's Desk Reference (57th ed., 2003).

The toxicity and/or efficacy of the prophylactic and/or therapeutic protocols of the present invention can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Therapies that exhibit large therapeutic indices are preferred. While therapies that exhibit toxic side effects may be used, care should be taken to design a delivery system that targets such agents to the site of affected tissue in order to minimize potential damage to uninfected cells and, thereby, reduce side effects.

The data obtained from the cell culture assays and animal studies can be used in formulating a range of dosage of the prophylactic and/or therapeutic agents for use in humans. The dosage of such agents lies preferably within a range of circulating concentrations that include the ED₅₀ with little or no toxicity. The dosage may vary within this range depending upon the dosage form employed and the route of administration utilized. For any therapy used in the method of the invention, the therapeutically effective dose can be estimated initially from cell culture assays. A dose may be formulated in animal models to achieve a circulating plasma concentration range that includes the IC₅₀ (i.e., the concentration of the test compound that achieves a half-maximal inhibition of symptoms) as determined in cell culture. Such information can be used to more accurately determine useful doses in humans. Levels in plasma may be measured, for example, by high performance liquid chromatography.

For peptides, polypeptides, proteins, fusion proteins, and antibodies, the dosage administered to a patient is typically 0.01 mg/kg to 100 mg/kg of the patient's body weight. Preferably, the dosage administered to a patient is between 0.1 mg/kg and 20 mg/kg of the patient's body weight, more preferably 1 mg/kg to 10 mg/kg of the patient's body weight. Generally, human and humanized antibodies have a longer half-life within the human body than antibodies from other species due to the immune response to the foreign polypeptides. Thus, lower dosages of human antibodies and less frequent administration is often possible.

Exemplary doses of a small molecule include milligram or microgram amounts of the small molecule per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram).

The dosages of prophylactic or therapeutically agents are described in the Physicians' Desk Reference (56th ed., 2002).

### 5.10. Biological Assays

Antibodies of the present invention or fragments thereof may be characterized in a variety of ways well-known to one of skill in the art. In particular, antibodies of the invention or fragments thereof may be assayed for the ability to immunospecifically bind to an antigen. Such an assay may be performed in solution (e.g., Houghten, 1992, Bio/Techniques 13:412 421), on beads (Lam, 1991, Nature 354:82 84), on chips (Fodor, 1993, Nature 364:555 556), on bacteria (U.S. Pat. No. 5,223,409), on spores (U.S. Pat. Nos. 5,571,698; 5,403,484; and 5,223,409), on plasmids (Cull et al., 1992, Proc. Natl. Acad. Sci. USA 89:1865 1869) or on phage (Scott and Smith, 1990, Science 249:386 390; Cwirla et al., 1990, Proc. Natl. Acad. Sci. USA 87:6378 6382; and Felici, 1991, J. Mol. Biol. 222:301 310) (each of these references is incorporated herein in its entirety by reference). Antibodies or fragments thereof that have been identified can then be assayed for specificity and affinity.

The antibodies of the invention or fragments thereof may be assayed for immunospecific binding to a specific antigen and cross-reactivity with other antigens by any method known in the art. Immunoassays which can be used to analyze immunospecific binding and cross-reactivity include, but are not limited to, competitive and non-competitive assay systems using techniques such as western blots, radioimmunoassays, ELISA (enzyme linked immunosorbent assay), “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays, protein A immunoassays, to name but a few. Such assays are routine and well-known in the art (see, e.g., Ausubel et al., eds., 1994, Current Protocols in Molecular Biology, Vol. 1, John Wiley & Sons, Inc., New York, which is incorporated by reference herein in its entirety). Exemplary immunoassays are described briefly in Section 5.6.

The antibodies of the invention or fragments thereof can also be assayed for their ability to inhibit the binding of an antigen to its host cell receptor using techniques known to those of skill in the art. For example, cells expressing a receptor can be contacted with a ligand for that receptor in the presence or absence of an antibody or fragment thereof that is an antagonist of the ligand and the ability of the antibody or fragment thereof to inhibit the ligand's binding can measured by, for example, flow cytometry or a scintillation assay. The ligand or the antibody or antibody fragment can be labeled with a detectable compound such as a radioactive label (e.g., ³²P, ³⁵S, and ¹²⁵I) or a fluorescent label (e.g., fluorescein isothiocyanate, rhodamine, phycoerythrin, phycocyanin, allophycocyanin, o-phthaldehyde and fluorescamine) to enable detection of an interaction between the ligand and its receptor. Alternatively, the ability of antibodies or fragments thereof to inhibit a ligand from binding to its receptor can be determined in cell-free assays. For example, a ligand can be contacted with an antibody or fragment thereof that is an antagonist of the ligand and the ability of the antibody or antibody fragment to inhibit the ligand from binding to its receptor can be determined. Preferably, the antibody or the antibody fragment that is an antagonist of the ligand is immobilized on a solid support and the ligand is labeled with a detectable compound. Alternatively, the ligand is immobilized on a solid support and the antibody or fragment thereof is labeled with a detectable compound. A ligand may be partially or completely purified (e.g., partially or completely free of other polypeptides) or part of a cell lysate. Alternatively, a ligand can be biotinylated using techniques well known to those of skill in the art (e.g., biotinylation kit, Pierce Chemicals; Rockford, Ill.).

An antibody or a fragment thereof constructed and/or identified in accordance with the present invention can be tested in vitro and/or in vivo for its ability to modulate the biological activity of cells. Such ability can be assessed by, e.g., detecting the expression of antigens and genes; detecting the proliferation of cells; detecting the activation of signaling molecules (e.g., signal transduction factors and kinases); detecting the effector function of cells; or detecting the differentiation of cells. Techniques known to those of skill in the art can be used for measuring these activities. For example, cellular proliferation can be assayed by ³H-thymidine incorporation assays and trypan blue cell counts. Antigen expression can be assayed, for example, by immunoassays including, but are not limited to, competitive and non-competitive assay systems using techniques such as western blots, immunohistochemistry radioimmunoassays, ELISA (enzyme linked immunosorbent assay), “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays, protein A immunoassays, and FACS analysis. The activation of signaling molecules can be assayed, for example, by kinase assays and electrophoretic shift assays (EMSAs).

The antibodies, fragments thereof, or compositions of the invention are preferably tested in vitro and then in vivo for the desired therapeutic or prophylactic activity prior to use in humans. For example, assays which can be used to determine whether administration of a specific pharmaceutical composition is indicated include cell culture assays in which a patient tissue sample is grown in culture and exposed to, or otherwise contacted with, a pharmaceutical composition, and the effect of such composition upon the tissue sample is observed. The tissue sample can be obtained by biopsy from the patient. This test allows the identification of the therapeutically most effective therapy (e.g., prophylactic or therapeutic agent) for each individual patient. In various specific embodiments, in vitro assays can be carried out with representative cells of cell types involved a particular disorder to determine if a pharmaceutical composition of the invention has a desired effect upon such cell types. For example, in vitro asssay can be carried out with cell lines.

The effect of an antibody, a fragment thereof, or a composition of the invention on peripheral blood lymphocyte counts can be monitored/assessed using standard techniques known to one of skill in the art. Peripheral blood lymphocytes counts in a subject can be determined by, e.g., obtaining a sample of peripheral blood from said subject, separating the lymphocytes from other components of peripheral blood such as plasma using, e.g., Ficoll-Hypaque (Pharmacia) gradient centrifugation, and counting the lymphocytes using trypan blue. Peripheral blood T-cell counts in subject can be determined by, e.g., separating the lymphocytes from other components of peripheral blood such as plasma using, e.g., a use of Ficoll-Hypaque (Pharmacia) gradient centrifugation, labeling the T-cells with an antibody directed to a T-cell antigen which is conjugated to FITC or phycoerythrin, and measuring the number of T-cells by FACS.

The antibodies, fragments, or compositions of the invention used to treat, manage, prevent, or ameliorate a viral infection or one or more symptoms thereof can be tested for their ability to inhibit viral replication or reduce viral load in in vitro assays. For example, viral replication can be assayed by a plaque assay such as described, e.g., by Johnson et al., 1997, Journal of Infectious Diseases 176:1215-1224 176:1215-1224. The antibodies or fragments thereof administered according to the methods of the invention can also be assayed for their ability to inhibit or downregulate the expression of viral polypeptides. Techniques known to those of skill in the art, including, but not limited to, western blot analysis, northern blot analysis, and RT-PCR can be used to measure the expression of viral polypeptides.

The antibodies, fragments, or compositions of the invention used to treat, manage, prevent, or ameliorate a bacterial infection or one or more symptoms thereof can be tested in in vitro assays that are well-known in the art. In vitro assays known in the art can also be used to test the existence or development of resistance of bacteria to a therapy. Such in vitro assays are described in Gales et al., 2002, Diag. Nicrobiol. Infect. Dis. 44(3):301-311; Hicks et al., 2002, Clin. Microbiol. Infect. 8(11): 753-757; and Nicholson et al., 2002, Diagn. Microbiol. Infect. Dis. 44(1): 101-107.

The antibodies, fragments, or compositions of the invention used to treat, manage, prevent, or ameliorate a fungal infection or one or more symptoms thereof can be tested for anti-fungal activity against different species of fungus. Any of the standard anti-fungal assays well-known in the art can be used to assess the anti-fungal activity of a therapy. The anti-fungal effect on different species of fungus can be tested. The tests recommended by the National Committee for Clinical Laboratories (NCCLS) (See National Committee for Clinical Laboratories Standards. 1995, Proposed Standard M27T. Villanova, Pa., all of which is incorporated herein by reference in its entirety) and other methods known to those skilled in the art (Pfaller et al., 1993, Infectious Dis. Clin. N. Am. 7: 435-444) can be used to assess the anti-fungal effect of a therapy. The antifungal properties of a therapy may also be determined from a fungal lysis assay, as well as by other methods, including, inter alia, growth inhibition assays, fluorescence-based fungal viability assays, flow cytometry analyses, and other standard assays known to those skilled in the art.

For example, the anti-fungal activity of a therapy can be tested using macrodilution methods and/or microdilution methods using protocols well-known to those skilled in the art (see, e.g., Clancy et al., 1997 Journal of Clinical Microbiology, 35(11): 2878-82; Ryder et al., 1998, Antimicrobial Agents and Chemotherapy, 42(5): 1057-61; U.S. Pat. No. 5,521,153; U.S. Pat. No. 5,883,120, U.S. Pat. No. 5,521,169, all of which are incorporated by reference in their entirety). Briefly, a fungal strain is cultured in an appropriate liquid media, and grown at an appropriate temperature, depending on the particular fungal strain used for a determined amount of time, which is also depends on the particular fungal strain used. An innoculum is then prepared photometrically and the turbidity of the suspension is matched to that of a standard, e.g., a McFarland standard. The effect of a therapy on the turbidity of the inoculum is determined visually or spectrophotometrically. The minimal inhibitory concentration (“MIC”) of the therapy is determined, which is defined as the lowest concentration of the lead compound which prevents visible growth of an inoculum as measured by determining the culture turbidity.

The anti-fungal activity of a therapy can also be determined utilizing colorimetric based assays well-known to one of skill in the art. One exemplary colorimetric assay that can be used to assess the anti-fungal activity of a therapy is described by Pfaller et al. (1994, Journal of Clinical Microbiology, 32(8): 1993-6, which is incorporated herein by reference in its entirety; also see Tiballi et al., 1995, Journal of Clinical Microbiology, 33(4): 915-7). This assay employs a colorimetric endpoint using an oxidation-reduction indicator (Alamar Biosciences, Inc., Sacramento Calif.).

The anti-fungal activity of a therapy can also be determined utilizing photometric assays well-known to one of skill in the art (see, e.g., Clancy et al., 1997 Journal of Clinical Microbiology, 35(11): 2878-82; Jahn et al., 1995, Journal of Clinical Microbiology, 33(3): 661-667, each of which is incorporated herein by reference in its entirety). This photometric assay is based on quantifying mitochondrial respiration by viable fungi through the reduction of 3-(4,5-dimethyl-2thiazolyl)-2,5,-diphenyl-2H-tetrazolium bromide (MTT) to formazan. MIC's determined by this assay are defined as the highest concentration of the test therapy associated with the first precipitous drop in optical density. In some embodiments, the therapy is assayed for anti-fungal activity using macrodilution, microdilution and MTT assays in parallel.

Further, any in vitro assays known to those skilled in the art can be used to evaluate the prophylactic and/or therapeutic utility of an antibody therapy disclosed herein for a particular disorder or one or more symptoms thereof.

The antibodies, compositions, or combination therapies of the invention can be tested in suitable animal model systems prior to use in humans. Such animal model systems include, but are not limited to, rats, mice, chicken, cows, monkeys, pigs, dogs, rabbits, etc. Any animal system well-known in the art may be used. Several aspects of the procedure may vary; said aspects include, but are not limited to, the temporal regime of administering the therapies (e.g., prophylactic and/or therapeutic agents) whether such therapies are administered separately or as an admixture, and the frequency of administration of the therapies.

Animal models can be used to assess the efficacy of the antibodies, fragments thereof, or compositions of the invention for treating, managing, preventing, or ameliorating a particular disorder or one or more symptom thereof.

The administration of antibodies, compositions, or combination therapies according to the methods of the invention can be tested for their ability to decrease the time course of a particular disorder by at least 25%, preferably at least 50%, at least 60%, at least 75%, at least 85%, at least 95%, or at least 99%. The antibodies, compositions, or combination therapies of the invention can also be tested for their ability to increase the survival period of humans suffering from a particular disorder by at least 25%, preferably at least 50%, at least 60%, at least 75%, at least 85%, at least 95%, or at least 99%. Further, antibodies, compositions, or combination therapies of the invention can be tested for their ability reduce the hospitalization period of humans suffering from viral respiratory infection by at least 60%, preferably at least 75%, at least 85%, at least 95%, or at least 99%. Techniques known to those of skill in the art can be used to analyze the function of the antibodies, compositions, or combination therapies of the invention in vivo.

Further, any in vivo assays known to those skilled in the art can be used to evaluate the prophylactic and/or therapeutic utility of an antibody, a fragment thereof, a composition, a combination therapy disclosed herein for a particular disorder or one or more symptoms thereof.

The toxicity and/or efficacy of the prophylactic and/or therapeutic protocols of the instant invention can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Therapies that exhibit large therapeutic indices are preferred. While therapies that exhibit toxic side effects may be used, care should be taken to design a delivery system that targets such agents to the site of affected tissue in order to minimize potential damage to uninfected cells and, thereby, reduce side effects.

The data obtained from the cell culture assays and animal studies can be used in formulating a range of dosage of the prophylactic and/or therapeutic agents for use in humans. The dosage of such agents lies preferably within a range of circulating concentrations that include the ED50 with little or no toxicity. The dosage may vary within this range depending upon the dosage form employed and the route of administration utilized. For any therapy used in the method of the invention, the therapeutically effective dose can be estimated initially from cell culture assays. A dose may be formulated in animal models to achieve a circulating plasma concentration range that includes the IC50 (i.e., the concentration of the test compound that achieves a half-maximal inhibition of symptoms) as determined in cell culture. Such information can be used to more accurately determine useful doses in humans. Levels in plasma may be measured, for example, by high performance liquid chromatography.

### 5.11. Kits

The invention provides kits comprising combinatorial libraries that comprises plurality of nucleic acid sequences comprising nucleotide sequences, each nucleotide sequence encoding the framework regions and CDRs fused in-frame (e.g., FR1+CDR1+FR2+CDR2+FR3+CDR3+FR4).

The invention also provides a pharmaceutical pack or kit comprising one or more containers filled with a humanized antibody of the invention. The pharmaceutical pack or kit may further comprises one or more other prophylactic or therapeutic agents useful for the treatment of a particular disease. The invention also provides a pharmaceutical pack or kit comprising one or more containers filled with one or more of the ingredients of the pharmaceutical compositions of the invention. Optionally associated with such container(s) can be a notice in the form prescribed by a governmental agency regulating the manufacture, use or sale of pharmaceuticals or biological products, which notice reflects approval by the agency of manufacture, use or sale for human administration.

### 5.12. Article of Manufacture

The present invention also encompasses a finished packaged and labeled pharmaceutical product. This article of manufacture includes the appropriate unit dosage form in an appropriate vessel or container such as a glass vial or other container that is hermetically sealed. In the case of dosage forms suitable for parenteral administration the active ingredient is sterile and suitable for administration as a particulate free solution. In other words, the invention encompasses both parenteral solutions and lyophilized powders, each being sterile, and the latter being suitable for reconstitution prior to injection. Alternatively, the unit dosage form may be a solid suitable for oral, transdermal, topical or mucosal delivery.

In a preferred embodiment, the unit dosage form is suitable for intravenous, intramuscular or subcutaneous delivery. Thus, the invention encompasses solutions, preferably sterile, suitable for each delivery route.

As with any pharmaceutical product, the packaging material and container are designed to protect the stability of the product during storage and shipment. Further, the products of the invention include instructions for use or other informational material that advise the physician, technician or patient on how to appropriately prevent or treat the disease or disorder in question. In other words, the article of manufacture includes instruction means indicating or suggesting a dosing regimen including, but not limited to, actual doses, monitoring procedures (such as methods for monitoring mean absolute lymphocyte counts, tumor cell counts, and tumor size) and other monitoring information.

More specifically, the invention provides an article of manufacture comprising packaging material, such as a box, bottle, tube, vial, container, sprayer, insufflator, intravenous (i.v.) bag, envelope and the like; and at least one unit dosage form of a pharmaceutical agent contained within said packaging material. The invention further provides an article of manufacture comprising packaging material, such as a box, bottle, tube, vial, container, sprayer, insufflator, intravenous (i.v.) bag, envelope and the like; and at least one unit dosage form of each pharmaceutical agent contained within said packaging material.

In a specific embodiment, an article of manufacture comprises packaging material and a pharmaceutical agent and instructions contained within said packaging material, wherein said pharmaceutical agent is a humanized antibody and a pharmaceutically acceptable carrier, and said instructions indicate a dosing regimen for preventing, treating or managing a subject with a particular disease. In another embodiment, an article of manufacture comprises packaging material and a pharmaceutical agent and instructions contained within said packaging material, wherein said pharmaceutical agent is a humanized antibody, a prophylactic or therapeutic agent other than the humanized antibody and a pharmaceutically acceptable carrier, and said instructions indicate a dosing regimen for preventing, treating or managing a subject with a particular disease. In another embodiment, an article of manufacture comprises packaging material and two pharmaceutical agents and instructions contained within said packaging material, wherein said first pharmaceutical agent is a humanized antibody and a pharmaceutically acceptable carrier and said second pharmaceutical agent is a prophylactic or therapeutic agent other than the humanized antibody, and said instructions indicate a dosing regimen for preventing, treating or managing a subject with a particular disease.

The present invention provides that the adverse effects that may be reduced or avoided by the methods of the invention are indicated in informational material enclosed in an article of manufacture for use in preventing, treating or ameliorating one or more symptoms associated with a disease. Adverse effects that may be reduced or avoided by the methods of the invention include but are not limited to vital sign abnormalities (e.g., fever, tachycardia, bardycardia, hypertension, hypotension), hematological events (e.g., anemia, lymphopenia, leukopenia, thrombocytopenia), headache, chills, dizziness, nausea, asthenia, back pain, chest pain (e.g., chest pressure), diarrhea, myalgia, pain, pruritus, psoriasis, rhinitis, sweating, injection site reaction, and vasodilatation. Since some of the therapies may be immunosuppressive, prolonged immunosuppression may increase the risk of infection, including opportunistic infections. Prolonged and sustained immunosuppression may also result in an increased risk of developing certain types of cancer.

Further, the information material enclosed in an article of manufacture for use in preventing, treating or ameliorating one or more symptoms with a skin condition characterized by increased T cell activation and/or abnormal antigen presentation can indicate that foreign proteins may also result in allergic reactions, including anaphylaxis, or cytosine release syndrome. The information material should indicate that allergic reactions may exhibit only as mild pruritic rashes or they may be severe such as erythroderma, Stevens Johnson syndrome, vasculitis, or anaphylaxis. The information material should also indicate that anaphylactic reactions (anaphylaxis) are serious and occasionally fatal hypersensitivity reactions. Allergic reactions including anaphylaxis may occur when any foreign protein is injected into the body. They may range from mild manifestations such as urticaria or rash to lethal systemic reactions. Anaphylactic reactions occur soon after exposure, usually within 10 minutes. Patients may experience paresthesia, hypotension, laryngeal edema, mental status changes, facial or pharyngeal angioedema, airway obstruction, bronchospasm, urticaria and pruritus, serum sickness, arthritis, allergic nephritis, glomerulonephritis, temporal arthritis, or eosinophilia.

The information material can also indicate that cytokine release syndrome is an acute clinical syndrome, temporally associated with the administration of certain activating anti T cell antibodies. Cytokine release syndrome has been attributed to the release of cytokines by activated lymphocytes or monocytes. The clinical manifestations for cytokine release syndrome have ranged from a more frequently reported mild, self limited, “flu like” illness to a less frequently reported severe, life threatening, shock like reaction, which may include serious cardiovascular, pulmonary and central nervous system manifestations. The syndrome typically begins approximately 30 to 60 minutes after administration (but may occur later) and may persist for several hours. The frequency and severity of this symptom complex is usually greatest with the first dose. With each successive dose, both the incidence and severity of the syndrome tend to diminish. Increasing the amount of a dose or resuming treatment after a hiatus may result in a reappearance of the syndrome. As mentioned above, the invention encompasses methods of treatment and prevention that avoid or reduce one or more of the adverse effects discussed herein.

### 5.13 Exemplary Embodiments

1. A library of nucleic acid sequences comprising nucleotide sequences encoding humanized heavy chain variable regions, each nucleotide sequence produced by fusing together in frame nucleic acid sequences encoding CDRs from a donor antibody heavy chain variable region and nucleic acid sequences encoding acceptor heavy chain variable framework regions that are together less than 65% identical to the donor antibody heavy chain variable framework regions together at the amino acid level.

2. A library of nucleic acid sequences comprising nucleotide sequences encoding humanized heavy chain variable regions, each nucleotide sequence produced by fusing together in frame nucleic acid sequences encoding CDRs from a donor antibody heavy chain variable region and nucleic acid sequences encoding acceptor heavy chain variable framework regions that are together less than 65% identical to the donor antibody heavy chain variable framework regions together at the amino acid level and contain one or more mutations at amino acid residues designated key residues, said key residues not including amino acid residues 2, 4, 24, 35, 36, 39, 43, 45, 64, 69, 70, 73, 74, 75, 76, 78, 92 and 93 according to the Kabat numbering system.

3. A library of nucleic acid sequences comprising nucleotide sequences encoding humanized light chain variable regions, each nucleotide sequence produced by fusing together in frame nucleic acid sequences encoding CDRs from a donor antibody light chain variable region and nucleic acid sequences encoding acceptor light chain variable framework regions together that are less than 65% identical to the donor antibody light chain variable framework regions together at the amino acid level.

4. A library of nucleic acid sequences comprising nucleotide sequences encoding humanized light chain variable regions, each nucleotide sequence produced by fusing together in frame nucleic acid sequences encoding CDRs from a donor antibody light chain variable region and nucleic acid sequences encoding acceptor light chain variable framework regions together that are less than 65% identical to the donor antibody light chain variable framework regions at the amino acid level and contain one or more mutations at amino acid residues designated key residues, said key residues not including amino acid residues 4, 38, 43, 44, 46, 58, 62, 65, 66, 67, 68, 69, 73, 85 and 98 according to the Kabat numbering system.

5. A library of nucleic acid sequences comprising (i) a first set of nucleotide sequences encoding humanized heavy chain variable regions, each nucleotide sequence in the first set of nucleotide sequences produced by fusing together in frame nucleic acid sequences encoding CDRs from a donor antibody heavy chain variable region and nucleic acid sequences encoding acceptor heavy chain variable framework regions together that are less than 65% identical to the donor antibody heavy chain variable framework regions together at the amino acid level; and (ii) a second set of nucleotide sequences encoding humanized light chain variable regions, each nucleotide sequence in the second set of nucleotide sequences produced by fusing together in frame nucleic acid sequences encoding CDRs from a donor antibody light chain variable region and nucleic acid sequences encoding acceptor light chain variable framework regions.

6. A library of nucleic acid sequences comprising (i) a first set of nucleotide sequences encoding humanized heavy chain variable regions, each nucleotide sequence in the first set of nucleotide sequences produced by fusing together in frame nucleic acid sequences encoding CDRs from a donor antibody heavy chain variable region and nucleic acid sequences encoding acceptor heavy chain variable framework regions together that are less than 65% identical to the donor antibody heavy chain variable framework regions together at the amino acid level and contain one or more mutations at amino acid residues designated key residues, said key residues not including amino acid residues 2, 4, 24, 35, 36, 39, 43, 45, 64, 69, 70, 73, 74, 75, 76, 78, 92 and 93 according to the Kabat numbering system; and (ii) a second set of nucleotide sequences encoding humanized light chain variable regions, each nucleotide sequence in the second set of nucleotide sequences produced by fusing together in frame nucleic acid sequences encoding CDRs from a donor antibody light chain variable region and nucleic acid sequences encoding acceptor light chain variable framework regions.

7. A library of nucleic acid sequences comprising: (i) a first set of nucleotide sequences encoding humanized heavy chain variable regions, each nucleotide sequence in the first set of nucleotide sequences produced by fusing together in frame nucleic acid sequences encoding CDRs from a donor antibody heavy chain variable region and nucleic acid sequences encoding acceptor heavy chain variable framework regions; and (ii) a second set of nucleotide sequences encoding humanized light chain variable regions, each nucleotide sequence in the second set of nucleotide sequences produced by fusing together in frame nucleic acid sequences encoding CDRs from a donor antibody light chain variable region and nucleic acid sequences encoding acceptor light chain variable framework regions together that are less than 65% identical to the donor antibody light chain variable framework regions together at the amino acid level.

8. A library of nucleic acid sequences comprising: (i) a first set of nucleotide sequences encoding humanized heavy chain variable regions, each nucleotide sequence in the first set of nucleotide sequences produced by fusing together in frame nucleic acid sequences encoding CDRs from a donor antibody heavy chain variable region and nucleic acid sequences encoding acceptor heavy chain variable framework regions; and (ii) a second set of nucleotide sequences encoding humanized light chain variable regions, each nucleotide sequence in the second set of nucleotide sequences produced by fusing together in frame nucleic acid sequences encoding CDRs from a donor antibody light chain variable region and nucleic acid sequences encoding acceptor light chain variable framework regions together that are less than 65% identical to the donor antibody light chain variable framework regions together at the amino acid level and contain one or more mutations at amino acid residues designated key residues, said key residues not including amino acid residues 4, 38, 43, 44, 46, 58, 62, 65, 66, 67, 68, 69, 73, 85 and 98 according to the Kabat numbering system.

9. A library of nucleic acid sequences comprising: (i) a first set of nucleotide sequences encoding humanized heavy chain variable regions, each nucleotide sequence in the first set of nucleotide sequences produced by fusing together in frame nucleic acid sequences encoding CDRs from a donor antibody heavy chain variable region and nucleic acid sequences encoding acceptor heavy chain variable framework regions together that are less than 65% identical to the donor antibody heavy chain variable framework regions together at the amino acid level; and (ii) a second set of nucleotide sequences encoding humanized light chain variable regions, each nucleotide sequence in the second set of nucleotide sequences produced by fusing together in frame nucleic acid sequences encoding CDRs from a donor antibody light chain variable region and nucleic acid sequences encoding acceptor light chain variable framework regions together that are less than 65% identical to the donor antibody light chain variable framework regions together at the amino acid level.

10. A library of nucleic acid sequences comprising: (i) a first set of nucleotide sequences encoding humanized heavy chain variable regions, each nucleotide sequence in the first set of nucleotide sequences produced by fusing together in frame nucleic acid sequences encoding CDRs from a donor antibody heavy chain variable region and nucleic acid sequences encoding acceptor heavy chain variable framework regions together that are less than 65% identical to the donor antibody heavy chain variable framework regions together at the amino acid level and contain one or more mutations at amino acid residues designated key residues, said key residues not including amino acid residues 2, 4, 24, 35, 36, 39, 43, 45, 64, 69, 70, 73, 74, 75, 76, 78, 92 and 93 according to the Kabat numbering system; and (ii) a second set of nucleotide sequences encoding humanized light chain variable regions, each nucleotide sequence in the second set of nucleotide sequences produced by fusing together in frame nucleic acid sequences encoding CDRs from a donor antibody light chain variable region and nucleic acid sequences encoding acceptor light chain variable framework regions together that are less than 65% identical to the donor antibody light chain variable framework regions together at the amino acid level.

11. A library of nucleic acid sequences comprising: (i) a first set of nucleotide sequences encoding humanized heavy chain variable regions, each nucleotide sequence in the first set of nucleotide sequences produced by fusing together in frame nucleic acid sequences encoding CDRs from a donor antibody heavy chain variable region and nucleic acid sequences encoding acceptor heavy chain variable framework regions together that are less than 65% identical to the donor antibody heavy chain variable framework regions together at the amino acid level; and (ii) a second set of nucleotide sequences encoding humanized light chain variable regions, each nucleotide sequence in the second set of nucleotide sequences produced by fusing together in frame nucleic acid sequences encoding CDRs from a donor antibody light chain variable region and nucleic acid sequences encoding acceptor light chain variable framework regions together that are less than 65% identical to the donor antibody light chain variable framework regions together at the amino acid level and contain one or more mutations at amino acid residues designated key residues, said key residues not including amino acid residues 4, 38, 43, 44, 46, 58, 62, 65, 66, 67, 68, 69, 73, 85 and 98 according to the Kabat numbering system.

12. A library of nucleic acid sequences comprising: (i) a first set of nucleotide sequences encoding humanized heavy chain variable regions, each nucleotide sequence in the first set of nucleotide sequences produced by fusing together in frame nucleic acid sequences encoding CDRs from a donor antibody heavy chain variable region and nucleic acid sequences encoding acceptor heavy chain variable framework regions together that are less than 65% identical to the donor antibody heavy chain variable framework regions together at the amino acid level and contain one or more mutations at amino acid residues designated key residues, said key residues not including amino acid residues 2, 4, 24, 35, 36, 39, 43, 45, 64, 69, 70, 73, 74, 75, 76, 78, 92 and 93 according to the Kabat numbering system; and (ii) a second set of nucleotide sequences encoding humanized light chain variable regions, each nucleotide sequence in the second set of nucleotide sequences produced by fusing together in frame nucleic acid sequences encoding CDRs from a donor antibody light chain variable region and nucleic acid sequences encoding acceptor light chain variable framework regions together that are less than 65% identical to the donor antibody light chain variable framework regions together at the amino acid level and contain one or more mutations at amino acid residues designated key residues, said key residues not including amino acid residues 4, 38, 43, 44, 46, 58, 62, 65, 66, 67, 68, 69, 73, 85 and 98 according to the Kabat numbering system.

13. The library of any of the embodiments 1 to 12, wherein said acceptor is human.

14. The library of any of the embodiments 1 to 12, wherein said acceptor contains at least one amino acid residue that does not occur at a specific position of a human antibody.

15. The library of embodiment 1, 2, 5, 6, 9, 10, 11 or 12, wherein the acceptor heavy chain variable framework regions contain at least one amino acid residue at amino acid residues 6, 23, 24 or 49 according to the Kabat numbering system that is not identical to the corresponding residue in the donor antibody.

16. The library of embodiment 2, 4, 6, 8, 10, 11 or 12, wherein the residues designated key are one or more of the following: a residue adjacent to a CDR, a potential glycosylation site, a rare residue, a residue capable of interacting with the antigen, a residue capable of interacting with a CDR, a canonical residue, a contact residue between the variable heavy region and variable light region, a residue within the Vernier zone, and a residue within the region which overlaps between the Chothia definition of the heavy chain variable region CDR1 and the Kabat definition of the first heavy chain framework.

17. A population of cells comprising the nucleic acid sequences of any one of embodiments 1-12.

18. A population of cells comprising the nucleic acid sequences of embodiment 15.

19. A method of identifying a humanized antibody that immunospecifically binds to an antigen, said method comprising expressing the nucleic acid sequences in the cells of embodiment 17 and screening for a humanized antibody that has an affinity of 1×10⁶ M⁻¹ or above for said antigen.

20. A method of identifying a humanized antibody that immunospecifically binds to an antigen, said method comprising expressing the nucleic acid sequences in the cells of embodiment 18 and identifying a humanized antibody that has an affinity of 1×10⁶ M⁻¹ or above for said antigen.

21. A humanized antibody identified by the method of embodiment 19.

22. A humanized antibody identified by the method of embodiment 20.

23. A composition comprising the humanized antibody of embodiment 21 and a carrier, diluent or excipient.

24. A composition comprising the humanized antibody of embodiment 22 and a carrier, diluent or excipient.

25. A cell containing nucleic acid sequences encoding a humanized antibody that immunospecifically binds to an antigen, said cell produced by the process comprising:
(a) selecting an acceptor heavy chain variable framework region less than 65% identical globally to a donor antibody heavy chain variable framework region at the amino acid level, which acceptor heavy chain variable framework region contains at least one amino acid residue at amino acid residues 6, 23, 24 or 49 according to the Kabat numbering system that is not identical to the corresponding residue in the donor antibody, and wherein the acceptor heavy chain framework region and donor antibody heavy chain framework region each comprises FR1, FR2, FR3 and FR4;
(b) synthesizing a nucleic acid sequence comprising a nucleotide sequence encoding a humanized heavy chain variable region, said nucleotide sequence comprising nucleic acid sequences encoding complementarity determining regions (CDRs) from the donor antibody heavy chain variable region and nucleic acid sequences encoding the acceptor heavy chain variable framework regions; and
(c) introducing the nucleic acid sequence comprising the nucleotide sequence encoding the humanized heavy chain variable region into a cell.

26. A cell containing nucleotide sequences encoding a humanized antibody that immunospecifically binds to an antigen, said cell produced by the process comprising:
(a) selecting an acceptor heavy chain variable framework region less than 65% identical to a donor antibody heavy chain variable framework region at the amino acid level, which acceptor heavy chain variable framework region contains at least one amino acid residue at amino acid residues 6, 23, 24 or 49 according to the Kabat numbering system that is not identical to the corresponding residue in the donor antibody, wherein the acceptor heavy chain framework region and donor antibody heavy chain framework region each comprises FR1, FR2, FR3 and FR4;
(b) synthesizing a nucleic acid sequence comprising a nucleotide sequence encoding a humanized heavy chain variable region with a framework region that remains less than 65% identical to the donor antibody heavy chain variable framework region at the amino acid level, said nucleotide sequence comprising nucleic acid sequences encoding CDRs from the donor antibody heavy chain variable region and nucleic acid sequences encoding the acceptor heavy chain variable framework regions with one or more mutations introduced at amino acid residues designated key residues, said key residues not including amino acid residues 2, 4, 24, 35, 36, 39, 43, 45, 64, 69, 70, 73, 74, 75, 76, 78, 92 and 93 according to the Kabat numbering system; and
(c) introducing the nucleic acid sequence comprising the nucleotide sequence encoding the humanized heavy chain variable region into a cell.

27. A cell containing nucleic acid sequences encoding a humanized antibody that immunospecifically binds to an antigen, said cell produced by the process comprising:
(a) selecting an acceptor light chain variable framework region less than 65% identical to a donor antibody light chain variable framework region at the amino acid level, wherein the acceptor light chain framework region and donor antibody light chain framework region each comprises FR1, FR2, FR3 and FR4;
(b) synthesizing a nucleic acid sequence comprising a nucleotide sequence encoding a humanized light chain variable region, said nucleotide sequence comprising nucleic acid sequences encoding complementarity determining regions (CDRs) from the donor antibody light chain variable region and nucleic acid sequences encoding the acceptor light chain variable framework regions; and
(c) introducing the nucleic acid sequence comprising the nucleotide sequence encoding the humanized light chain variable region into a cell.

28. A cell containing nucleotide sequences encoding a humanized antibody that immunospecifically binds to an antigen, said cell produced by the process comprising:
(a) selecting an acceptor light chain variable framework region less than 65% identical to a donor antibody heavy chain variable framework region at the amino acid level, wherein the acceptor light chain framework region and donor antibody light chain framework region each comprises FR1, FR2, FR3 and FR4;
(b) synthesizing a nucleic acid sequence comprising a nucleotide sequence encoding a humanized light chain variable region, said nucleotide sequence comprising nucleic acid sequences encoding CDRs from the donor antibody light chain variable region and nucleic acid sequences encoding the acceptor light chain variable framework regions with one or more mutations introduced at amino acid residues designated key residues, said key residues not including amino acid residues 4, 38, 43, 44, 46, 58, 62, 65, 66, 67, 68, 69, 73, 85, and 98 according to the Kabat numbering system; and
(c) introducing the nucleic acid sequence comprising the nucleotide sequence encoding the humanized light chain variable region into a cell.

29. A cell containing a nucleic acid sequence encoding a humanized antibody that immunospecifically binds to an antigen, said cell produced by the process comprising:
(a) selecting an acceptor heavy chain variable framework region less than 65% identical to a donor antibody heavy chain variable framework region at the amino acid level, which acceptor heavy chain variable framework region contains at least one amino acid residue at amino acid residues 6, 23, 24 or 49 according to the Kabat numbering system that is not identical to the corresponding residue in the donor antibody, wherein the acceptor heavy chain framework region and donor antibody heavy chain framework region each comprises FR1, FR2, FR3 and FR4;
(b) synthesizing a nucleic acid sequence comprising: (i) a first nucleotide sequence encoding a light chain variable region, and (ii) a second nucleotide sequence encoding a humanized heavy chain variable region with a framework region comprising FR1, FR2, FR3 and FR4 that remains globally less than 65% identical to the donor antibody heavy chain variable framework region at the amino acid level, said second nucleotide sequence comprising nucleic acid sequences encoding complementarity determining regions (CDRs) from the donor antibody heavy chain variable region and nucleic acid sequences encoding the acceptor heavy chain variable framework regions; and
(c) introducing the nucleic acid sequence comprising the first nucleotide sequence and second nucleotide sequence into a cell.

30. A cell containing a nucleotide sequence encoding a humanized antibody that immunospecifically binds to an antigen, said cell produced by the process comprising:
(a) selecting an acceptor heavy chain variable framework region less than 65% identical to a donor antibody heavy chain variable framework region at the amino acid level, which acceptor heavy chain variable framework region contains at least one amino acid residue at amino acid residues 6, 23, 24 or 49 according to the Kabat numbering system that is not identical to the corresponding residue in the donor antibody, wherein the acceptor heavy chain framework region and donor antibody heavy chain framework region each comprises FR1, FR2, FR3 and FR4;
(b) synthesizing a nucleic acid sequence comprising: (i) a first nucleotide sequence encoding a light chain variable region, and (ii) a second nucleotide sequence encoding a humanized heavy chain variable region with a framework region comprising FR1, FR2, FR3 and FR4 that remains globally less than 65% identical to the donor antibody heavy chain variable framework region at the amino acid level, said second nucleotide sequence comprising nucleic acid sequences encoding CDRs from the donor antibody heavy chain variable region and nucleic acid sequences encoding the acceptor heavy chain variable framework regions with one or more mutations introduced at amino acid residues designated key residues, said key residues not including amino acid residues 2, 4, 24, 35, 36, 39, 43, 45, 64, 69, 70, 73, 74, 75, 76, 78, 92 and 93 according to the Kabat numbering system; and
(c) introducing the nucleic acid sequence comprising the first nucleotide sequence and the second nucleotide sequence into a cell.

31. A cell containing a nucleic acid sequence encoding a humanized antibody that immunospecifically binds to an antigen, said cell produced by the process comprising:
(a) selecting an acceptor heavy chain variable framework region less than 65% identical to a donor antibody heavy chain variable framework region at the amino acid level, which acceptor heavy chain variable framework region contains at least one amino acid residue at amino acid residues 6, 23, 24 or 49 according to the Kabat numbering system that is not identical to the corresponding residue in the donor antibody, wherein the acceptor heavy chain framework region and donor antibody heavy chain framework region each comprises FR1, FR2, FR3 and FR4;
(b) selecting an acceptor light chain variable framework region less than 65% identical to a donor antibody light chain variable framework region at the amino acid level, wherein the acceptor light chain framework region and donor antibody light chain framework region each comprises FR1, FR2, FR3 and FR4;
(c) synthesizing a nucleic acid sequence comprising: (i) a first nucleotide sequence encoding a humanized light chain variable region, said first nucleotide sequence comprising nucleic acid sequences encoding CDRs from the donor antibody light chain variable region and nucleic acid sequences encoding the acceptor light chain variable framework regions with one or more mutations introduced at amino acid residues designated key residues, said key residues not including amino acid residues 4, 38, 43, 44, 46, 58, 62, 65, 66, 67, 68, 69, 73, 85, and 98 according to the Kabat numbering system, and (ii) a second nucleotide sequence encoding a humanized heavy chain variable region with a framework region comprising FR1, FR2, FR3 and FR4 that remains globally less than 65% identical to the donor antibody heavy chain variable framework region at the amino acid level, said second nucleotide sequence comprising nucleic acid sequences encoding complementarity determining regions (CDRs) from the donor antibody heavy chain variable region and nucleic acid sequences encoding the acceptor heavy chain variable framework regions; and
(d) introducing the nucleic acid sequence comprising the first nucleotide sequence and second nucleotide sequence into a cell.

32. A cell containing a nucleotide sequence encoding a humanized antibody that immunospecifically binds to an antigen, said cell produced by the process comprising:
(a) selecting an acceptor heavy chain variable framework region less than 65% identical to a donor antibody heavy chain variable framework region at the amino acid level, which acceptor heavy chain variable framework region contains at least one amino acid residue at amino acid residues 6, 23, 24 or 49 according to the Kabat numbering system that is not identical to the corresponding residue in the donor antibody, wherein the acceptor heavy chain framework region and donor antibody heavy chain framework region each comprises FR1, FR2, FR3 and FR4;
(b) selecting an acceptor light chain variable framework region less than 65% identical to a donor antibody light chain variable framework region at the amino acid level, wherein the acceptor light chain framework region and donor antibody light chain framework region each comprises FR1, FR2, FR3 and FR4;
(c) synthesizing a nucleic acid sequence comprising: (i) a first nucleotide sequence encoding a humanized light chain variable region, said first nucleotide sequence comprising nucleic acid sequences encoding CDRs from the donor antibody light chain variable region and nucleic acid sequences encoding the acceptor light chain variable framework regions with one or more mutations introduced at amino acid residues designated key residues, said key residues not including amino acid residues 4, 38, 43, 44, 46, 58, 62, 65, 66, 67, 68, 69, 73, 85, and 98 according to the Kabat numbering system, and (ii) a second nucleotide sequence encoding a humanized heavy chain variable region with a framework region comprising FR1, FR2, FR3 and FR4 that remains globally less than 65% identical to the donor antibody heavy chain variable framework region at the amino acid level, said second nucleotide sequence comprising nucleic acid sequences encoding CDRs from the donor antibody heavy chain variable region and nucleic acid sequences encoding the acceptor heavy chain variable framework regions with one or more mutations introduced at amino acid residues designated key residues, said key residues not including amino acid residues 2, 4, 24, 35, 36, 39, 43, 45, 64, 69, 70, 73, 74, 75, 76, 78, 92 and 93 according to the Kabat numbering system; and
(d) introducing the nucleic acid sequence comprising the first nucleotide sequence and the second nucleotide sequence into a cell.

33. The cell of embodiment 25, wherein the cell further contains a nucleic acid sequence comprising a nucleotide sequence encoding a light chain variable region.

34. The cell of embodiment 26, wherein the cell further contains a nucleic acid sequence comprising a nucleotide sequence encoding a light chain variable region.

35. The cell of embodiment 33 or 34, wherein the light chain is humanized.

36. The cell of embodiment 29 or 30, wherein the light chain is humanized.

37. The cell of embodiment 26, wherein the residues designated key are one or more of the following: a residue adjacent to a CDR, a potential glycosylation site, a rare residue, a residue capable of interacting with the antigen, a residue capable of interacting with a CDR, a canonical residue, a contact residue between the variable heavy region and variable light region, a residue within the Vernier zone, and a residue within the region which overlaps between the Chothia definition of the heavy chain variable region CDR1 and the Kabat definition of the first heavy chain framework.

38. The cell of embodiment 28, wherein the residues designated key are one or more of the following: a residue adjacent to a CDR, a potential glycosylation site, a rare residue, a residue capable of interacting with the antigen, a residue capable of interacting with a CDR, a canonical residue, a contact residue between the variable heavy region and variable light region, and a residue within the Vernier zone.

39. The cell of embodiment 30, wherein the residues designated key are one or more of the following: a residue adjacent to a CDR, a potential glycosylation site, a rare residue, a residue capable of interacting with the antigen, a residue capable of interacting with a CDR, a canonical residue, a contact residue between the variable heavy region and variable light region, a residue within the Vernier zone, and a residue within the region which overlaps between the Chothia definition of the heavy chain variable region CDR1 and the Kabat definition of the first heavy chain framework.

40. The cell of embodiment 31, wherein the residues designated key are one or more of the following: a residue adjacent to a CDR, a potential glycosylation site, a rare residue, a residue capable of interacting with the antigen, a residue capable of interacting with a CDR, a canonical residue, a contact residue between the variable heavy region and variable light region, and a residue within the Vernier zone.

41. The cell of embodiment 32, wherein the residues designated key are one or more of the following: a residue adjacent to a CDR, a potential glycosylation site, a rare residue, a residue capable of interacting with the antigen, a residue capable of interacting with a CDR, a canonical residue, a contact residue between the variable heavy region and variable light region, a residue within the Vernier zone, and a residue within the region which overlaps between the Chothia definition of the heavy chain variable region CDR1 and the Kabat definition of the first heavy chain framework.

42. The cell of embodiment 33, wherein the residues designated key are one or more of the following: a residue adjacent to a CDR, a potential glycosylation site, a rare residue, a residue capable of interacting with the antigen, a residue capable of interacting with a CDR, a canonical residue, a contact residue between the variable heavy region and variable light region, and a residue within the Vernier zone.

43. The cell of embodiment 26, wherein the mutations are substitutions.

44. The cell of embodiment 28, wherein the mutations are substitutions.

45. The cell of embodiment 30, wherein the mutations are substitutions.

46. The cell of embodiment 31, wherein the mutations are substitutions.

47. The cell of embodiment 32, wherein the mutations are substitutions.

48. The cell of embodiment 43, wherein the substitutions replace the acceptor amino acid residues in the heavy chain variable framework region with the corresponding amino acid residues in the donor heavy chain variable framework region.

49. The cell of embodiment 44, wherein the substitutions replace the acceptor amino acid residues in the light chain variable framework region with the corresponding amino acid residues in the donor light chain variable framework region.

50. The cell of embodiment 45, wherein the substitutions replace the acceptor amino acid residues in the heavy chain variable framework region with the corresponding amino acid residues in the donor heavy chain variable framework region.

51. The cell of embodiment 46, wherein the substitutions replace the acceptor amino acid residues in the light chain variable framework region with the corresponding amino acid residues in the donor light chain variable framework region.

52. The cell of embodiment 47, wherein the substitutions replace the acceptor amino acid residues in the heavy chain variable framework region with the corresponding amino acid residues in the donor heavy chain variable framework region.

53. The cell of embodiment 48, wherein the substitutions replace the acceptor amino acid residues in the light chain variable framework region with the corresponding amino acid residues in the donor light chain variable framework region.

54. The cell of embodiment 47, wherein the substitutions replace the acceptor amino acid residues in the heavy and light chain variable framework regions with the corresponding amino acid residues in the donor heavy and light chain variable framework regions.

55. The cell of embodiment 26, 30 or 31, wherein the acceptor heavy chain variable framework region contains donor antibody amino acid residues at amino acid residues 6 and 23.

56. The cell of embodiment 26, 30 or 31, wherein the acceptor heavy chain variable framework region contains donor antibody amino acid residues at amino acid residues 6 and 24.

57. The cell of embodiment 26, 30 or 31, wherein the acceptor heavy chain variable framework region contains donor antibody amino acid residues at amino acid residues 6 and 49.

58. The cell of embodiment 26, 30 or 31, wherein the acceptor heavy chain variable framework region contains donor antibody amino acid residues at amino acid residues 23 and 49.

59. The cell of embodiment 26, 30 or 31, wherein the acceptor heavy chain variable framework region contains donor antibody amino acid residues at amino acid residues 24 and 49.

60. The cell of embodiment 26, 30 or 31, wherein the acceptor heavy chain variable framework region contains donor antibody amino acid residues at amino acid residues 23 and 24.

61. The cell of embodiment 55, wherein the acceptor heavy chain variable framework region further contains donor antibody amino acid residues at amino acid residue 49.

62. The cell of embodiment 56, wherein the acceptor heavy chain variable framework region further contains donor antibody amino acid residues at amino acid residue 49.

63. The cell of embodiment 60, wherein the acceptor heavy chain variable framework region further contains donor antibody amino acid residues at amino acid residue 49.

64. The cell of embodiment 55, wherein the acceptor heavy chain variable framework region further contains donor antibody amino acid residues at amino acid residues 24.

65. The cell of embodiment 64, wherein the acceptor heavy chain variable framework region further contains donor antibody amino acid residues at amino acid residue 49.

66. The cell of embodiment 26, 30 or 32, wherein the amino acid residues designated key are not heavy chain variable framework region amino acid residues 6, 23, 24 and 49 according to the Kabat numbering system.

67. The cell of embodiment 25, 26, 29, 30, 31 or 32, wherein the acceptor heavy chain variable framework region is less than 60% identical to the donor antibody heavy chain variable framework region.

68. The cell of embodiment 67, wherein the acceptor heavy chain variable framework region is less than 55% identical to the donor antibody heavy chain variable framework region.

69. The cell of embodiment 68, wherein the acceptor heavy chain variable framework region is less than 50% identical to the donor antibody heavy chain variable framework region.

70. The cell of embodiment 27, 28, 31 or 32, wherein the acceptor light chain variable framework region is less than 65% identical to the donor antibody light chain variable framework region at the amino acid level.

71. The cell of embodiment 70, wherein the acceptor light chain variable framework region is less than 60% identical to the donor antibody light chain variable framework region.

72. The cell of embodiment 71, wherein the acceptor light chain variable framework region is less than 55% identical to the donor antibody light chain variable framework region.

73. The cell of embodiment 25, 26, 29, 30, 31 or 32, wherein a donor antibody amino acid residue in the humanized heavy chain variable framework region is not within 6 Å of a CDR.

74. The cell of embodiment 26, 30 or 32, wherein a donor antibody amino acid residue in the humanized light chain variable framework region is not within 6 Å of a CDR.

75. The cell of any of the embodiments 25 to 32, wherein said acceptor is human.

76. The cell of any of the embodiments 25 to 32, wherein said acceptor contains at least one amino acid residue that does not occur at a specific position of a human antibody.

77. A population of cells engineered to contain nucleotide sequences encoding a plurality of humanized antibodies produced by a process comprising:
(a) selecting acceptor heavy chain variable framework regions less than 65% identical to a donor antibody heavy chain variable framework region at the amino acid level, which acceptor heavy chain variable framework regions contain amino acid residues at amino acid residues 6, 23, 24 or 49 according to the Kabat numbering system that are not conserved between the framework region of the donor antibody and the acceptor heavy chain variable framework region, wherein the acceptor heavy chain framework region and donor antibody heavy chain framework region each comprises FR1, FR2, FR3 and FR4;
(b) synthesizing a nucleic acid sequences comprising nucleotide sequences encoding humanized heavy chain variable regions, said nucleotide sequences comprising nucleic acid sequences encoding complementarity determining regions (CDRs) from the donor antibody heavy chain variable region and nucleic acid sequences encoding the acceptor heavy chain variable framework regions; and
(c) introducing the nucleic acid sequences comprising the nucleotide sequences encoding the humanized heavy chain variable regions into cells.

78. A population of cells engineered to contain nucleotide sequences encoding a plurality of humanized antibodies produced by a process comprising:
(a) selecting acceptor heavy chain variable framework regions less than 65% identical to a donor antibody heavy chain variable framework region at the amino acid level, which acceptor heavy chain variable framework regions contain amino acid residues at amino acid residues 6, 23, 24 or 49 according to the Kabat numbering system that are not conserved between the framework region of the donor antibody and the acceptor heavy chain variable framework region, wherein the acceptor heavy chain framework region and donor antibody heavy chain framework region each comprises FR1, FR2, FR3 and FR4;
(b) synthesizing nucleic acid sequences comprising nucleotide sequences encoding humanized heavy chain variable regions with framework regions comprising FR1, FR2, FR3 and FR4 that remain globally less than 65% identical to the donor antibody heavy chain variable framework region at the amino acid level, said nucleotide sequences comprising nucleic acid sequences encoding CDRs from the donor antibody heavy chain variable region and nucleic acid sequences encoding the acceptor heavy chain variable framework regions with one or more mutations introduced at amino acid residues designated key residues, said key residues not including amino acid residues 2, 4, 24, 35, 36, 39, 43, 45, 64, 69, 70, 73, 74, 75, 76, 78, 92 and 93 according to the Kabat numbering system; and
(c) introducing the nucleic acid sequences comprising the nucleotide sequences encoding the humanized heavy chain variable regions into cells.

79. A population of cells engineered to contain nucleotide sequences encoding a plurality of humanized antibodies produced by a process comprising:
(a) selecting acceptor light chain variable framework regions less than 65% identical to a donor antibody light chain variable framework region at the amino acid level, wherein the acceptor light chain framework region and donor antibody light chain framework region each comprises FR1, FR2, FR3 and FR4;
(b) synthesizing nucleic acid sequences comprising nucleotide sequences encoding humanized light chain variable regions, said nucleotide sequences comprising nucleic acid sequences encoding complementarity determining regions (CDRs) from the donor antibody light chain variable region and nucleic acid sequences encoding the acceptor light chain variable framework regions; and
(c) introducing the nucleic acid sequences comprising the nucleotide sequences encoding the humanized light chain variable regions into cells.

80. A population of cells engineered to contain nucleotide sequences encoding a plurality of humanized antibodies produced by a process comprising:
(a) selecting acceptor light chain variable framework regions less than 65% identical to a donor antibody heavy chain variable framework region at the amino acid level, wherein the acceptor light chain framework region and donor antibody light chain framework region each comprises FR1, FR2, FR3 and FR4;
(b) synthesizing nucleic acid sequences comprising nucleotide sequences encoding humanized light chain variable regions, said nucleotide sequences comprising nucleic acid sequences encoding CDRs from the donor antibody light chain variable region and nucleic acid sequences encoding the acceptor light chain variable framework regions with one or more mutations introduced at amino acid residues designated key residues, said key residues not including amino acid residues 4, 38, 43, 44, 46, 58, 62, 65, 66, 67, 68, 69, 73, 85, and 98 according to the Kabat numbering system; and
(c) introducing the nucleic acid sequences comprising the nucleotide sequences encoding the humanized light chain variable regions into cells.

81. A population of cells engineered to contain nucleotide sequences encoding a plurality of humanized antibodies produced by a process comprising:
(a) selecting acceptor heavy chain variable framework regions less than 65% identical to a donor antibody heavy chain variable framework region at the amino acid level, which acceptor heavy chain variable framework regions contain amino acid residues at amino acid residues 6, 23, 24 or 49 according to the Kabat numbering system that are not conserved between the framework region of the donor antibody and the acceptor heavy chain variable framework region, wherein the acceptor heavy chain framework region and donor antibody heavy chain framework region each comprises FR1, FR2, FR3 and FR4;
(b) synthesizing nucleic acid sequences comprising: (i) a first set of nucleotides sequence encoding light chain variable regions, and (ii) a second set of nucleotide sequences encoding humanized heavy chain variable regions with framework regions comprising FR1, FR2, FR3 and FR4 that remain globally less than 65% identical to the donor antibody heavy chain variable framework region at the amino acid level, said second set of nucleotide sequences comprising nucleic acid sequences encoding complementarity determining regions (CDRs) from the donor antibody heavy chain variable region and nucleic acid sequences encoding the acceptor heavy chain variable framework regions; and
(c) introducing the nucleic acid sequences comprising the first set of nucleotide sequences and second set of nucleotide sequences into a cell.

82. A population of cells engineered to contain nucleotide sequences encoding a plurality of humanized antibodies produced by a process comprising:
(a) selecting acceptor heavy chain variable framework regions less than 65% identical to a donor antibody heavy chain variable framework region at the amino acid level, which acceptor heavy chain variable framework regions contain amino acid residues at amino acid residues 6, 23, 24 or 49 according to the Kabat numbering system that are not conserved between the framework region of the donor antibody and the acceptor heavy chain variable framework region, wherein the acceptor heavy chain framework region and donor antibody heavy chain framework region each comprises FR1, FR2, FR3 and FR4;
(b) synthesizing a nucleic acid sequence comprising: (i) a first set of nucleotide sequences encoding light chain variable regions, and (ii) a second set of nucleotide sequences encoding humanized heavy chain variable regions with framework regions comprising FR1, FR2, FR3 and FR4 that remain globally less than 65% identical to the donor antibody heavy chain variable framework region at the amino acid level, said second set of nucleotide sequence comprising nucleic acid sequences encoding CDRs from the donor antibody heavy chain variable region and nucleic acid sequences encoding the acceptor heavy chain variable framework regions with one or more mutations introduced at amino acid residues designated key residues, said key residues not including amino acid residues 2, 4, 24, 35, 36, 39, 43, 45, 64, 69, 70, 73, 74, 75, 76, 78, 92 and 93 according to the Kabat numbering system; and
(c) introducing the nucleic acid sequences comprising the first set of nucleotide sequences and the second set of nucleotide sequences into cells.

83. A population of cells engineered to contain nucleotide sequences encoding a plurality of humanized antibodies produced by a process comprising:
(a) selecting acceptor heavy chain variable framework regions less than 65% identical to a donor antibody heavy chain variable framework region at the amino acid level, which acceptor heavy chain variable framework regions contain amino acid residues at amino acid residues 6, 23, 24 or 49 according to the Kabat numbering system that are not conserved between the framework region of the donor antibody and the acceptor heavy chain variable framework region, wherein the acceptor heavy chain framework region and donor antibody heavy chain framework region each comprises FR1, FR2, FR3 and FR4;
(c) selecting acceptor light chain variable framework regions less than 65% identical to a donor antibody light chain variable framework region at the amino acid level, wherein the acceptor light chain framework region and donor antibody light chain framework region each comprises FR1, FR2, FR3 and FR4;
(c) synthesizing nucleic acid sequences comprising: (i) a first set of nucleotide sequences encoding humanized light chain variable regions, said first set of nucleotide sequences comprising nucleic acid sequences encoding CDRs from the donor antibody light chain variable region and nucleic acid sequences encoding the acceptor light chain variable framework regions with one or more mutations introduced at amino acid residues designated key residues, said key residues not including amino acid residues 4, 38, 43, 44, 46, 58, 62, 65, 66, 67, 68, 69, 73, 85, and 98 according to the Kabat numbering system, and (ii) a second set of nucleotide sequences encoding humanized heavy chain variable regions with framework regions comprising FR1, FR2, FR3 and FR4 that remain globally less than 65% identical to the donor antibody heavy chain variable framework region at the amino acid level, said second set of nucleotide sequences comprising nucleic acid sequences encoding complementarity determining regions (CDRs) from the donor antibody heavy chain variable region and nucleic acid sequences encoding the acceptor heavy chain variable framework regions; and
(d) introducing the nucleic acid sequences comprising the first set of nucleotide sequences and second set of nucleotide sequences into cells.

84. A population of cells engineered to contain nucleotide sequences encoding a plurality of humanized antibodies produced by a process comprising:
(a) selecting acceptor heavy chain variable framework regions less than 65% identical to a donor antibody heavy chain variable framework region at the amino acid level, which acceptor heavy chain variable framework regions contain amino acid residues at amino acid residues 6, 23, 24 or 49 according to the Kabat numbering system that are not conserved between the framework region of the donor antibody and the acceptor heavy chain variable framework region, wherein the acceptor heavy chain framework region and donor antibody heavy chain framework region each comprises FR1, FR2, FR3 and FR4;
(b) selecting acceptor light chain variable framework regions less than 65% identical to a donor antibody light chain variable framework region at the amino acid level, wherein the acceptor light chain framework region and donor antibody light chain framework region each comprises FR1, FR2, FR3 and FR4;
(c) synthesizing nucleic acid sequences comprising: (i) a first set of nucleotide sequences encoding humanized light chain variable regions, said first set of nucleotide sequences comprising nucleic acid sequences encoding CDRs from the donor antibody light chain variable region and nucleic acid sequences encoding the acceptor light chain variable framework regions with one or more mutations introduced at amino acid residues designated key residues, said key residues not including amino acid residues 4, 38, 43, 44, 46, 58, 62, 65, 66, 67, 68, 69, 73, 85, and 98 according to the Kabat numbering system, and (ii) a second set of nucleotide sequences encoding humanized heavy chain variable regions with framework regions comprising FR1, FR2, FR3 and FR4 that remain globally less than 65% identical to the donor antibody heavy chain variable framework region at the amino acid level, said second set of nucleotide sequences comprising nucleic acid sequences encoding CDRs from the donor antibody heavy chain variable region and nucleic acid sequences encoding the acceptor heavy chain variable framework regions with one or more mutations introduced at amino acid residues designated key residues, said key residues not including amino acid residues 2, 4, 24, 35, 36, 39, 43, 45, 64, 69, 70, 73, 74, 75, 76, 78, 92 and 93 according to the Kabat numbering system; and
(d) introducing the nucleic acid sequences comprising the first set of nucleotide sequences and the second set of nucleotide sequences into cells.

85. The cells of embodiment 77, wherein the cells further contains a nucleic acid sequence comprising a nucleotide sequence encoding a light chain variable region.

86. The cells of embodiment 78, wherein the cells further contains a nucleotide sequence encoding a light chain variable region.

87. The cells of embodiment 81 or 82, wherein the light chain is humanized.

88. The cells of embodiment 85 or 87, wherein the light chain is humanized.

89. The cells of embodiment 78, wherein the residues designated key are one or more of the following: a residue adjacent to a CDR, a potential glycosylation site, a rare residue, a residue capable of interacting with the antigen, a residue capable of interacting with a CDR, a canonical residue, a contact residue between the variable heavy region and variable light region, a residue within the Vernier zone, and a residue within the region which overlaps between the Chothia definition of the heavy chain variable region CDR1 and the Kabat definition of the first heavy chain framework.

90. The cells of embodiment 80, wherein the residues designated key are one or more of the following: a residue adjacent to a CDR, a potential glycosylation site, a rare residue, a residue capable of interacting with the antigen, a residue capable of interacting with a CDR, a canonical residue, a contact residue between the variable heavy region and variable light region, and a residue within the Vernier zone.

91. The cells of embodiment 82, wherein the residues designated key are one or more of the following: a residue adjacent to a CDR, a potential glycosylation site, a rare residue, a residue capable of interacting with the antigen, a residue capable of interacting with a CDR, a canonical residue, a contact residue between the variable heavy region and variable light region, a residue within the Vernier zone, and a residue within the region which overlaps between the Chothia definition of the heavy chain variable region CDR1 and the Kabat definition of the first heavy chain framework.

92. The cells of embodiment 83, wherein the residues designated key are one or more of the following: a residue adjacent to a CDR, a potential glycosylation site, a rare residue, a residue capable of interacting with the antigen, a residue capable of interacting with a CDR, a canonical residue, a contact residue between the variable heavy region and variable light region, and a residue within the Vernier zone.

93. The cells of embodiment 84, wherein the residues designated key are one or more of the following: a residue adjacent to a CDR, a potential glycosylation site, a rare residue, a residue capable of interacting with the antigen, a residue capable of interacting with a CDR, a canonical residue, a contact residue between the variable heavy region and variable light region, a residue within the Vernier zone, and a residue within the region which overlaps between the Chothia definition of the heavy chain variable region CDR1 and the Kabat definition of the first heavy chain framework.

94. The cells of embodiment 85, wherein the residues designated key are one or more of the following: a residue adjacent to a CDR, a potential glycosylation site, a rare residue, a residue capable of interacting with the antigen, a residue capable of interacting with a CDR, a canonical residue, a contact residue between the variable heavy region and variable light region, a residue within the Vernier zone, and a residue within the region which overlaps between the Chothia definition of the heavy chain variable region CDR1 and the Kabat definition of the first heavy chain framework.

95. The cells of embodiment 78, wherein the mutations are substitutions.

96. The cells of embodiment 80, wherein the mutations are substitutions.

97. The cells of embodiment 82, wherein the mutations are substitutions.

98. The cells of embodiment 83, wherein the mutations are substitutions.

99. The cells of embodiment 84, wherein the mutations are substitutions.

100. The cells of embodiment 95, wherein the substitutions replace the acceptor amino acid residues in the heavy chain variable framework regions with the corresponding amino acid residues in the donor heavy chain variable framework region.

101. The cells of embodiment 96, wherein the substitutions replace the acceptor amino acid residues in the light chain variable framework regions with the corresponding amino acid residues in the donor light chain variable framework region.

102. The cells of embodiment 97, wherein the substitutions replace the acceptor amino acid residues in the heavy chain variable framework regions with the corresponding amino acid residues in the donor heavy chain variable framework region.

103. The cells of embodiment 98, wherein the substitutions replace the acceptor amino acid residues in the light chain variable framework regions with the corresponding amino acid residues in the donor light chain variable framework region.

104. The cells of embodiment 99, wherein the substitutions replace the acceptor amino acid residues in the heavy chain variable framework regions with the corresponding amino acid residues in the donor heavy chain variable framework region.

105. The cells of embodiment 99, wherein the substitutions replace the acceptor amino acid residues in the light chain variable framework regions with the corresponding amino acid residues in the donor light chain variable framework region.

106. The cells of embodiment 99, wherein the substitutions replace the acceptor amino acid residues in the heavy and light chain variable framework regions with the corresponding amino acid residues in the donor heavy and light chain variable framework regions.

107. The cells of embodiment 78, 82 or 83, wherein the acceptor heavy chain variable framework region contains donor antibody amino acid residues at amino acid residues 6 and 23.

108. The cells of embodiment 78, 82 or 83, wherein the acceptor heavy chain variable framework region contains donor antibody amino acid residues at amino acid residues 6 and 24.

109. The cells of embodiment 78, 82 or 83, wherein the acceptor heavy chain variable framework region contains donor antibody amino acid residues at amino acid residues 6 and 49.

110. The cells of embodiment 78, 82 or 83, wherein the acceptor heavy chain variable framework region contains donor antibody amino acid residues at amino acid residues 23 and 49.

111. The cells of embodiment 78, 82 or 83, wherein the acceptor heavy chain variable framework region contains donor antibody amino acid residues at amino acid residues 24 and 49.

112. The cells of embodiment 78, 82 or 83, wherein the acceptor heavy chain variable framework region contains donor antibody amino acid residues at amino acid residues 23 and 24.

113. The cells of embodiment 107, wherein the acceptor heavy chain variable framework region further contains donor antibody amino acid residues at amino acid residue 49.

114. The cells of embodiment 108, wherein the acceptor heavy chain variable framework region further contains donor antibody amino acid residues at amino acid residue 49.

115. The cells of embodiment 112, wherein the acceptor heavy chain variable framework region further contains donor antibody amino acid residues at amino acid residue 49.

116. The cells of embodiment 107, wherein the acceptor heavy chain variable framework region further contains donor antibody amino acid residues at amino acid residues 24.

117. The cells of embodiment 116, wherein the acceptor heavy chain variable framework region further contains donor antibody amino acid residues at amino acid residues 49.

118. The cells of embodiment 78, 82 or 83, wherein the amino acid residues designated key are not heavy chain variable framework region amino acid residues 6, 23, 24 or 49 according to the Kabat numbering system.

119. The cells of embodiment 77, 78, 79, 80, 81, 82, 83 or 84, wherein the acceptor heavy chain variable framework regions are less than 60% identical to the donor antibody heavy chain variable framework region.

120. The cells of any of the embodiments 77 to 84, wherein said acceptor is human.

121. The cells of any of the embodiments 77 to 84, wherein said acceptor contains at least one amino acid residue that does not occur at a specific position of a human antibody.

122. A method of producing a humanized antibody that immunospecifically binds to an antigen, said method comprising expressing nucleic acid sequences encoding the humanized antibody contained in the cell of embodiment 25.

123. A method of producing a humanized antibody that immunospecifically binds to an antigen, said method comprising expressing nucleic acid sequences encoding the humanized antibody contained in the cell of embodiment 26.

124. A method of producing a humanized antibody that immunospecifically binds to an antigen, said method comprising expressing nucleic acid sequences encoding the humanized antibody contained in the cell of embodiment 27.

125. A method of producing a humanized antibody that immunospecifically binds to an antigen, said method comprising expressing the nucleic acid sequence encoding the humanized antibody contained in the cell of embodiment 29.

126. A method of producing a humanized antibody that immunospecifically binds to an antigen, said method comprising expressing the nucleic acid sequence encoding the humanized antibody contained in the cell of embodiment 30.

127. A method of producing a humanized antibody that immunospecifically binds to an antigen, said method comprising expressing the nucleic acid sequence encoding the humanized antibody contained in the cell of embodiment 31.

128. A method of producing a humanized antibody that immunospecifically binds to an antigen, said method comprising expressing the nucleic acid sequence encoding the humanized antibody contained in the cell of embodiment 32.

129. A method of producing a humanized antibody that immunospecifically binds to an antigen, said method comprising providing a cell containing nucleotide sequences encoding humanized heavy chain and light chain variable regions and expressing the nucleotide sequences, wherein said cell containing the nucleotide sequences was produced by:
(a) comparing the nucleotide sequence of a donor antibody heavy chain variable region against a collection of sequences of acceptor heavy chain variable regions;
(b) selecting an acceptor heavy chain variable framework region less than 65% identical to the donor antibody heavy chain variable framework region at the amino acid level, which acceptor heavy chain variable framework region contains at least one amino acid residue at amino acid residues 6, 23, 24 or 49 according to the Kabat numbering system that is not identical to the corresponding residue in the donor antibody, wherein the acceptor heavy chain framework region and donor antibody heavy chain framework region each comprises FR1, FR2, FR3 and FR4;
(b) synthesizing a nucleotide sequence encoding a humanized heavy chain variable region, said nucleotide sequence comprising nucleic acid sequences encoding complementarity determining regions (CDRs) from the donor antibody heavy chain variable region and nucleic acid sequences encoding the acceptor heavy chain variable framework regions; and
(c) introducing the nucleotide sequence encoding the humanized heavy chain variable region into a cell.

130. A method of producing a humanized antibody that immunospecifically binds to an antigen, said method comprising providing a cell containing nucleotide sequences encoding humanized heavy chain and light chain variable regions and expressing nucleotide sequences, wherein said cell containing the nucleotide sequences was produced by:
(a) comparing the nucleotide sequence of a donor antibody heavy chain variable region against a collection of sequences of acceptor heavy chain variable regions;
(b) selecting an acceptor heavy chain variable framework region less than 65% identical to the donor antibody heavy chain variable framework region at the amino acid level, which acceptor heavy chain variable framework region contains at least one amino acid residue at amino acid residues 6, 23, 24 or 49 according to the Kabat numbering system that is not identical to the corresponding residue in the donor antibody, wherein the acceptor heavy chain framework region and donor antibody heavy chain framework region each comprises FR1, FR2, FR3 and FR4;
(c) synthesizing a nucleic acid sequence comprising nucleotide sequence encoding a humanized heavy chain variable region, said nucleotide sequence comprising nucleic acid sequences encoding complementarity determining regions (CDRs) from the donor antibody heavy chain variable region and nucleic acid sequences encoding the acceptor heavy chain variable framework regions with one or more mutations introduced at residues designated key residues; and
(d) introducing the nucleic acid sequence comprising the nucleotide sequence encoding the humanized heavy chain variable region into a cell.

131. The method of embodiment 129, wherein the residues designated key are one or more of the following: a residue adjacent to a CDR, a potential glycosylation site, a rare residue, a residue capable of interacting with the antigen, a residue capable of interacting with a CDR, a canonical residue, a contact residue between the variable heavy region and variable light region, and a residue within the Vernier zone.

132. The method of embodiment 130, wherein the residues designated key are one or more of the following: a residue adjacent to a CDR, a potential glycosylation site, a rare residue, a residue capable of interacting with the antigen, a residue capable of interacting with a CDR, a canonical residue, a contact residue between the variable heavy region and variable light region, a residue within the Vernier zone, and a residue within the region which overlaps between the Chothia definition of the heavy chain variable region CDR1 and the Kabat definition of the first heavy chain framework.

133. The method of embodiment 131, wherein the mutations are substitutions.

134. The method of embodiment 132, wherein the mutations are substitutions.

135. The method of embodiment 133, wherein the substitutions replace the acceptor amino acid residues in the heavy chain variable framework region with the corresponding amino acid residues in the donor heavy chain variable framework region.

136. The method of embodiment 134, wherein the substitutions replace the acceptor amino acid residues in the heavy chain variable framework region with the corresponding amino acid residues in the donor heavy chain variable framework region.

137. The method of embodiment 129, wherein the amino acid residues designated key are not amino acid residues 6, 23, 24 or 49.

138. The method of embodiment 130, wherein the amino acid residues designated key are not amino acid residues 6, 23, 24 or 49.

139. The method of embodiment 129 or 130, wherein said acceptor is human.

140. The method of embodiment 129 or 130, wherein said acceptor contains at least one amino acid residue that does not occur at a specific position of a human antibody.

141. A humanized antibody produced by the method of embodiment 122, 123, 124, 125, 126, 127 or 128.

142. A humanized antibody produced by the method of embodiment 129 or 130.

143. A composition comprising the humanized antibody of embodiment 138, and a carrier, diluent or excipient.

144. A composition comprising the humanized antibody of embodiment 142, and a carrier, diluent or excipient.

145. A method of identifying a humanized antibody that immunospecifically binds to an antigen, said method comprising expressing the nucleic acid sequences in the cells of embodiment 53, 54, 55, 56, 57, 58 or 59 and screening for a humanized antibody that has an affinity of 1×10⁶ M⁻¹ or above for said antigen.

146. A humanized antibody identified by the method of embodiment 145.

147. A composition comprising the humanized antibody of embodiment 146, and a carrier, diluent or excipient.

### 6. EXAMPLE

### Humanization Of Anti-Interleukin-9 Antibodies

Interleukin-9 (“IL-9”) is member of the 4-helix bundle cytokine family, which includes IL-2, IL-3, IL-4, IL-5, IL-6, IL-7, IL-15, and IL-23. IL-9 plays a critical role in a number of antigen-induced responses in mice, such as bronchial hyperresponsiveness, epithelial mucin production, eosinophilia, elevated T cells, B cells, mast cells, neutrophils, and other inflammatory cell counts in the bronchial lavage, histologic changes in the lung associated with inflammation, and elevated serum total IgE. See U.S. Application Ser. Nos. 60/477,797 and 60/477,801 (both filed Jun. 10, 2003, MedImmune, Inc., incorporated herein by reference). IL-9 is expressed by activated T cells and mast cells and functions as a T cell growth factor. Further, IL-9 mediates the growth of erythroid progenitors, B cells, mast cells, eosinophils, and fetal thymocytes, acts synergistically with interleukin-3 (“IL-3”) to induce mast cell activation and proliferation, and promotes the production of mucin by lung epithelium.

Structural similarity has been observed for the human and murine IL-9 genes, suggesting that human IL-9 would be expected to play a similar role in the indication of asthmatic immune responses in human. It would be valuable for human patients suffering from diseases or conditions associated with IL-9 expression such as asthma if antibodies having a low immunogenicity and a high binding affinity for human IL-9 could be designed for use in human therapy. This example demonstrates how such antibodies can be constructed according to the present invention.

### 6.1. Selection of Human Framework

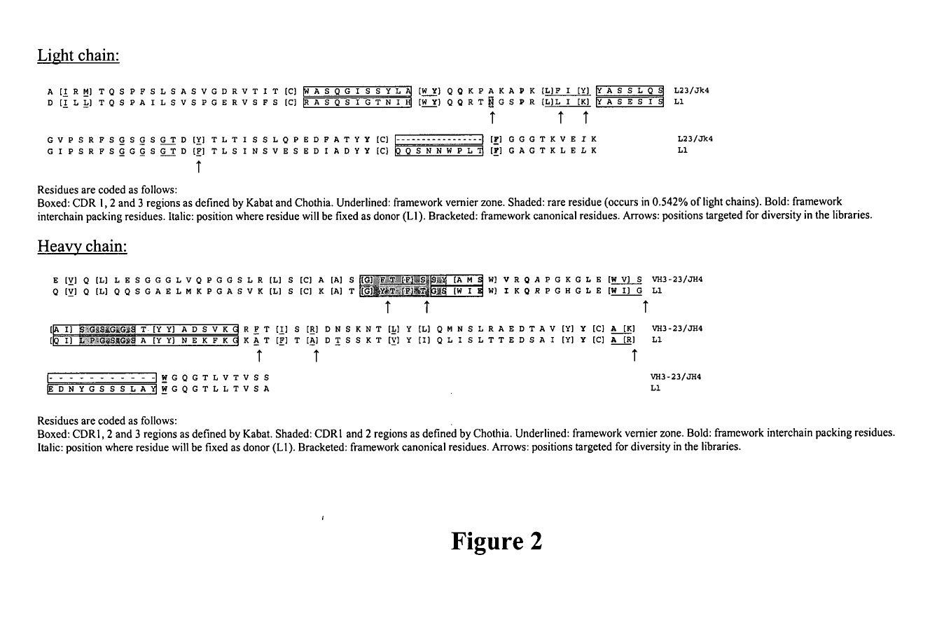

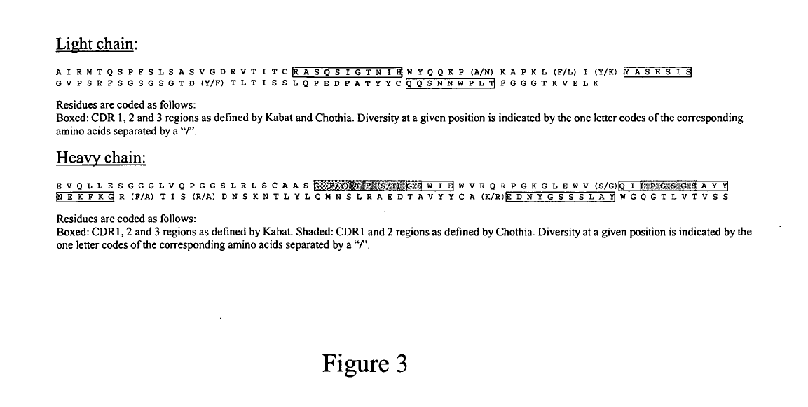

According to the rules of design (see Section 5.1), human germline V_{H}3-23 in combination with JH4 was used to graft the donor heavy chain CDR loops and human germline L23 in combination with Jκ4 was used to graft the donor light chain CDR loops (see FIG. 2). Using those combinations, homologies between donor antibody and acceptor antibody frameworks were 60% and 56.3% for the light chain and the heavy chain according to Kabat definition, respectively. In the humanized light chain, diversity was introduced at four positions (41, 47, 49 and 71 according to Kabat numbering). In the humanized heavy chain, four (49, 67, 71 and 94 according to Kabat numbering) or six (27, 30, 49, 67, 71 and 94 according to Kabat numbering) positions were diversified, depending on what definition of the heavy chain CDR1 and 2 (i.e., Chothia or Kabat, respectively), is used (see FIG. 3). Briefly, mutagenesis was carried out using the Polymerase Chain Reaction by overlap extension in order to synthesize the humanized L1-light and L1-heavy chains where all mouse residues were substituted by their human counterparts except in regions where diversity was introduced (see FIG. 3 and Rule (6)(a)-(f) in Section 5.1) or where a donor residue was fixed (see FIG. 3 and Rule (5)). This was carried out with degenerated oligonucleotides encoding the codons for both the human and mouse residues (wobbles).

### 6.2. Construction of Combinatorial Libraries

Two libraries were constructed: library 1 comprised a heavy chain combinatorial library (with CDRs definition according to Kabat) and a light chain combinatorial library using oligonucleotides whose length ranged from 47 to 80 mers (see Table 7 and 8). Library 2 comprised a heavy chain combinatorial library (with CDRs definition according to Chothia) and a light chain combinatorial library using oligonucleotides whose length ranged from 39 to 60 mers (see Table 9 and 10. In Table 7-10, all oligonucleotides are shown in the 5′ to 3′ orientation, name followed by sequence, wherein K=G or T, M=A or C, R=A or G, S=C or G, W=A or T, and Y=C or T).

TABLE 7
Library 1-Heavy chain (CDRs defined according to Kabat):

| | |
|---|---|
| 414 | 1K Biotin-GATTCCGCTGGTGGTGCCGTTCTATAGCCATAGCGAGGTGCAGCTG TGG |
| | AGTCTGGGGGAGGCTTGGTACAGCCTGGG |
| 415 | 2K CAGAGGCTGCACAGGAGAGTCTCAGGGACCCCCCAGGCTGTACCAAGCC |
| 416 | 3K CTCCTGTGCAGCCTCTGGATWCACCTTTASCGGCTCCTGGATAGAGTGGGTCC |
| | GCCAGCGTCCAGGGAAGGGGCTG (C) |
| 417 | 4K CCTTGAACTTCTCATTGTAGTAAGCACTACCACTTCCAGGTAAAATCTGGCYGAC |
| | CCACT CCAGCCCCTTCCCTGGA |
| 418 | 5K CTACAATGAGAAGTTCAAGGGCCGGTTCACCATCTCCAGAGACAATTCCAAGA A |
| | CACGCTGTATCTGCAAATGAACAGCC |
| 419 | 6K CTACAATGAGAAGTTCAAGGGCCGGTTCACCATCTCCGCAGACAATTCCAAGAA |
| | CACGCTGTATCTGCAAATGAACAGCC |
| 420 | 7K CTACAATGAGAAGTTCAAGGGCCGGGCCACCATCTCCAGAGACAATTCCAAGAA |
| | CACGCTGTATCTGCAAATGAACAGCC |
| 421 | 8K CTACAATGAGAAGTTCAAGGGCCGGGCCACCATCTCCGCAGACAATTCCAAGAA |
| | CACGCTGTATCTGCAAATGAACAGCC |
| 422 | 9K GTTATCCTCTYTCGCACAGTAATATACGGCCGTGTCCTCGGCTCTCAGGCTGTTC |
| | ATTTGCAGATA |
| 423 | 10K CTGTGCGARAGAGGATAACTACGGTAGTAGCTCGTTAGCTTACTGGGGCCAAGG |
| | AACCCTGGTCAC |
| 424 | 11K GGGGGAAGACCGATGGGCCCTTGGTGGAGGCTGAGGAGACGGTGACCAGG GT T |
| | CCTTG |

TABLE 8
Library 1-Light chain:

| | |
|---|---|
| 425 | 1′K Biotin-GGTCGTTCCATTTTACTCCCACTCCGCCATCCGGATGACCCAGTCTCCATT |
| | CTC CCTGTCTGCAT |
| 426 | 2′K TTGTGCCAATGCTCTGACTGGCCCTGCAAGTGATGGTGACTCTGTCTCCTACAGA |
| | TGCAGACAGGGAGAATG |
| 427 | 3′K GTCAGAGCATTGGCACAAACATTCACTGGTATCAGCAAAAACCAGCAAAAGCCCCTAAGCTC |
| | YTCAT |
| 428 | 4′K GTCAGAGCATTGGCACAAACATTCACTGGTATCAGCAAAAACCAAATAAAGCC |
| | CCTAAGCTCYTCAT |
| 429 | 5′K CGCTGAACCTTGATGGGACCCCAGAGATAGACTCAGAAGCATAATAGATGARG |
| | AGCTTAGGGGCT |
| 430 | 6′K CGCTGAACCTTGATGGGACCCCAGAGATAGACTCAGAAGCATACTTGATGARGA |
| | GCTTAGGGGCT |
| 431 | 7′K CCCATCAAGGTTCAGCGGCAGTGGATCTGGGACGGATTWCACTCTCACCATCAG |
| | CAGCCTGCAG |
| 432 | 8′K CGGCCAGTTATTACTTTGTTGACAGTAATAAGTTGCAAAATCTTCAGGCTGCAG |
| | GCTGCTGATGG |
| 433 | 9′K CAACAAAGTAATAACTGGCCGCTCACGTTCGGCGGAGGGACCAAGGT |
| 434 | 10′K GATGAAGACAGATGGTGCAGCCACAGTACGTTTGAGCTCCACCTTGGTCCCTCC |
| | GCCGAACG |

TABLE 9
Library 2-Heavy chain (CDRs defined according to Chothia):

| | |
|---|---|
| 435 | 1C Biotin-TTCCGCTGGTGGTGCCGTTCTATAGCCATAGCGAGGTGCAGCTGTTGGAG |
| 436 | 2C GGACCCCCCAGGCTGTACCAAGCCTCCCCCAGACTCCAACAGCTGCACCTC |
| 437 | 3C TACAGCCTGGGGGGTCCCTGAGACTCTCCTGTGCAGCCTCTGGATTCACC |
| 438 | 4C TGGCGGACCCACTCTATCCAGGAGCCGCTAAAGGTGAATCCAGAGGCTGC |
| 439 | 5C GATAGAGTGGGTCCGCCAGCGTCCAGGGAAGGGGCTGGAGTGGGTCRGCCAGAT |
| 440 | 6C CTTGAACTTCTCATTGTAGTAAGCACTACCACTTCCAGGTAAAATCTGGCYGACC |
| | CACTC |
| 441 | 7C ACTACAATGAGAAGTTCAAGGGCCGGTTCACCATCTCCAGAGACAATTCCAAGAA |
| | CACGC |
| 442 | 8C ACTACAATGAGAAGTTCAAGGGCCGGTTCACCATCTCCGCAGACAATTCCAAGAA |
| | CACGC |
| 443 | 9C ACTACAATGAGAAGTTCAAGGGCCGGGCCACCATCTCCAGAGACAATTCCAAG |
| | AACACGC |
| 444 | 10C ACTACAATGAGAAGTTCAAGGGCCGGGCCACCATCTCCGCAGACAATTCCAAG |
| | AACACGC |
| 445 | 11C CCTCGGCTCTCAGGCTGTTCATTTGCAGATACAGCGTGTTCTTGGAATTG |
| 446 | 12C CAGCCTGAGAGCCGAGGACACGGCCGTATATTACTGTGCGARAGAGG |
| 447 | 13C TAAGCTAACGAGCTACTACCGTAGTTATCCTCTYTCGCACAGTAATATAC |
| 448 | 14C GGTAGTAGCTCGTTAGCTTACTGGGGCCAAGGAACCCTGGTCACCGTCTC |
| 449 | 15C GGGGGAAGACCGATGGGCCCTTGGTGGAGGCTGAGGAGACGGTGACCAGGGT |

TABLE 10
Library 2-Light chain:

450
1′C Biotin-
GGTCGTTCCATTTTACTCCCACTCCGCCATCCGGATGACCCAGTCTCC
451
2′C
TCTGTCTCCTACAGATGCAGACAGGGAGAATGGAGACTGGGTCATCCGG
452
3′C
TGCATCTGTAGGAGACAGAGTCACCATCACTTGCAGGGCCAGTCAGAGC
453
4′C
TTTGCTGATACCAGTGAATGTTTGTGCCAATGCTCTGACTGGCCCTGCA
454
5′C
CACTGGTATCAGCAAAAACCAGCAAAAGCCCCTAAGCTCYTCA
455
6′C
CACTGGTATCAGCAAAAACCAAATAAAGCCCCTAAGCTCYTCA
456
7′C
GACCCCAGAGATAGACTCAGAAGCATACTTGATGARGAGCTTAGGGGCT
457
8′C
GACCCCAGAGATAGACTCAGAAGCATAATAGATGARGAGCTTAGGGGCT
458
9′C
GAGTCTATCTCTGGGGTCCCATCAAGGTTCAGCGGCAGTGGATCTGGGA
459
10′C
CTGCAGGCTGCTGATGGTGAGAGTGWAATCCGTCCCAGATCCACTGCCG
460
11′C
CCATCAGCAGCCTGCAGCCTGAAGATTTTGCAACTTATTACTGTCAACA
461
12′C
CGCCGAACGTGAGCGGCCAGTTATTACTTTGTTGACAGTAATAAGTTGC
462
13′C
CCGCTCACGTTCGGCGGAGGGACCAAGGTGGAGCTCAAA
463
14′C
GATGAAGACAGATGGTGCAGCCACAGTACGTTTGAGCTCCACCTTGGTC

The heavy and light chains libraries were assembled as described in Wu, 2003, Methods Mol. Biol., 207, 197-212 using the following oligonucleotide combinations: Library 1 heavy chain: 1K to 11K; Library 1 light chain: 1′K to 10′K; Library 2 heavy chain: IC to 15C; and Library 2 light chain: 1° C. to 14° C.

The V_{H} and V_{L} genes were subsequently amplified as described in Wu, 2003, Methods Mol. Biol., 207, 197-212 using the following oligonucleotide combinations: Library 1 heavy chain: 1K/11K; Library 1 light chain: 1′K/10′K; Library 2 heavy chain: 1C/15C; and Library 2 light chain: 1° C./14° C.

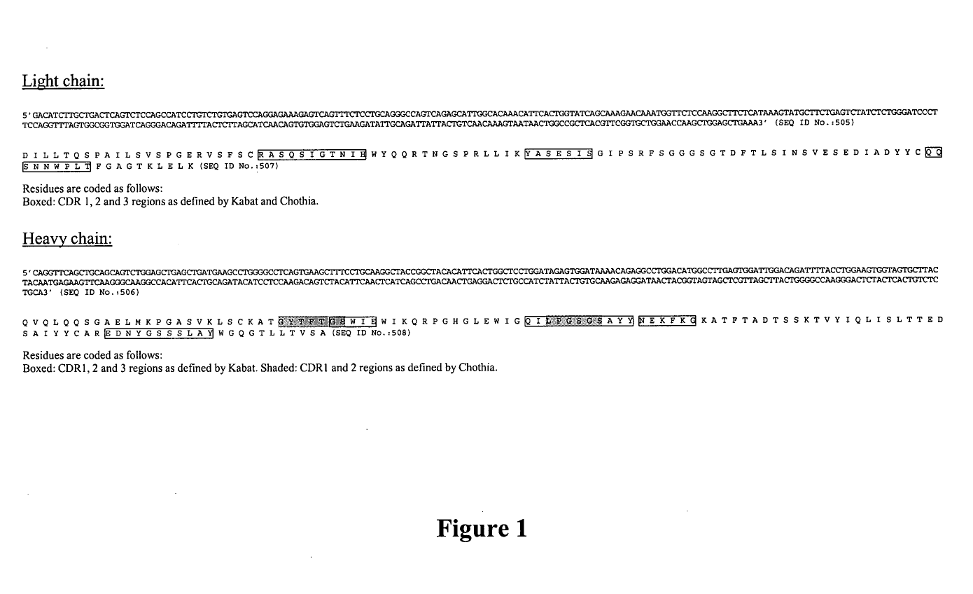

A chimeric Fab (mouse VH and VL regions fused to the corresponding human constant regions) was also constructed after amplification of the genes coding for L1-V_{L} and L1-V_{H} (see FIG. 1) with the CmH/CmH′ and CmL/CmL′ oligonucleotides combinations, respectively (see below and Section 6.3).

(SEQ ID No: 497)

CmH BIOTIN-
GATTCCGCTGGTGGTGCCGTTCTATAGCCATAGCCAGGTTCA
GCTGCAGCAGTCTGGAG

(SEQ ID No: 498)

CmH′
GGGGGAAGACCGATGGGCCCTTGGTGGAGGCTGCAGAGAC
AGTGAGTAGAGTCCC

(SEQ ID No: 499)

CmL BIOTIN-
GGTCGTTCCATTTTACTCCCACTCCGACATCTTGCTGAC
TCAGTCTCC

(SEQ ID No: 500)

CmL′
GATGAAGACAGATGGTGCAGCCACAGTACGTTTCAGCTCCAG
CTTGGTTCCAGC

The minus single-stranded DNA was purified by ethanol precipitation after dissociation of the double-stranded PCR product using sodium hydroxide and elimination of the biotinylated strand by streptavidin-coated magnetic beads as described in Wu & An, 2003, Methods Mol. Biol., 207, 213-233 and Wu, 2003, Methods Mol. Biol., 207, 197-212.

### 6.3. Cloning of Combinatorial Libraries into a Expression System

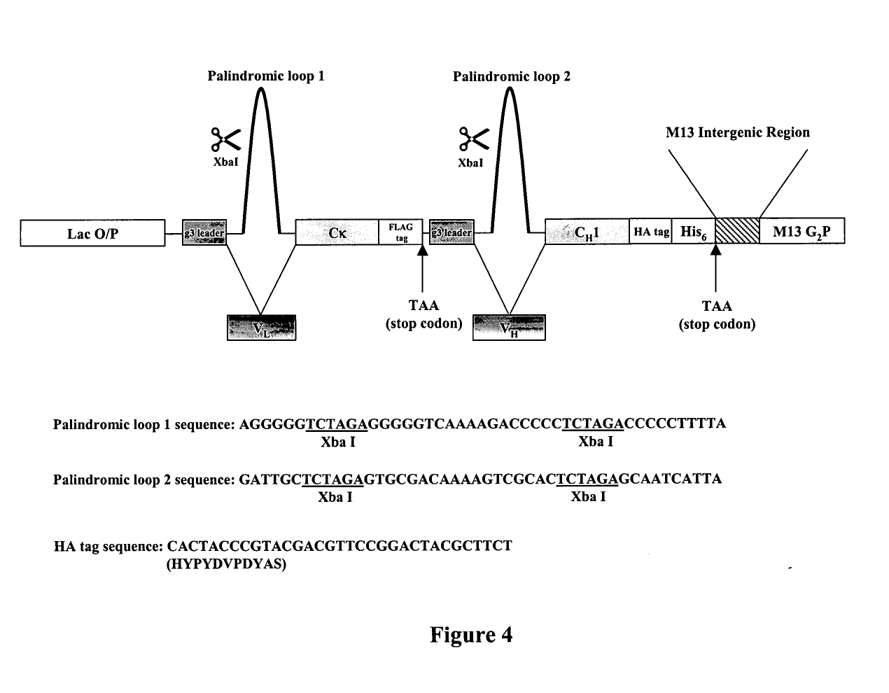

Libraries 1 and 2 as well as the chimeric construct were cloned into a M13-based phage vector. This vector allows the expression of Fab fragments that contain the first constant domain of the human γ1 heavy chain and the constant domain of the human kappa (κ) light chain under the control of the lacZ promoter (see FIG. 4). This was carried out by hybridization mutagenesis essentially as described in Wu & An, 2003, Methods Mol. Biol., 207, 213-233, Wu, 2003, Methods Mol. Biol., 207, 197-212 and Kunkel et al., 1987, Methods Enzymol. 154, 367-382. Briefly, purified minus strands corresponding to the heavy and light chains to be cloned were annealed to two regions containing each one palindromic loop. Those loops contain a unique XbaI site which allows for the selection of the vectors that contain both VL and VH chains fused in frame with the human kappa (κ) constant and first human γ1 constant regions, respectively (Wu & An, 2003, Methods Mol. Biol., 207, 213-233, Wu, 2003, Methods Mol. Biol., 207, 197-212). Synthesized DNA was then electroporated into XL1-blue for plaque formation on XL1-blue bacterial lawn or production of Fab fragments as described in Wu, 2003, Methods Mol. Biol., 207, 197-212.

### 6.4. Screening of the Libraries

To screen the libraries, a primary screen using a capture lift assay was performed followed by a single point ELISA (SPE) secondary screen. However, the SPE can also be used for the initial screening of the libraries.

Primary Screening of Libraries 1 and 2:

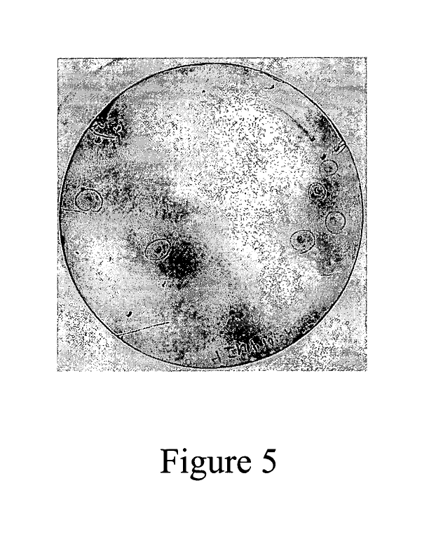

Libraries 1 and 2 were screened by a capture lift assay essentially as described in Wu, 2003, Methods Mol. Biol., 207, 197-212. IL-9 binders were identified after incubation of the filter with biotinylated human IL-9 followed by development with a streptavidin-alkaline phosphatase conjugate. Six and forty positive clones from library 1 and 2, respectively, were selected for secondary screening (see FIG. 5).

Secondary Screening of Libraries 1 and 2:

The secondary screening was carried out by ELISA on supernatant-expressed Fab fragments in order to confirm the clones identified by the capture lift assay. Using supernatants prepared from 1 ml-bacterial culture grown in 96 deep-well plates, two ELISAs were carried out, a quantification ELISA and a functional ELISA.

Quantification ELISA: This was performed essentially as described in Wu, 2003, Methods Mol. Biol., 207, 197-212. Briefly, concentrations were determined by an anti-human Fab ELISA in which individual wells of a 96-well Immulon Immunoplate were coated with 50 ng of a goat anti-human Fab antibody and then incubated with samples (supernatant-expressed Fabs) or standard (human IgG Fab). Incubation with a goat anti-human kappa horseradish peroxidase (HRP) conjugate then followed. HRP activity was detected with tetramethylbenzidine (TMB) substrate and the reaction quenched with 0.2 M H₂SO₄. Plates were read at 450 nm. 4 and 32 clones from library 1 and 2, respectively, expressed detectable amounts of Fab. Those clones were then selected for the next part of the secondary screening (see below).

Functional ELISA: briefly, IL-9 binding activity was determined by an IL-9-based ELISA in which individual wells of a 96-well Maxisorp Immunoplate were coated with 50 ng of human IL9, blocked with 1% BSA/0.1% Tween 20 and then incubated with samples (supernatant-expressed Fabs). Incubation with a goat anti-human kappa horseradish peroxidase (HRP) conjugate then followed. HRP activity was detected with TMB substrate and the reaction quenched with 0.2 M H₂SO₄. Plates were read at 450 nm.

### 6.5. Characterization and Analysis of Selected Humanized Clones

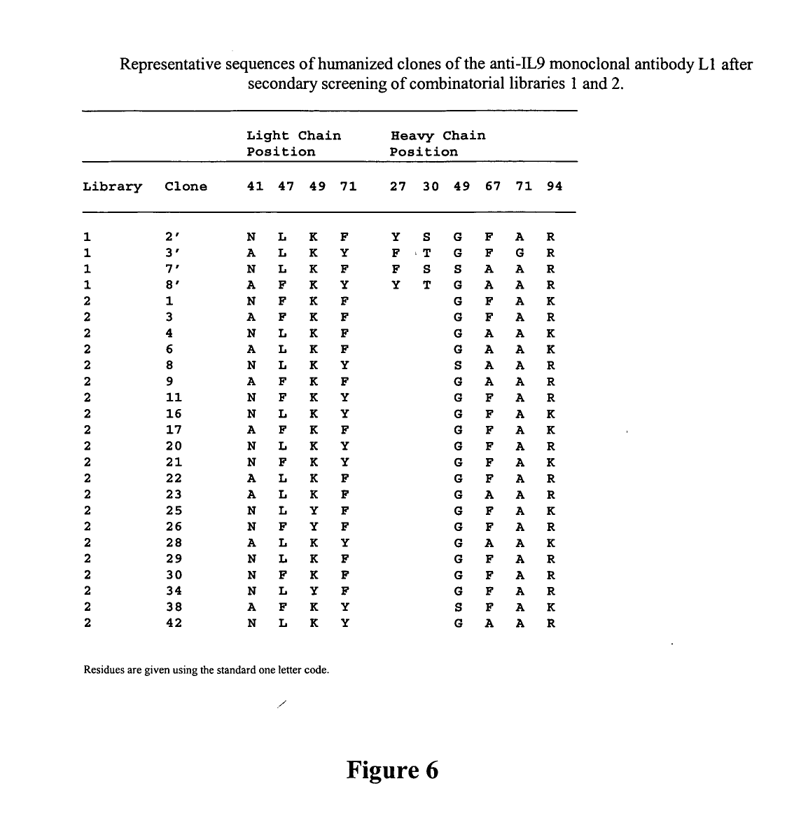

Clones that tested positive after the secondary screening were characterized by dideoxynucleotide sequencing using a ABI300 genomic analyzer. Four and twenty-one unique sequences were found for library 1 and 2, respectively (see FIG. 6). Those different humanized versions of the anti-IL9 monoclonal L1 contain from 2 to 5 and from 3 to 7 mouse residues in the light and heavy chains, respectively. Overall, the number of mouse residues ranged from 5 to 10. Those numbers include the two non-human residues that were fixed in each of the light and heavy chains (see Rule (5) in Section 5.1). Interestingly, position 49 in the light chain and positions 49 and 71 in the heavy chain almost exclusively retain the corresponding non-human residues. This suggests that those framework residues play a critical role in maintaining binding to IL9.

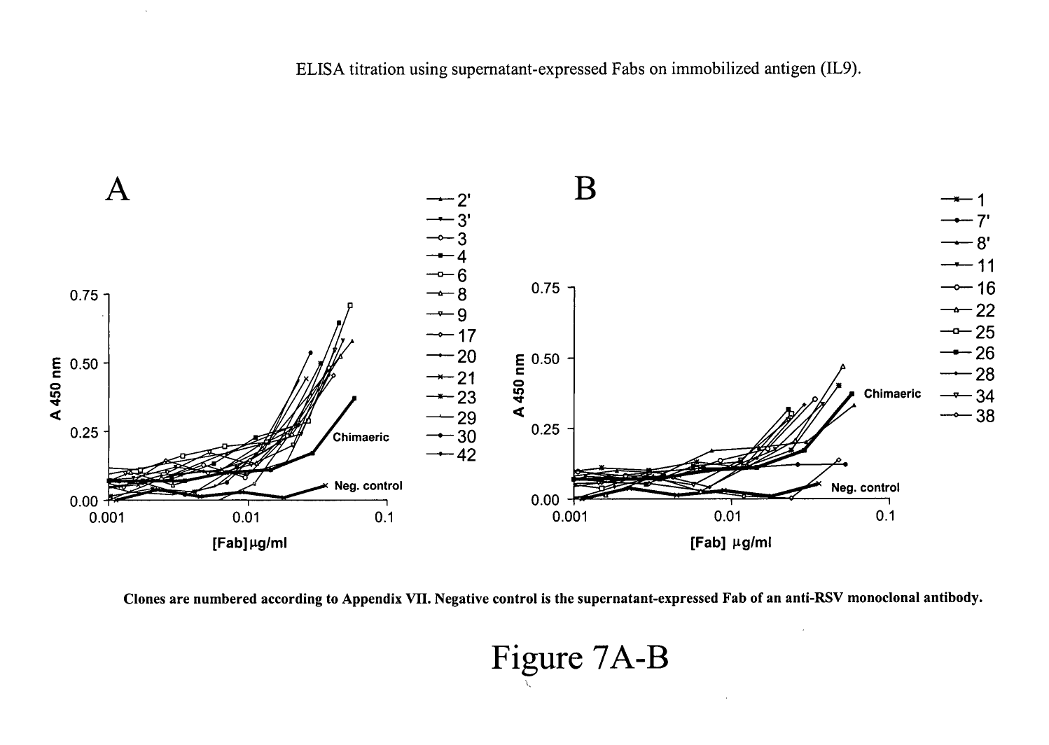

The two-part secondary ELISA screen allowed us to compare the clones to each other and to the chimeric Fab of L1 in terms of binding to human IL-9 (see FIG. 7). As shown in FIG. 7, most of the humanized molecules retained good binding to IL9 as compared with the chimeric Fab of L1. In particular, several humanized clones exhibited better binding to IL9 than the chimeric molecule (clones 2′, 3′, 3, 4, 6, 8, 9, 17, 20, 21, 23, 29, 30 and 42, see FIG. 7(A)). Others exhibited binding to IL9 as good as the chimeric molecule (clones 8′, 1, 11, 16, 22, 25, 26, 28 and 34, see FIG. 7(B)) whereas two false-positive clones (7′ and 38) did not display any significant binding activity (see FIG. 7(B)).

Thus, the strategy of the present invention has allowed the generation of different humanized versions of a non-human antibody which retain good binding to its cognate antigen.

### 7. EXAMPLE

### Humanization of Anti-EphA2 Antibodies

EphA2 is a 130 kDa receptor tyrosine kinase that is expressed in adult epithelia, where it is found at low levels and is enriched within sites of cell-cell adhesion (Zantek et al, Cell Growth & Differentiation 10:629, 1999; R. A. Lindberg et al., Molecular & Cellular Biology 10: 6316, 1990). The subcellular localization of EphA2 is important because EphA2 binds ligands (known as EphrinsA1 to A5) that are anchored to the cell membrane (Eph Nomenclature Committee, Cell 90:403. 1997; Gale et al., Cell & Tissue Research 290: 227, 1997). The primary consequence of ligand binding is EphA2 autophosphorylation (Lindberg et al., Molecular & Cellular Biology 10: 6316, 1990). However, unlike other receptor tyrosine kinases, EphA2 retains enzymatic activity in the absence of ligand binding or phosphotyrosine content (Zantek et al., Cell Growth & Differentiation 10:629, 1999).

Antibodies to EphA2 have been made and shown to be useful: (1) in the prevention, treatment, management and/or amelioration of cancer (see e.g., U.S. application Ser. No. 10/436,782, which is incorporated herein by reference in its entirety); (2) in the prevention, treatment, management and/or amelioration of disorders involving non-neoplastic hyperproliferative cells, particularly hyperproliferative epithelial and endothelial cells (see e.g., U.S. Provisional Application Ser. No. 60/462,024, which is incorporated herein by reference in its entirety); and (3) as diagnostic or screening tools (see e.g., U.S. application Ser. No. 10/436,782 and U.S. Provisional Application Ser. No. 60/462,024, each of which is incorporated herein by reference in its entirety).

### 7.1 Selection of Human Framework

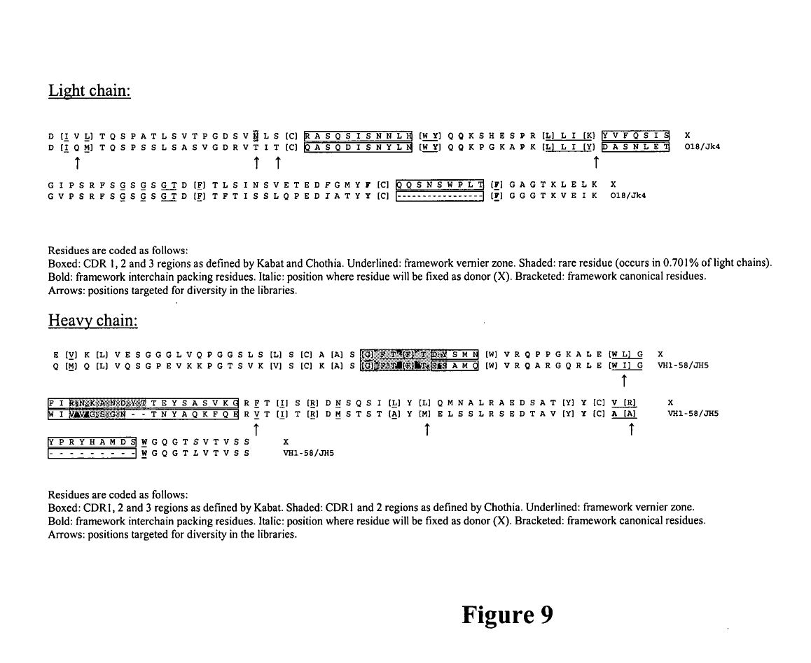

According to the rules of design (see Section 5.1), human germline VH1-58 in combination with JH5 was used to graft the donor heavy chain CDR loops and human germline O18 in combination with Jκ4 was used to graft the donor light chain CDR loops (see FIG. 9).

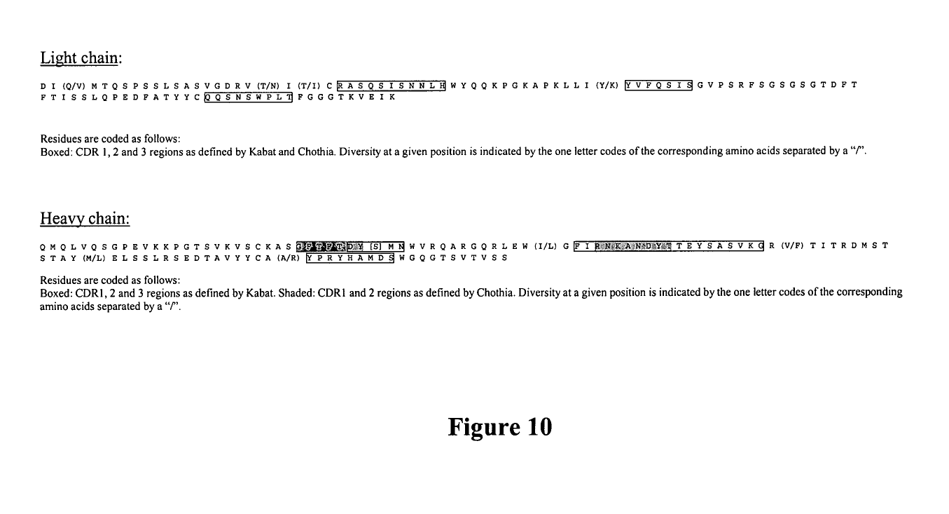

Diversity was introduced at four positions (3, 20, 22 and 49 according to Kabat numbering) in the humanized light chain (see FIG. 10). More precisely, the generation of diversity at position 22 arose from the investigation of the importance of a potential glycosylation site and consists of a wobble between the corresponding mouse residue and a human residue found in human germline L22. In the humanized heavy chain, four positions (48, 67, 80 and 94 according to Kabat numbering) were diversified (see FIG. 10). In both cases, mutagenesis was carried out using the Polymerase Chain Reaction by overlap extension in order to synthesize humanized anti-EphA2 antibody light chains and anti-EphA2 antibody heavy chains in which all of the murine residues were substituted by their human counterparts, except in regions where diversity was introduced (see FIG. 10 and § 5.1) or where a donor residue was fixed (see FIG. 10 and § 5.1). The polymerase chain reaction was performed using degenerate oligonucleotides encoding the codons for both the human and murine residues (wobbles).

### 7.2 Construction of Combinatorial Libraries

One main humanization library (library “A”) was constructed that included two sub-libraries: (1) Sub-library 1 was a heavy chain combinatorial library with CDRs defined according to Kabat; and (2) Sub-library 2 was a light chain combinatorial library with CDRs defined according to Kabat.

The oligonucleotides in Table 11 and 12, infra, were used to construct the sub-libraries (all shown in the 5′ to 3′ orientation, name followed by sequence, where K=G or T, M=A or C, R=A or G, S=C or G, W=A or T and Y=C or T).

TABLE 11
Sub-library 1-Heavy chain (CDRs defined according to Kabat):

| | |
|---|---|
| 464 | 1K BIOTIN-CGCTGGTGGTGCCGTTCTATAGCCATAGCCAAATGCAGCTGGTGCAGTCTG |
| | GGCCTGAG |
| 465 | 2K CTATGGACTCCTGGGGCCAAGGAACCTCGGTCACCGTCTCCTCAGCCTCCAC |
| 466 | 3K CCCAGGAGTCCATAGCATGATACCTAGGGTATctCGCACAGTAATACAC |
| 467 | 4K TCCGAGGACACGGCCGTGTATTACTGTGCGAGATACCCTAGGTATCATG |
| 468 | 5K GGCCGTGTCCTCGGATCTCAGGCTGCTCAGCTCCAWGTAGGCTGTGCT |
| 469 | 6K CAGGGACATGTCCACAAGCACAGCCTACWTGGAGCTGAGCAGCCTGAGA |
| 470 | 7K TGTGGACATGTCCCTGGTAATGGTGAMTCTACCCTTCA |
| 471 | 8K TACACAACAGAGTACAGTGCATCTGTGAAGGGTAGAKTCACCATTAC |
| 472 | 9K CAGATGCACTGTACTCTGTTGTGTAATCATTAGCTTTGTTTCTAA |
| 473 | 10K TAAATCCTAKCCACTCAAGGCGTTGTCCACGAGCCTGTCGCACC |
| 474 | 11K GACAACGCCTTGAGTGGMTAGGATTTATTAGAAACAAAGCTAATGAT |
| 475 | 12K TCACCTTTACTGATTACTCCATGAACTGGGTGCGACAGGCTCGTG |
| 476 | 13K GACCTTCACTGAGGTCCCAGGCTTCTTCACCTCAGGCCCAGACTG |
| 477 | 14K GTGAAGAAGCCTGGGACCTCAGTGAAGGTCTCCTGCAAGGCTTCTGGAT |
| 478 | 15K CAGTTCATGGAGTAATCAGTAAAGGTGAATCCAGAAGCCTTGCAGGA |
| 479 | 16K CACCAGCTGCATTTGGCTATGGCTATAGAACGGCACCACCAGCG |
| 480 | 17K GGAAGACCGATGGGCCCTTGGTGGAGGCTGAGGAGACGGTGACCGAGGTTCCTTGGC |

TABLE 12
Sub-library 2-Light chain (CDRs defined according to Kabat):

| | |
|---|---|
| 481 | 1′K BIOTIN-GGTCGTTCCATTTTACTCCCACTCCGACATCGTGATGACCCAGTCTCC |
| 482 | 2′K CGCTCACGTTCGGCGGAGGGACCAAGGTGGAGATCAAACGTACTGTGGC |
| 483 | 3′K CCTCCGCCGAACGTGAGCGGCCAGCTGTTACTCTGTTGACA |
| 484 | 4′K AGCCTGAAGATTTTGCAACATATTACTGTCAACAGAGTAACAGCTGGC |
| 485 | 5′K GTAATATGTTGCAAAATCTTCAGGCTGCAGGCTGCTGATGGT |
| 486 | 6′K GATCTGGGACAGATTTTACTTTCACCATCAGCAGCCTGC |
| 487 | 7′K GAAAGTAAAATCTGTCCCAGATCCACTTCCACTGAACCTTGATGG |
| 488 | 8′K GTCCATCTCTGGGGTCCCATCAAGGTTCAGTGGAAGTG |
| 489 | 9′K GACCCCAGAGATGGACTGGAAAACATACTTGATCAGGAGCTTAGG |
| 490 | 10′K AGAAACCAGGGAAAGCCCCTAAGCTCCTGATCAAGTATGTTTTCCA |
| 491 | 11′K GGCTTTCCCTGGTTTCTGCTGATACCAGTGTAGGTTGTTGCTAA |
| 492 | 12′K CAGGGCCAGCCAAAGTATTAGCAACAACCTACACTGGTATCAGC |
| 493 | 13′K TACTTTGGCTGGCCCTGCAARTGATGKTGACTCTGTCTCCTACAGATG |
| 494 | 14′K ATCCTCCCTGTCTGCATCTGTAGGAGACAGAGTCAMCATCAYTTG |
| 495 | 15′K CAGACAGGGAGGATGGAGACTGGGTCATCACGATGTCGGAGTGGGAGTA |
| 496 | 16′K GATGAAGACAGATGGTGCAGCCACAGTACGTTTGATCTCCACCTTGGTC |

The heavy and light chains libraries were assembled by fusion essentially as described in Wu, Methods Mol. Biol., 207:197-212, 2003 using the following oligonucleotide combinations: Sub-library 1 (heavy chain): 1K to 17K; and Sub-library 2 (light chain): 1′K to 16′K.

The V_{H} and V_{L} genes were subsequently amplified as described in Wu, 2003, Methods Mol. Biol., 207, 197-212 using the following oligonucleotide combinations: Sub-library 1 (heavy chain): 1K/17K; and Sub-library 2 (light chain): 1′K/16′K.

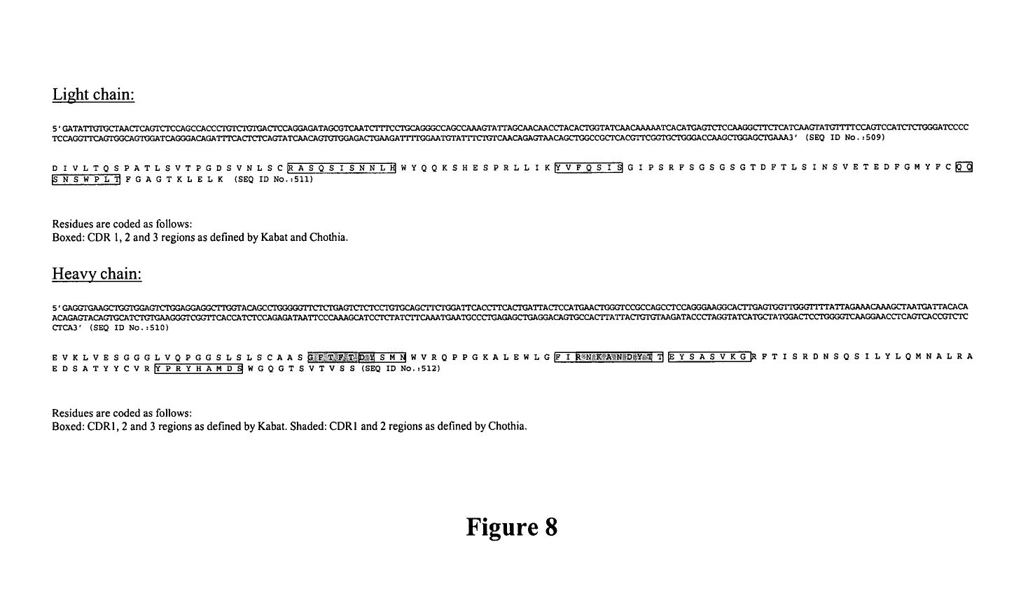

A chimeric Fab (mouse V_{H} and V_{L} regions fused to the corresponding human constant regions) was also constructed after amplification of the genes coding for X-V_{H} and X-V_{L} (see FIG. 8) with the ChimH/ChimH′ and ChimL/ChimL′ oligonucleotides combinations, respectively (see below and § 7.3).

| | |
|---|---|
| ChimH BIOTIN- | |
| GCTGGTGGTGCCGTTCTATAGCCATAGCGAGGTGAAGCTGGTGGAGTCTGGAGGAG | (SEQ ID NO.: 501) |
| ChimH′ | |
| GGAAGACCGATGGGCCCTTGGTGGAGGCTGAGGAGACGGTGACTGAGGTTCCTTG | (SEQ ID No: 502) |
| ChimL BIOTIN- | |
| GGTCGTTCCATTTTACTCCCACTCCGATATTGTGCTAACTCAGTCTCCAGCCACCCTG | (SEQ ID No: 503) |
| ChimL′ | |
| GATGAAGACAGATGGTGCAGCCACAGTACGTTTCAGCTCCAGCTTGGTCCCAGCAC | (SEQ ID No: 504) |
| CGAACG | |

In every case, the minus single-stranded DNA was purified by ethanol precipitation after dissociation of the double-stranded PCR product using sodium hydroxide and elimination of the biotinylated strand by streptavidin-coated magnetic beads as described in Wu & An, 2003, Methods Mol. Biol., 207, 213-233 and Wu, 2003, Methods Mol. Biol., 207, 197-212.

7.3. Cloning of Combinatorial Libraries into a Expression System Library A (see above) as well as the chimaeric construct (see above) were cloned into a M13-based phage vector. This vector allows the expression of Fab fragments that contain the first constant domain of the human γ1 heavy chain and the constant domain of the human kappa (κ) light chain under the control of the lacZ promoter (see FIG. 4). This was carried out by hybridization mutagenesis essentially as described in Wu & An, Methods Mol. Biol., 207:213-233, 2003; Wu, Methods Mol. Biol., 207:197-212, 2003; and Kunkel et al., Methods Enzymol. 154:367-382, 1987. Briefly, purified minus strands corresponding to the heavy and light chains to be cloned (see § 7.2) were annealed to two regions, each containing one palindromic loop. Those loops contain a unique XbaI site which allows for the selection of the vectors that contain both V_{L} and V_{H} chains fused in frame with the human kappa (κ) constant and first human γ1 constant regions, respectively (Wu & An, Methods Mol. Biol., 207:213-233, 2003; Wu, Methods Mol. Biol., 207:197-212, 2003). Synthesized DNA was then electroporated into XL1-blue for plaque formation on XL1-blue bacterial lawn or production of Fab fragments as described in Wu, Methods Mol. Biol., 207:197-212, 2003.

### 7.4. Screening of the Libraries

To screen the libraries, a primary screen using a single point ELISA (SPE) was performed followed by a functional ELISA and Quantification ELISA secondary screen.

Primary Screening:

The primary screen consisted of a single point ELISA (SPE) which was carried out essentially as described in Wu, Methods Mol. Biol., 207:197-212, 2003. Briefly, individual wells of a 96-well Maxisorp Immunoplate were coated with 100 ng of a goat anti-human Fab antibody and then incubated with samples (periplasm-expressed Fabs) for 1 hour at room temperature. After blocking with 3% BSA/PBS for 2 hours at 37° C., 100 ng/well of biotinylated human EphA2-Fc were added and incubated for 1 hour at room temperature. This was followed by incubation with neutravidin-horseradish peroxidase (HRP) conjugate for 40 minutes at room temperature. HRP activity was detected with TMB substrate and the reaction quenched with 0.2 M H₂SO₄. Plates were read at 450 nm. Out of approximately 180 clones from library A that were screened, 12 exhibited a significant signal (OD₄₅₀ ranging from 0.1-0.3). Those clones were then selected for confirmation by a secondary screening (see below).

Secondary Screening:

The secondary screening was performed by ELISA on periplasm-expressed Fab fragments in order to confirm the clones identified by the SPE assay (see above). More precisely, using periplasmic extracts prepared from 1 ml-bacterial culture grown in 96 deep-well plates, two ELISAs were carried out, a functional ELISA and a quantification ELISA.

Functional ELISA: Briefly, individual wells of a 96-well Maxisorp Immunoplate were coated with 500 ng of human EphA2-Fc and blocked with 3% BSA/PBS for 2 hours at 37° C. Samples (periplasm-expressed Fabs) were added and incubated for 1 hour at room temperature. Incubation with a goat anti-human kappa horseradish peroxidase (HRP) conjugate then followed. HRP activity was detected with TMB substrate and the reaction quenched with 0.2 M H₂SO₄. Plates were read at 450 nm.

Quantification ELISA: This was performed essentially as described in Wu, Methods Mol. Biol., 207:197-212, 2003. Briefly, concentrations were determined by an anti-human Fab ELISA in which individual wells of a 96-well Immulon Immunoplate were coated with 50 ng of a goat anti-human Fab antibody and then incubated with samples (periplasm-expressed Fabs) or standard (human IgG Fab). Incubation with a goat anti-human kappa horseradish peroxidase (HRP) conjugate then followed. HRP activity was detected with TMB substrate and the reaction quenched with 0.2 M H₂SO₄. Plates were read at 450 nm.

### 7.5. Characterization and Analysis of Selected Humanized Clones

Clones that tested positive after the secondary screening were characterized by dideoxynucleotide sequencing using a ABI300 genomic analyzer. Three different antibody sequences (named I, II and III thereafter) were identified, which contained from 4 to 6 murine residues per antibody, including the two non-human residues that were fixed in each of the light and heavy chains (see § 5.1). Within those three antibodies, two unique sequences were found for the heavy chains and two unique sequences were found for the light chains (see FIG. 10). Interestingly, position 49 in the light chain and position 94 in the heavy chain exclusively retain the corresponding non-human residues. This suggests that those framework residues play a critical role in maintaining binding of the anti-EphA2 antibody EP101 to human EphA2.

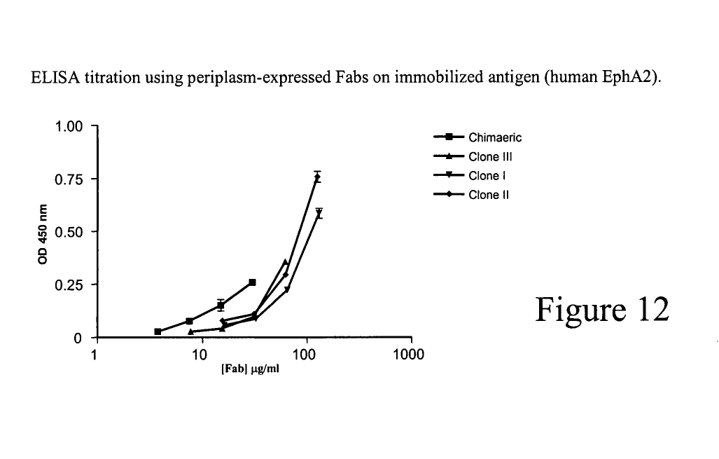

The two-part secondary ELISA screen (see § 7.4) allowed us to compare Fab clones I, II and III to each other and to the chimaeric Fab of anti-EphA2 antibody in terms of binding to human EphA2 (see FIG. 12). As shown in FIG. 12, Fab clones I, II and III retain good binding to human EphA2 as compared with the chimeric Fab of anti-EphA2 antibody. In order to further characterize the different humanized versions of anti-EphA2 antibody, Fab clones I, II and III as well as the chimeric Fab were then cloned and expressed as a full length human IgG1. A BIAcore analysis allowed us to compare the different molecules to each other.

| kₒₙ (s⁻¹ · M⁻¹) | k_{off} (s⁻¹) | K_{D} | Molecule |
|---|---|---|---|
| 3.3 × 10⁵ | 1.01 × 10⁻⁴ | 0.3 nM | Mouse version of EP101 |
| | | | (mouse IgG) |
| 2.42 × 10⁵ | 8.04 × 10⁻⁵ | 0.3 nM | Chimaeric version of EP101 |
| | | | (hu IgG1) |
| 5.32 × 10⁴ | 3.76 × 10⁻⁵ | 0.7 nM | Humanized version I of EP101 |
| | | | (hu IgG1) |
| 3.56 × 10⁴ | 4.13 × 10⁻⁵ | 1.2 nM | Humanized version II of EP101 |
| | | | (hu IgG1) |
| 6.00 × 10⁴ | 7.62 × 10⁻⁵ | 1.3 nM | Humanized version III of EP101 |
| | | | (hu IgG1) |

As shown above, the three different humanized antibodies exhibit affinities towards human EphA2 which are similar to those of the chimeric version of anti-EphA2 antibody and the parental murine antibody.

Thus, our strategy has allowed the generation of different humanized versions of a non-human antibody which retain good binding to its cognate antigen. Altogether, the data validate the choice of the “rules of design” and more generally of the approach to humanization of antibodies in accordance with the present invention.

References Cited and Equivalents

All references cited herein are incorporated herein by reference in their entirety and for all purposes to the same extent as if each individual publication or patent or patent application was specifically and individually indicated to be incorporated by reference in its entirety for all purposes.

Many modifications and variations of this invention can be made without departing from its spirit and scope, as will be apparent to those skilled in the art.

1. A library of nucleic acid sequences comprising nucleotide sequences encoding humanized heavy chain variable regions, each nucleotide sequence produced by fusing together in frame nucleic acid sequences encoding CDRs from a donor antibody heavy chain variable region and nucleic acid sequences encoding acceptor heavy chain variable framework regions that are together less than 65% identical to the donor antibody heavy chain variable framework regions together at the amino acid level.

2. A library of nucleic acid sequences comprising nucleotide sequences encoding humanized heavy chain variable regions, each nucleotide sequence produced by fusing together in frame nucleic acid sequences encoding CDRs from a donor antibody heavy chain variable region and nucleic acid sequences encoding acceptor heavy chain variable framework regions that are together less than 65% identical to the donor antibody heavy chain variable framework regions together at the amino acid level and contain one or more mutations at amino acid residues designated key residues, said key residues not including amino acid residues 2, 4, 24, 35, 36, 39, 43, 45, 64, 69, 70, 73, 74, 75, 76, 78, 92 and 93 according to the Kabat numbering system.

3. A library of nucleic acid sequences comprising nucleotide sequences encoding humanized light chain variable regions, each nucleotide sequence produced by fusing together in frame nucleic acid sequences encoding CDRs from a donor antibody light chain variable region and nucleic acid sequences encoding acceptor light chain variable framework regions together that are less than 65% identical to the donor antibody light chain variable framework regions together at the amino acid level.

4. A library of nucleic acid sequences comprising nucleotide sequences encoding humanized light chain variable regions, each nucleotide sequence produced by fusing together in frame nucleic acid sequences encoding CDRs from a donor antibody light chain variable region and nucleic acid sequences encoding acceptor light chain variable framework regions together that are less than 65% identical to the donor antibody light chain variable framework regions at the amino acid level and contain one or more mutations at amino acid residues designated key residues, said key residues not including amino acid residues 4, 38, 43, 44, 46, 58, 62, 65, 66, 67, 68, 69, 73, 85 and 98 according to the Kabat numbering system.

5. A library of nucleic acid sequences comprising (i) a first set of nucleotide sequences encoding humanized heavy chain variable regions, each nucleotide sequence in the first set of nucleotide sequences produced by fusing together in frame nucleic acid sequences encoding CDRs from a donor antibody heavy chain variable region and nucleic acid sequences encoding acceptor heavy chain variable framework regions together that are less than 65% identical to the donor antibody heavy chain variable framework regions together at the amino acid level; and (ii) a second set of nucleotide sequences encoding humanized light chain variable regions, each nucleotide sequence in the second set of nucleotide sequences produced by fusing together in frame nucleic acid sequences encoding CDRs from a donor antibody light chain variable region and nucleic acid sequences encoding acceptor light chain variable framework regions.

6. A library of nucleic acid sequences comprising (i) a first set of nucleotide sequences encoding humanized heavy chain variable regions, each nucleotide sequence in the first set of nucleotide sequences produced by fusing together in frame nucleic acid sequences encoding CDRs from a donor antibody heavy chain variable region and nucleic acid sequences encoding acceptor heavy chain variable framework regions together that are less than 65% identical to the donor antibody heavy chain variable framework regions together at the amino acid level and contain one or more mutations at amino acid residues designated key residues, said key residues not including amino acid residues 2, 4, 24, 35, 36, 39, 43, 45, 64, 69, 70, 73, 74, 75, 76, 78, 92 and 93 according to the Kabat numbering system; and (ii) a second set of nucleotide sequences encoding humanized light chain variable regions, each nucleotide sequence in the second set of nucleotide sequences produced by fusing together in frame nucleic acid sequences encoding CDRs from a donor antibody light chain variable region and nucleic acid sequences encoding acceptor light chain variable framework regions.

7. A library of nucleic acid sequences comprising: (i) a first set of nucleotide sequences encoding humanized heavy chain variable regions, each nucleotide sequence in the first set of nucleotide sequences produced by fusing together in frame nucleic acid sequences encoding CDRs from a donor antibody heavy chain variable region and nucleic acid sequences encoding acceptor heavy chain variable framework regions; and (ii) a second set of nucleotide sequences encoding humanized light chain variable regions, each nucleotide sequence in the second set of nucleotide sequences produced by fusing together in frame nucleic acid sequences encoding CDRs from a donor antibody light chain variable region and nucleic acid sequences encoding acceptor light chain variable framework regions together that are less than 65% identical to the donor antibody light chain variable framework regions together at the amino acid level.

8. A library of nucleic acid sequences comprising: (i) a first set of nucleotide sequences encoding humanized heavy chain variable regions, each nucleotide sequence in the first set of nucleotide sequences produced by fusing together in frame nucleic acid sequences encoding CDRs from a donor antibody heavy chain variable region and nucleic acid sequences encoding acceptor heavy chain variable framework regions; and (ii) a second set of nucleotide sequences encoding humanized light chain variable regions, each nucleotide sequence in the second set of nucleotide sequences produced by fusing together in frame nucleic acid sequences encoding CDRs from a donor antibody light chain variable region and nucleic acid sequences encoding acceptor light chain variable framework regions together that are less than 65% identical to the donor antibody light chain variable framework regions together at the amino acid level and contain one or more mutations at amino acid residues designated key residues, said key residues not including amino acid residues 4, 38, 43, 44, 46, 58, 62, 65, 66, 67, 68, 69, 73, 85 and 98 according to the Kabat numbering system.

9. A library of nucleic acid sequences comprising: (i) a first set of nucleotide sequences encoding humanized heavy chain variable regions, each nucleotide sequence in the first set of nucleotide sequences produced by fusing together in frame nucleic acid sequences encoding CDRs from a donor antibody heavy chain variable region and nucleic acid sequences encoding acceptor heavy chain variable framework regions together that are less than 65% identical to the donor antibody heavy chain variable framework regions together at the amino acid level; and (ii) a second set of nucleotide sequences encoding humanized light chain variable regions, each nucleotide sequence in the second set of nucleotide sequences produced by fusing together in frame nucleic acid sequences encoding CDRs from a donor antibody light chain variable region and nucleic acid sequences encoding acceptor light chain variable framework regions together that are less than 65% identical to the donor antibody light chain variable framework regions together at the amino acid level.

10. The library of any of the claims 1 to 9, wherein said acceptor is human.

11. A cell containing nucleic acid sequences encoding a humanized antibody that immunospecifically binds to an antigen, said cell produced by the process comprising:
(a) selecting an acceptor heavy chain variable framework region less than 65% identical globally to a donor antibody heavy chain variable framework region at the amino acid level, which acceptor heavy chain variable framework region contains at least one amino acid residue at amino acid residues 6, 23, 24 or 49 according to the Kabat numbering system that is not identical to the corresponding residue in the donor antibody, and wherein the acceptor heavy chain framework region and donor antibody heavy chain framework region each comprises FR1, FR2, FR3 and FR4;
(b) synthesizing a nucleic acid sequence comprising a nucleotide sequence encoding a humanized heavy chain variable region, said nucleotide sequence comprising nucleic acid sequences encoding complementarity determining regions (CDRs) from the donor antibody heavy chain variable region and nucleic acid sequences encoding the acceptor heavy chain variable framework regions; and
(c) introducing the nucleic acid sequence comprising the nucleotide sequence encoding the humanized heavy chain variable region into a cell.

12. The cell of claim 11, wherein the cell further contains a nucleic acid sequence comprising a nucleotide sequence encoding a light chain variable region.

13. A method of producing a humanized antibody that immunospecifically binds to an antigen, said method comprising expressing nucleic acid sequences encoding the humanized antibody contained in the cell of claim 11.

14. A cell containing nucleotide sequences encoding a humanized antibody that immunospecifically binds to an antigen, said cell produced by the process comprising:
(a) selecting an acceptor heavy chain variable framework region less than 65% identical to a donor antibody heavy chain variable framework region at the amino acid level, which acceptor heavy chain variable framework region contains at least one amino acid residue at amino acid residues 6, 23, 24 or 49 according to the Kabat numbering system that is not identical to the corresponding residue in the donor antibody, wherein the acceptor heavy chain framework region and donor antibody heavy chain framework region each comprises FR1, FR2, FR3 and FR4;
(b) synthesizing a nucleic acid sequence comprising a nucleotide sequence encoding a humanized heavy chain variable region with a framework region that remains less than 65% identical to the donor antibody heavy chain variable framework region at the amino acid level, said nucleotide sequence comprising nucleic acid sequences encoding CDRs from the donor antibody heavy chain variable region and nucleic acid sequences encoding the acceptor heavy chain variable framework regions with one or more mutations introduced at amino acid residues designated key residues, said key residues not including amino acid residues 2, 4, 24, 35, 36, 39, 43, 45, 64, 69, 70, 73, 74, 75, 76, 78, 92 and 93 according to the Kabat numbering system; and
(c) introducing the nucleic acid sequence comprising the nucleotide sequence encoding the humanized heavy chain variable region into a cell.

15. The cell of claim 14, wherein the cell further contains a nucleic acid sequence comprising a nucleotide sequence encoding a light chain variable region.

16. A method of producing a humanized antibody that immunospecifically binds to an antigen, said method comprising expressing nucleic acid sequences encoding the humanized antibody contained in the cell of claim 14.

17. A cell containing nucleic acid sequences encoding a humanized antibody that immunospecifically binds to an antigen, said cell produced by the process comprising:
(a) selecting an acceptor light chain variable framework region less than 65% identical to a donor antibody light chain variable framework region at the amino acid level, wherein the acceptor light chain framework region and donor antibody light chain framework region each comprises FR1, FR2, FR3 and FR4;
(b) synthesizing a nucleic acid sequence comprising a nucleotide sequence encoding a humanized light chain variable region, said nucleotide sequence comprising nucleic acid sequences encoding complementarity determining regions (CDRs) from the donor antibody light chain variable region and nucleic acid sequences encoding the acceptor light chain variable framework regions; and
(c) introducing the nucleic acid sequence comprising the nucleotide sequence encoding the humanized light chain variable region into a cell.

18. The cell of claim 17, wherein the cell further contains a nucleic acid sequence comprising a nucleotide sequence encoding a heavy chain variable region.

19. A method of producing a humanized antibody that immunospecifically binds to an antigen, said method comprising expressing nucleic acid sequences encoding the humanized antibody contained in the cell of claim 17.

20. A cell containing nucleotide sequences encoding a humanized antibody that immunospecifically binds to an antigen, said cell produced by the process comprising:
(a) selecting an acceptor light chain variable framework region less than 65% identical to a donor antibody heavy chain variable framework region at the amino acid level, wherein the acceptor light chain framework region and donor antibody light chain framework region each comprises FR1, FR2, FR3 and FR4;
(b) synthesizing a nucleic acid sequence comprising a nucleotide sequence encoding a humanized light chain variable region, said nucleotide sequence comprising nucleic acid sequences encoding CDRs from the donor antibody light chain variable region and nucleic acid sequences encoding the acceptor light chain variable framework regions with one or more mutations introduced at amino acid residues designated key residues, said key residues not including amino acid residues 4, 38, 43, 44, 46, 58, 62, 65, 66, 67, 68, 69, 73, 85, and 98 according to the Kabat numbering system; and
(c) introducing the nucleic acid sequence comprising the nucleotide sequence encoding the humanized light chain variable region into a cell.

21. The cell of claim 20, wherein the cell further contains a nucleic acid sequence comprising a nucleotide sequence encoding a heavy chain variable region.

22. A method of producing a humanized antibody that immunospecifically binds to an antigen, said method comprising expressing nucleic acid sequences encoding the humanized antibody contained in the cell of claim 20.

23. A cell containing a nucleic acid sequence encoding a humanized antibody that immunospecifically binds to an antigen, said cell produced by the process comprising:
(a) selecting an acceptor heavy chain variable framework region less than 65% identical to a donor antibody heavy chain variable framework region at the amino acid level, which acceptor heavy chain variable framework region contains at least one amino acid residue at amino acid residues 6, 23, 24 or 49 according to the Kabat numbering system that is not identical to the corresponding residue in the donor antibody, wherein the acceptor heavy chain framework region and donor antibody heavy chain framework region each comprises FR1, FR2, FR3 and FR4;
(b) selecting an acceptor light chain variable framework region less than 65% identical to a donor antibody light chain variable framework region at the amino acid level, wherein the acceptor light chain framework region and donor antibody light chain framework region each comprises FR1, FR2, FR3 and FR4;
(c) synthesizing a nucleic acid sequence comprising: (i) a first nucleotide sequence encoding a humanized light chain variable region, said first nucleotide sequence comprising nucleic acid sequences encoding CDRs from the donor antibody light chain variable region and nucleic acid sequences encoding the acceptor light chain variable framework regions with one or more mutations introduced at amino acid residues designated key residues, said key residues not including amino acid residues 4, 38, 43, 44, 46, 58, 62, 65, 66, 67, 68, 69, 73, 85, and 98 according to the Kabat numbering system, and (ii) a second nucleotide sequence encoding a humanized heavy chain variable region with a framework region comprising FR1, FR2, FR3 and FR4 that remains globally less than 65% identical to the donor antibody heavy chain variable framework region at the amino acid level, said second nucleotide sequence comprising nucleic acid sequences encoding complementarity determining regions (CDRs) from the donor antibody heavy chain variable region and nucleic acid sequences encoding the acceptor heavy chain variable framework regions; and
(d) introducing the nucleic acid sequence comprising the first nucleotide sequence and second nucleotide sequence into a cell.

24. A method of producing a humanized antibody that immunospecifically binds to an antigen, said method comprising expressing the nucleic acid sequence encoding the humanized antibody contained in the cell of claim 23.

25. A population of cells engineered to contain nucleotide sequences encoding a plurality of humanized antibodies produced by a process comprising:
(a) selecting acceptor heavy chain variable framework regions less than 65% identical to a donor antibody heavy chain variable framework region at the amino acid level, which acceptor heavy chain variable framework regions contain amino acid residues at amino acid residues 6, 23, 24 or 49 according to the Kabat numbering system that are not conserved between the framework region of the donor antibody and the acceptor heavy chain variable framework region, wherein the acceptor heavy chain framework region and donor antibody heavy chain framework region each comprises FR1, FR2, FR3 and FR4;
(b) synthesizing a nucleic acid sequences comprising nucleotide sequences encoding humanized heavy chain variable regions, said nucleotide sequences comprising nucleic acid sequences encoding complementarity determining regions (CDRs) from the donor antibody heavy chain variable region and nucleic acid sequences encoding the acceptor heavy chain variable framework regions; and
(c) introducing the nucleic acid sequences comprising the nucleotide sequences encoding the humanized heavy chain variable regions into cells.

26. A population of cells engineered to contain nucleotide sequences encoding a plurality of humanized antibodies produced by a process comprising:
(a) selecting acceptor light chain variable framework regions less than 65% identical to a donor antibody light chain variable framework region at the amino acid level, wherein the acceptor light chain framework region and donor antibody light chain framework region each comprises FR1, FR2, FR3 and FR4;
(b) synthesizing nucleic acid sequences comprising nucleotide sequences encoding humanized light chain variable regions, said nucleotide sequences comprising nucleic acid sequences encoding complementarity determining regions (CDRs) from the donor antibody light chain variable region and nucleic acid sequences encoding the acceptor light chain variable framework regions; and
(c) introducing the nucleic acid sequences comprising the nucleotide sequences encoding the humanized light chain variable regions into cells.

27. A method of producing a humanized antibody that immunospecifically binds to an antigen, said method comprising providing a cell containing nucleotide sequences encoding humanized heavy chain and light chain variable regions and expressing the nucleotide sequences, wherein said cell containing the nucleotide sequences was produced by:
(a) comparing the nucleotide sequence of a donor antibody heavy chain variable region against a collection of sequences of acceptor heavy chain variable regions;
(b) selecting an acceptor heavy chain variable framework region less than 65% identical to the donor antibody heavy chain variable framework region at the amino acid level, which acceptor heavy chain variable framework region contains at least one amino acid residue at amino acid residues 6, 23, 24 or 49 according to the Kabat numbering system that is not identical to the corresponding residue in the donor antibody, wherein the acceptor heavy chain framework region and donor antibody heavy chain framework region each comprises FR1, FR2, FR3 and FR4;
(b) synthesizing a nucleotide sequence encoding a humanized heavy chain variable region, said nucleotide sequence comprising nucleic acid sequences encoding complementarity determining regions (CDRs) from the donor antibody heavy chain variable region and nucleic acid sequences encoding the acceptor heavy chain variable framework regions; and
(c) introducing the nucleotide sequence encoding the humanized heavy chain variable region into a cell.

28. A method of producing a humanized antibody that immunospecifically binds to an antigen, said method comprising providing a cell containing nucleotide sequences encoding humanized heavy chain and light chain variable regions and expressing nucleotide sequences, wherein said cell containing the nucleotide sequences was produced by:
(a) comparing the nucleotide sequence of a donor antibody heavy chain variable region against a collection of sequences of acceptor heavy chain variable regions;
(b) selecting an acceptor heavy chain variable framework region less than 65% identical to the donor antibody heavy chain variable framework region at the amino acid level, which acceptor heavy chain variable framework region contains at least one amino acid residue at amino acid residues 6, 23, 24 or 49 according to the Kabat numbering system that is not identical to the corresponding residue in the donor antibody, wherein the acceptor heavy chain framework region and donor antibody heavy chain framework region each comprises FR1, FR2, FR3 and FR4;
(c) synthesizing a nucleic acid sequence comprising nucleotide sequence encoding a humanized heavy chain variable region, said nucleotide sequence comprising nucleic acid sequences encoding complementarity determining regions (CDRs) from the donor antibody heavy chain variable region and nucleic acid sequences encoding the acceptor heavy chain variable framework regions with one or more mutations introduced at residues designated key residues; and
(d) introducing the nucleic acid sequence comprising the nucleotide sequence encoding the humanized heavy chain variable region into a cell.

29. The method of claim 27, wherein the residues designated key are one or more of the following: a residue adjacent to a CDR, a potential glycosylation site, a rare residue, a residue capable of interacting with the antigen, a residue capable of interacting with a CDR, a canonical residue, a contact residue between the variable heavy region and variable light region, and a residue within the Vernier zone.

30. The method of claim 28, wherein the residues designated key are one or more of the following: a residue adjacent to a CDR, a potential glycosylation site, a rare residue, a residue capable of interacting with the antigen, a residue capable of interacting with a CDR, a canonical residue, a contact residue between the variable heavy region and variable light region, a residue within the Vernier zone, and a residue within the region which overlaps between the Chothia definition of the heavy chain variable region CDR1 and the Kabat definition of the first heavy chain framework.

31. A humanized antibody produced by the method of claim 27 or 28.

32. A composition comprising the humanized antibody of claim 31, and a carrier, diluent or excipient.

33. A method of identifying a humanized antibody that immunospecifically binds to an antigen, said method comprising expressing the nucleic acid sequences in the cells of claim 11, 14, 17, or 20 and screening for a humanized antibody that has an affinity of 1×10⁶ M⁻¹ or above for said antigen.

34. A humanized antibody identified by the method of claim 33.

35. A composition comprising the humanized antibody of claim 34, and a carrier, diluent or excipient.


SEQUENCE LISTING (518 sequences)

SEQ ID NO: 1 - protein, 23 aa, Homo sapiens
DVVMTQSPLSLPVTLGQPASISC

SEQ ID NO: 2 - protein, 15 aa, Homo sapiens
WFQQRPGQSPRRLIY

SEQ ID NO: 3 - protein, 32 aa, Homo sapiens
GVPDRFSGSGSGTDFTLKISRVEAEDVGVYYC

SEQ ID NO: 4 - protein, 23 aa, Homo sapiens
EIVLTQSPDFQSVTPKEKVTITC

SEQ ID NO: 5 - protein, 15 aa, Homo sapiens
WYQQKPDQSPKLLIK

SEQ ID NO: 6 - protein, 32 aa, Homo sapiens
GVPSRFSGSGSGTDFTLTINSLEAEDAATYYC

SEQ ID NO: 7 - protein, 23 aa, Homo sapiens
EIVLTQSPATLSLSPGERATLSC

SEQ ID NO: 8 - protein, 15 aa, Homo sapiens
WYQQKPGLAPRLLIY

SEQ ID NO: 9 - protein, 32 aa, Homo sapiens
GIPDRFSGSGSGTDFTLTISRLEPEDFAVYYC

SEQ ID NO: 10 - protein, 23 aa, Homo sapiens
DVVMTQSPAFLSVTPGEKVTITC

SEQ ID NO: 11 - protein, 15 aa, Homo sapiens
WYQQKPDQAPKLLIK

SEQ ID NO: 12 - protein, 32 aa, Homo sapiens
GVPSRFSGSGSGTDFTFTISSLEAEDAATYYC

SEQ ID NO: 13 - protein, 23 aa, Homo sapiens
DVVMTQSPLSLPVTLGQPASISC

SEQ ID NO: 14 - protein, 15 aa, Homo sapiens
WFQQRPGQSPRRLIY

SEQ ID NO: 15 - protein, 32 aa, Homo sapiens; identical to SEQ ID NO: 3

SEQ ID NO: 16 - protein, 23 aa, Homo sapiens
DIVMTQTPLSLSVTPGQPASISC

SEQ ID NO: 17 - protein, 15 aa, Homo sapiens
WYLQKPGQSPQLLIY

SEQ ID NO: 18 - protein, 32 aa, Homo sapiens; identical to SEQ ID NO: 3

SEQ ID NO: 19 - protein, 23 aa, Homo sapiens
DIVMTQSPLSLPVTPGEPASISC

SEQ ID NO: 20 - protein, 15 aa, Homo sapiens
WYLQKPGQSPQLLIY

SEQ ID NO: 21 - protein, 32 aa, Homo sapiens; identical to SEQ ID NO: 3

SEQ ID NO: 22 - protein, 23 aa, Homo sapiens
DIVMTQTPLSLSVTPGQPASISC

SEQ ID NO: 23 - protein, 15 aa, Homo sapiens
WYLQKPGQPPQLLIY

SEQ ID NO: 24 - protein, 32 aa, Homo sapiens; identical to SEQ ID NO: 3

SEQ ID NO: 25 - protein, 23 aa, Homo sapiens
DIQMTQSPSSLSASVGDRVTITC

SEQ ID NO: 26 - protein, 15 aa, Homo sapiens
WYQQKPGKVPKLLIY

SEQ ID NO: 27 - protein, 32 aa, Homo sapiens
GVPSRFSGSGSGTDFTLTISSLQPEDVATYYC

SEQ ID NO: 28 - protein, 23 aa, Homo sapiens
DIVMTQTPLSSPVTLGQPASISC

SEQ ID NO: 29 - protein, 15 aa, Homo sapiens
WLQQRPGQPPRLLIY

SEQ ID NO: 30 - protein, 32 aa, Homo sapiens
GVPDRFSGSGAGTDFTLKISRVEAEDVGVYYC

SEQ ID NO: 31 - protein, 23 aa, Homo sapiens
EIVLTQSPDFQSVTPKEKVTITC

SEQ ID NO: 32 - protein, 15 aa, Homo sapiens
WYQQKPDQSPKLLIK

SEQ ID NO: 33 - protein, 32 aa, Homo sapiens; identical to SEQ ID NO: 6

SEQ ID NO: 34 - protein, 23 aa, Homo sapiens
EIVLTQSPGTLSLSPGERATLSC

SEQ ID NO: 35 - protein, 15 aa, Homo sapiens
WYQQKPGQAPRLLIY

SEQ ID NO: 36 - protein, 32 aa, Homo sapiens; identical to SEQ ID NO: 9

SEQ ID NO: 37 - protein, 23 aa, Homo sapiens
DIVMTQSPLSLPVTPGEPASISC

SEQ ID NO: 38 - protein, 15 aa, Homo sapiens
WYLQKPGQSPQLLIY

SEQ ID NO: 39 - protein, 32 aa, Homo sapiens; identical to SEQ ID NO: 3

SEQ ID NO: 40 - protein, 23 aa, Homo sapiens
DIQMTQSPSSLSASVGDRVTITC

SEQ ID NO: 41 - protein, 15 aa, Homo sapiens
WYQQKPGKAPKRLIY

SEQ ID NO: 42 - protein, 32 aa, Homo sapiens
GVPSRFSGSGSGTEFTLTISSLQPEDFATYYC

SEQ ID NO: 43 - protein, 23 aa, Homo sapiens
EIVMTQTPLSLSITPGEQASISC

SEQ ID NO: 44 - protein, 15 aa, Homo sapiens
WFLQKARPVSTLLIY

SEQ ID NO: 45 - protein, 32 aa, Homo sapiens
GVPDRFSGSGSGTDFTLKISRVEAEDFGVYYC

SEQ ID NO: 46 - protein, 23 aa, Homo sapiens
DIVMTQTPLSSPVTLGQPASISF

SEQ ID NO: 47 - protein, 15 aa, Homo sapiens
WLQQRPGQPPRLLIY

SEQ ID NO: 48 - protein, 32 aa, Homo sapiens; identical to SEQ ID NO: 30

SEQ ID NO: 49 - protein, 23 aa, Homo sapiens
ETTLTQSPAFMSATPGDKVNISC

SEQ ID NO: 50 - protein, 15 aa, Homo sapiens
WYQQKPGEAAIFIIQ

SEQ ID NO: 51 - protein, 32 aa, Homo sapiens
GIPPRFSGSGYGTDFTLTINNIESEDAAYYFC

SEQ ID NO: 52 - protein, 23 aa, Homo sapiens
DIVMTQSPDSLAVSLGERATINC

SEQ ID NO: 53 - protein, 15 aa, Homo sapiens
WYQQKPGQPPKLLIY

SEQ ID NO: 54 - protein, 32 aa, Homo sapiens
GVPDRFSGSGSGTDFTLTISSLQAEDVAVYYC

SEQ ID NO: 55 - protein, 23 aa, Homo sapiens
DIQMTQSPSSLSASVGDRVTITC

SEQ ID NO: 56 - protein, 15 aa, Homo sapiens
WFQQKPGKAPKSLIY

SEQ ID NO: 57 - protein, 32 aa, Homo sapiens
GVPSRFSGSGSGTDFTLTISSLQPEDFATYYC

SEQ ID NO: 58 - protein, 23 aa, Homo sapiens
EIVMTQSPPTLSLSPGERVTLSC

SEQ ID NO: 59 - protein, 15 aa, Homo sapiens
WYQQKPGQAPRLLIY

SEQ ID NO: 60 - protein, 32 aa, Homo sapiens
SIPARFSGSGSGTDFTLTISSLQPEDFAVYYC

SEQ ID NO: 61 - protein, 23 aa, Homo sapiens
AIQMTQSPSSLSASVGDRVTITC

SEQ ID NO: 62 - protein, 15 aa, Homo sapiens
WYQQKPGKAPKLLIY

SEQ ID NO: 63 - protein, 32 aa, Homo sapiens; identical to SEQ ID NO: 57

SEQ ID NO: 64 - protein, 23 aa, Homo sapiens
DIQMTQSPSTLSASVGDRVTITC

SEQ ID NO: 65 - protein, 15 aa, Homo sapiens
WYQQKPGKAPKLLIY

SEQ ID NO: 66 - protein, 32 aa, Homo sapiens
GVPSRFSGSGSGTEFTLTISSLQPDDFATYYC

SEQ ID NO: 67 - protein, 23 aa, Homo sapiens
NIQMTQSPSAMSASVGDRVTITC

SEQ ID NO: 68 - protein, 15 aa, Homo sapiens
WFQQKPGKVPKHLIY

SEQ ID NO: 69 - protein, 32 aa, Homo sapiens; identical to SEQ ID NO: 42

SEQ ID NO: 70 - protein, 23 aa, Homo sapiens
DIQMTQSPSSLSASVGDRVTITC

SEQ ID NO: 71 - protein, 15 aa, Homo sapiens
WYQQKPEKAPKSLIY

SEQ ID NO: 72 - protein, 32 aa, Homo sapiens; identical to SEQ ID NO: 57

SEQ ID NO: 73 - protein, 23 aa, Homo sapiens
EIVMTQSPATLSVSPGERATLSC

SEQ ID NO: 74 - protein, 15 aa, Homo sapiens
WYQQKPGQAPRLLIY

SEQ ID NO: 75 - protein, 32 aa, Homo sapiens
GIPARFSGSGSGTEFTLTISSLQSEDFAVYYC

SEQ ID NO: 76 - protein, 23 aa, Homo sapiens
AIQLTQSPSSLSASVGDRVTITC

SEQ ID NO: 77 - protein, 15 aa, Homo sapiens
WYQQKPGKAPKLLIY

SEQ ID NO: 78 - protein, 32 aa, Homo sapiens; identical to SEQ ID NO: 57

SEQ ID NO: 79 - protein, 23 aa, Homo sapiens
DIQMTQSPSSVSASVGDRVTITC

SEQ ID NO: 80 - protein, 15 aa, Homo sapiens
WYQQKPGKAPKLLIY

SEQ ID NO: 81 - protein, 32 aa, Homo sapiens; identical to SEQ ID NO: 57

SEQ ID NO: 82 - protein, 23 aa, Homo sapiens
EIVMTQSPATLSVSPGERATLSC

SEQ ID NO: 83 - protein, 15 aa, Homo sapiens
WYQQKPGQAPRLLIY

SEQ ID NO: 84 - protein, 32 aa, Homo sapiens; identical to SEQ ID NO: 75

SEQ ID NO: 85 - protein, 23 aa, Homo sapiens
EIVLTQSPATLSLSPGERATLSC

SEQ ID NO: 86 - protein, 15 aa, Homo sapiens
WYQQKPGQAPRLLIY

SEQ ID NO: 87 - protein, 32 aa, Homo sapiens
GIPARFSGSGPGTDFTLTISSLEPEDFAVYYC

SEQ ID NO: 88 - protein, 23 aa, Homo sapiens
DIQMIQSPSFLSASVGDRVSIIC

SEQ ID NO: 89 - protein, 15 aa, Homo sapiens
WYLQKPGKSPKLFLY

SEQ ID NO: 90 - protein, 32 aa, Homo sapiens
GVSSRFSGRGSGTDFTLTIISLKPEDFAAYYC

SEQ ID NO: 91 - protein, 23 aa, Homo sapiens
AIRMTQSPFSLSASVGDRVTITC

SEQ ID NO: 92 - protein, 15 aa, Homo sapiens
WYQQKPAKAPKLFIY

SEQ ID NO: 93 - protein, 32 aa, Homo sapiens
GVPSRFSGSGSGTDYTLTISSLQPEDFATYYC

SEQ ID NO: 94 - protein, 23 aa, Homo sapiens
VIWMTQSPSLLSASTGDRVTISC

SEQ ID NO: 95 - protein, 15 aa, Homo sapiens
WYQQKPGKAPELLIY

SEQ ID NO: 96 - protein, 32 aa, Homo sapiens
GVPSRFSGSGSGTDFTLTISCLQSEDFATYYC

SEQ ID NO: 97 - protein, 23 aa, Homo sapiens
EIVMTQSPATLSLSPGERATLSC

SEQ ID NO: 98 - protein, 15 aa, Homo sapiens
WYQQKPGQAPRLLIY

SEQ ID NO: 99 - protein, 32 aa, Homo sapiens
GIPARFSGSGSGTDFTLTISSLQPEDFAVYYC

SEQ ID NO: 100 - protein, 23 aa, Homo sapiens
AIQLTQSPSSLSASVGDRVTITC

SEQ ID NO: 101 - protein, 15 aa, Homo sapiens
WYQQKPGKAPKLLIY

SEQ ID NO: 102 - protein, 32 aa, Homo sapiens; identical to SEQ ID NO: 57

SEQ ID NO: 103 - protein, 23 aa, Homo sapiens
DIQMTQSPSSVSASVGDRVTITC

SEQ ID NO: 104 - protein, 15 aa, Homo sapiens
WYQQKPGKAPKLLIY

SEQ ID NO: 105 - protein, 32 aa, Homo sapiens; identical to SEQ ID NO: 57

SEQ ID NO: 106 - protein, 23 aa, Homo sapiens
EIVLTQSPATLSLSPGERATLSC

SEQ ID NO: 107 - protein, 15 aa, Homo sapiens
WYQQKPGQAPRLLIY

SEQ ID NO: 108 - protein, 32 aa, Homo sapiens
GIPARFSGSGSGTDFTLTISSLEPEDFAVYYC

SEQ ID NO: 109 - protein, 23 aa, Homo sapiens
DIQLTQSPSFLSASVGDRVTITC

SEQ ID NO: 110 - protein, 15 aa, Homo sapiens
WYQQKPGKAPKLLIY

SEQ ID NO: 111 - protein, 32 aa, Homo sapiens; identical to SEQ ID NO: 42

SEQ ID NO: 112 - protein, 23 aa, Homo sapiens
AIRMTQSPSSFSASTGDRVTITC

SEQ ID NO: 113 - protein, 15 aa, Homo sapiens
WYQQKPGKAPKLLIY

SEQ ID NO: 114 - protein, 32 aa, Homo sapiens; identical to SEQ ID NO: 96

SEQ ID NO: 115 - protein, 23 aa, Homo sapiens
DIVMTQTPLSLPVTPGEPASISC

SEQ ID NO: 116 - protein, 15 aa, Homo sapiens
WYLQKPGQSPQLLIY

SEQ ID NO: 117 - protein, 32 aa, Homo sapiens; identical to SEQ ID NO: 3

SEQ ID NO: 118 - protein, 23 aa, Homo sapiens
DIVMTQTPLSLPVTPGEPASISC

SEQ ID NO: 119 - protein, 15 aa, Homo sapiens
WYLQKPGQSPQLLIY

SEQ ID NO: 120 - protein, 32 aa, Homo sapiens; identical to SEQ ID NO: 3

SEQ ID NO: 121 - protein, 23 aa, Homo sapiens
DIQMTQSPSSLSASVGDRVTITC

SEQ ID NO: 122 - protein, 15 aa, Homo sapiens
WYQQKPGKAPKLLIY

SEQ ID NO: 123 - protein, 32 aa, Homo sapiens; identical to SEQ ID NO: 57

SEQ ID NO: 124 - protein, 23 aa, Homo sapiens
DIQLTQSPSSLSASVGDRVTITC

SEQ ID NO: 125 - protein, 15 aa, Homo sapiens
WYRQKPGKVPKLLIY

SEQ ID NO: 126 - protein, 32 aa, Homo sapiens
GVPSRFSGSGSGTDFTLTISSLQPEDVATYYG

SEQ ID NO: 127 - protein, 23 aa, Homo sapiens
DIQMTQSPSSLSASVGDRVTITC

SEQ ID NO: 128 - protein, 15 aa, Homo sapiens
WYQQKPGKAPKLLIY

SEQ ID NO: 129 - protein, 32 aa, Homo sapiens
GVPSRFSGSGSGTDFTFTISSLQPEDIATYYC

SEQ ID NO: 130 - protein, 23 aa, Homo sapiens
DIQMTQSPSSLSASVGDRVTITC

SEQ ID NO: 131 - protein, 15 aa, Homo sapiens
WYQQKPGKAPKLLIY

SEQ ID NO: 132 - protein, 32 aa, Homo sapiens; identical to SEQ ID NO: 57

SEQ ID NO: 133 - protein, 23 aa, Homo sapiens
DIQLTQSPSSLSASVGDRVTITC

SEQ ID NO: 134 - protein, 15 aa, Homo sapiens
WYRQKPGKVPKLLIY

SEQ ID NO: 135 - protein, 32 aa, Homo sapiens; identical to SEQ ID NO: 126

SEQ ID NO: 136 - protein, 23 aa, Homo sapiens
DIQMTQSPSSLSASVGDRVTITC

SEQ ID NO: 137 - protein, 15 aa, Homo sapiens
WYQQKPGKAPKLLIY

SEQ ID NO: 138 - protein, 32 aa, Homo sapiens; identical to SEQ ID NO: 129

SEQ ID NO: 139 - protein, 12 aa, Homo sapiens
WTFGQGTKVEIK

SEQ ID NO: 140 - protein, 12 aa, Homo sapiens
YTFGQGTKLEIK

SEQ ID NO: 141 - protein, 12 aa, Homo sapiens
FTFGPGTKVDIK

SEQ ID NO: 142 - protein, 12 aa, Homo sapiens
LTFGGGTKVEIK

SEQ ID NO: 143 - protein, 12 aa, Homo sapiens
ITFGQGTRLEIK

SEQ ID NO: 144 - protein, 30 aa, Homo sapiens
QVQLVQSGAEVKKPGASVKVSCKASGYTFT

SEQ ID NO: 145 - protein, 14 aa, Homo sapiens
WVRQAPGQGLEWMG

SEQ ID NO: 146 - protein, 32 aa, Homo sapiens
RVTMTTDTSTSTAYMELRSLRSDDTAVYYCAR

SEQ ID NO: 147 - protein, 30 aa, Homo sapiens; identical to SEQ ID NO: 144

SEQ ID NO: 148 - protein, 14 aa, Homo sapiens
WVRQAPGQGLEWMG

SEQ ID NO: 149 - protein, 32 aa, Homo sapiens
RVTMTRDTSISTAYMELSRLRSDDTAVYYCAR

SEQ ID NO: 150 - protein, 30 aa, Homo sapiens
QVQLVQSGAEVKKPGASVKVSCKVSGYTLT

SEQ ID NO: 151 - protein, 14 aa, Homo sapiens
WVRQAPGKGLEWMG

SEQ ID NO: 152 - protein, 32 aa, Homo sapiens
RVTMTEDTSTDTAYMELSSLRSEDTAVYYCAT

SEQ ID NO: 153 - protein, 30 aa, Homo sapiens; identical to SEQ ID NO: 144

SEQ ID NO: 154 - protein, 14 aa, Homo sapiens
WVRQAPGQRLEWMG

SEQ ID NO: 155 - protein, 32 aa, Homo sapiens
RVTITRDTSASTAYMELSSLRSEDMAVYYCAR

SEQ ID NO: 156 - protein, 30 aa, Homo sapiens
QMQLVQSGAEVKKTGSSVKVSCKASGYTFT

SEQ ID NO: 157 - protein, 14 aa, Homo sapiens
WVRQAPGQALEWMG

SEQ ID NO: 158 - protein, 32 aa, Homo sapiens
RVTITRDRSMSTAYMELSSLRSEDTAMYYCAR

SEQ ID NO: 159 - protein, 30 aa, Homo sapiens; identical to SEQ ID NO: 144

SEQ ID NO: 160 - protein, 14 aa, Homo sapiens
WVRQAPGQGLEWMG

SEQ ID NO: 161 - protein, 32 aa, Homo sapiens
RVTMTRDTSTSTVYMELSSLRSEDTAVYYCAR

SEQ ID NO: 162 - protein, 30 aa, Homo sapiens
QMQLVQSGPEVKKPGTSVKVSCKASGFTFT

SEQ ID NO: 163 - protein, 14 aa, Homo sapiens
WVRQARGQRLEWIG

SEQ ID NO: 164 - protein, 32 aa, Homo sapiens
RVTITRDMSTSTAYMELSSLRSEDTAVYYCAA

SEQ ID NO: 165 - protein, 30 aa, Homo sapiens
QVQLVQSGAEVKKPGSSVKVSCKASGGTFS

SEQ ID NO: 166 - protein, 14 aa, Homo sapiens
WVRQAPGQGLEWMG

SEQ ID NO: 167 - protein, 32 aa, Homo sapiens
RVTITADKSTSTAYMELSSLRSEDTAVYYCAR

SEQ ID NO: 168 - protein, 30 aa, Homo sapiens; identical to SEQ ID NO: 144

SEQ ID NO: 169 - protein, 14 aa, Homo sapiens
WVRQATGQGLEWMG

SEQ ID NO: 170 - protein, 32 aa, Homo sapiens
RVTMTRNTSISTAYMELSSLRSEDTAVYYCAR

SEQ ID NO: 171 - protein, 30 aa, Homo sapiens
QVTLKESGPVLVKPTETLTLTCTVSGFSLS

SEQ ID NO: 172 - protein, 14 aa, Homo sapiens
WIRQPPGKALEWLA

SEQ ID NO: 173 - protein, 32 aa, Homo sapiens
RLTISKDTSKSQVVLTMTNMDPVDTATYYCAR

SEQ ID NO: 174 - protein, 30 aa, Homo sapiens
QITLKESGPTLVKPTQTLTLTCTFSGFSLS

SEQ ID NO: 175 - protein, 14 aa, Homo sapiens
WIRQPPGKALEWLA

SEQ ID NO: 176 - protein, 32 aa, Homo sapiens
RLTITKDTSKNQVVLTMTNMDPVDTATYYCAH

SEQ ID NO: 177 - protein, 30 aa, Homo sapiens
QVTLRESGPALVKPTQTLTLTCTFSGFSLS

SEQ ID NO: 178 - protein, 14 aa, Homo sapiens
WIRQPPGKALEWLA

SEQ ID NO: 179 - protein, 32 aa, Homo sapiens
RLTISKDTSKNQVVLTMTNMDPVDTATYYCAR

SEQ ID NO: 180 - protein, 30 aa, Homo sapiens
QVQLVESGGGLVKPGGSLRLSCAASGFTFS

SEQ ID NO: 181 - protein, 14 aa, Homo sapiens
WIRQAPGKGLEWVS

SEQ ID NO: 182 - protein, 32 aa, Homo sapiens
RFTISRDNAKNSLYLQMNSLRAEDTAVYYCAR

SEQ ID NO: 183 - protein, 30 aa, Homo sapiens
EVQLVESGGGLVQPGGSLRLSCAASGFTFS

SEQ ID NO: 184 - protein, 14 aa, Homo sapiens
WVRQATGKGLEWVS

SEQ ID NO: 185 - protein, 32 aa, Homo sapiens
RFTISRENAKNSLYLQMNSLRAGDTAVYYCAR

SEQ ID NO: 186 - protein, 30 aa, Homo sapiens
EVQLVESGGGLVKPGGSLRLSCAASGFTFS

SEQ ID NO: 187 - protein, 14 aa, Homo sapiens
WVRQAPGKGLEWVG

SEQ ID NO: 188 - protein, 32 aa, Homo sapiens
RFTISRDDSKNTLYLQMNSLKTEDTAVYYCTT

SEQ ID NO: 189 - protein, 30 aa, Homo sapiens; identical to SEQ ID NO: 183

SEQ ID NO: 190 - protein, 14 aa, Homo sapiens
WARKAPGKGLEWVS

SEQ ID NO: 191 - protein, 32 aa, Homo sapiens
RFIISRDNSRNSLYLQKNRRRAEDMAVYYCVR

SEQ ID NO: 192 - protein, 30 aa, Homo sapiens
EVQLVESGGGVVRPGGSLRLSCAASGFTFD

SEQ ID NO: 193 - protein, 14 aa, Homo sapiens
WVRQAPGKGLEWVS

SEQ ID NO: 194 - protein, 32 aa, Homo sapiens
RFTISRDNAKNSLYLQMNSLRAEDTALYHCAR

SEQ ID NO: 195 - protein, 30 aa, Homo sapiens; identical to SEQ ID NO: 186

SEQ ID NO: 196 - protein, 14 aa, Homo sapiens
WVRQAPGKGLEWVS

SEQ ID NO: 197 - protein, 32 aa, Homo sapiens; identical to SEQ ID NO: 182

SEQ ID NO: 198 - protein, 30 aa, Homo sapiens
EVQLLESGGGLVQPGGSLRLSCAASGFTFS

SEQ ID NO: 199 - protein, 14 aa, Homo sapiens
WVRQAPGKGLEWVS

SEQ ID NO: 200 - protein, 32 aa, Homo sapiens
RFTISRDNSKNTLYLQMNSLRAEDTAVYYCAK

SEQ ID NO: 201 - protein, 30 aa, Homo sapiens
QVQLVESGGGVVQPGRSLRLSCAASGFTFS

SEQ ID NO: 202 - protein, 14 aa, Homo sapiens
WVRQAPGKGLEWVA

SEQ ID NO: 203 - protein, 32 aa, Homo sapiens
RFTISRDNSKNTLYLQMNSLRAEDTAVYYCAR

SEQ ID NO: 204 - protein, 30 aa, Homo sapiens; identical to SEQ ID NO: 201

SEQ ID NO: 205 - protein, 14 aa, Homo sapiens
WVRQAPGKGLEWVA

SEQ ID NO: 206 - protein, 32 aa, Homo sapiens; identical to SEQ ID NO: 203

SEQ ID NO: 207 - protein, 30 aa, Homo sapiens; identical to SEQ ID NO: 183

SEQ ID NO: 208 - protein, 14 aa, Homo sapiens
WVHQAPGKGLEWVS

SEQ ID NO: 209 - protein, 32 aa, Homo sapiens
RFIISRDNSRNTLYLQTNSLRAEDTAVYYCVR

SEQ ID NO: 210 - protein, 30 aa, Homo sapiens
EVQLVESGGGLVQPRGSLRLSCAASGFTVS

SEQ ID NO: 211 - protein, 14 aa, Homo sapiens
WIRQAPGKGLEWVS

SEQ ID NO: 212 - protein, 32 aa, Homo sapiens
RFTISRDNSKNTLYLQMNNLRAEGTAVYYCAR

SEQ ID NO: 213 - protein, 30 aa, Homo sapiens
EVQLVESGGVVVQPGGSLRLSCAASGFTFD

SEQ ID NO: 214 - protein, 14 aa, Homo sapiens
WVRQAPGKGLEWVS

SEQ ID NO: 215 - protein, 32 aa, Homo sapiens
RFTISRDNSKNSLYLQMNSLRTEDTALYYCAK

SEQ ID NO: 216 - protein, 30 aa, Homo sapiens; identical to SEQ ID NO: 183

SEQ ID NO: 217 - protein, 14 aa, Homo sapiens
WVRQAPGKGLEWVS

SEQ ID NO: 218 - protein, 32 aa, Homo sapiens
RFTISRDNAKNSLYLQMNSLRDEDTAVYYCAR

SEQ ID NO: 219 - protein, 30 aa, Homo sapiens
EVQLVESGGGLVQPGRSLRLSCTASGFTFG

SEQ ID NO: 220 - protein, 14 aa, Homo sapiens
WFRQAPGKGLEWVG

SEQ ID NO: 221 - protein, 32 aa, Homo sapiens
RFTISRDDSKSIAYLQMNSLKTEDTAVYYCTR

SEQ ID NO: 222 - protein, 30 aa, Homo sapiens
EVQLVESGGGLIQPGGSLRLSCAASGFTVS

SEQ ID NO: 223 - protein, 14 aa, Homo sapiens
WVRQAPGKGLEWVS

SEQ ID NO: 224 - protein, 32 aa, Homo sapiens; identical to SEQ ID NO: 203

SEQ ID NO: 225 - protein, 30 aa, Homo sapiens
EVQLVESGEGLVQPGGSLRLSCAASGFTFS

SEQ ID NO: 226 - protein, 14 aa, Homo sapiens
WVRQAPGKGLEYVS

SEQ ID NO: 227 - protein, 32 aa, Homo sapiens
RFTISRDNSKNTLYLQMGSLRAEDMAVYYCAR

SEQ ID NO: 228 - protein, 30 aa, Homo sapiens; identical to SEQ ID NO: 222

SEQ ID NO: 229 - protein, 14 aa, Homo sapiens
WVRQAPGKGLEWVS

SEQ ID NO: 230 - protein, 32 aa, Homo sapiens; identical to SEQ ID NO: 203

SEQ ID NO: 231 - protein, 30 aa, Homo sapiens; identical to SEQ ID NO: 183

SEQ ID NO: 232 - protein, 14 aa, Homo sapiens
WVRQAPGKGLEWVA

SEQ ID NO: 233 - protein, 32 aa, Homo sapiens; identical to SEQ ID NO: 182

SEQ ID NO: 234 - protein, 30 aa, Homo sapiens; identical to SEQ ID NO: 183

SEQ ID NO: 235 - protein, 14 aa, Homo sapiens
WVRQAPGKGLEWVG

SEQ ID NO: 236 - protein, 32 aa, Homo sapiens
RFTISRDDSKNSLYLQMNSLKTEDTAVYYCAR

SEQ ID NO: 237 - protein, 30 aa, Homo sapiens
EVQLVESGGGLVQPGGSLKLSCAASGFTFS

SEQ ID NO: 238 - protein, 14 aa, Homo sapiens
WVRQASGKGLEWVG

SEQ ID NO: 239 - protein, 32 aa, Homo sapiens
RFTISRDDSKNTAYLQMNSLKTEDTAVYYCTR

SEQ ID NO: 240 - protein, 30 aa, Homo sapiens; identical to SEQ ID NO: 183

SEQ ID NO: 241 - protein, 14 aa, Homo sapiens
WVRQAPGKGLVWVS

SEQ ID NO: 242 - protein, 32 aa, Homo sapiens
RFTISRDNAKNTLYLQMNSLRAEDTAVYYCAR

SEQ ID NO: 243 - protein, 30 aa, Homo sapiens
EVQLVESGGGLVQPGRSLRLSCAASGFTFD

SEQ ID NO: 244 - protein, 14 aa, Homo sapiens
WVRQAPGKGLEWVS

SEQ ID NO: 245 - protein, 32 aa, Homo sapiens
RFTISRDNAKNSLYLQMNSLRAEDTALYYCAK

SEQ ID NO: 246 - protein, 30 aa, Homo sapiens
QVQLQESGPGLVKPSDTLSLTCAVSGYSIS

SEQ ID NO: 247 - protein, 14 aa, Homo sapiens
WIRQPPGKGLEWIG

SEQ ID NO: 248 - protein, 32 aa, Homo sapiens
RVTMSVDTSKNQFSLKLSSVTAVDTAVYYCAR

SEQ ID NO: 249 - protein, 30 aa, Homo sapiens
QVQLQESGPGLVKPSQTLSLTCTVSGGSIS

SEQ ID NO: 250 - protein, 14 aa, Homo sapiens
WIRQHPGKGLEWIG

SEQ ID NO: 251 - protein, 32 aa, Homo sapiens
RVTISVDTSKNQFSLKLSSVTAADTAVYYCAR

SEQ ID NO: 252 - protein, 30 aa, Homo sapiens
QVQLQQWGAGLLKPSETLSLTCAVYGGSFS

SEQ ID NO: 253 - protein, 14 aa, Homo sapiens
WIRQPPGKGLEWIG

SEQ ID NO: 254 - protein, 32 aa, Homo sapiens; identical to SEQ ID NO: 251

SEQ ID NO: 255 - protein, 30 aa, Homo sapiens
QLQLQESGPGLVKPSETLSLTCTVSGGSIS

SEQ ID NO: 256 - protein, 14 aa, Homo sapiens
WIRQPPGKGLEWIG

SEQ ID NO: 257 - protein, 32 aa, Homo sapiens; identical to SEQ ID NO: 251

SEQ ID NO: 258 - protein, 30 aa, Homo sapiens
QVQLQESGPGLVKPSETLSLTCTVSGGSIS

SEQ ID NO: 259 - protein, 14 aa, Homo sapiens
WIRQPAGKGLEWIG

SEQ ID NO: 260 - protein, 32 aa, Homo sapiens
RVTMSVDTSKNQFSLKLSSVTAADTAVYYCAR

SEQ ID NO: 261 - protein, 30 aa, Homo sapiens; identical to SEQ ID NO: 258

SEQ ID NO: 262 - protein, 14 aa, Homo sapiens
WIRQPPGKGLEWIG

SEQ ID NO: 263 - protein, 32 aa, Homo sapiens; identical to SEQ ID NO: 251

SEQ ID NO: 264 - protein, 30 aa, Homo sapiens
QVQLQESGPGLVKPSETLSLTCTVSGGSVS

SEQ ID NO: 265 - protein, 14 aa, Homo sapiens
WIRQPPGKGLEWIG

SEQ ID NO: 266 - protein, 32 aa, Homo sapiens; identical to SEQ ID NO: 251

SEQ ID NO: 267 - protein, 30 aa, Homo sapiens
EVQLVQSGAEVKKPGESLKISCKGSGYSFT

SEQ ID NO: 268 - protein, 14 aa, Homo sapiens
WVRQMPGKGLEWMG

SEQ ID NO: 269 - protein, 32 aa, Homo sapiens
QVTISADKSISTAYLQWSSLKASDTAMYYCAR

SEQ ID NO: 270 - protein, 30 aa, Homo sapiens
QVQLQQSGPGLVKPSQTLSLTCAISGDSVS

SEQ ID NO: 271 - protein, 14 aa, Homo sapiens
WIRQSPSRGLEWLG

SEQ ID NO: 272 - protein, 32 aa, Homo sapiens
RITINPDTSKNQFSLQLNSVTPEDTAVYYCAR

SEQ ID NO: 273 - protein, 30 aa, Homo sapiens
QVQLVQSGHEVKQPGASVKVSCKASGYSFT

SEQ ID NO: 274 - protein, 14 aa, Homo sapiens
WVPQAPGQGLEWMG

SEQ ID NO: 275 - protein, 32 aa, Homo sapiens
RFVFSMDTSASTAYLQISSLKAEDMAMYYCAR

SEQ ID NO: 276 - protein, 25 aa, Homo sapiens [sequence omitted]

SEQ ID NO: 277 - protein, 17 aa, Homo sapiens
GISWVRQAPGQGLEWMG

SEQ ID NO: 278 - protein, 32 aa, Homo sapiens; identical to SEQ ID NO: 146

SEQ ID NO: 279 - protein, 25 aa, Homo sapiens [sequence omitted]

SEQ ID NO: 280 - protein, 17 aa, Homo sapiens
YMHWVRQAPGQGLEWMG

SEQ ID NO: 281 - protein, 32 aa, Homo sapiens; identical to SEQ ID NO: 149

SEQ ID NO: 282 - protein, 25 aa, Homo sapiens
QVQLVQSGAEVKKPGASVKVSCKVS

SEQ ID NO: 283 - protein, 17 aa, Homo sapiens
SMHWVRQAPGKGLEWMG

SEQ ID NO: 284 - protein, 32 aa, Homo sapiens; identical to SEQ ID NO: 152

SEQ ID NO: 285 - protein, 25 aa, Homo sapiens [sequence omitted]

SEQ ID NO: 286 - protein, 17 aa, Homo sapiens
AMHWVRQAPGQRLEWMG

SEQ ID NO: 287 - protein, 32 aa, Homo sapiens; identical to SEQ ID NO: 155

SEQ ID NO: 288 - protein, 25 aa, Homo sapiens
QMQLVQSGAEVKKTGSSVKVSCKAS

SEQ ID NO: 289 - protein, 17 aa, Homo sapiens
YLHWVRQAPGQALEWMG

SEQ ID NO: 290 - protein, 32 aa, Homo sapiens; identical to SEQ ID NO: 158

SEQ ID NO: 291 - protein, 25 aa, Homo sapiens [sequence omitted]

SEQ ID NO: 292 - protein, 17 aa, Homo sapiens
YMHWVRQAPGQGLEWMG

SEQ ID NO: 293 - protein, 32 aa, Homo sapiens; identical to SEQ ID NO: 161

SEQ ID NO: 294 - protein, 25 aa, Homo sapiens
QMQLVQSGPEVKKPGTSVKVSCKAS

SEQ ID NO: 295 - protein, 17 aa, Homo sapiens
AMQWVRQARGQRLEWIG

SEQ ID NO: 296 - protein, 32 aa, Homo sapiens; identical to SEQ ID NO: 164

SEQ ID NO: 297 - protein, 25 aa, Homo sapiens [sequence omitted]

SEQ ID NO: 298 - protein, 17 aa, Homo sapiens
AISWVRQAPGQGLEWMG

SEQ ID NO: 299 - protein, 32 aa, Homo sapiens; identical to SEQ ID NO: 167

SEQ ID NO: 300 - protein, 25 aa, Homo sapiens [sequence omitted]

SEQ ID NO: 301 - protein, 17 aa, Homo sapiens
DINWVRQATGQGLEWMG

SEQ ID NO: 302 - protein, 33 aa, Homo sapiens
RVTMTRNTSISTAYMELSSLRSEDTAVYYCARG

SEQ ID NO: 303 - protein, 25 aa, Homo sapiens
QVTLKESGPVLVKPTETLTLTCTVS

SEQ ID NO: 304 - protein, 17 aa, Homo sapiens
GVSWIRQPPGKALEWLA

SEQ ID NO: 305 - protein, 32 aa, Homo sapiens; identical to SEQ ID NO: 173

SEQ ID NO: 306 - protein, 25 aa, Homo sapiens
QITLKESGPTLVKPTQTLTLTCTFS

SEQ ID NO: 307 - protein, 17 aa, Homo sapiens
GVGWIRQPPGKALEWLA

SEQ ID NO: 308 - protein, 33 aa, Homo sapiens
RLTITKDTSKNQVVLTMTNMDPVDTATYYCAHR

SEQ ID NO: 309 - protein, 25 aa, Homo sapiens
QVTLRESGPALVKPTQTLTLTCTFS

SEQ ID NO: 310 - protein, 17 aa, Homo sapiens
CVSWIRQPPGKALEWLA

SEQ ID NO: 311 - protein, 32 aa, Homo sapiens; identical to SEQ ID NO: 179

SEQ ID NO: 312 - protein, 25 aa, Homo sapiens
QVQLVESGGGLVKPGGSLRLSCAAS

SEQ ID NO: 313 - protein, 17 aa, Homo sapiens
YMSWIRQAPGKGLEWVS

SEQ ID NO: 314 - protein, 32 aa, Homo sapiens; identical to SEQ ID NO: 182

SEQ ID NO: 315 - protein, 25 aa, Homo sapiens [sequence omitted]

SEQ ID NO: 316 - protein, 17 aa, Homo sapiens
DMHWVRQATGKGLEWVS

SEQ ID NO: 317 - protein, 32 aa, Homo sapiens; identical to SEQ ID NO: 185

SEQ ID NO: 318 - protein, 25 aa, Homo sapiens
EVQLVESGGGLVKPGGSLRLSCAAS

SEQ ID NO: 319 - protein, 17 aa, Homo sapiens
WMSWVRQAPGKGLEWVG

SEQ ID NO: 320 - protein, 32 aa, Homo sapiens; identical to SEQ ID NO: 188

SEQ ID NO: 321 - protein, 25 aa, Homo sapiens [sequence omitted]

SEQ ID NO: 322 - protein, 17 aa, Homo sapiens
DMNWARKAPGKGLEWVS

SEQ ID NO: 323 - protein, 32 aa, Homo sapiens; identical to SEQ ID NO: 191

SEQ ID NO: 324 - protein, 25 aa, Homo sapiens
EVQLVESGGGVVRPGGSLRLSCAAS

SEQ ID NO: 325 - protein, 17 aa, Homo sapiens
GMSWVRQAPGKGLEWVS

SEQ ID NO: 326 - protein, 32 aa, Homo sapiens; identical to SEQ ID NO: 194

SEQ ID NO: 327 - protein, 25 aa, Homo sapiens
EVQLVESGGGLVKPGGSLRLSCAAS

SEQ ID NO: 328 - protein, 17 aa, Homo sapiens
SMNWVRQAPGKGLEWVS

SEQ ID NO: 329 - protein, 32 aa, Homo sapiens; identical to SEQ ID NO: 182

SEQ ID NO: 330 - protein, 25 aa, Homo sapiens
EVQLLESGGGLVQPGGSLRLSCAAS

SEQ ID NO: 331 - protein, 17 aa, Homo sapiens
AMSWVRQAPGKGLEWVS

SEQ ID NO: 332 - protein, 32 aa, Homo sapiens; identical to SEQ ID NO: 200

SEQ ID NO: 333 - protein, 25 aa, Homo sapiens
QVQLVESGGGVVQPGRSLRLSCAAS

SEQ ID NO: 334 - protein, 17 aa, Homo sapiens
GMHWVRQAPGKGLEWVA

SEQ ID NO: 335 - protein, 32 aa, Homo sapiens; identical to SEQ ID NO: 203

SEQ ID NO: 336 - protein, 25 aa, Homo sapiens
QVQLVESGGGVVQPGRSLRLSCAAS

SEQ ID NO: 337 - protein, 17 aa, Homo sapiens
GMHWVRQAPGKGLEWVA

SEQ ID NO: 338 - protein, 32 aa, Homo sapiens; identical to SEQ ID NO: 203

SEQ ID NO: 339 - protein, 25 aa, Homo sapiens [sequence omitted]

SEQ ID NO: 340 - protein, 17 aa, Homo sapiens
DMNWVHQAPGKGLEWVS

SEQ ID NO: 341 - protein, 32 aa, Homo sapiens; identical to SEQ ID NO: 209

SEQ ID NO: 342 - protein, 25 aa, Homo sapiens
EVQLVESGGGLVQPRGSLRLSCAAS

SEQ ID NO: 343 - protein, 17 aa, Homo sapiens
EMSWIRQAPGKGLEWVS

SEQ ID NO: 344 - protein, 32 aa, Homo sapiens; identical to SEQ ID NO: 212

SEQ ID NO: 345 - protein, 25 aa, Homo sapiens
EVQLVESGGVVVQPGGSLRLSCAAS

SEQ ID NO: 346 - protein, 17 aa, Homo sapiens
TMHWVRQAPGKGLEWVS

SEQ ID NO: 347 - protein, 33 aa, Homo sapiens
RFTISRDNSKNSLYLQMNSLRTEDTALYYCAKD

SEQ ID NO: 348 - protein, 25 aa, Homo sapiens [sequence omitted]

SEQ ID NO: 349 - protein, 17 aa, Homo sapiens
SMNWVRQAPGKGLEWVS

SEQ ID NO: 350 - protein, 32 aa, Homo sapiens; identical to SEQ ID NO: 218

SEQ ID NO: 351 - protein, 25 aa, Homo sapiens
EVQLVESGGGLVQPGRSLRLSCTAS

SEQ ID NO: 352 - protein, 17 aa, Homo sapiens
AMSWFRQAPGKGLEWVG

SEQ ID NO: 353 - protein, 32 aa, Homo sapiens; identical to SEQ ID NO: 221

SEQ ID NO: 354 - protein, 25 aa, Homo sapiens
EVQLVESGGGLIQPGGSLRLSCAAS

SEQ ID NO: 355 - protein, 17 aa, Homo sapiens
YMSWVRQAPGKGLEWVS

SEQ ID NO: 356 - protein, 32 aa, Homo sapiens; identical to SEQ ID NO: 203

SEQ ID NO: 357 - protein, 25 aa, Homo sapiens
EVQLVESGEGLVQPGGSLRLSCAAS

SEQ ID NO: 358 - protein, 17 aa, Homo sapiens
AMHWVRQAPGKGLEYVS

SEQ ID NO: 359 - protein, 32 aa, Homo sapiens; identical to SEQ ID NO: 227

SEQ ID NO: 360 - protein, 25 aa, Homo sapiens
EVQLVESGGGLIQPGGSLRLSCAAS

SEQ ID NO: 361 - protein, 17 aa, Homo sapiens
YMSWVRQAPGKGLEWVS

SEQ ID NO: 362 - protein, 32 aa, Homo sapiens; identical to SEQ ID NO: 203

SEQ ID NO: 363 - protein, 25 aa, Homo sapiens [sequence omitted]

SEQ ID NO: 364 - protein, 17 aa, Homo sapiens
WMSWVRQAPGKGLEWVA

SEQ ID NO: 365 - protein, 32 aa, Homo sapiens; identical to SEQ ID NO: 182

SEQ ID NO: 366 - protein, 25 aa, Homo sapiens [sequence omitted]

SEQ ID NO: 367 - protein, 17 aa, Homo sapiens
YMDWVRQAPGKGLEWVG

SEQ ID NO: 368 - protein, 32 aa, Homo sapiens; identical to SEQ ID NO: 236

SEQ ID NO: 369 - protein, 25 aa, Homo sapiens
EVQLVESGGGLVQPGGSLKLSCAAS

SEQ ID NO: 370 - protein, 17 aa, Homo sapiens
AMHWVRQASGKGLEWVG

SEQ ID NO: 371 - protein, 32 aa, Homo sapiens; identical to SEQ ID NO: 239

SEQ ID NO: 372 - protein, 25 aa, Homo sapiens [sequence omitted]

SEQ ID NO: 373 - protein, 17 aa, Homo sapiens
WMHWVRQAPGKGLVWVS

SEQ ID NO: 374 - protein, 32 aa, Homo sapiens; identical to SEQ ID NO: 242

SEQ ID NO: 375 - protein, 25 aa, Homo sapiens [sequence omitted]

SEQ ID NO: 376 - protein, 17 aa, Homo sapiens
AMHWVRQAPGKGLEWVS

SEQ ID NO: 377 - protein, 32 aa, Homo sapiens; identical to SEQ ID NO: 245

SEQ ID NO: 378 - protein, 25 aa, Homo sapiens
QVQLQESGPGLVKPSDTLSLTCAVS

SEQ ID NO: 379 - protein, 17 aa, Homo sapiens
WWGWIRQPPGKGLEWIG

SEQ ID NO: 380 - protein, 32 aa, Homo sapiens; identical to SEQ ID NO: 248

SEQ ID NO: 381 - protein, 25 aa, Homo sapiens
QVQLQESGPGLVKPSQTLSLTCTVS

SEQ ID NO: 382 - protein, 17 aa, Homo sapiens
YWSWIRQHPGKGLEWIG

SEQ ID NO: 383 - protein, 32 aa, Homo sapiens; identical to SEQ ID NO: 251

SEQ ID NO: 384 - protein, 25 aa, Homo sapiens [sequence omitted]

SEQ ID NO: 385 - protein, 17 aa, Homo sapiens
YWSWIRQPPGKGLEWIG

SEQ ID NO: 386 - protein, 32 aa, Homo sapiens; identical to SEQ ID NO: 251

SEQ ID NO: 387 - protein, 25 aa, Homo sapiens
QLQLQESGPGLVKPSETLSLTCTVS

SEQ ID NO: 388 - protein, 17 aa, Homo sapiens
YWGWIRQPPGKGLEWIG

SEQ ID NO: 389 - protein, 32 aa, Homo sapiens; identical to SEQ ID NO: 251

SEQ ID NO: 390 - protein, 25 aa, Homo sapiens
QVQLQESGPGLVKPSETLSLTCTVS

SEQ ID NO: 391 - protein, 17 aa, Homo sapiens
YWSWIRQPAGKGLEWIG

SEQ ID NO: 392 - protein, 32 aa, Homo sapiens; identical to SEQ ID NO: 260

SEQ ID NO: 393 - protein, 25 aa, Homo sapiens
QVQLQESGPGLVKPSETLSLTCTVS

SEQ ID NO: 394 - protein, 17 aa, Homo sapiens
YWSWIRQPPGKGLEWIG

SEQ ID NO: 395 - protein, 32 aa, Homo sapiens; identical to SEQ ID NO: 251

SEQ ID NO: 396 - protein, 25 aa, Homo sapiens
QVQLQESGPGLVKPSETLSLTCTVS

SEQ ID NO: 397 - protein, 17 aa, Homo sapiens
YWSWIRQPPGKGLEWIG

SEQ ID NO: 398 - protein, 32 aa, Homo sapiens; identical to SEQ ID NO: 251

SEQ ID NO: 399 - protein, 25 aa, Homo sapiens
EVQLVQSGAEVKKPGESLKISCKGS

SEQ ID NO: 400 - protein, 17 aa, Homo sapiens
WIGWVRQMPGKGLEWMG

SEQ ID NO: 401 - protein, 32 aa, Homo sapiens; identical to SEQ ID NO: 269

SEQ ID NO: 402 - protein, 25 aa, Homo sapiens
QVQLQQSGPGLVKPSQTLSLTCAIS

SEQ ID NO: 403 - protein, 17 aa, Homo sapiens
AWNWIRQSPSRGLEWLG

SEQ ID NO: 404 - protein, 32 aa, Homo sapiens; identical to SEQ ID NO: 272

SEQ ID NO: 405 - protein, 25 aa, Homo sapiens
QVQLVQSGHEVKQPGASVKVSCKAS

SEQ ID NO: 406 - protein, 17 aa, Homo sapiens
GMNWVPQAPGQGLEWMG

SEQ ID NO: 407 - protein, 32 aa, Homo sapiens; identical to SEQ ID NO: 275

SEQ ID NO: 408 - protein, 11 aa, Homo sapiens
WGQGTLVTVSS

SEQ ID NO: 409 - protein, 11 aa, Homo sapiens
WGRGTLVTVSS

SEQ ID NO: 410 - protein, 11 aa, Homo sapiens
WGQGTMVTVSS

SEQ ID NO: 411 - protein, 11 aa, Homo sapiens
WGQGTLVTVSS

SEQ ID NO: 412 - protein, 11 aa, Homo sapiens
WGQGTLVTVSS

SEQ ID NO: 413 - protein, 11 aa, Homo sapiens
WGQGTTVTVSS

SEQ ID NO: 414 - DNA, 79 bp, Artificial Sequence
  Oligonucleotide used for constructing combinatorial library
gattccgctggtggtgccgttctatagccatagcgaggtgcagctgttggagtctgggggaggcttggtacagcctggg

SEQ ID NO: 415 - DNA, 49 bp, Artificial Sequence
  Oligonucleotide used for constructing combinatorial library
cagaggctgcacaggagagtctcagggaccccccaggctgtaccaagcc

SEQ ID NO: 416 - DNA, 77 bp, Artificial Sequence
  Oligonucleotide used for constructing combinatorial library
ctcctgtgcagcctctggatwcacctttascggctcctggatagagtgggtccgccagcgtccagggaaggggctgc

SEQ ID NO: 417 - DNA, 77 bp, Artificial Sequence
  Oligonucleotide used for constructing combinatorial library
ccttgaacttctcattgtagtaagcactaccacttccaggtaaaatctggcygacccactccagccccttccctgga

SEQ ID NO: 418 - DNA, 80 bp, Artificial Sequence
  Oligonucleotide used for constructing combinatorial library
ctacaatgagaagttcaagggccggttcaccatctccagagacaattccaagaacacgctgtatctgcaaatgaacagcc

SEQ ID NO: 419 - DNA, 80 bp, Artificial Sequence
  Oligonucleotide used for constructing combinatorial library
ctacaatgagaagttcaagggccggttcaccatctccgcagacaattccaagaacacgctgtatctgcaaatgaacagcc

SEQ ID NO: 420 - DNA, 80 bp, Artificial Sequence
  Oligonucleotide used for constructing combinatorial library
ctacaatgagaagttcaagggccgggccaccatctccagagacaattccaagaacacgctgtatctgcaaatgaacagcc

SEQ ID NO: 421 - DNA, 80 bp, Artificial Sequence
  Oligonucleotide used for constructing combinatorial library
ctacaatgagaagttcaagggccgggccaccatctccgcagacaattccaagaacacgctgtatctgcaaatgaacagcc

SEQ ID NO: 422 - DNA, 66 bp, Artificial Sequence
  Oligonucleotide used for constructing combinatorial library
gttatcctctytcgcacagtaatatacggccgtgtcctcggctctcaggctgttcatttgcagata

SEQ ID NO: 423 - DNA, 66 bp, Artificial Sequence
  Oligonucleotide used for constructing combinatorial library
ctgtgcgaragaggataactacggtagtagctcgttagcttactggggccaaggaaccctggtcac

SEQ ID NO: 424 - DNA, 58 bp, Artificial Sequence
  Oligonucleotide used for constructing combinatorial library
gggggaagaccgatgggcccttggtggaggctgaggagacggtgaccagggttccttg

SEQ ID NO: 425 - DNA, 65 bp, Artificial Sequence
  Oligonucleotide used for constructing combinatorial library
ggtcgttccattttactcccactccgccatccggatgacccagtctccattctccctgtctgcat

SEQ ID NO: 426 - DNA, 72 bp, Artificial Sequence
  Oligonucleotide used for constructing combinatorial library
ttgtgccaatgctctgactggccctgcaagtgatggtgactctgtctcctacagatgcagacagggagaatg

SEQ ID NO: 427 - DNA, 67 bp, Artificial Sequence
  Oligonucleotide used for constructing combinatorial library
gtcagagcattggcacaaacattcactggtatcagcaaaaaccagcaaaagcccctaagctcytcat

SEQ ID NO: 428 - DNA, 67 bp, Artificial Sequence
  Oligonucleotide used for constructing combinatorial library
gtcagagcattggcacaaacattcactggtatcagcaaaaaccaaataaagcccctaagctcytcat

SEQ ID NO: 429 - DNA, 65 bp, Artificial Sequence
  Oligonucleotide used for constructing combinatorial library
cgctgaaccttgatgggaccccagagatagactcagaagcataatagatgargagcttaggggct

SEQ ID NO: 430 - DNA, 65 bp, Artificial Sequence
  Oligonucleotide used for constructing combinatorial library
cgctgaaccttgatgggaccccagagatagactcagaagcatacttgatgargagcttaggggct

SEQ ID NO: 431 - DNA, 64 bp, Artificial Sequence
  Oligonucleotide used for constructing combinatorial library
cccatcaaggttcagcggcagtggatctgggacggattwcactctcaccatcagcagcctgcag

SEQ ID NO: 432 - DNA, 65 bp, Artificial Sequence
  Oligonucleotide used for constructing combinatorial library
cggccagttattactttgttgacagtaataagttgcaaaatcttcaggctgcaggctgctgatgg

SEQ ID NO: 433 - DNA, 47 bp, Artificial Sequence
  Oligonucleotide used for constructing combinatorial library
caacaaagtaataactggccgctcacgttcggcggagggaccaaggt

SEQ ID NO: 434 - DNA, 62 bp, Artificial Sequence
  Oligonucleotide used for constructing combinatorial library
gatgaagacagatggtgcagccacagtacgtttgagctccaccttggtccctccgccgaacg

SEQ ID NO: 435 - DNA, 50 bp, Artificial Sequence
  Oligonucleotide used for constructing combinatorial library
ttccgctggtggtgccgttctatagccatagcgaggtgcagctgttggag

SEQ ID NO: 436 - DNA, 51 bp, Artificial Sequence
  Oligonucleotide used for constructing combinatorial library
ggaccccccaggctgtaccaagcctcccccagactccaacagctgcacctc

SEQ ID NO: 437 - DNA, 50 bp, Artificial Sequence
  Oligonucleotide used for constructing combinatorial library
tacagcctggggggtccctgagactctcctgtgcagcctctggattcacc

SEQ ID NO: 438 - DNA, 50 bp, Artificial Sequence
  Oligonucleotide used for constructing combinatorial library
tggcggacccactctatccaggagccgctaaaggtgaatccagaggctgc

SEQ ID NO: 439 - DNA, 54 bp, Artificial Sequence
  Oligonucleotide used for constructing combinatorial library
gatagagtgggtccgccagcgtccagggaaggggctggagtgggtcrgccagat

SEQ ID NO: 440 - DNA, 60 bp, Artificial Sequence
  Oligonucleotide used for constructing combinatorial library
cttgaacttctcattgtagtaagcactaccacttccaggtaaaatctggcygacccactc

SEQ ID NO: 441 - DNA, 60 bp, Artificial Sequence
  Oligonucleotide used for constructing combinatorial library
actacaatgagaagttcaagggccggttcaccatctccagagacaattccaagaacacgc

SEQ ID NO: 442 - DNA, 60 bp, Artificial Sequence
  Oligonucleotide used for constructing combinatorial library
actacaatgagaagttcaagggccggttcaccatctccgcagacaattccaagaacacgc

SEQ ID NO: 443 - DNA, 60 bp, Artificial Sequence
  Oligonucleotide used for constructing combinatorial library
actacaatgagaagttcaagggccgggccaccatctccagagacaattccaagaacacgc

SEQ ID NO: 444 - DNA, 60 bp, Artificial Sequence
  Oligonucleotide used for constructing combinatorial library
actacaatgagaagttcaagggccgggccaccatctccgcagacaattccaagaacacgc

SEQ ID NO: 445 - DNA, 50 bp, Artificial Sequence
  Oligonucleotide used for constructing combinatorial library
cctcggctctcaggctgttcatttgcagatacagcgtgttcttggaattg

SEQ ID NO: 446 - DNA, 47 bp, Artificial Sequence
  Oligonucleotide used for constructing combinatorial library
cagcctgagagccgaggacacggccgtatattactgtgcgaragagg

SEQ ID NO: 447 - DNA, 50 bp, Artificial Sequence
  Oligonucleotide used for constructing combinatorial library
taagctaacgagctactaccgtagttatcctctytcgcacagtaatatac

SEQ ID NO: 448 - DNA, 50 bp, Artificial Sequence
  Oligonucleotide used for constructing combinatorial library
ggtagtagctcgttagcttactggggccaaggaaccctggtcaccgtctc

SEQ ID NO: 449 - DNA, 52 bp, Artificial Sequence
  Oligonucleotide used for constructing combinatorial library
gggggaagaccgatgggcccttggtggaggctgaggagacggtgaccagggt

SEQ ID NO: 450 - DNA, 48 bp, Artificial Sequence
  Oligonucleotide used for constructing combinatorial library
ggtcgttccattttactcccactccgccatccggatgacccagtctcc

SEQ ID NO: 451 - DNA, 49 bp, Artificial Sequence
  Oligonucleotide used for constructing combinatorial library
tctgtctcctacagatgcagacagggagaatggagactgggtcatccgg

SEQ ID NO: 452 - DNA, 49 bp, Artificial Sequence
  Oligonucleotide used for constructing combinatorial library
tgcatctgtaggagacagagtcaccatcacttgcagggccagtcagagc

SEQ ID NO: 453 - DNA, 49 bp, Artificial Sequence
  Oligonucleotide used for constructing combinatorial library
tttgctgataccagtgaatgtttgtgccaatgctctgactggccctgca

SEQ ID NO: 454 - DNA, 43 bp, Artificial Sequence
  Oligonucleotide used for constructing combinatorial library
cactggtatcagcaaaaaccagcaaaagcccctaagctcytca

SEQ ID NO: 455 - DNA, 43 bp, Artificial Sequence
  Oligonucleotide used for constructing combinatorial library
cactggtatcagcaaaaaccaaataaagcccctaagctcytca

SEQ ID NO: 456 - DNA, 49 bp, Artificial Sequence
  Oligonucleotide used for constructing combinatorial library
gaccccagagatagactcagaagcatacttgatgargagcttaggggct

SEQ ID NO: 457 - DNA, 49 bp, Artificial Sequence
  Oligonucleotide used for constructing combinatorial library
gaccccagagatagactcagaagcataatagatgargagcttaggggct

SEQ ID NO: 458 - DNA, 49 bp, Artificial Sequence
  Oligonucleotide used for constructing combinatorial library
gagtctatctctggggtcccatcaaggttcagcggcagtggatctggga

SEQ ID NO: 459 - DNA, 49 bp, Artificial Sequence
  Oligonucleotide used for constructing combinatorial library
ctgcaggctgctgatggtgagagtgwaatccgtcccagatccactgccg

SEQ ID NO: 460 - DNA, 49 bp, Artificial Sequence
  Oligonucleotide used for constructing combinatorial library
ccatcagcagcctgcagcctgaagattttgcaacttattactgtcaaca

SEQ ID NO: 461 - DNA, 49 bp, Artificial Sequence
  Oligonucleotide used for constructing combinatorial library
cgccgaacgtgagcggccagttattactttgttgacagtaataagttgc

SEQ ID NO: 462 - DNA, 39 bp, Artificial Sequence
  Oligonucleotide used for constructing combinatorial library
ccgctcacgttcggcggagggaccaaggtggagctcaaa

SEQ ID NO: 463 - DNA, 49 bp, Artificial Sequence
  Oligonucleotide used for constructing combinatorial library
gatgaagacagatggtgcagccacagtacgtttgagctccaccttggtc

SEQ ID NO: 464 - DNA, 59 bp, Artificial Sequence
  Oligonucleotide used for constructing combinatorial library
cgctggtggtgccgttctatagccatagccaaatgcagctggtgcagtctgggcctgag

SEQ ID NO: 465 - DNA, 52 bp, Artificial Sequence
  Oligonucleotide used for constructing combinatorial library
ctatggactcctggggccaaggaacctcggtcaccgtctcctcagcctccac

SEQ ID NO: 466 - DNA, 49 bp, Artificial Sequence
  Oligonucleotide used for constructing combinatorial library
cccaggagtccatagcatgatacctagggtatctcgcacagtaatacac

SEQ ID NO: 467 - DNA, 49 bp, Artificial Sequence
  Oligonucleotide used for constructing combinatorial library
tccgaggacacggccgtgtattactgtgcgagataccctaggtatcatg

SEQ ID NO: 468 - DNA, 48 bp, Artificial Sequence
  Oligonucleotide used for constructing combinatorial library
ggccgtgtcctcggatctcaggctgctcagctccawgtaggctgtgct

SEQ ID NO: 469 - DNA, 49 bp, Artificial Sequence
  Oligonucleotide used for constructing combinatorial library
cagggacatgtccacaagcacagcctacwtggagctgagcagcctgaga

SEQ ID NO: 470 - DNA, 38 bp, Artificial Sequence
  Oligonucleotide used for constructing combinatorial library
tgtggacatgtccctggtaatggtgamtctacccttca

SEQ ID NO: 471 - DNA, 47 bp, Artificial Sequence
  Oligonucleotide used for constructing combinatorial library
tacacaacagagtacagtgcatctgtgaagggtagaktcaccattac

SEQ ID NO: 472 - DNA, 45 bp, Artificial Sequence
  Oligonucleotide used for constructing combinatorial library
cagatgcactgtactctgttgtgtaatcattagctttgtttctaa

SEQ ID NO: 473 - DNA, 44 bp, Artificial Sequence
  Oligonucleotide used for constructing combinatorial library
taaatcctakccactcaaggcgttgtccacgagcctgtcgcacc

SEQ ID NO: 474 - DNA, 47 bp, Artificial Sequence
  Oligonucleotide used for constructing combinatorial library
gacaacgccttgagtggmtaggatttattagaaacaaagctaatgat

SEQ ID NO: 475 - DNA, 45 bp, Artificial Sequence
  Oligonucleotide used for constructing combinatorial library
tcacctttactgattactccatgaactgggtgcgacaggctcgtg

SEQ ID NO: 476 - DNA, 45 bp, Artificial Sequence
  Oligonucleotide used for constructing combinatorial library
gaccttcactgaggtcccaggcttcttcacctcaggcccagactg

SEQ ID NO: 477 - DNA, 49 bp, Artificial Sequence
  Oligonucleotide used for constructing combinatorial library
gtgaagaagcctgggacctcagtgaaggtctcctgcaaggcttctggat

SEQ ID NO: 478 - DNA, 47 bp, Artificial Sequence
  Oligonucleotide used for constructing combinatorial library
cagttcatggagtaatcagtaaaggtgaatccagaagccttgcagga

SEQ ID NO: 479 - DNA, 44 bp, Artificial Sequence
  Oligonucleotide used for constructing combinatorial library
caccagctgcatttggctatggctatagaacggcaccaccagcg

SEQ ID NO: 480 - DNA, 57 bp, Artificial Sequence
  Oligonucleotide used for constructing combinatorial library
ggaagaccgatgggcccttggtggaggctgaggagacggtgaccgaggttccttggc

SEQ ID NO: 481 - DNA, 48 bp, Artificial Sequence
  Oligonucleotide used for constructing combinatorial library
ggtcgttccattttactcccactccgacatcgtgatgacccagtctcc

SEQ ID NO: 482 - DNA, 49 bp, Artificial Sequence
  Oligonucleotide used for constructing combinatorial library
cgctcacgttcggcggagggaccaaggtggagatcaaacgtactgtggc

SEQ ID NO: 483 - DNA, 41 bp, Artificial Sequence
  Oligonucleotide used for constructing combinatorial library
cctccgccgaacgtgagcggccagctgttactctgttgaca

SEQ ID NO: 484 - DNA, 48 bp, Artificial Sequence
  Oligonucleotide used for constructing combinatorial library
agcctgaagattttgcaacatattactgtcaacagagtaacagctggc

SEQ ID NO: 485 - DNA, 42 bp, Artificial Sequence
  Oligonucleotide used for constructing combinatorial library
gtaatatgttgcaaaatcttcaggctgcaggctgctgatggt

SEQ ID NO: 486 - DNA, 39 bp, Artificial Sequence
  Oligonucleotide used for constructing combinatorial library
gatctgggacagattttactttcaccatcagcagcctgc

SEQ ID NO: 487 - DNA, 45 bp, Artificial Sequence
  Oligonucleotide used for constructing combinatorial library
gaaagtaaaatctgtcccagatccacttccactgaaccttgatgg

SEQ ID NO: 488 - DNA, 38 bp, Artificial Sequence
  Oligonucleotide used for constructing combinatorial library
gtccatctctggggtcccatcaaggttcagtggaagtg

SEQ ID NO: 489 - DNA, 45 bp, Artificial Sequence
  Oligonucleotide used for constructing combinatorial library
gaccccagagatggactggaaaacatacttgatcaggagcttagg

SEQ ID NO: 490 - DNA, 46 bp, Artificial Sequence
  Oligonucleotide used for constructing combinatorial library
agaaaccagggaaagcccctaagctcctgatcaagtatgttttcca

SEQ ID NO: 491 - DNA, 44 bp, Artificial Sequence
  Oligonucleotide used for constructing combinatorial library
ggctttccctggtttctgctgataccagtgtaggttgttgctaa

SEQ ID NO: 492 - DNA, 44 bp, Artificial Sequence
  Oligonucleotide used for constructing combinatorial library
cagggccagccaaagtattagcaacaacctacactggtatcagc

SEQ ID NO: 493 - DNA, 48 bp, Artificial Sequence
  Oligonucleotide used for constructing combinatorial library
tactttggctggccctgcaartgatgktgactctgtctcctacagatg

SEQ ID NO: 494 - DNA, 45 bp, Artificial Sequence
  Oligonucleotide used for constructing combinatorial library
atcctccctgtctgcatctgtaggagacagagtcamcatcayttg

SEQ ID NO: 495 - DNA, 49 bp, Artificial Sequence
  Oligonucleotide used for constructing combinatorial library
cagacagggaggatggagactgggtcatcacgatgtcggagtgggagta

SEQ ID NO: 496 - DNA, 49 bp, Artificial Sequence
  Oligonucleotide used for constructing combinatorial library
gatgaagacagatggtgcagccacagtacgtttgatctccaccttggtc

SEQ ID NO: 497 - DNA, 59 bp, Artificial Sequence
  Oligonucleotide used for constructing combinatorial library
gattccgctggtggtgccgttctatagccatagccaggttcagctgcagcagtctggag

SEQ ID NO: 498 - DNA, 55 bp, Artificial Sequence
  Oligonucleotide used for constructing combinatorial library
gggggaagaccgatgggcccttggtggaggctgcagagacagtgagtagagtccc

SEQ ID NO: 499 - DNA, 48 bp, Artificial Sequence
  Oligonucleotide used for constructing combinatorial library
ggtcgttccattttactcccactccgacatcttgctgactcagtctcc

SEQ ID NO: 500 - DNA, 54 bp, Artificial Sequence
  Oligonucleotide used for constructing combinatorial library
gatgaagacagatggtgcagccacagtacgtttcagctccagcttggttccagc

SEQ ID NO: 501 - DNA, 56 bp, Artificial Sequence
  Oligonucleotide used for constructing combinatorial library
gctggtggtgccgttctatagccatagcgaggtgaagctggtggagtctggaggag

SEQ ID NO: 502 - DNA, 55 bp, Artificial Sequence
  Oligonucleotide used for constructing combinatorial library
ggaagaccgatgggcccttggtggaggctgaggagacggtgactgaggttccttg

SEQ ID NO: 503 - DNA, 58 bp, Artificial Sequence
  Oligonucleotide used for constructing combinatorial library
ggtcgttccattttactcccactccgatattgtgctaactcagtctccagccaccctg

SEQ ID NO: 504 - DNA, 62 bp, Artificial Sequence
  Oligonucleotide used for constructing combinatorial library
gatgaagacagatggtgcagccacagtacgtttcagctccagcttggtcccagcaccgaacg

SEQ ID NO: 505 - DNA, 321 bp, Mus musculus
gacatcttgctgactcagtctccagccatcctgtctgtgagtccaggagaaagagtcagtttctcctgcagggccagtcagagcattggcacaaacattcactggtatcagcaaagaacaaatggttctccaaggcttctcataaagtatgcttctgagtctatctctgggatcccttccaggtttagtggcggtggatcagggacagattttactcttagcatcaacagtgtggagtctgaagatattgcagattattactgtcaacaaagtaataactggccgctcacgttcggtgctggaaccaagctggagctgaaa

SEQ ID NO: 506 - DNA, 360 bp, Mus musculus
caggttcagctgcagcagtctggagctgagctgatgaagcctggggcctcagtgaagctttcctgcaaggctaccggctacacattcactggctcctggatagagtggataaaacagaggcctggacatggccttgagtggattggacagattttacctggaagtggtagtgcttactacaatgagaagttcaagggcaaggccacattcactgcagatacatcctccaagacagtctacattcaactcatcagcctgacaactgaggactctgccatctattactgtgcaagagaggataactacggtagtagctcgttagcttactggggccaagggactctactcactgtctctgca

SEQ ID NO: 507 - protein, 107 aa, Mus musculus
DILLTQSPAILSVSPGERVSFSCRASQSIGTNIHWYQQRTNGSPRLLIKYASESISGIPSRFSGGGSGTDFTLSINSVESEDIADYYCQQSNNWPLTFGAGTKLELK

SEQ ID NO: 508 - protein, 120 aa, Mus musculus
QVQLQQSGAELMKPGASVKLSCKATGYTFTGSWIEWIKQRPGHGLEWIGQILPGSGSAYYNEKFKGKATFTADTSSKTVYIQLISLTTEDSAIYYCAREDNYGSSSLAYWGQGTLLTVSA

SEQ ID NO: 509 - protein, 321 aa, Mus musculus
GATATTGTGCTAACTCAGTCTCCAGCCACCCTGTCTGTGACTCCAGGAGATAGCGTCAATCTTTCCTGCAGGGCCAGCCAAAGTATTAGCAACAACCTACACTGGTATCAACAAAAATCACATGAGTCTCCAAGGCTTCTCATCAAGTATGTTTTCCAGTCCATCTCTGGGATCCCCTCCAGGTTCAGTGGCAGTGGATCAGGGACAGATTTCACTCTCAGTATCAACAGTGTGGAGACTGAAGATTTTGGAATGTATTTCTGTCAACAGAGTAACAGCTGGCCGCTCACGTTCGGTGCTGGGACCAAGCTGGAGCTGAAA

SEQ ID NO: 510 - DNA, 360 bp, Mus musculus
gaggtgaagctggtggagtctggaggaggcttggtacagcctgggggttctctgagtctctcctgtgcagcttctggattcaccttcactgattactccatgaactgggtccgccagcctccagggaaggcacttgagtggttgggttttattagaaacaaagctaatgattacacaacagagtacagtgcatctgtgaagggtcggttcaccatctccagagataattcccaaagcatcctctatcttcaaatgaatgccctgagagctgaggacagtgccacttattactgtgtaagataccctaggtatcatgctatggactcctggggtcaaggaacctcagtcaccgtctcctca

SEQ ID NO: 511 - protein, 107 aa, Mus musculus
DIVLTQSPATLSVTPGDSVNLSCRASQSISNNLHWYQQKSHESPRLLIKYVFQSISGIPSRFSGSGSGTDFTLSINSVETEDFGMYFCQQSNSWPLTFGAGTKLELK

SEQ ID NO: 512 - protein, 120 aa, Mus musculus
EVKLVESGGGLVQPGGSLSLSCAASGFTFTDYSMNWVRQPPGKALEWLGFIRNKANDYTTEYSASVKGRFTISRDNSQSILYLQMNALRAEDSATYYCVRYPRYHAMDSWGQGTSVTVSS

SEQ ID NO: 513 - protein, 107 aa, Artificial
  Humanized Variable Region
DIVMTQSPSSLSASVGDRVTIICRASQSISNNLHWYQQKPGKAPKLLIKYVFQSISGVPSRFSGSGSGTDFTFTISSLQPEDFATYYCQQSNSWPLTFGGGTKVEIK

SEQ ID NO: 514 - protein, 107 aa, Artificial
  Humanized Variable Region
DIQMTQSPSSLSASVGDRVTITCRASQSISNNLHWYQQKPGKAPKLLIKYVFQSISGVPSRFSGSGSGTDFTFTISSLQPEDFATYYCQQSNSWPLTFGGGTKVEIK

SEQ ID NO: 515 - protein, 107 aa, Artificial; identical to SEQ ID NO: 513
  Humanized Variable Region

SEQ ID NO: 516 - protein, 120 aa, Artificial
  Humanized Variable Region
QMQLVQSGPEVKKPGTSVKVSCKASGFTFTDYSMNWVRQARGQRLEWLGFIRNKANDYTTEYSASVKGRVTITRDMSTSTAYMELSSLRSEDTAVYYCARYPRYHAMDSWGQGTSVTVSS

SEQ ID NO: 517 - protein, 120 aa, Artificial
  Humanized Variable Region
QMQLVQSGPEVKKPGTSVKVSCKASGFTFTDYSMNWVRQARGQRLEWIGFIRNKANDYTTEYSASVKGRVTITRDMSTSTAYMELSSLRSEDTAVYYCARYPRYHAMDSWGQGTSVTVSS

SEQ ID NO: 518 - protein, 120 aa, Artificial; identical to SEQ ID NO: 517
  Humanized Variable Region